(19) United States
(12) Patent Application Publication
(10) Pub. No.: US 2009/0093454 A1
(43) Pub. Date: Apr. 9, 2009

(54) Chemical Compounds

(76) Inventors: Kristjan Gudmundsson, Research Triangle Park, NC (US); John G. Catalano, Research Triangle Park, NC (US); Angilique Svolto, Research Triangle Park, NC (US)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(21) Appl. No.: 11/817,521

(22) PCT Filed: Mar. 1, 2006
(86) PCT No.: PCT/US2006/007395

§ 371 Date: Aug. 31, 2007

Related U.S. Application Data
(60) Provisional application No. 60/658,530, filed on Mar. 4, 2005.

(51) Int. Cl. A61K 31/397; C07D 487/02; A61K 31/496; A61K 31/5377; A61P 29/00; A61P 31/00; A61K 31/473; A61K 31/551

(52) U.S. Cl. 514/210.21; 544/361; 514/253.03; 546/84; 514/292; 540/575; 514/218; 514/234.5; 544/126

(57) ABSTRACT

The present invention provides compounds of Formula (I) comprising:

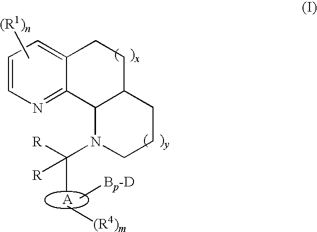

including salts, solvates, and physiologically functional derivatives thereof, pharmaceutical formulations containing them, processes for their preparation, and methods of treatment using them.

### FIELD OF THE INVENTION

The present invention provides novel compounds that demonstrate protective effects on target cells from HIV infection in a manner as to bind specifically to the chemokine receptor, and which affect the binding of the natural ligand or chemokine to a receptor such as CXCR4 of a target cell.

### BACKGROUND OF THE INVENTION

HIV gains entry into host cells by means of the CD4 receptor and at least one co-receptor expressed on the surface of the cell membrane. M-tropic strains of HIV utilize the chemokine receptor CCR5, whereas T-tropic strains of HIV mainly use CXCR4 as the co-receptor. HIV co-receptor usage largely depends on hyper-variable regions of the V3 loop located on the viral envelope protein gp120. Binding of gp120 with CD4 and the appropriate co-receptor results in a conformational change and unmasking of a second viral envelope protein called gp41. The protein gp41 subsequently interacts with the host cell membrane resulting in fusion of the viral envelop with the cell. Subsequent transfer of viral genetic information into the host cell allows for the continuation of viral replication. Thus infection of host cells with HIV is usually associated with the virus gaining entry into the cell via the formation of the ternary complex of CCR5 or CXCR4, CD4, and gp120.

A pharmacological agent that would inhibit the interaction of gp120 with either CCR5/CD4 or CXCR4/CD4 would be a useful therapeutic in the treatment of a disease, disorder, or condition characterized by infection with M-tropic or T-tropic strains, respectively, either alone or in combination therapy.

Evidence that administration of a selective CXCR4 antagonist could result in an effective therapy comes from in vitro studies that have demonstrated that addition of ligands selective for CXCR4 as well as CXCR4-neutralizing antibodies to cells can block HIV viral/host cell fusion. In addition, human studies with the selective CXCR4 antagonist AMD-3100, have demonstrated that such compounds can significantly reduce T-tropic HIV viral load in those patients that are either dual tropic or those where only the T-tropic form of the virus is present.

In addition to serving as a co-factor for HIV entry, it has been recently suggested that the direct interaction of the HIV viral protein gp120 with CXCR4 could be a possible cause of CD8⁺ T-cell apoptosis and AIDS-related dementia via induction of neuronal cell apoptosis.

The signal provided by SDF-1 on binding to CXCR4 may also play an important role in tumor cell proliferation and regulation of angiogenesis associated with tumor growth; the known angiogenic growth factors VEG-F and bFGF up-regulate levels of CXCR4 in endothelial cells and SDF-1 can induce neovascularization in vivo. In addition, leukemia cells that express CXCR4 migrate and adhere to lymph nodes and bone marrow stromal cells that express SDF-1.

The binding of SDF-1 to CXCR4 has also been implicated in the pathogenesis of atherosclerosis, renal allograft rejection, asthma and allergic airway inflammation, Alzheimer's disease, and arthritis.

The present invention is directed to compounds that can act as agents that modulate chemokine receptor activity. Such chemokine receptors include, but are not limited to, CCR1, CCR2, CCR3, CCR4, CCR5, CCR6, CCR7, CCR8, CXCR1, CXCR2, CXCR3, CXCR4, and CXCR5.

The present invention provides novel compounds that demonstrate protective effects on target cells from HIV infection in a manner as to bind specifically to the chemokine receptor, and which affect the binding of the natural ligand or chemokine to a receptor, such as CXCR4 of a target cell.

### SUMMARY OF THE INVENTION

The present invention includes compounds of Formula (I):

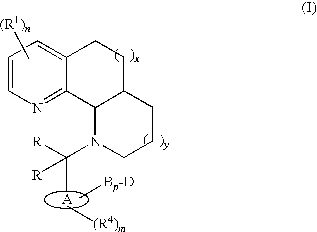

or a salt, a solvate, and a physiologically functional derivative thereof, wherein
x and y are each independently 0, 1, or 2;
each R independently is H, alkyl, alkenyl, alkynyl, haloalkyl, cycloalkyl, —R^{a}Ay, —R^{a}OR¹⁰, —R^{a}NR⁶R⁷, R^{a}C(O)R¹⁰, —C(O)R¹⁰, —CO₂R¹⁰, —R^{a}CO₂R¹⁰ or —R^{a}S(O)_{q}R¹⁰;
n is 0, 1, 2, or 3, such that an R¹ may be substituted throughout the depicted tricyclic ring system (for example, 1,2,3,4,4a,5,6,10b-octahydro-1,10-phenanthroline when x=1 and y=1);
each R¹ independently is halogen, haloalkyl, alkyl, alkenyl, alkynyl, cycloalkyl, cycloalkenyl, -Ay, —NHAy, -Het, —NHHet, —OR¹⁰, —OAy, —OHet, —R^{a}OR¹⁰, —NR⁶R⁷, —R^{a}NR⁶R⁷, —R^{a}C(O)R¹⁰, —C(O)R¹⁰, —CO₂R¹⁰, —R^{a}CO₂R¹⁰, —C(O)NR⁶R⁷, —C(O)Ay, —C(O)Het, —S(O)₂NR⁶R⁷, —R^{a}S(O)₂NR⁶R⁷, —S(O)_{q}R¹⁰, —S(O)_{q}Ay, —S(O)_{q}Het, —R^{a}S(O)_{q}R¹⁰, cyano, nitro, or azido;

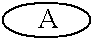

is heteroaryl;
each R⁴ independently is halogen, haloalkyl, alkyl, alkenyl, alkynyl, cycloalkyl, cycloalkenyl, -Ay, —NHAy, -Het, —NHHet, —OR¹⁰, —OAy, —OHet, —R^{a}OR¹⁰, —NR⁶R⁷, —R^{a}NR⁶R⁷, —R^{a}C(O)R¹⁰, —C(O)R¹⁰, —CO₂R¹⁰, —R^{a}CO₂R¹⁰, —C(O)NR⁶R⁷, C(O)Ay, —C(O)Het, —S(O)₂NR⁶R⁷, —R^{a}S(O)₂NR⁶R⁷, —S(O)_{q}R¹⁰, —S(O)_{q}Ay, —S(O)_{q}Het, cyano, nitro, or azido;
m is 0, 1, or 2, such that R⁴ can be substituted anywhere on heteroaryl A (that is, m does not exceed the number of open valences on the heteroaryl A);
p is 0 or 1;
B is —NR¹⁰—, —O—, —C(O)NR¹⁰—, —NR¹⁰C(O)—, —C(O)—, —C(O)O—, —NR¹⁰C(O)N(R¹⁰)—, —S(O)_{q}—, —S(O)_{q}NR¹⁰—, or —NR¹⁰S(O)_{q}—;
D is —N(R¹⁰)₂, —R^{a}N(R¹⁰)₂, -AyN(R¹⁰)₂, R^{a}AyN(R¹⁰)₂, AyR^{a}N(R¹⁰)₂, —R^{a}AyR^{a}N(R¹⁰)₂, -Het, —R^{a}Het, -HetN(R¹⁰)₂, —R^{a}HetN(R¹⁰)₂, -HetR^{a}N(R¹⁰)₂, —R^{a}HetR^{a}N(R¹⁰)₂, -HetR^{a}Ay, or -HetR^{a}Het;
each R^{a} independently is alkylene, cycloalkylene, alkenylene, cycloalkenylene, or alkynylene, where each may be substituted with C₁-C₈ alkyl, hydroxyl, or oxo;
each R¹⁰ independently is H, alkyl, cycloalkyl, alkenyl, alkynyl, cycloalkenyl, —R^{a}cycloalkyl, —R^{a}OH, —R^{a}OR⁸, —R^{a}NR⁸R⁹, or —R^{a}Het;
each of R⁶ and R⁷ independently are selected from H, alkyl, alkenyl, alkynyl, cycloalkyl, cycloalkenyl, —R^{a}cycloalkyl, —R^{a}OH, —R^{a}OR¹⁰, —R^{a}NR⁸R⁹, -Ay, -Het, —R^{a}Ay, —R^{a}Het, or —S(O)_{q}R¹⁰;
each of R⁸ and R⁹ independently are selected from H or alkyl;
each q independently is 0, 1, or 2;
each Ay independently represents an unsubstituted or substituted aryl group; and
each Het independently represents a substituted or unsubstituted 4-, 5-, or 6-membered heterocyclyl or heteroaryl group. In Formula (I) x and y are each independently 0, 1, or 2. That means the ring in which ( )ₓ and ( )_{y} are depicted is 5-, 6-, or 7-membered ring(s), respectively. This is due to the absence or presence of carbon atom(s).

In one embodiment x and y are both 1. In another embodiment x is 1 and y is 2. In still another embodiment x is 1 and y is 0. In a further embodiment x is 0 and y is 0. In another embodiment x is 0 and y is 1. And in another embodiment x is 0 and y is 2. Also in one embodiment x is 2 and y is 0. In another embodiment is x is 2 and y is 1. Or, still a further embodiment occurs when x is 2 and y is 2.

In one embodiment R is H or alkyl. Preferably R is H.

In one embodiment n is 0.

In one embodiment n is 1 and R¹ is halogen, haloalkyl, alkyl, OR¹⁰, NR⁶R⁷, CO₂R¹⁰, CONR⁶R⁷, or cyano.

In one embodiment the heteroaryl A is a benzimidazole.

In one embodiment the heteroaryl A is an imidazole.

In one embodiment the heteroaryl A is an imidazopyridine

In one embodiment m is 0.

In one embodiment m is 1 or 2. Preferably m is 1.

When m is 1 or 2, R⁴ preferably is one or more of halogen, haloalkyl, alkyl, OR¹⁰, NR⁶R⁷, CO₂R¹⁰, CONR⁶R⁷, or cyano.

In one embodiment p is 0 and D is —R^{a}N(R¹⁰)₂, -AyR^{a}N(R¹⁰)₂, —R^{a}AyR^{a}N(R¹⁰)₂, -Het, —R^{a}Het, -HetN(R¹⁰)₂, —R^{a}HetN(R¹⁰)₂, or -HetR^{a}N(R¹⁰)₂. Preferably D is —R^{a}N(R¹⁰)₂, -Het, —R^{a}Het, -HetN(R¹⁰)₂, —R^{a}HetN(R¹⁰)₂, or

-HetR^{a}N(R¹⁰)₂. More preferably D is R^{a}N(R¹⁰)₂, -Het, —R^{a}Het, or -HetN(R¹⁰)₂. Most preferably, D is Het, and Het is heterocyclyl.

In one embodiment p is 1; B is —N(R¹⁰)—, —O—, —S—, —CONR¹⁰—, —NR¹⁰CO—, or —S(O)_{q}NR¹⁰—; and D is —R^{a}N(R¹⁰)₂, -AyR^{a}N(R¹⁰)₂, —R^{a}AyR^{a}N(R¹⁰)₂, -Het, —R^{a}Het, -HetN(R¹⁰)₂, —R^{a}HetN(R¹⁰)₂, or -HetR^{a}N(R¹⁰)₂. Preferably B is —N(R¹⁰)—, —O—, —CONR¹⁰—, —NR¹⁰CO— and D is —R^{a}N(R¹⁰)₂, -Het, —R^{a}Het, or -HetN(R¹⁰)₂. More preferably B is —N(R¹⁰)—, and D is Het.

Preferably Het is piperidine, piperazine, azetidine, pyrrolidine, imidazole, pyridine, and the like, including substituted versions thereof. Most preferably Het is piperazine.

In one embodiment p is 0 and D is -Het. Preferably -Het is unsubstituted or substituted with one or more C₁-C₆ alkyl or cycloalkyl.

In one embodiment heteroaryl A is imidazole or benzimidazole and substituent —Bₚ-D is attached via imidazole nitrogen as shown by formula 1-A and 1-B:

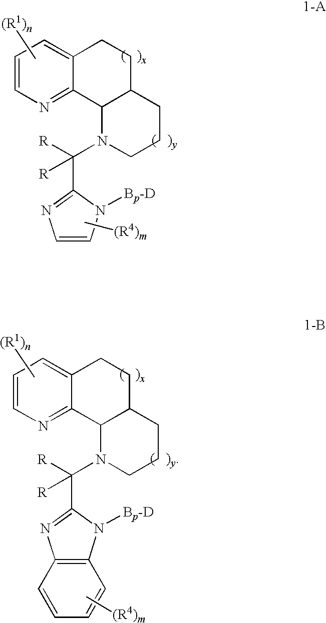

In one embodiment heteroaryl A is benzimidazole or imidazopyridine and substituent —Bₚ-D is attached via carbon as shown by formula 1-C and 1-D:

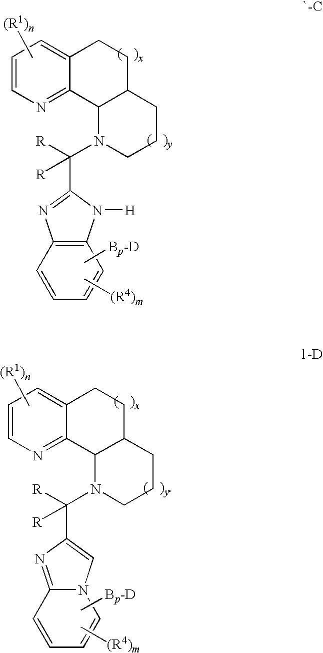

One aspect of the invention includes compounds of formula 1-A where n is 0, x and y are 1, R is H, p is 0, D is R^{a}N(R¹⁰)₂, -Het, —R^{a}Het, or -HetN(R¹⁰)₂ where R¹⁰ is H or C₁-C₈ alkyl, and m is 0.

One aspect of the invention includes compounds of formula 1-B where n is 0, x and y are 1, R is H, p is 0, D is R^{a}N(R¹⁰)₂, -Het, —R^{a}Het, or -HetN(R¹⁰)₂ where R¹⁰ is H or C₁-C₈ alkyl, and m is 0.

One aspect of the invention includes compounds of formula 1-C where n is 0, x and y are 1, R is H, p is 0, D is R^{a}N(R¹⁰)₂, -Het, —R^{a}Het, or -HetN(R¹⁰)₂ where R¹⁰ is H or C₁-C₈ alkyl, and m is 0.

One aspect of the invention includes compounds of formula 1-D where n is 0, x and y are 1, R is H, p is 0, D is R^{a}N(R¹⁰)₂, -Het, —R^{a}Het, or -HetN(R¹⁰)₂ where R¹⁰ is H or C₁-C₈ alkyl, and m is 0.

One aspect of the present invention includes the compounds as herein defined with reference to any one of the Examples.

One aspect of the present invention includes a pharmaceutical composition comprising one or more compounds of the present invention and a pharmaceutically acceptable carrier.

One aspect of the present invention includes one or more compounds of the present invention for use as an active therapeutic substance.

One aspect of the present invention includes one or more compounds of the present invention for use in the treatment (including prophylaxis) of diseases and conditions caused by inappropriate activity of CXCR4.

One aspect of the present invention includes one or more compounds of the present invention for use in the treatment (including prophylaxis) of HIV infection, diseases associated with hematopoiesis, myocardial infarction, controlling the side effects of chemotherapy, enhancing the success of bone marrow transplantation, enhancing wound healing and burn treatment, combating bacterial infections in leukemia, inflammation, inflammatory or allergic diseases, asthma, allergic rhinitis, hypersensitivity lung diseases, hypersensitivity pneumonitis, eosinophilic pneumonitis, delayed-type hypersensitivity, interstitial lung disease (ILD), idiopathic pulmonary fibrosis, systemic lupus erythematosus, ankylosing spondylitis, systemic sclerosis, Sjogren's syndrome, polymyositis or dermatomyositis, systemic anaphylaxis or hypersensitivity responses, drug allergies, insect sting allergies, autoimmune diseases, rheumatoid arthritis, psoriatic arthritis, systemic lupus erythematosus, myastenia gravis, juvenile onset diabetes, glomerulonephritis, autoimmune throiditis, graft rejection, allograft rejection, graft-versus-host disease, inflammatory bowel diseases, Crohn's disease, ulcerative colitus; spondylo-arthropathies, scleroderma; psoriasis, T-cell-mediated psoriasis, inflammatory dermatoses, dermatitis, eczema, atopic dermatitis, allergic contact dermatitis, urticaria, vasculitis, necrotizing, cutaneous, hypersensitivity vasculitis, eosinophilic myotis, eosinophilic fasciitis, and brain, breast, prostate, lung, or haematopoetic tissue cancers. Preferably the condition or disease is HIV infection, rheumatoid arthritis, inflammation, or cancer.

One aspect of the present invention includes the use of one or more compounds of the present invention in the manufacture of a medicament for use in the treatment (including prophylaxis) of a condition or disease modulated by a chemokine receptor. Preferably the chemokine receptor is CXCR4.

One aspect of the present invention includes use of one or more compounds of the present invention in the manufacture of a medicament for use in the treatment (including prophylaxis) of HIV infection, diseases associated with hematopoiesis, myocardial infarction, controlling the side effects of chemotherapy, enhancing the success of bone marrow transplantation, enhancing wound healing and burn treatment, combating bacterial infections in leukemia, inflammation, inflammatory or allergic diseases, asthma, allergic rhinitis, hypersensitivity lung diseases, hypersensitivity pneumonitis, eosinophilic pneumonitis, delayed-type hypersensitivity, interstitial lung disease (ILD), idiopathic pulmonary fibrosis, systemic lupus erythematosus, ankylosing spondylitis, systemic sclerosis, Sjogren's syndrome, polymyositis or dermatomyositis, systemic anaphylaxis or hypersensitivity responses, drug allergies, insect sting allergies, autoimmune diseases, rheumatoid arthritis, psoriatic arthritis, systemic lupus erythematosus, myastenia gravis, juvenile onset diabetes, glomerulonephritis, autoimmune throiditis, graft rejection, allograft rejection, graft-versus-host disease, inflammatory bowel diseases, Crohn's disease, ulcerative colitus; spondylo-arthropathies, scleroderma; psoriasis, T-cell-mediated psoriasis, inflammatory dermatoses, dermatitis, eczema, atopic dermatitis, allergic contact dermatitis, urticaria, vasculitis, necrotizing, cutaneous, hypersensitivity vasculitis, eosinophilic myotis, eosinophilic fasciitis, and brain, breast, prostate, lung, or haematopoetic tissue cancers. Preferably the use relates to a medicament wherein the condition or disorder is HIV infection rheumatoid arthritis, inflammation, or cancer.

One aspect of the present invention includes a method for the treatment (including prophylaxis) of a condition or disease modulated by a chemokine receptor comprising the administration of one or more compounds of the present invention. Preferably the chemokine receptor is CXCR4.

One aspect of the present invention includes a method for the treatment (including prophylaxis) of HIV infection, diseases associated with hematopoiesis, myocardial infarction, controlling the side effects of chemotherapy, enhancing the success of bone marrow transplantation, enhancing wound healing and burn treatment, combating bacterial infections in leukemia, inflammation, inflammatory or allergic diseases, asthma, allergic rhinitis, hypersensitivity lung diseases, hypersensitivity pneumonitis, eosinophilic pneumonitis, delayed-type hypersensitivity, interstitial lung disease (ILD), idiopathic pulmonary fibrosis, systemic lupus erythematosus, ankylosing spondylitis, systemic sclerosis, Sjogren's syndrome, polymyositis or dermatomyositis, systemic anaphylaxis or hypersensitivity responses, drug allergies, insect sting allergies, autoimmune diseases, rheumatoid arthritis, psoriatic arthritis, systemic lupus erythematosus, myastenia gravis, juvenile onset diabetes, glomerulonephritis, autoimmune throiditis, graft rejection, allograft rejection, graft-versus-host disease, inflammatory bowel diseases, Crohn's disease, ulcerative colitus; spondylo-arthropathies, scleroderma; psoriasis, T-cell-mediated psoriasis, inflammatory dermatoses, dermatitis, eczema, atopic dermatitis, allergic contact dermatitis, urticaria, vasculitis, necrotizing, cutaneous, hypersensitivity vasculitis, eosinophilic myotis, eosinophilic fasciitis, and brain, breast, prostate, lung, or haematopoetic tissue cancers comprising the administration of one or more compounds of the present invention.

One aspect of the present invention includes a method for the treatment (including prophylaxis) of HIV infection rheumatoid arthritis, inflammation, or cancer comprising the administration of a therapeutically effective amount of one or more compounds of the present invention.

One aspect of the invention includes a pharmaceutical composition comprising a compound of claims 1-37 and one or more additional therapeutic agent selected from the group consisting of nucleotide reverse transcriptase inhibitors, non-nucleotide reverse transcriptase inhibitors, protease inhibitors, entry inhibitors, integrase inhibitors, budding inhibitors, CXCR4 inhibitors, and CCR5 inhibitors.

One aspect of the invention includes a pharmaceutical composition wherein the nucleotide reverse transcriptase inhibitor is selected from zidovudine, didanosine, lamivudine, zalcitabine, abacavir, stavidine, adefovir, adefovir dipivoxil, fozivudine, todoxil, emtricitabine, alovudine, amdoxovir, and elvucitabine; the non-nucleotide reverse transcriptase inhibitor is selected from nevirapine, delavirdine, efavirenz, loviride, immunocal, oltipraz, capravirine, TMC-278, TMC-125, and etravirine; the protease inhibitor is selected from saquinavir, ritonavir, indinavir, nelfinavir, amprenavir, fosamprenavir, brecanavir, palinavir, lasinavir, atazanavir, and tipranavir; the entry inhibitor is selected from enfuvirtide (T-20), T-1249, PRO-542, PRO-140, TNX-355, BMS-806, and 5-Helix; the integrase inhibitor is selected from L-870,180; the budding inhibitor is selected from PA-344 and PA-457; and the CXCR4/CCR5 inhibitor is selected from vicriviroc (Sch-C), Sch-D, TAK779, maraviroc (UK 427,857), and TAK449. In one embodiment of the present invention a pharmaceutical composition of the present invention contains an additional therapeutic agent that is a CXCR4 inhibitor or CCR5 inhibitor.

### DETAILED DESCRIPTION OF THE INVENTION

Terms are used within their accepted meanings. The following definitions are meant to clarify, but not limit, the terms defined.

As used herein the term “alkyl” refers to a straight or branched chain hydrocarbon, preferably having from one to twelve carbon atoms. Examples of “alkyl” as used herein include, but are not limited to, methyl, ethyl, propyl, isopropyl, isobutyl, n-butyl, tert-butyl, isopentyl, n-pentyl.

As used throughout this specification, the preferred number of atoms, such as carbon atoms, will be represented by, for example, the phrase “Cₓ-C_{y} alkyl,” which refers to an alkyl group, as herein defined, containing the specified number of carbon atoms. Similar terminology will apply for other preferred terms and ranges as well.

As used herein the term “alkenyl” refers to a straight or branched chain aliphatic hydrocarbon containing one or more carbon-to-carbon double bonds. Examples include, but are not limited to, vinyl, allyl, and the like.

As used herein the term “alkynyl” refers to a straight or branched chain aliphatic hydrocarbon containing one or more carbon-to-carbon triple bonds. Examples include, but are not limited to, ethynyl and the like.

As used herein, the term “alkylene” refers to a straight or branched chain divalent hydrocarbon radical, preferably having from one to ten carbon atoms. Examples of “alkylene” as used herein include, but are not limited to, methylene, ethylene, n-propylene, n-butylene, and the like.

As used herein, the term “alkenylene” refers to a straight or branched chain divalent hydrocarbon radical, preferably having from one to ten carbon atoms, containing one or more carbon-to-carbon double bonds. Examples include, but are not limited to, vinylene, allylene or 2-propenylene, and the like.

As used herein, the term “alkynylene” refers to a straight or branched chain divalent hydrocarbon radical, preferably having from one to ten carbon atoms, containing one or more carbon-to-carbon triple bonds. Examples include, but are not limited to, ethynylene and the like.

As used herein, the term “cycloalkyl” refers to an optionally substituted non-aromatic cyclic hydrocarbon ring. Exemplary “cycloalkyl” groups include, but are not limited to, cyclopropyl, cyclobutyl, cyclopentyl, cyclohexyl, and cycloheptyl. As used herein, the term “cycloalkyl” includes an optionally substituted fused polycyclic hydrocarbon saturated ring and aromatic ring system, namely polycyclic hydrocarbons with less than maximum number of non-cumulative double bonds, for example where a saturated hydrocarbon ring (such as a cyclopentyl ring) is fused with an aromatic ring (herein “aryl,” such as a benzene ring) to form, for example, groups such as indane. Preferred substituent groups include alkyl, alkenyl, alkynyl, alkoxy, hydroxyl, halogen, haloalkyl, cycloalkyl, cycloalkoxy, cyano, amide, amino, and alkylamino.

As used herein, the term “cycloalkenyl” refers to an optionally substituted non-aromatic cyclic hydrocarbon ring containing one or more carbon-to-carbon double bonds which optionally includes an alkylene linker through which the cycloalkenyl may be attached. Exemplary “cycloalkenyl” groups include, but are not limited to, cyclopropenyl, cyclobutenyl, cyclopentenyl, cyclohexenyl, and cycloheptenyl. Preferred substituent groups include alkyl, alkenyl, alkynyl, alkoxy, hydroxyl, halogen, haloalkyl, cycloalkyl, cycloalkoxy, cyano, amide, amino, and alkylamino.

As used herein, the term “cycloalkylene” refers to a divalent, optionally substituted non-aromatic cyclic hydrocarbon ring. Exemplary “cycloalkylene” groups include, but are not limited to, cyclopropylene, cyclobutylene, cyclopentylene, cyclohexylene, and cycloheptylene.

As used herein, the term “cycloalkenylene” refers to a divalent optionally substituted non-aromatic cyclic hydrocarbon ring containing one or more carbon-to-carbon double bonds. Exemplary “cycloalkenylene” groups include, but are not limited to, cyclopropenylene, cyclobutenylene, cyclopentenylene, cyclohexenylene, and cycloheptenylene.

As used herein, the term “heterocycle” or “heterocyclyl” refers to an optionally substituted mono- or polycyclic ring system containing one or more degrees of unsaturation and also containing one or more heteroatoms. Preferred heteroatoms include N, O, and/or S, including N-oxides, sulfur oxides, and dioxides. More preferably, the heteroatom is N.

Preferably the heterocyclyl ring is three to twelve-membered and is either fully saturated or has one or more degrees of unsaturation. Such rings may be optionally fused to one or more of another “heterocyclic” ring(s) or cycloalkyl ring(s). Examples of “heterocyclic” groups include, but are not limited to, tetrahydrofuran, pyran, 1,4-dioxane, 1,3-dioxane, piperidine, piperazine, pyrrolidine, morpholine, tetrahydrothiopyran, aziridine, azetidine and tetrahydrothiophene. Preferred substituent groups include alkyl, alkenyl, alkynyl, alkoxy, hydroxyl, halogen, haloalkyl, cycloalkyl, cycloalkoxy, cyano, amide, amino, and alkylamino.

As used herein, the term “aryl” refers to an optionally substituted benzene ring or to an optionally substituted fused benzene ring system, for example anthracene, phenanthrene, or naphthalene ring systems. Examples of “aryl” groups include, but are not limited to, phenyl, 2-naphthyl, and 1-naphthyl. Preferred substituent groups include alkyl, alkenyl, alkynyl, alkoxy, hydroxyl, halogen, haloalkyl, cycloalkyl, cycloalkoxy, cyano, amide, amino, and alkylamino.

As used herein, the term “heteroaryl” refers to an optionally substituted monocyclic five to seven membered aromatic ring, or to an optionally substituted fused bicyclic aromatic ring system comprising two of such aromatic rings. These heteroaryl rings contain one or more nitrogen, sulfur, and/or oxygen atoms, where N-oxides, sulfur oxides, and dioxides are permissible heteroatom substitutions. Preferably, the heteroatom is N.

Examples of “heteroaryl” groups used herein include, but should not be limited to, furan, thiophene, pyrrole, imidazole, pyrazole, triazole, tetrazole, thiazole, oxazole, isoxazole, oxadiazole, thiadiazole, isothiazole, pyridine, pyridazine, pyrazine, pyrimidine, quinoline, isoquinoline, benzofuran, benzothiophene, indole, indazole, benzimidazolyl, imidazopyridinyl, pyrazolopyridinyl, and pyrazolopyrimidinyl. Preferred substituent groups include alkyl, alkenyl, alkynyl, alkoxy, hydroxyl, halogen, haloalkyl, cycloalkyl, cycloalkoxy, cyano, amide, amino, and alkylamino.

As used herein the term “halogen” refers to fluorine, chlorine, bromine, or iodine.

As used herein the term “haloalkyl” refers to an alkyl group, as defined herein, which is substituted with at least one halogen. Examples of branched or straight chained “haloalkyl” groups useful in the present invention include, but are not limited to, methyl, ethyl, propyl, isopropyl, n-butyl, and t-butyl substituted independently with one or more halogens, e.g., fluoro, chloro, bromo, and iodo. The term “haloalkyl” should be interpreted to include such substituents as perfluoroalkyl groups and the like.

As used herein the term “hydroxy” or “hydroxyl” refers to a group —OH.

As used herein the term “alkoxy” refers to a group —OR′, where R′ is alkyl as defined.

As used herein the term “cycloalkoxy” refers to a group —OR′, where R′ is cycloalkyl as defined.

As used herein the term “alkoxycarbonyl” refers to groups such as:

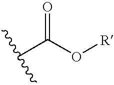

where the R′ represents an alkyl group as herein defined.

As used herein the term “aryloxycarbonyl” refers to groups such as:

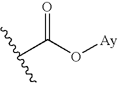

where the Ay represents an aryl group as herein defined.

As used herein the term “oxo” refers to a group ═O.

As used herein the term “nitro” refers to a group —NO₂.

As used herein the term “cyano” refers to a group —CN.

As used herein the term “azido” refers to a group —N₃.

As used herein the term amino refers to a group —NR′R″, where R′ and R″ independently represent H, alkyl, alkenyl, alkynyl, cycloalkyl, heterocyclyl, aryl, or heteroaryl. Similarly, the term “alkylamino” includes an alkylene linker through which the amino group is attached. Examples of “alkylamino” as used herein include groups such as —(CH₂)ₓNH₂, where x is preferably 1 to 6.

As used herein the term “amide” refers to a group —C(O)NR′R″, where R′ and R″ independently represent H, alkyl, alkenyl, alkynyl, cycloalkyl, heterocyclyl, aryl, or heteroaryl. Examples of “amide” as used herein include groups such as —C(O)NH₂, —C(O)NH(CH₃), —C(O)N(CH₃)₂, and the like.

As used herein throughout the present specification, the phrase “optionally substituted” or variations thereof denote an optional substitution, including multiple degrees of substitution, with one or more substituent group. The phrase should not be interpreted so as to be imprecise or duplicative of substitution patterns herein described or depicted specifically. Rather, those of ordinary skill in the art will appreciate that the phrase is included to provide for obvious modifications, which are encompassed within the scope of the appended claims.

The compounds of Formula (I) may crystallize in more than one form, a characteristic known as polymorphism, and such polymorphic forms (“polymorphs”) are within the scope of Formula (I). Polymorphism generally can occur as a response to changes in temperature, pressure, or both. Polymorphism can also result from variations in the crystallization process. Polymorphs can be distinguished by various physical characteristics known in the art such as x-ray diffraction patterns, solubility, and melting point. Though a crystalline form of compounds of Formula (I) are generally preferred, the invention also contemplates amorphous forms of the compounds produced by methods known in the art (e.g., spray drying, milling, freeze drying, and so forth).

Certain of the compounds described herein contain one or more chiral centers, or may otherwise be capable of existing as multiple stereoisomers. The scope of the present invention includes mixtures of stereoisomers as well as purified enantiomers or enantiomerically and/or diastereomerically enriched mixtures. Also included within the scope of the invention are the individual isomers of the compounds represented by Formula (I), as well as any wholly or partially equilibrated mixtures thereof. The present invention also includes the individual isomers of the compounds represented by the formulas above as mixtures with isomers thereof in which one or more chiral centers are inverted.

Typically, but not absolutely, the salts of the present invention are pharmaceutically acceptable salts. Salts encompassed within the term “pharmaceutically acceptable salts” refer to non-toxic salts of the compounds of this invention. Salts of the compounds of the present invention may comprise acid addition salts. Representative salts include acetate, benzenesulfonate, benzoate, bicarbonate, bisulfate, bitartrate, borate, calcium edetate, camsylate, carbonate, clavulanate, citrate, dihydrochloride, edisylate, estolate, esylate, fumarate, gluceptate, gluconate, glutamate, glycollylarsanilate, hexylresorcinate, hydrabamine, hydrobromide, hydrochloride, hydroxynaphthoate, hydroiodide (HI), isethionate, lactate, lactobionate, laurate, malate, maleate, mandelate, mesylate, methylsulfate, monopotassium maleate, mucate, napsylate, nitrate, N-methylglucamine, oxalate, pamoate (embonate), palmitate, pantothenate, phosphate/diphosphate, polygalacturonate, potassium, salicylate, sodium, stearate, subacetate, succinate, sulfate, tannate, tartrate, teoclate, tosylate, triethiodide, trimethylammonium, and valerate salts. Other salts, which are not pharmaceutically acceptable, may be useful in the preparation of compounds of this invention and these should be considered to form a further aspect of the invention.

As used herein, the term “solvate” refers to a complex of variable stoichiometry formed by a solute (in this invention, a compound of Formula I, or a salt or physiologically functional derivative thereof) and a solvent. Such solvents, for the purpose of the invention, should not interfere with the biological activity of the solute. Non-limiting examples of suitable solvents include, but are not limited to water, methanol, ethanol, ethyl acetate, acetone, acetonitrile, and acetic acid. Preferably the solvent used is a pharmaceutically acceptable solvent. Non-limiting examples of suitable pharmaceutically acceptable solvents include water, ethanol, and acetic acid. Most preferably the solvent used is water.

As used herein, the term “physiologically functional derivative” refers to any pharmaceutically acceptable derivative of a compound of the present invention that, upon administration to a mammal, is capable of providing (directly or indirectly) a compound of the present invention or an active metabolite thereof. Such derivatives, for example, esters and amides, will be clear to those skilled in the art, without undue experimentation. Reference may be made to the teaching of Burger's Medicinal Chemistry And Drug Discovery, 5^{th} Edition, Vol 1: Principles and Practice, which is incorporated herein by reference to the extent that it teaches physiologically functional derivatives.

As used herein, the term “effective amount” means that amount of a drug or pharmaceutical agent that will elicit the biological or medical response of a tissue, system, animal, or human that is being sought, for instance, by a researcher or clinician. The term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

The term “modulators” as used herein is intended to encompass antagonist, agonist, inverse agonist, partial agonist or partial antagonist, inhibitors and activators. In one preferred embodiment of the present invention, the compounds demonstrate protective effects against HIV infection by inhibiting binding of HIV to a chemokine receptor such as CXCR4 of a target cell. The invention includes a method that comprises contacting the target cell with an amount of the compound that is effective at inhibiting the binding of the virus to the chemokine receptor.

In addition to the role chemokine receptors play in HIV infection this receptor class has also been implicated in a wide variety of diseases. Thus CXCR4 modulators may also have a therapeutic role in the treatment of diseases associated with hematopoiesis, myocardial infarction, including but not limited to, controlling the side effects of chemotherapy, enhancing the success of bone marrow transplantation, enhancing wound healing and burn treatment, as well as combating bacterial infections in leukemia. In addition, compounds may also have a therapeutic role in diseases associated with inflammation, including but not limited to inflammatory or allergic diseases such as asthma, allergic rhinitis, hypersensitivity lung diseases, hypersensitivity pneumonitis, eosinophilic pneumonitis, delayed-type hypersensitivity, interstitial lung disease (ILD) (e.g. idiopathic pulmonary fibrosis, or ILD associated with rheumatoid arthritis, systemic lupus erythematosus, ankylosing spondylitis, systemic sclerosis, Sjogren's syndrome, polymyositis or dermatomyositis); systemic anaphylaxis or hypersensitivity responses, drug allergies, insect sting allergies; autoimmune diseases such as rheumatoid arthritis, psoriatic arthritis, systemic lupus erythematosus, myastenia gravis, juvenile onset diabetes; glomerulonephritis, autoimmune throiditis, graft rejection, including allograft rejection or graft-versus-host disease; inflammatory bowel diseases, such as Crohn's disease and ulcerative colitus; spondyloarthropathies; scleroderma; psoriasis (including T-cell-mediated psoriasis) and inflammatory dermatoses such as dermatitis, eczema, atopic dermatitis, allergic contact dermatitis, urticaria, vasculitis (e.g. necrotizing, cutaneous, and hypersensitivity vasculitis); eosinophilic myotis, eosinophilic fasciitis; and cancers.

For use in therapy, therapeutically effective amounts of a compound of Formula (I), as well as salts, solvates, and physiological functional derivatives thereof, may be administered as the raw chemical. Additionally, the active ingredient may be presented as a pharmaceutical composition.

Accordingly, the invention further provides pharmaceutical compositions that include effective amounts of compounds of the Formula (I) and salts, solvates, and physiological functional derivatives thereof, and one or more pharmaceutically acceptable carriers, diluents, or excipients. The compounds of Formula (I) and salts, solvates, and physiologically functional derivatives thereof, are as herein described. The carrier(s), diluent(s) or excipient(s) must be acceptable, in the sense of being compatible with the other ingredients of the formulation and not deleterious to the recipient of the pharmaceutical composition.

In accordance with another aspect of the invention there is also provided a process for the preparation of a pharmaceutical formulation including admixing a compound of the Formula (I) or salts, solvates, and physiological functional derivatives thereof, with one or more pharmaceutically acceptable carriers, diluents or excipients.

A therapeutically effective amount of a compound of the present invention will depend upon a number of factors. For example, the species, age, and weight of the recipient, the precise condition requiring treatment and its severity, the nature of the formulation, and the route of administration are all factors to be considered. The therapeutically effective amount ultimately should be at the discretion of the attendant physician or veterinarian. Regardless, an effective amount of a compound of Formula (I) for the treatment of humans suffering from frailty, generally, should be in the range of 0.1 to 100 mg/kg body weight of recipient (mammal) per day. More usually the effective amount should be in the range of 0.1 to 10 mg/kg body weight per day. Thus, for a 70 kg adult mammal one example of an actual amount per day would usually be from 7 to 700 mg. This amount may be given in a single dose per day or in a number (such as two, three, four, five, or more) of sub-doses per day such that the total daily dose is the same. An effective amount of a salt, solvate, or physiologically functional derivative thereof, may be determined as a proportion of the effective amount of the compound of Formula (I) per se. Similar dosages should be appropriate for treatment of the other conditions referred to herein.

Pharmaceutical formulations may be presented in unit dose forms containing a predetermined amount of active ingredient per unit dose. Such a unit may contain, as a non-limiting example, 0.5 mg to 1 g of a compound of the Formula (I), depending on the condition being treated, the route of administration, and the age, weight, and condition of the patient. Preferred unit dosage formulations are those containing a daily dose or sub-dose, as herein above recited, or an appropriate fraction thereof, of an active ingredient. Such pharmaceutical formulations may be prepared by any of the methods well known in the pharmacy art.

Pharmaceutical formulations may be adapted for administration by any appropriate route, for example by an oral (including buccal or sublingual), rectal, nasal, topical (including buccal, sublingual or transdermal), vaginal, or parenteral (including subcutaneous, intramuscular, intravenous or intradermal) route. Such formulations may be prepared by any method known in the art of pharmacy, for example by bringing into association the active ingredient with the carrier(s) or excipient(s). By way of example, and not meant to limit the invention, with regard to certain conditions and disorders for which the compounds of the present invention are believed useful certain routes will be preferable to others.

Pharmaceutical formulations adapted for oral administration may be presented as discrete units such as capsules or tablets; powders or granules; solutions or suspensions, each with aqueous or non-aqueous liquids; edible foams or whips; or oil-in-water liquid emulsions or water-in-oil liquid emulsions. For instance, for oral administration in the form of a tablet or capsule, the active drug component can be combined with an oral, non-toxic pharmaceutically acceptable inert carrier such as ethanol, glycerol, water, and the like. Generally, powders are prepared by comminuting the compound to a suitable fine size and mixing with an appropriate pharmaceutical carrier such as an edible carbohydrate, as, for example, starch or mannitol. Flavorings, preservatives, dispersing agents, and coloring agents can also be present.

Capsules are made by preparing a powder, liquid, or suspension mixture and encapsulating with gelatin or some other appropriate shell material. Glidants and lubricants such as colloidal silica, talc, magnesium stearate, calcium stearate, or solid polyethylene glycol can be added to the mixture before the encapsulation. A disintegrating or solubilizing agent such as agar-agar, calcium carbonate or sodium carbonate can also be added to improve the availability of the medicament when the capsule is ingested. Moreover, when desired or necessary, suitable binders, lubricants, disintegrating agents, and coloring agents can also be incorporated into the mixture. Examples of suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants useful in these dosage forms include, for example, sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

Tablets are formulated, for example, by preparing a powder mixture, granulating or slugging, adding a lubricant and disintegrant, and pressing into tablets. A powder mixture may be prepared by mixing the compound, suitably comminuted, with a diluent or base as described above. Optional ingredients include binders such as carboxymethylcellulose, aliginates, gelatins, or polyvinyl pyrrolidone, solution retardants such as paraffin, resorption accelerators such as a quaternary salt, and/or absorption agents such as bentonite, kaolin, or dicalcium phosphate. The powder mixture can be wet-granulated with a binder such as syrup, starch paste, acadia mucilage or solutions of cellulosic or polymeric materials, and forcing through a screen. As an alternative to granulating, the powder mixture can be run through the tablet machine and the result is imperfectly formed slugs broken into granules. The granules can be lubricated to prevent sticking to the tablet-forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material, and a polish coating of wax can be provided. Dyestuffs can be added to these coatings to distinguish different unit dosages.

Oral fluids such as solutions, syrups, and elixirs can be prepared in dosage unit form so that a given quantity contains a predetermined amount of the compound. Syrups can be prepared, for example, by dissolving the compound in a suitably flavored aqueous solution, while elixirs are prepared through the use of a non-toxic alcoholic vehicle. Suspensions can be formulated generally by dispersing the compound in a non-toxic vehicle. Solubilizers and emulsifiers such as ethoxylated isostearyl alcohols and polyoxy ethylene sorbitol ethers, preservatives; flavor additives such as peppermint oil, or natural sweeteners, saccharin, or other artificial sweeteners; and the like can also be added.

Where appropriate, dosage unit formulations for oral administration can be microencapsulated. The formulation can also be prepared to prolong or sustain the release as for example by coating or embedding particulate material in polymers, wax or the like.

The compounds of Formula (I) and salts, solvates, and physiological functional derivatives thereof, can also be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

The compounds of Formula (I) and salts, solvates, and physiologically functional derivatives thereof may also be delivered by the use of monoclonal antibodies as individual carriers to which the compound molecules are coupled.

The compounds may also be coupled with soluble polymers as targetable drug carriers. Such polymers can include polyvinylpyrrolidone (PVP), pyran copolymer, polyhydroxypropylmethacrylamide-phenol, polyhydroxyethyl-aspartamidephenol, or polyethyleneoxidepolylysine substituted with palmitoyl residues. Furthermore, the compounds may be coupled to a class of biodegradable polymers useful in achieving controlled release of a drug; for example, polylactic acid, polyepsilon caprolactone, polyhydroxy butyric acid, polyorthoesters, polyacetals, polydihydropyrans, polycyanoacrylates, and cross-linked or amphipathic block copolymers of hydrogels.

Pharmaceutical formulations adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research, 3(6), 318 (1986), incorporated herein by reference as related to such delivery systems.

Pharmaceutical formulations adapted for topical administration may be formulated as ointments, creams, suspensions, lotions, powders, solutions, pastes, gels, sprays, aerosols, or oils.

For treatments of the eye or other external tissues, for example mouth and skin, the formulations may be applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base.

Pharmaceutical formulations adapted for topical administrations to the eye include eye drops wherein the active ingredient is dissolved or suspended in a suitable carrier, especially an aqueous solvent.

Pharmaceutical formulations adapted for topical administration in the mouth include lozenges, pastilles, and mouthwashes.

Pharmaceutical formulations adapted for nasal administration, where the carrier is a solid, include a coarse powder having a particle size for example in the range 20 to 500 microns. The powder is administered in the manner in which snuff is taken, i.e., by rapid inhalation through the nasal passage from a container of the powder held close up to the nose. Suitable formulations wherein the carrier is a liquid, for administration as a nasal spray or as nasal drops, include aqueous or oil solutions of the active ingredient.

Pharmaceutical formulations adapted for administration by inhalation include fine particle dusts or mists, which may be generated by means of various types of metered dose pressurized aerosols, nebulizers, or insufflators.

Pharmaceutical formulations adapted for rectal administration may be presented as suppositories or as enemas.

Pharmaceutical formulations adapted for vaginal administration may be presented as pessaries, tampons, creams, gels, pastes, foams, or spray formulations.

Pharmaceutical formulations adapted for parenteral administration include aqueous and non-aqueous sterile injection solutions which may contain anti-oxidants, buffers, bacteriostats, and solutes that render the formulation isotonic with the blood of the intended recipient; and aqueous and non-aqueous sterile suspensions which may include suspending agents and thickening agents. The formulations may be presented in unit-dose or multi-dose containers, for example sealed ampules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules, and tablets.

In addition to the ingredients particularly mentioned above, the formulations may include other agents conventional in the art having regard to the type of formulation in question. For example, formulations suitable for oral administration may include flavoring or coloring agents.

The compounds of the present invention and their salts, solvates, and physiologically functional derivatives thereof, may be employed alone or in combination with other therapeutic agents. The compound(s) of Formula (I) and the other pharmaceutically active agent(s) may be administered together or separately and, when administered separately, administration may occur simultaneously or sequentially, in any order. The amounts of the compound(s) of Formula (I) and the other pharmaceutically active agent(s) and the relative timings of administration will be selected in order to achieve the desired combined therapeutic effect. The administration in combination of a compound of Formula (I) salts, solvates, or physiologically functional derivatives thereof with other treatment agents may be in combination by administration concomitantly in: (1) a unitary pharmaceutical composition including both compounds; or (2) separate pharmaceutical compositions each including one of the compounds. Alternatively, the combination may be administered separately in a sequential manner wherein one treatment agent is administered first and the other second or vice versa. Such sequential administration may be close in time or remote in time.

The compounds of the present invention may be used in the treatment of a variety of disorders and conditions and, as such, the compounds of the present invention may be used in combination with a variety of other suitable therapeutic agents useful in the treatment or prophylaxis of those disorders or conditions.

The compounds may be used in combination with any other pharmaceutical composition where such combined therapy may be useful to modulate chemokine receptor activity and thereby prevent and treat inflammatory and/or immunoregulatory diseases.

The present invention may be used in combination with one or more agents useful in the prevention or treatment of HIV. Examples of such agents include:

Nucleotide reverse transcriptase inhibitors such as zidovudine, didanosine, lamivudine, zalcitabine, abacavir, stavidine, adefovir, adefovir dipivoxil, fozivudine, todoxil, and similar agents;

Non-nucleotide reverse transcriptase inhibitors (including an agent having anti-oxidation activity such as immunocal, oltipraz, etc.) such as nevirapine, delavirdine, efavirenz, loviride, immunocal, oltipraz, and similar agents;

Protease inhibitors such as saquinavir, ritonavir, indinavir, nelfinavir, amprenavir, fosamprenavir, brecanavir, atazanavir, tipranavir, palinavir, lasinavir, and similar agents;

Entry inhibitors such as T-20, T-1249, PRO-542, PRO-140, TNX-355, BMS-806, 5-Helix and similar agents;

Integrase inhibitors such as L-870,180 and similar agents and/or those set forth in WO 2004/101512 and U.S. 60/543,670;

Budding inhibitors such as PA-344 and PA-457, and similar agents; and

Other CXCR4 and/or CCR5 inhibitors such as Sch-C, Sch-D, TAK779, UK 427,857, TAK449, as well as those disclosed in WO 02/74769, PCT/US03/39644, PCT/US03/39975, PCT/US03/39619, PCT/US03/39618, PCT/US03/39740, and PCT/US03/39732, and similar agents.

The scope of combinations of compounds of this invention with HIV agents is not limited to those mentioned above, but includes in principle any combination with any pharmaceutical composition useful for the treatment of HIV. As noted, in such combinations the compounds of the present invention and other HIV agents may be administered separately or in conjunction. In addition, one agent may be prior to, concurrent to, or subsequent to the administration of other agent(s).

The compounds of this invention may be made by a variety of methods, including well-known standard synthetic methods. Illustrative general synthetic methods are set out below and then specific compounds of the invention are prepared in the working Examples.

In all of the examples described below, protecting groups for sensitive or reactive groups are employed where necessary in accordance with general principles of synthetic chemistry. Protecting groups are manipulated according to standard methods of organic synthesis (T. W. Green and P. G. M. Wuts (1991) Protecting Groups in Organic Synthesis, John Wiley & Sons, incorporated by reference with regard to protecting groups). These groups are removed at a convenient stage of the compound synthesis using methods that are readily apparent to those skilled in the art. The selection of processes as well as the reaction conditions and order of their execution shall be consistent with the preparation of compounds of Formula (I).

Those skilled in the art will recognize if a stereocenter exists in compounds of Formula (I). Accordingly, the scope of the present invention includes all possible stereoisomers and includes not only racemic compounds but the individual enantiomers as well. When a compound is desired as a single enantiomer, such may be obtained by stereospecific synthesis, by resolution of the final product or any convenient intermediate, or by chiral chromatographic methods as are known in the art. Resolution of the final product, an intermediate, or a starting material may be affected by any suitable method known in the art. See, for example, Stereochemistry of Organic Compounds by E. L. Eliel, S. H. Wilen, and L. N. Mander (Wiley-Interscience, 1994), incorporated by reference with regard to stereochemistry.

### Experimental Section

### Abbreviations:

As used herein the symbols and conventions used in these processes, schemes and examples are consistent with those used in the contemporary scientific literature, for example, the Journal of the American Chemical Society or the Journal of Biological Chemistry. Specifically, the following abbreviations may be used in the examples and throughout the specification:

| | |
|---|---|
| g (grams); | mg (milligrams); |
| L (liters); | mL (milliliters); |
| μL (microliters); | psi (pounds per square inch); |
| M (molar); | mM (millimolar); |
| Hz (Hertz); | MHz (megahertz); |
| mol (moles); | mmol (millimoles); |
| RT (room temperature); | h (hours); |
| min (minutes); | TLC (thin layer chromatography); |
| mp (melting point); | RP (reverse phase); |
| Tᵣ (retention time); | TFA (trifluoroacetic acid); |
| TEA (triethylamine); | THF (tetrahydrofuran); |
| TFAA (trifluoroacetic anhydride); | CD₃OD (deuterated methanol); |
| CDCl₃ (deuterated chloroform); | DMSO (dimethylsulfoxide); |
| SiO₂ (silica); | atm (atmosphere); |
| EtOAc (ethyl acetate); | CHCl₃ (chloroform); |
| HCl (hydrochloric acid); | Ac (acetyl); |
| DMF (N,N-dimethylformamide); | Me (methyl); |
| Cs₂CO₃ (cesium carbonate); | EtOH (ethanol); |
| Et (ethyl); | tBu (tert-butyl); |
| MeOH (methanol) | p-TsOH (p-toluenesulfonic acid); |

MP-TsOH (polystyrene resin bound equivalent of p-TsOH from Argonaut
Technologies).

Unless otherwise indicated, all temperatures are expressed in ° C. (degrees Centigrade). All reactions conducted at room temperature unless otherwise noted.

¹H-NMR spectra were recorded on a Varian instrument (e.g., VXR-300, a Varian Unity-300, a Varian Unity-400), or a General Electric QE-300. Chemical shifts are expressed in parts per million (ppm, δ units). Coupling constants are in units of hertz (Hz). Splitting patterns describe apparent multiplicities and are designated as s (singlet), d (doublet), t (triplet), q (quartet), m (multiplet), or br (broad).

Mass spectra were obtained on Micromass Platform or ZMD mass spectrometers from Micromass Ltd., Altricham, UK, using either Atmospheric Chemical Ionization (APCI) or Electrospray Ionization (ESI).

Analytical thin layer chromatography was used to verify the purity of intermediate(s) which could not be isolated or which were too unstable for full characterization as well as to follow the progress of reaction(s).

The absolute configuration of compounds was assigned by Ab Initio Vibrational Circular Dichroism (VCD) Spectroscopy. The experimental VCD spectra were acquired in CDCl₃ using a Bomem Chiral RTM VCD spectrometer operating between 2000 and 800 cm⁻¹. The Gaussian 98 Suite of computational programs was used to calculate model VCD spectrums. The stereochemical assignments were made by comparing this experimental spectrum to the VCD spectrum calculated for a model structure. Incorporated by reference with regard to such spectroscopy are: J. R. Chesseman, M. J. Frisch, F. J. Devlin and P. J. Stephens, Chem. Phys. Lett. 252 (1996) 211; P. J. Stephens and F. J. Devlin, Chirality 12 (2000) 172; and Gaussian 98, Revision A.11.4, M. J. Frisch et al., Gaussian, Inc., Pittsburgh Pa., 2002.

Compounds of formula (V) where all variables are as defined herein can be prepared according to Scheme 1 (where m¹ is 1-3):

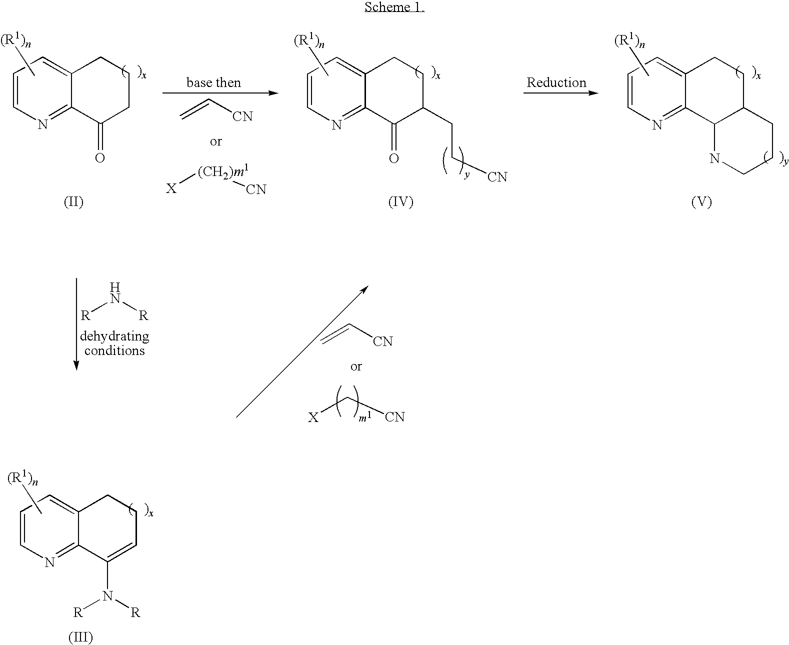

Generally, the process for preparing compounds of formula (V) comprises the steps of:
a) reacting a compound of formula (II) with a secondary amine to prepare a compound of formula (III);
b) reacting a compound of formula (III) with an appropriate alkylating agent to prepare a compound of formula (IV);

### Alternatively,

c) treating a compound of formula (II) with a suitable base and alkylating with an alkylating reagent to prepare a compound of formula (IV);
d) Forming a compound of formula (V) via reduction of a compound of formula (IV).

More specifically, treatment of a compound of formula (II) with a suitable secondary amine, in a suitable solvent, with a suitable acid catalyst, optionally with heating, and a suitable means of water removal gives a compound of formula (III). Suitable secondary amines include, but are not limited to, diethylamine, pyrrolidine, piperidine, morpholine, and the like. Optionally a chiral secondary amine can be used. Suitable solvents include benzene, toluene, and the like. The reaction can optionally be heated to between about 25° C. and 150° C. Suitable acid catalysts include, but are not limited to, p-toluenesulfonic acid, hydrogen chloride, and the like. Suitable means of water removal include, but are not limited to, Dean-Stark apparatus, molecular sieves, and the like.

Reaction of a compound (III) with a suitable alkylating reagent, in a suitable solvent, optionally with heating gives a compound of formula (IV). Suitable alkylating reagents include, but are not limited to acrylonitrile, bromoacetonitrile, and the like. Suitable solvents include, but are not limited to, methanol, ethanol, dioxane, and the like. The reaction can optionally be heated to between about 30° C. and 100° C.

Treatment of a compound (II) with a suitable base, in a suitable solvent, followed by treatment with a suitable alkylating reagent, optionally with heating, gives a compound of formula (IV). Suitable bases include, but are not limited to, lithium diisopropylamide, n-butyl lithium, lithium bis(trimethylsilyl)amide and the like. Suitable solvents include tetrahydrofuran, diethyl ether and the like. Suitable alkylating reagents include, but are not limited to, acrylonitrile, bromoacetonitrile, and the like.

Reduction of a compound (formula IV) in a suitable solvent, with an appropriate metal catalyst, under a hydrogen atomosphere of suitable pressure, optionally with heating, gives a compound of formula (V). Suitable solvents include, but are not limited to, methanol, ethanol, tetrahydrofuran, and the like. Appropriate metal catalysts include, but are not limited to, raney nickel, palladium, platinum, and the like. Suitable hydrogen pressures are 1-100 psi. The reaction may optionally be heated to between 30° C. and 100° C.

Compounds of formula (Va) where all variables are as defined herein can be prepared according to Scheme 2.

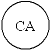

is a suitable chiral auxiliary.

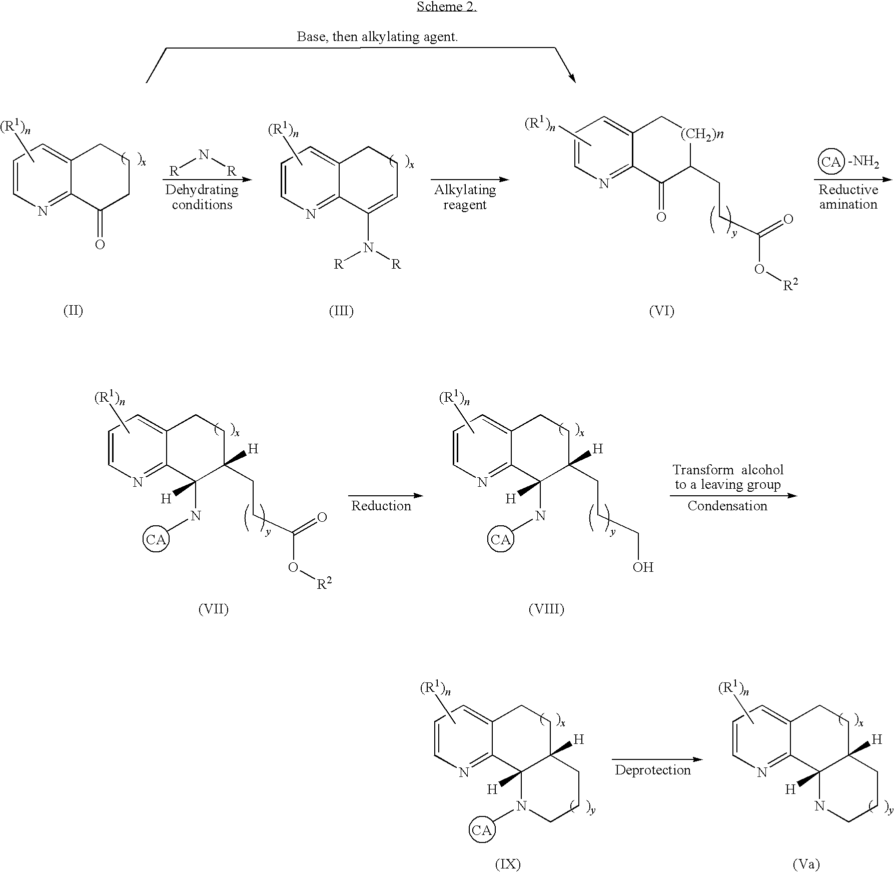

Generally, the process for preparing compounds of formula (Va) comprises the steps of:
a) reacting a compound of formula (II) with a secondary amine to prepare a compound of formula (III);
b) reacting a compound of formula (III) with an appropriate alkylating reagent to prepare a compound of formula (VI);
c) optionally forming compound of formula (VI) directly from a compound of formula (II) by treatment with base followed by an appropriate alkylating agent
d) reacting a compound of formula (VI) with a chiral auxiliary amine (CA-NH₂) and a reducing reagent to prepare a compound of formula (VII);
e) treating a compound (VII) with a reducing reagent to prepare a compound of formula (VIII);
f) transforming a compound of formula (VII) to prepare a compound of formula (IX);
g) cleavage of the chiral auxiliary of a compound (IX) to prepare a compound of formula (Va) in a chiral form.
   For Scheme 2 the alkylating agent depicted for transformation of compound of formula (II) or (III) to a compound of formula (VI) is an ester. Optionally, protected or unprotected aldehydes or alcohols could be used as alkylating agent as is evident to one skilled in the art.

More specifically, treatment of a compound of formula (II) with a suitable secondary amine (optionally chiral), in a suitable solvent, with a suitable acid catalyst, optionally with heating, and a with suitable means of water removal gives a compound of formula (III). Suitable secondary amines include, but are not limited to, diethylamine, pyrrolidine, piperidine, morpholine, and the like. Suitable solvents include benzene, toluene, and the like. The reaction can optionally be heated to between about 25° C. and 150° C. Suitable acid catalysts include, but are not limited to, p-toluenesulfonic acid, hydrogen chloride, and the like. Suitable means of water removal include, but are not limited to, azeotrope via Dean-Stark apparatus, molecular sieves, and the like.

Reaction of a compound (III) with a suitable alkylating reagent, in a suitable solvent, optionally with heating gives a compound of formula (VI). Suitable alkylating reagents include, but are not limited to, methyl acrylate, ethyl acrylate, methyl bromoacetate, ethyl bromoacetate, and the like. Suitable solvents include, but are not limited to, methanol, ethanol, dioxane, and the like. The reaction can optionally be heated to between about 30° C. and reflux.

A compound of formula (VII) can be prepared by reacting a compound of formula (VI) with a chiral auxiliary amine (CA-NH₂) and suitable reducing agent. The reaction may be carried out as a two step process by adding the chiral auxiliary amine to first form an imine, which is subsequently treated with a reductive agent to form a compound of formula (VII). Alternatively, this reaction can be carried out in one pot by adding the chiral auxiliary amine and reductive agent, either sequentially or at the same time.

For the two step process, a compound of formula (VI) is reacted with a suitable chiral auxiliary amine, in a suitable solvent, with a suitable acid catalyst, with heating, and with a suitable means of water removal to prepare a compound of formula (VII). Suitable chiral auxiliary amines include, but are not limited to, (1R)-1-[4-(methyloxy)phenyl]ethanamine, (1S)-1-[4-(methyloxy)phenyl]ethanamine, (1R)-1-[2,4-bis(methyloxy)phenyl]ethanamine, (1S)-1-[2,4-bis(methyloxy)phenyl]ethanamine, (1R)-1-phenylethanamine, (1S)-1-phenylethanamine, and the like. Suitable solvents include, but are not limited to, benzene, toluene, and the like. Suitable acid catalysts include, but are not limited to, p-toluenesulfonic acid, hydrogen chloride, and the like. Suitable means of water removal include, but are not limited to, azeotrope via Dean-Stark apparatus, molecular sieves, and the like. The imine thus formed is dissolved in a suitable solvent and treated with a suitable reducing agent. Suitable solvents include, but are not limited to, 1,2-dichloroethane, dichloromethane, methanol, ethanol, tetrahydrofuran, and the like. Suitable reducing reagents include, but are not limited to, sodium triacetoxyborohydride, sodium cyanoborohydride, sodium borohydride, and the like.

For a one pot process, a suitable chiral auxiliary amine, and a suitable reducing reagent are added to a compound of formula (VI) in a suitable solvent to give a compound of formula (VII). The reaction may optionally incorporate a suitable acid catalyst, and may optionally be heated between room temperature and 150° C. Suitable chiral auxiliary amines include, but are not limited to, (1R)-1-[4-(methyloxy)phenyl]ethanamine, (1S)-1-[4-(methyloxy)phenyl]ethanamine, (1R)-1-[2,4-bis(methyloxy)phenyl]ethanamine, (1S)-1-[2,4-bis(methyloxy)phenyl]ethanamine, (1R)-1-phenylethanamine, (1S)-1-phenylethanamine, and the like. Suitable solvents include, but are not limited to, 1,2-dichloroethane, dichloromethane, and the like. Suitable acid catalysts include, but are not limited to, acetic acid, and the like.

A compound of formula (VII) can be treated with a suitable reducing reagent, in a suitable solvent, optionally with heating, to prepare a compound of formula (VII). Suitable reducing reagents include, but are not limited to, lithium aluminum hydride, lithium borohydride, borane dimethylsulfide complex, diisobutylaluminum hydride, and the like. Suitable solvents include, but are not limited to, tetrahydrofuran, diethyl ether, and the like. The reaction may optionally be heated between about 25° C. and 100° C.

A compound of formula (VIII) can be treated with a suitable activating reagent, in a suitable solvent, with a suitable base, optionally with a suitable catalyst, and optionally with heating to promote condensation to a compound of formula (IX). Suitable activating reagents are those that convert the hydroxyl moiety of compound (VIII) to a leaving group and include, but are not limited to, methanesulfonyl chloride, p-toluenesulfonyl, chloride, thionyl chloride, and the like. Suitable solvents include, but are not limited to, dichloromethane, tetrahydrofuran, acetonitrile, N,N-dimethylformamide and the like. Suitable bases include, but are not limited to, N,N-diisopropylethylamine, triethylamine, DBU, 4-dimethylaminopyridine, cesium carbonate, potassium carbonate, sodium hydride and the like. Suitable catalysts include, but are not limited to, 4-dimethylaminopyridine, sodium iodide, tetrabutylammonium iodide and the like. The reaction may optionally be heated to between about 25° C. and 150° C.

A compound of formula (Va) can be prepared from a compound of formula (IX) via removal of the chiral auxiliary. For an acid sensitive chiral auxiliary, suitable acids include, but are not limited to trifluoroacetic acid, hydrogen chloride, hydrogen bromide, and the like. Suitable solvents include, but are not limited to, dichloromethane, methanol, ethanol, tetrahydrofuran, and the like.

Alternatively, a compound of formula (IX) can be converted to a compound of formula (Va) under reducing conditions, where CA is reductively labile. Suitable reducing conditions include, but are not limited to, catalytic hydrogenation using a suitable metal catalyst. Suitable metal catalysts include, but are not limited to, palladium, platinum, and the like.

Scheme 2 shows a suggested order of steps. As evident to one skilled in the art, the sequence of several of these steps can be changed.

Compounds of Formula (I) can be prepared from a compound of formula (V) and a compound of formula (X) as outlined in Scheme 3.

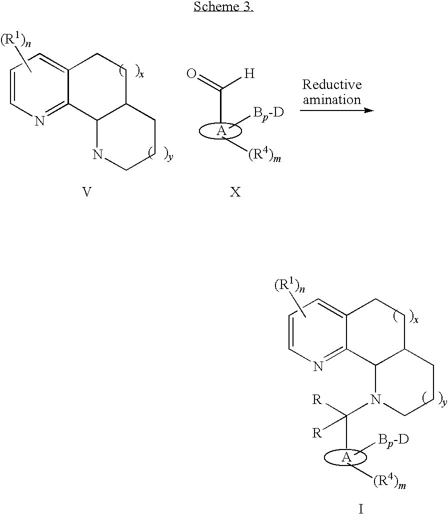

More specifically, compounds of Formula (I) can be prepared by reacting a compound of formula (X) with a compound (V) under reductive conditions. The reductive amination can be carried out in an inert solvent in the presence of a reducing agent. The reaction may be heated to 50-150° C. or performed at ambient temperature. Suitable solvents include dichloromethane, dichloroethane, tetrahydrofuran, acetonitrile, toluene, and the like. The reducing agent is typically sodium borohydride, sodium cyanoborohydride, sodium triacetoxyborohydride, and the like. Optionally the reaction can be run in presence of acid, such as acetic acid and the like.

Compounds of formula (X) can be prepared by methods well known to those skilled in the art of organic synthesis.

Compound of Formula (I) can be prepared from a compound of formula (V) and a compound of formula (XI) as outlined in Scheme 4.

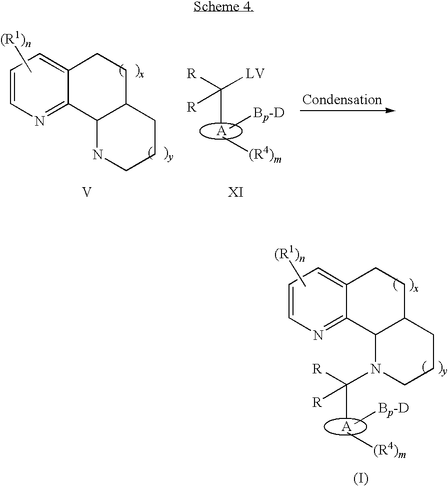

Compound of Formula (I) can be prepared by reacting a compound of formula (V) with a compound of formula (XI) where LV is a leaving group (e.g., halogen, mesylate, tosylate, or the like). This condensation is typically carried out in a suitable solvent optionally in the presence of base, optionally with heating. Suitable solvents include tetrahydrofuran, dioxane, acetonitrile, nitromethane, N,N-dimethylformamide, and the like. Suitable bases include triethylamine, pyridine, dimethylaminopyridine, N,N-diisopropylethylamine, potassium carbonate, sodium carbonate, cesium carbonate and the like. The reaction can be carried out at room temperature or optionally heated to 30-200° C. Optionally the reaction can be carried out in a microwave. A catalyst, such as potassium iodide, tertbutylammonium iodide, or the like, can optionally be added to the reaction mixture. Compounds of formula (XI) can be prepared by methods similar to those described in the literature (e.g. for imidazopyridines: Chem. Pharm. Bull. 2000, 48, 935; Tetrahedron, 1991, 47, 5173; Tetrahedron Lett 1990, 31, 3013; J. Heterocyclic Chemistry, 1988, 25, 129; Chemistry of Heterocyclic Compounds, 2002, 38, 590; each incorporated by reference with regard to such synthesis).

Compounds of Formula (I) where heterocycle A is a benzimidazole can be prepared as outlined in Scheme 5, where all variables are as defined in connection with Formula (I) and R³ is H or alkyl.

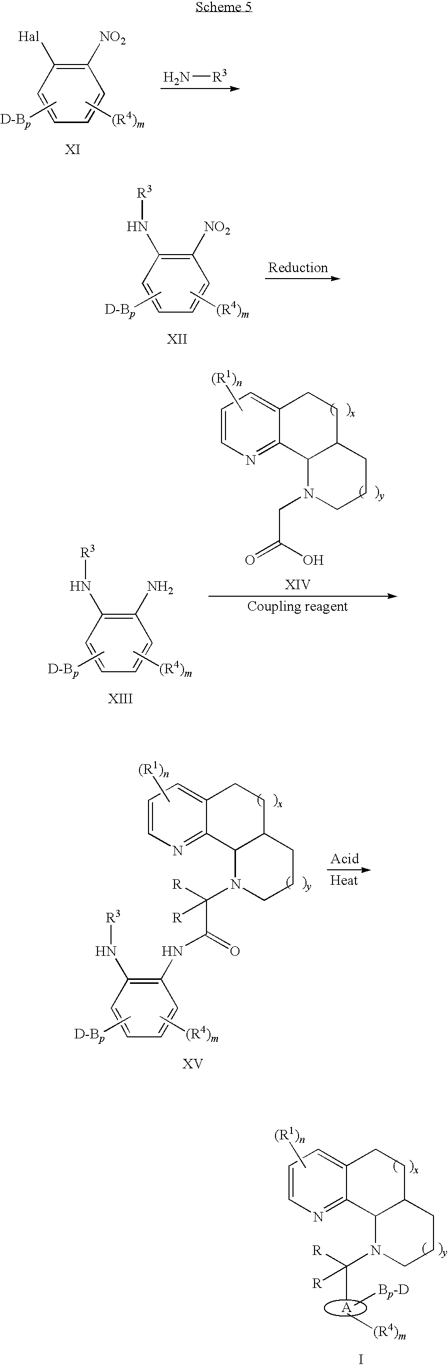

Compounds of Formula (I) where heterocycle

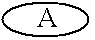

is a benzimidazole can be prepared by treatment of compound of formula (XV) under acidic conditions optionally with heating. The reaction can be carried out by treating the compound of formula (XV) with a suitable acid optionally in the presence of an inert solvent. The reaction may be heated to 50-200° C. or performed at ambient temperature. Suitable acids include acetic acid, trifluoroacetic acid, hydrochloric acid, and the like. The reaction can be carried out using the acid as a solvent. Other suitable solvents include tetrahydrofuran, acetonitrile, toluene, and the like. More specifically, as illustrated, compounds of formula (XV) can be prepared by coupling of a compound of formula (XIII) with a compound of formula (XIV). This coupling can be carried out using a variety of coupling reagent well know to those skilled in the art of organic synthesis (e.g., EDC, HOBt/HBTu; BOPCl). The reaction can be carried out with heating or at ambient temperature. Suitable solvents for this reaction include acetonitrile, tetrahydrofuran, and the like. Compounds of formula (XIII) are commercially available or can be prepared by methods similar to those known in the literature. Compound of formula (XIV) can be prepared from compound of formula (V).

Compound of formula (XXIV) where n is 0, x and y are 1, R is H, A is imidazopyridine, p is 0 and D is Het, m is 0 can be prepared according to scheme 6.

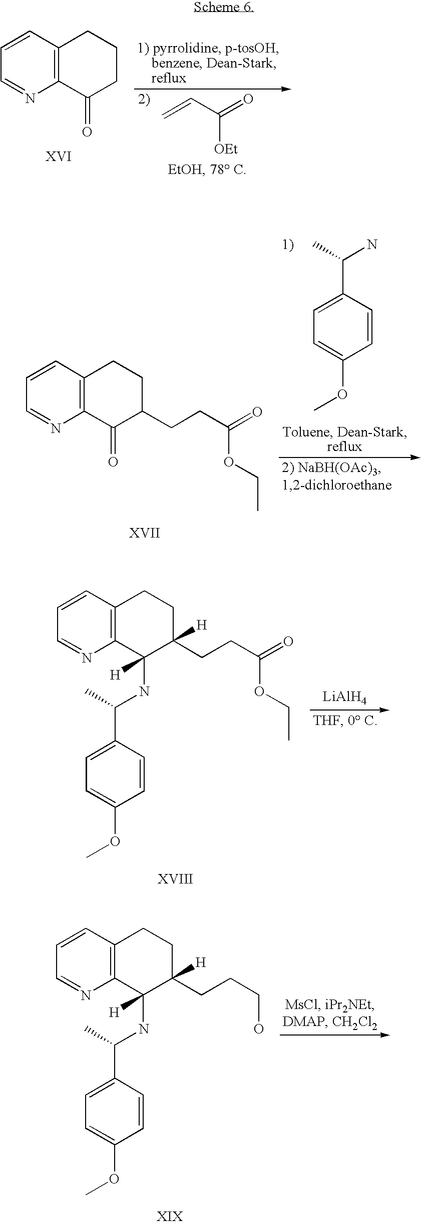

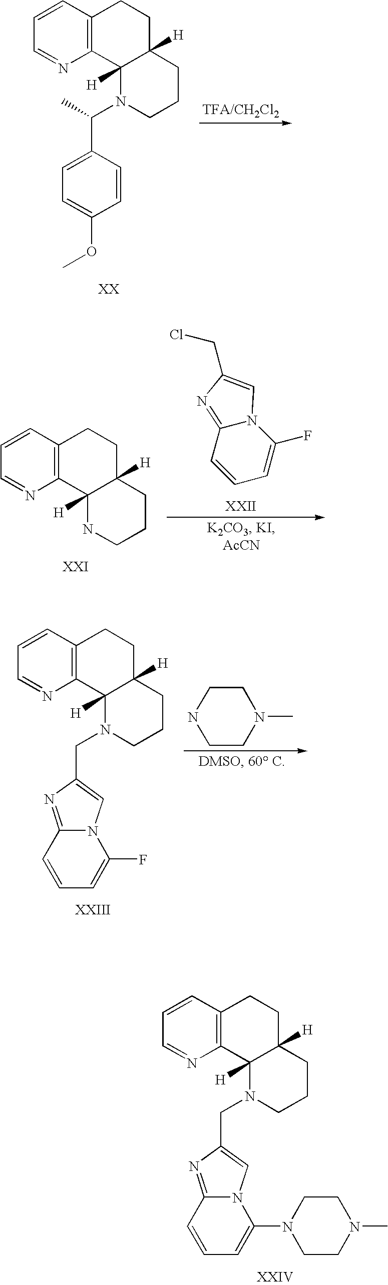

Generally, a process for preparing compound of (XXIV) comprises the steps of:
e) reacting compound (XVI) with pyrrolidine to form an enamine, which is treated with ethyl acrylate to prepare compound (XVII);
f) reacting compound (XVII) with (1S)-1-[4-methyloxy)phenyl]ethanamine to form an enamine, which is reduced with sodium triacetoxyborohydride to prepare compound (XVIII);
g) treating compound (XVIII) with lithium aluminum hydride to prepare compound (XIX);
h) reacting (XIX) with methanesulfonyl chloride in the presence of N,N-diisopropylethylamine and 4-dimethylaminopyridine to prepare compound (XX);
i) treating (XX) with trifluoroacetic acid to prepare compound (XXI);
j) reacting (XXI) with 2-(chloromethyl)-5-fluoroimidazo[1,2-a]pyridine (XXII) in the presence of potassium carbonate and potassium iodide to prepare compound (XXIII);
k) reacting (XXIII) with 1-methylpiperazine to prepare (XIV);

### EXAMPLES

### Example 1

### 3-(8-Oxo-5,6,7,8-tetrahydro-7-quinolinyl)propanenitrile (intermediate)

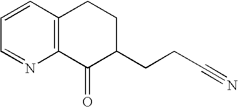

To a −78° C. solution of diisopropylamine (4.5 g, 45 mmol) in 100 mL of tetrahydrofuran was added 1.6M n-butyllithium (28 mL, 45 mmol) dropwise. The mixture was stirred for 1.5 hours before a solution of 6,7-dihydro-8(5H)-quinolinone (6.0 g, 41 mmol) in 50 mL of tetrahydrofuran was added dropwise. The mixture was stirred for 3 hours and acrylonitrile (2.6 g, 49 mmol) was added. The reaction was allowed to warm to room temperature and then heated to 40° C. for 16 hours. The mixture was allowed to cool to room temperature and was concentrated to a volume of 50 mL. The remainder was quenched with 100 mL of water and extracted with dichloromethane (3×100 mL). The combined organic extracts were dried over anhydrous sodium sulfate and concentrated. The residue was purified by silica chromatography eluting acetonitrile to yield 5.88 g (72%) of 3-(8-oxo-5,6,7,8-tetrahydro-7-quinolinyl)propanenitrile. ¹H NMR (300 MHz, METHANOL-D4) δ ppm 1.9 (m, 1H), 2.0 (m, 1H), 2.4 (m, 2H), 2.7 (t, J=7.5 Hz, 2H), 2.9 (m, J=12.8, 6.3, 6.3, 4.5 Hz, 1H), 3.2 (m, 2H), 7.6 (d, J=7.9, 4.6 Hz, 1H), 7.9 (d, J=7.9, 1H), 8.6 (d, J=3.9 Hz, 1H); MS m/z 201 (M+1).

### Example 2

### 1,2,3,4,4a,5,6,10b-Octahydro-1,10-phenanthroline (intermediate)

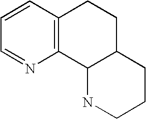

3-(8-Oxo-5,6,7,8-tetrahydro-7-quinolinyl)propanenitrile (1.54 g, 7.7 mmol) was dissolved in 100 mL of absolute ethanol, then Raney nickel (250 mg) was added. The reaction mixture was hydrogenated at 70° C. under 60 psi H₂ for 16 hours. The reaction mixture was allowed to cool to room temperature. The Raney nickel catalyst was removed by filtration over celite and the wash concentrated. The residue was purified by silica chromatography eluting with a 0% to 10% gradient of 30% aqueous ammonium hydroxide in acetonitrile to yield 0.63 g (43%) of 1,2,3,4,4a,5,6,10b-octahydro-1,10-phenanthroline. ¹H NMR (400 MHz, METHANOL-D4) δ ppm 1.3 (m, 1 H), 1.6 (m, 3H), 1.8 (m, 2H), 2.1 (m, 1H), 2.9 (m, 3H), 3.2 (m, 1H), 3.4 and 3.8 (d, J=3.8 Hz, and s, 1H), 7.2 (m, 1H), 7.5 (m, 1H), 8.4 (m, 1H); MS m/z 189 (M+1).

### Example 3A

### (trans)-1,2,3,4,4a,5,6,10b-Octahydro-1,10-phenanthroline (intermediate)

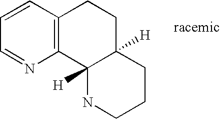

### Example 3B

### (cis)-1,2,3,4,4a,5,6,10b-Octahydro-1,10-phenanthroline (intermediate)

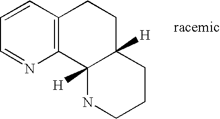

Trans and cis diastereomers of 1,2,3,4,4a,5,6,10b-octahydro-1,10-phenanthroline were separated by silica chromatography eluting with a 0% to 10% gradient of 2M ammonia in methanol with dichloromethane as co-solvent to give (trans)-1,2,3,4,4a,5,6,10b-octahydro-1,10-phenanthroline and (cis)-1,2,3,4,4a,5,6,10b-octahydro-1,10-phenanthroline. 3A: ¹H NMR (400 MHz, METHANOL-D4) δ ppm 1.3 (m, 1H), 1.6 (m, 3H), 1.7 (m, 1H), 1.8 (m, 2H), 2.9 (m, 3H), 3.2 (m, 1H), 3.3 (d, J=9.2 Hz, 1H), 7.2 (dd, J=7.3, 5.1 Hz, 1H), 7.5 (d, J=7.7, 1H), 8.3 (m, 1H); MS m/z 189 (M+1). 3B: ¹H NMR (300 MHz, METHANOL-D4) δ ppm 1.6 (m, 1H), 1.7 (m, 3H), 1.8 (m, 1H), 2.2 (m, 2H), 2.9 (m, 4H), 3.9 (d, J=3.7 Hz, 1H), 7.3 (dd, J=7.7, 4.8 Hz, 1H), 7.6 (d, J=7.7 Hz, 1H), 8.4 (d, J=4.8 Hz, 1H); MS m/z 189 (M+1).

### Example 4

### 1,1-Dimethylethyl 2-(chloromethyl)-1H-benzimidazole-1-carboxylate (intermediate)

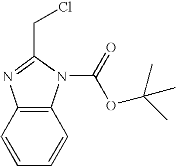

To a solution of 2(chloromethyl)-1H-benzimidazole (4.55 g, 27 mmol) in 250 mL of dichloromethane was added N,N-dimethyl-4-pyridinamine (0.16 g, 1.4 mmol), and di-tert-butyl-dicarbonate (7.2 g, 33 mmol). The reaction mixture was stirred at room temperature for 16 hours. The mixture was concentrated and the residue purified by silica gel chromatography eluting with ethyl acetate and hexanes to yield 6.1 g (38%) of 1,1-dimethylethyl 2-(chloromethyl)-1H-benzimidazole-1-carboxylate. ¹H NMR (400 MHz, CHLOROFORM-D) δ ppm 1.7 (s, 9H), 5.1 (s, 2H), 7.4 (m, 2H), 7.8 (m, 1H), 8.0 (ddd, J=7.7, 1.1, 0.7 Hz, 1H); MS m/z 167 (M+1).

### Example 5A

### (trans)-1,1-Dimethylethyl 2-(3,4,4a,5,6,10b-hexahydro-1,10-phenanthrolin-1(2H)-ylmethyl)-1H-benzimidazole-1-carboxylate (intermediate)

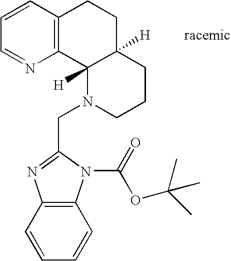

### Example 5B

### (cis)-1,1-Dimethylethyl 2-(3,4,4a,5,6,10b-hexahydro-1,10-phenanthrolin-1(2H)-ylmethyl)-1H-benzimidazole-1-carboxylate (intermediate)

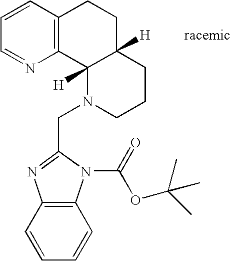

1,2,3,4,4a,5,6,10b-Octahydro-1,10-phenanthroline (0.64 g, 3.4 mmol), potassium carbonate (2.3 g, 17 mmol), 1,1-dimethylethyl 2-(chloromethyl)-1H-benzimidazole-1-carboxylate (0.99 g, 3.7 mmol), and potassium iodide (20 mg) were stirred in 10 mL of acetonitrile for 48 hours. The mixture was quenched with 25 mL of water and extracted 3 times with 20 mL of dichloromethane. The combined organic layers were concentrated and the residue purified by silica chromatography eluting with a 0% to 10% gradient of 2M ammonia in isopropanol with dichloromethane as co-solvent to yield 0.39 g (27%) of (trans) 1,1-dimethylethyl 2-(3,4,4a,5,6,10b-hexahydro-1,10-phenanthrolin-1(2H)-ylmethyl)-1H-benzimidazole-1-carboxylate and 0.44 g (31%) of (cis)-1,1-dimethylethyl 2-(3,4,4a,5,6,10b-hexahydro-1,10-phenanthrolin-1(2H)-ylmethyl)-1H-benzimidazole-1-carboxylate. 5A: ¹H NMR (400 MHz, METHANOL-D4) δ ppm 1.5 (m, 3H), 1.6 (s, 9H), 1.9 (m, 1H), 2.0 (m, 2H), 2.2 (q, J=11.7 Hz, 1H), 2.7 (m, 1H), 3.0 (m, 1H), 3.1 (t, J=13.5 Hz, 1H), 3.6 (d, J=14.5 Hz, 1H), 3.9 (d, J=10.4 Hz, 1H), 4.2 (d, J=15.9 Hz, 1H), 4.4 (m, 1H), 6.8 (dd, J=7.6, 4.7 Hz, 1H), 7.3 (m, 2H), 7.4 (m, 2H), 7.7 (d, J=4.0 Hz, 1H), 7.8 (d, J=7.5 Hz, 1H); MS m/z 419 (M+1). 5B: ¹H NMR (400 MHz, METHANOL-D4) δ ppm 1.5 (m, 1H), 1.7 (s, 9H), 1.7 (m, 4H), 2.0 (m, 1H), 2.4 (m, 2H), 2.7 (m, 1H), 2.9 (m, 1H), 3.0 (m, 1H), 3.6 (s, 1 H), 3.8 (d, J=14.3 Hz, 1H), 4.6 (d, J=14.8 Hz, 1H), 7.1 (dd, J=7.9, 4.8 Hz, 1H), 7.3 (m, 2H), 7.4 (d, J=7.7 Hz, 1H), 7.5 (d, J=7.0 Hz, 1H), 7.8 (d, J=9.0 Hz, 1H), 8.3 (d, J=4.4 Hz, 1H); m/z 419 (M+1).

### Example 6

### (trans)-1-(1H-Benzimidazol-2-ylmethyl)-1,2,3,4,4a,5,6,10b-octahydro-1,10-phenanthroline (intermediate)

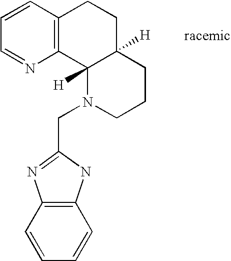

(trans)-1,1-Dimethylethyl 2-(3,4,4a,5,6,10b-hexahydro-1,10-phenanthrolin-1(2H)-ylmethyl)-1H-benzimidazole-1-carboxylate (0.34 g, 0.81 mmol) was dissolved in 5 mL of dichloromethane and treated with 5 mL of trifluoroacetic acid. The reaction mixture was stirred at room temperature for 2 hours and then concentrated to dryness. The residue was taken up in 15 mL of saturated sodium bicarbonate and extracted 3 times with 15 mL of dichloromethane. The combined organic layers were washed with 15 mL of saturated sodium chloride and dried over sodium sulfate before being concentrated and dried under vacuum to yield 0.23 g (88%) of (trans)-1-(1H-benzimidazol-2-ylmethyl)-1,2,3,4,4a,5,6,10b-octahydro-1,10-phenanthroline. ¹H NMR (400 MHz, METHANOL-D4) δ ppm 1.4 (m, 2H), 1.6 (td, J=12.6, 4.9 Hz, 1H), 1.8 (m, 1H), 2.0 (m, 2H), 2.1 (m, 1H), 2.8 (m, 2H), 3.0 (m, 2H), 3.4 (d, J=13.6 Hz, 1H), 3.9 (d, J=10.8 Hz, 1H), 4.1 (d, J=14.7 Hz, 1H), 7.2 (m, 3H), 7.5 (m, 2H), 7.6 (d, J=7.9 Hz, 1H), 8.4 (d, J=4.8 Hz, 1H); MS m/z 319 (M+1).

### Example 7

### (cis)-1-(1H-Benzimidazol-2-ylmethyl)-1,2,3,4,4a,5,6,10b-octahydro-1,10-phenanthroline (intermediate)

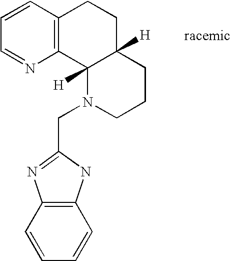

(cis)-1,1-Dimethylethyl 2-(3,4,4a,5,6,10b-hexahydro-1,10-phenanthrolin-1(2H)-ylmethyl)-1H-benzimidazole-1-carboxylate (0.76 g, 1.8 mmol) was dissolved in 10 mL of dichloromethane and treated with 10 mL of trifluoroacetic acid. The reaction mixture was stirred at room temperature for 2 hours and then concentrated to dryness. The residue was taken up in 30 mL of saturated sodium bicarbonate and extracted 3 times with 30 mL of dichloromethane. The combined organic layers were washed with 30 mL of saturated sodium chloride and dried over sodium sulfate before being concentrated and dried under vacuum to yield 0.46 g (80%) of (cis)-1-(1H-benzimidazol-2-ylmethyl)-1,2,3,4,4a,5,6,10b-octahydro-1,10-phenanthroline. ¹H NMR (400 MHz, METHANOL-D4) δ ppm 1.7 (m, 5H), 2.2 (m, 1H), 2.4 (m, 2H), 2.8 (m, 2H), 3.1 (m, 1H), 3.7 (m, 1H), 4.0 (d, J=15.2 Hz, 1H), 4.1 (d, J=15.3 Hz, 1H), 7.2 (m, 2H), 7.3 (dd, J=7.7, 4.9 Hz, 1H), 7.5 (dd, J=6.0, 3.2 Hz, 2H), 7.6 (d, J=7.9 Hz, 1H), 8.4 (d, J=5.1 Hz, 1H); MS m/z 319 (M+1).

### Example 8

### (trans)-3-[2-(3,4,4a,5,6,10b-Hexahydro-1,10-phenanthrolin-1(2H)-ylmethyl)-1H-benzimidazol-1-yl]-1-propanamine

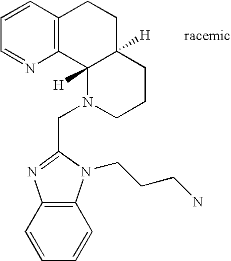

(trans)-1-(1H-Benzimidazol-2-ylmethyl)-1,2,3,4,4a,5,6,10b-octahydro-1,10-phenanthroline (0.045 g, 0.14 mmol), diisopropylethylamine (0.038 g, 0.30 mmol), 3-bromopropylphthalimide (0.056 g, 0.21 mmol) and 10 mg of potassium iodide were added to 4 mL of DMF and heated to 200° C. for 20 minutes in a microwave reactor. The reaction was allowed to cool to room temperature and was diluted with 10 mL of water. The mixture was extracted 3 times with 10 mL of diethyl ether. The combined organic layers were washed with 10 mL of brine, dried over sodium sulfate, and concentrated. The residue was dissolved in 5 mL of ethanol and treated with 0.2 mL of hydrazine. The reaction mixture was stirred at room temperature for 16 hours. The solids were filtered off and the filtrate concentrated. The residue was purified by silica chromatography eluting with a 0% to 10% gradient of 30% aqueous ammonium hydroxide in acetonitrile to yield 0.020 g (38%) of (trans)-3-[2-(3,4,4a,5,6,10b-hexahydro-1,10-phenanthrolin-1(2H)-ylmethyl)-1H-benzimidazol-1-yl]-1-propanamine. ¹H NMR (400 MHz, METHANOL-D4) δ ppm 1.4 (m, 1H), 1.6 (td, J=12.4, 5.5 Hz, 1H), 1.9 (m, 1H), 2.1 (m, 2H), 2.2 (m, 3H), 2.6 (m, 1H), 2.7 (m, 2 H), 2.8 (m, 2H), 3.0 (m, 2H), 3.6 (d, J=13.0 Hz, 1H), 3.9 (d, J=11.5 Hz, 1H), 4.2 (d, J=13.2 Hz, 1H), 4.6 (m, 1H), 5.1 (m, 1H), 7.2 (m, 3H), 7.6 (m, 3H), 8.5 (d, J=4.4 Hz, 1H); MS m/z 376 (M+1).

### Example 9

### (cis)-3-[2-(3,4,4a,5,6,10b-Hexahydro-1,10-phenanthrolin-1(2H)-ylmethyl)-1H-benzimidazol-1-yl]-1-propanamine

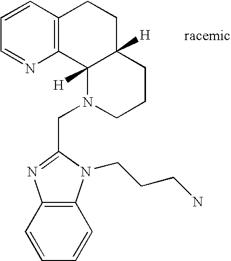

(cis)-1-(1H-Benzimidazol-2-ylmethyl)-1,2,3,4,4a,5,6,10b-octahydro-1,10-phenanthroline (0.053 g, 0.17 mmol), diisopropylethylamine (0.045 g, 0.30 mmol), 3-bromopropylphthalimide (0.068 g, 0.21 mmol) and 10 mg of potassium iodide were added to 4 mL of DMF and heated to 200° C. for 20 minutes in a microwave reactor. The reaction was allowed to cool to room temperature and was diluted with 10 mL of water. The mixture was extracted 3 times with 10 mL of diethyl ether. The combined organic layers were washed with 10 mL of brine, dried over sodium sulfate, and concentrated. The residue was dissolved in 5 mL of ethanol and treated with 0.2 mL of hydrazine. The reaction mixture was stirred at room temperature for 16 hours. The solids were filtered off and the wash concentrated. The residue was purified by silica chromatography eluting with a 0% to 10% gradient of 30% aqueous ammonium hydroxide in acetonitrile to yield 0.010 g (16%) of (cis)-3-[2-(3,4,4a,5,6,10b-hexahydro-1,10-phenanthrolin-1(2H)-ylmethyl)-1H-benzimidazol-1-yl]-1-propanamine. ¹H NMR (400 MHz, METHANOL-D4) δ ppm 1.6 (m, 4H), 1.8 (m, 3H), 2.1 (m, 1H), 2.3 (m, 3H), 2.5 (m, 1H), 2.8 (m, 1H), 2.9 (m, 1H), 3.1 (m, 1H), 3.5 (d, J=2.7 Hz, 1H), 3.5 (d, J=13.9 Hz, 1H), 3.9 (m, 1H), 4.1 (m, 1H), 4.2 (d, J=13.9 Hz, 1H), 7.2 (m, 2H), 7.3 (dd, J=7.7, 4.8 Hz, 1H), 7.4 (d, J=7.3 Hz, 1H), 7.5 (d, J=7.3 Hz, 1H), 7.7 (d, J=7.7 Hz, 1H), 8.4 (d, J=3.3 Hz, 1H); MS m/z 376 (M+1).

### Example 10A

### (3-{2-[(4aR,10bR)-3,4,4a,5,6,10b-Hexahydro-1,10-phenanthrolin-1(2H)-ylmethyl]-1H-benzimidazol-1-yl}propyl)dimethylamine

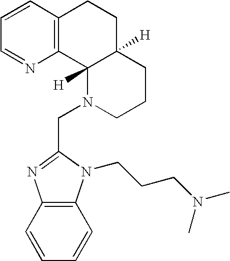

### Example 10B

### (3-{2-[(4aS,10bS)-3,4,4a,5,6,10b-Hexahydro-1,10-phenanthrolin-1(2H)-ylmethyl]-1H-benzimidazol-1-yl}propyl)dimethylamine

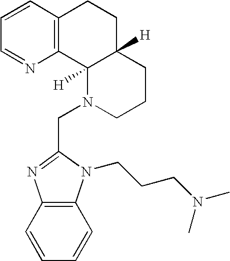

To a solution of (trans)-1-(1H-benzimidazol-2-ylmethyl)-1,2,3,4,4a,5,6,10b-octahydro-1,10-phenanthroline (0.061 g, 0.19 mmol) in 5 mL of N,N-dimethylformamide was added potassium carbonate (0.053 g, 0.38 mmol), potassium iodide (10 mg) and (3-chloropropyl)dimethylamine hydrochloride (0.033 g, 0.21 mmol). The reaction mixture was heated to 80° C. in a sealed tube for 16 hours. The mixture was diluted with 10 mL of water and extracted 3 times with 10 mL of dichloromethane. The combined organic layers were concentrated and the residue purified by silica chromatography eluting with a 0% to 10% gradient of 30% aqueous ammonium hydroxide in acetonitrile to yield 0.048 g (63%) of (trans) {3-[2-(3,4,4a,5,6,10b-hexahydro-1,10-phenanthrolin-1(2H)-ylmethyl)-1H-benzimidazol-1-yl]propyl}dimethylamine. Enantiomers were separated by chiral super critical fluid chromatography to yield (3-{2-[(4aS,10bS)-3,4,4a,5,6,10b-hexahydro-1,10-phenanthrolin-1(2H)-ylmethyl]-1H-benzimidazol-1-yl}propyl)dimethylamine and (3-{2-[(4aR,10bR)-3,4,4a,5,6,10b-hexahydro-1,10-phenanthrolin-1(2H)-ylmethyl]-1H-benzimidazol-1-yl}propyl)dimethylamine. Chromatography parameters: column=chiralpak AD-H; mobile phase=70% CO₂/30% isopropanol (0.5% diethylamine); flow rate=2 mL/min; pressure=1500 psi; temperature=27° C.; inject volume=10 μL; λ=230 nM. Enantiomer #1: Retention time=4.29 minutes; ¹H NMR (400 MHz, METHANOL-D4) δ ppm 1.4 (m, 2H), 1.5 (m, 1H), 1.9 (m, 1H), 2.1 (m, 4H), 2.3 (m, 6H), 2.4 (t, J=6.9 Hz, 2H), 2.9 (m, 4H), 3.3 (s, 1H), 3.7 (d, J=13.0 Hz, 1H), 3.9 (d, J=11.0 Hz, 1 H), 4.2 (d, J=13.2 Hz, 1H), 4.7 (dt, J=14.2, 7.1 Hz, 1H), 4.9 (m, 1H), 7.3 (m, 3H), 7.5 (m, 3H), 8.5 (d, J=4.6 Hz, 1H); MS m/z 404 (M+1). Enantiomer #2; Retention time=8.79 minutes; ¹H NMR (400 MHz, METHANOL-D4) δ ppm 1.4 (m, 2H), 1.6 (m, 1H), 1.9 (m, 1H), 2.0 (d, J=12.1 Hz, 1H), 2.2 (m, 4H), 2.4 (s, 6H), 2.6 (m, 2H), 2.9 (m, 4H), 3.6 (d, J=13.0 Hz, 1H), 3.9 (d, J=11.0 Hz, 1H), 4.2 (d, J=13.2 Hz, 1H), 4.7 (m, 2H), 7.3 (m, 3H), 7.6 (m, 3H), 8.5 (d, J=3.7 Hz, 1H); MS m/z 404 (M+1).

### Example 11A

### (3-{2-[(4aS,10bR)-3,4,4a,5,6,10b-Hexahydro-1,10-phenanthrolin-1(2H)-ylmethyl]-1H-benzimidazol-1-yl}propyl)dimethylamine

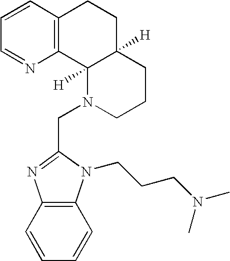

### Example 11B

### (3-{2-[(4aR,10bS)-3,4,4a,5,6,10b-Hexahydro-1,10-phenanthrolin-1(2H)-ylmethyl]-1H-benzimidazol-1-yl}propyl)dimethylamine

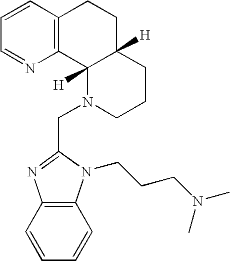

To a solution of (cis)-1-(1H-benzimidazol-2-ylmethyl)-1,2,3,4,4a,5,6,10b-octahydro-1,10-phenanthroline (0.054 g, 0.17 mmol) in 5 mL of N,N-dimethylformamide was added potassium carbonate (0.094 g, 0.68 mmol), potassium iodide (10 mg) and (3-chloropropyl)dimethylamine hydrochloride (0.030 g, 0.19 mmol). The reaction mixture was heated to 80° C. in a sealed tube for 16 hours. The mixture was diluted with 10 mL of water and extracted 3 times with 10 mL of dichloromethane. The combined organic layers were concentrated and the residue purified by silica chromatography eluting with a 0% to 10% gradient of 30% aqueous ammonium hydroxide in acetonitrile to yield 0.036 g (52%) of (cis) {3-[2-(3,4,4a,5,6,10b-hexahydro-1,10-phenanthrolin-1(2H)-ylmethyl)-1H-benzimidazol-1-yl]propyl}dimethylamine. Enantiomers were separated by chiral SFC chromatography to yield 3-{2-[(4aR,10bS)-3,4,4a,5,6,10b-hexahydro-1,10-phenanthrolin-1(2H)-ylmethyl]-1H-benzimidazol-1-yl}-N,N-dimethyl-1-propanamine and 3-{2-[(4aS,10bR)-3,4,4a,5,6,10b-hexahydro-1,10-phenanthrolin-1(2H)-ylmethyl]-1H-benzimidazol-1-yl}-N,N-dimethyl-1-propanamine. Chromatography parameters: column=chiralpak AD-H; mobile phase=70% CO₂/30% isopropanol (0.5% diethylamine); flow rate=2 mL/min; pressure=1500 psi; temperature=27° C.; inject volume=10 μL; λ=230 nM. Enantiomer #1: Retention time=4.79 minutes; ¹H NMR (400 MHz, METHANOL-D4) δ ppm 1.5 (m, 1H), 1.6 (m, 3H), 1.8 (m, 3H), 1.9 (m, 1H), 2.0 (m, 1H), 2.1 (s, 7 H), 2.3 (t, J=11.3 Hz, 1H), 2.6 (m, 1H), 2.8 (d, J=11.9 Hz, 1H), 2.9 (m, 1H), 3.1 (m, 1H), 3.5 (d, J=2.7 Hz, 1H), 3.6 (d, J=13.9 Hz, 1H), 4.0 (dt, J=14.4, 7.1 Hz, 1H), 4.2 (m, 2H), 7.2 (m, 2H), 7.3 (dd, J=7.9, 4.8 Hz, 1H), 7.4 (d, J=7.3 Hz, 1H), 7.6 (d, J=6.8 Hz, 1H), 7.7 (d, J=9.1 Hz, 1H), 8.4 (d, J=4.8 Hz, 1H); MS m/z 404 (M+1). Enantiomer #2: Retention time=8.61 minutes; ¹H NMR (400 MHz, METHANOL-D4) δ ppm 1.4 (m, 1H), 1.6 (m, 3H), 1.8 (m, 3H), 1.9 (m, 1H), 2.0 (m, 1H), 2.1 (m, 7H), 2.3 (t, J=11.2, 1H), 2.5 (m, 1H), 2.8 (d, J=11.9 Hz, 1H), 2.9 (m, 1H), 3.1 (m, 1H), 3.5 (d, J=2.7 Hz, 1H), 3.6 (d, J=13.9 Hz, 1H), 4.0 (dt, J=14.4, 7.1 Hz, 1H), 4.2 (m, 2 H), 7.2 (m, 2H), 7.3 (dd, J=7.9, 4.8 Hz, 1H), 7.4 (d, J=8.6 Hz, 1H), 7.6 (d, J=8.6 Hz, 1H), 7.7 (d, J=9.1 Hz, 1H), 8.4 (d, J=6.4 Hz, 1H); MS m/z 404 (M+1).

### Example 12

### (cis)-1-{[1-(4-Pyridinylmethyl)-1H-benzimidazol-2-yl]methyl}-1,2,3,4,4a,5,6,10b-octahydro-1,10-phenanthroline

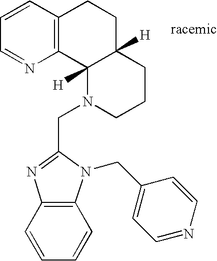

To a solution of (cis)-1-(1H-benzimidazol-2-ylmethyl)-1,2,3,4,4a,5,6,10b-octahydro-1,10-phenanthroline (0.032 g, 0.10 mmol) in 5 mL of N,N-dimethylformamide was added potassium carbonate (0.14 g, 1.0 mmol), potassium iodide (10 mg) and 4-(chloromethyl)pyridine hydrochloride (0.041 g, 0.25 mmol). The reaction mixture was heated to 80° C. in a sealed tube for 16 hours. The mixture was diluted with 10 mL of water and extracted 3 times with 10 mL of dichloromethane. The combined organic layers were concentrated and the residue purified by silica chromatography eluting with a 0% to 10% gradient of 30% aqueous ammonium hydroxide in acetonitrile to yield 0.0060 g (15%) of (cis)-1-{[1-(4-pyridinylmethyl)-1H-benzimidazol-2-yl]methyl}-1,2,3,4,4a,5,6,10b-octahydro-1,10-phenanthroline. ¹H NMR (400 MHz, METHANOL-D4) δ ppm 1.4 (m, 2H), 1.6 (m, 2H), 1.7 (m, 1H), 1.9 (m, 1H), 2.1 (m, 1H), 2.3 (m, 1 H), 2.8 (m, 2H), 2.9 (d, J=11.7 Hz, 1H), 3.4 (d, J=2.4 Hz, 1H), 3.5 (d, J=14.3 Hz, 1 H), 4.2 (d, J=14.3 Hz, 1H), 5.1 (d, J=17.8 Hz, 1H), 5.4 (d, J=16.1 Hz, 1H), 6.7 (d, J=6.0 Hz, 1H), 7.2 (m, 5H), 7.5 (d, J=7.7 Hz, 1H), 7.6 (d, J=7.9 Hz, 1H), 8.2 (d, J=4.8 Hz, 1H), 8.3 (d, J=6.2 Hz, 2H); MS m/z 410 (M+1).

### Example 13

### (cis)-1-({1-[(1-Methyl-3-piperidinyl)methyl]-1H-benzimidazol-2-yl}methyl)-1,2,3,4,4a,5,6,10b-octahydro-1,10-phenanthroline

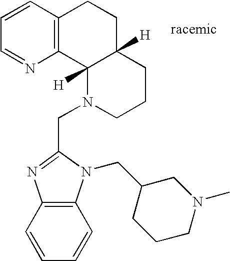

To a solution of (cis)-1-(1H-benzimidazol-2-ylmethyl)-1,2,3,4,4a,5,6,10b-octahydro-1,10-phenanthroline (0.032 g, 0.10 mmol) in 5 mL of N,N-dimethylformamide was added potassium carbonate (0.14 g, 1.0 mmol), potassium iodide (10 mg) and 4-(chloromethyl)pyridine hydrochloride (0.041 g, 0.25 mmol). The reaction mixture was heated to 80° C. in a sealed tube for 16 hours. The mixture was allowed to cool to room temperature, diluted with 10 mL of water, and extracted 3 times with 10 mL of dichloromethane. The combined organic layers were concentrated and the residue purified by silica chromatography eluting with a 0% to 10% gradient of 30% aqueous ammonium hydroxide in acetonitrile to yield 0.0090 g (21%) of (cis)-1-({1-[(1-methyl-3-piperidinyl)methyl]-1H-benzimidazol-2-yl}methyl)-1,2,3,4,4a,5,6,10b-octahydro-1,10-phenanthroline. ¹H NMR (400 MHz, METHANOL-D4) δ ppm 0.2 and 0.7 (2m, 1 H), 1.0 and 1.2 (2m, 1H), 1.5 (m, 2H), 1.7 (m, 2H), 1.8 (m, 5H), 2.0 (m, 5H), 2.2 (s, 3H), 2.6 (m, 2H), 2.8 (m, 2H), 3.0 (m, 1H), 3.2 (m, 1H), 3.4 (d, J=3.2 Hz, 1H), 3.5 (d, J=2.2 Hz, 1H), 4.0 (m, 1H), 4.2 (m, 2H), 7.2 (m, 2H), 7.4 (m, 2H), 7.6 (m, 1H), 7.7 (m, 1H), 8.4 (m, 1H); MS m/z 430 (M+1).

### Example 14

### 1-[(5-Fluoroimidazo[1,2-a]pyridin-2-yl)methyl]-1,2,3,4,4a,5,6,10b-octahydro-1,10-phenanthroline (intermediate)

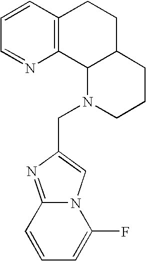

### Method A:

### A) 5-Fluoroimidazo-[1,2-a]pyridine-2-carbaldehyde

To a solution of 6-fluoro-2-pyridinamine (Tetrahedron, 2002, 58, 489, incorporated by reference with regard to such) (2.8 g, 25 mmol) in ethylene glycol dimethyl ether (28 mL) was added trichloroacetone (7.9 mL, 75 mmol). The mixture was stirred at room temperature for 15 hours and the resulting precipitate was collected by filtration and refluxed in ethyl alcohol (8 mL) for 4 hours. The reaction mixture was cooled to room temperature, concentrated, dissolved in dichloromethane and washed with saturated aqueous sodium bicarbonate. The organic layer was isolated, dried with magnesium sulfate, and concentrated. The resulting solid was refluxed in aqueous calcium carbonate for 2 hours, cooled to room temperature, and extracted with dichloromethane. The organic layer was dried with magnesium sulfate and concentrated to give 1.4 g (34% yield) 5-fluoroimidazo[1,2-a]pyridine-2-carbaldehyde as a tan solid. ¹H-NMR (CDCl₃): δ 10.16 (s, 1H), 8.22 (s, 1H), 7.54 (d, 1H), 7.34 (m, 1H), 6.59 (m, 1H); TLC (10% 2 M ammonia in methyl alcohol-ethyl acetate) R_{f}=0.60.

### B) 1-[(5-fluoroimidazo[1,2-a]pyridin-2-yl)methyl]-1,2,3,4,4a,5,6,10b-octahydro-1,10-phenanthroline

To a solution of 1,2,3,4,4a,5,6,10b-octahydro-1,10-phenanthroline (0.10 g, 0.53 mmol) in 10 mL of 1,2-dichloroethane was added 5-fluoroimidazo[1,2-a]pyridine-2-carbaldehyde (similar methods as described in J. Het. Chem. (1992), vol. 29, p. 691; 0.087 g, 0.53 mmol), acetic acid (0.039 g, 0.53 mmol), and sodium triacetoxyborohydride (0.33 g, 1.6 mmol). The reaction mixture was stirred at room temperature for 16 hours. The mixture was quenched with 10 mL of saturated sodium bicarbonate and extracted 3 times with 10 mL of dichloromethane. The combined organic layers were washed with 20 mL of brine, dried over anhydrous sodium sulfate, and concentrated. The residue was purified by silica chromatography eluting with a 0% to 10% gradient of 2M ammonia in methanol and dichloromethane as co-solvent to yield 0.065 g (57%) of 1-[(5-fluoroimidazo[1,2-a]pyridin-2-yl)methyl]-1,2,3,4,4a,5,6,10b-octahydro-1,10-phenanthroline.

### Method B:

### A) 2-(chloromethyl)-5-fluoroimidazo[1,2-a]pyridine

2,6-difluoropyridine (31.5 mL, 0.348 mol) was diluted with 30% ammonium hydroxide (200 mL) in a steel bomb and heated to 110° C. overnight. The bomb was cooled to room temperature over two hours then further cooled to 0° C. for two hours. The resulting solid was filtered and rinsed with water to obtain 26.39 g as a white solid. The filtrate was extracted with dichloromethane, dried over sodium sulfate and concentrated to afford an additional 9.3 g (92% overall yield) of 6-fluoro-2-pyridinamine. A portion of the solid (5 g, 0.044 mol) was dissolved in 1,2-dichloroethane (20 mL) and 1,3-dichloro-2-propanone (34.2 mL, 4.34 mol) was added in two portions. The reaction was stirred at 40° C. over two days. The resulting solid was collected by filtration, dissolved in absolute ethanol (100 mL), and refluxed at 90° C. overnight. Solvent was evaporated and dichloromethane and saturated aqueous sodium bicarbonate was added to the residue. The aqueous layer was extracted two times with dichloromethane and once with a 3:1 chloroform: isopropanol mixture. Combined organics were dried over sodium sulfate and concentrated to a 3.61 g (62%) of 2-(chloromethyl)-5-fluoroimidazo[1,2-a]pyridine as a black oil which solidified upon standing. ¹H NMR (400 MHz, DMSO-D6) δ 5.02 (s, 2H), 7.29 (d, 1H), 7.74 (d, 1H), 7.88 (m, 1H), 8.41 (s, 1H); MS m/z 185 (M+1).

### B) 1-[(5-fluoroimidazo[1,2-a]pyridin-2-yl)methyl]-1,2,3,4,4a,5,6,10b-octahydro-1,10-phenanthroline

To a solution of 1,2,3,4,4a,5,6,10b-octahydro-1,10-phenanthroline (0.63 g, 3.4 mmol) in 10 mL of acetonitrile was added potassium carbonate (2.3 g, 17 mmol), 2-(chloromethyl)-5-fluoroimidazo[1,2-a]pyridine (similar methods as described in Chem Pharm Bull 2000, 48, 935; Tetrahedron 1992, 47, 5173; Tet Lett 1990, 31, 3013; J. Het Chem 1998, 25, 129; Chemistry of Heterocyclic Compounds 2002, 38, 590; each incorporated by reference to such synthesis; 0.68 g, 3.7 mmol), and 10 mg of potassium iodide. The reaction mixture was stirred for 16 hours at room temperature and quenched with 10 mL of water. The mixture was extracted 3 times with 20 mL of dichloromethane. The combined organic layers were concentrated and the residue purified by silica chromatography eluting with a 0% to 10% gradient of 2M ammonia in methanol and dichloromethane as co-solvent to yield 0.96 g (84%) of 1-[(5-fluoroimidazo[1,2-a]pyridin-2-yl)methyl]-1,2,3,4,4a,5,6,10b-octahydro-1,10-phenanthroline. ¹H NMR (400 MHz, METHANOL-D4) δ ppm 1.4 (m, 1H), 1.6 (m, 3 H), 1.8 (m, 1H), 2.0 (m, 1H), 2.4 (m, 1H), 2.9 (m, 3H), 3.2 (m, 1H), 3.4 (m, 1H), 3.7 (m, 1H), 4.0 (m, 1H), 6.6 (m, 1H), 7.2 (m, 1H), 7.3 (m, 2H), 7.5 (m, 1H), 7.6 and 8.0 (2s, 1H), 8.3 (m, 1H); MS m/z 337 (M+1).

### Example 15A

### (trans)-1-{[5-(4-Methyl-1-piperazinyl)imidazo[1,2-a]pyridin-2-yl]methyl}-1,2,3,4,4a,5,6,10b-octahydro-1,10-phenanthroline

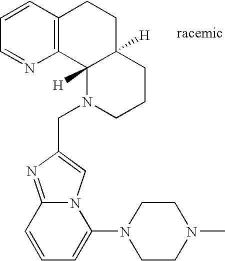

### Example 15B

### (cis)-1-{[5-(4-Methyl-1-piperazinyl)imidazo[1,2-a]pyridin-2-yl]methyl}-1,2,3,4,4a,5,6,10b-octahydro-1,10-phenanthroline

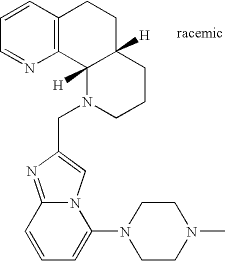

1-[(5-Fluoroimidazo[1,2-a]pyridin-2-yl)methyl]-1,2,3,4,4a,5,6,10b-octahydro-1,10-phenanthroline (0.065 g (0.19 mmol) was dissolved in 0.5 mL 1-methylpiperazine and heated to 200° C. in a microwave reactor for 20 minutes. The reaction was allowed to cool and diluted with 5 mL of brine. The mixture was extracted 3 times with 5 mL of dichloromethane. The combined organic layers were washed 3 times with 5 mL of water, dried over sodium sulfate, and concentrated. The residue purified by silica chromatography eluting with a 0% to 10% gradient of 30% aqueous ammonium hydroxide in acetonitrile to yield 0.0060 g (7%) of (trans)-1-{[5-(4-methyl-1-piperazinyl)imidazo[1,2-a]pyridin-2-yl]methyl}-1,2,3,4,4a,5,6,10b-octahydro-1,10-phenanthroline and 0.030 g (38%) of (cis)-1-{[5-(4-methyl-1-piperazinyl)imidazo[1,2-a]pyridin-2-yl]methyl}-1,2,3,4,4a,5,6,10b-octahydro-1,10-phenanthroline. 15A: ¹H NMR (400 MHz, METHANOL-D4) δ ppm 1.5 (m, 1H), 1.7 (m, 2H), 2.0 (m, 2H), 2.2 (m, 3H), 2.5 (s, 3H), 2.8 (m, 4H), 3.0 (m, 2H), 3.2 (m, 4H), 3.4 (d, J=14.1 Hz, 1H), 3.8 (d, J=15.2 Hz, 1H), 4.2 (m, 2H), 6.5 (d, J=7.1 Hz, 1H), 7.3 (m, 3H), 7.6 (d, J=7.5 Hz, 1H), 7.9 (s, 1H), 8.5 (d, J=4.4 Hz, 1H); MS m/z 417 (M+1). 15B: ¹H NMR (400 MHz, METHANOL-D4) δ ppm 1.7 (m, 5H), 2.1 (m, 1H), 2.4 (m, 5H), 2.8 (m, 5H), 2.9 (m, 1H), 3.1 (m, 5H), 3.6 (s, 1H), 3.7 (d, J=14.7 Hz, 1H), 4.0 (d, J=14.7 Hz, 1H), 6.4 (d, J=7.3 Hz, 1H), 7.2 (m, 2H), 7.2 (m, 1H), 7.5 (d, J=6.4 Hz, 1 H), 7.6 (s, 1H), 8.3 (d, J=4.8 Hz, 1H); MS m/z 417 (M+1).

### Example 16

### Ethyl 3-(8-oxo-5,6,7,8-tetrahydro-7-quinolinyl)propanoate (intermediate)

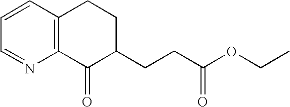

To a solution of 6,7-dihydro-8(5H)-quinolinone (3.0 g, 20 mmol) in 75 mL of benzene was added p-toluenesulfonic acid (0.34 g, 2 mmol) and pyrrolidine (2.8 g, 40 mmol). The mixture was heated to reflux under a Dean-Stark trap for 16 hours. The reaction mixture was allowed to cool to room temperature and was concentrated. The residue was taken up in toluene, concentrated, and dried under vacuum. The residue was dissolved in 50 mL of absolute ethanol before ethyl 2-propanoate (2.8 g, 28 mmol) was added. The mixture was heated to 78° C. for 3 hours before 25 mL of water was added. The reaction mixture was heated an additional 2 hours and then allowed to cool to room temperature. The mixture was diluted with 100 mL of water and extracted 3 times with 50 mL of dichloromethane. The combined organic layers were concentrated and the residue purified by silica chromatography eluting with acetonitrile to yield 3.1 g (62%) of ethyl 3-(8-oxo-5,6,7,8-tetrahydro-7-quinolinyl)propanoate. ¹H NMR (400 MHz, METHANOL-D4) δ ppm 1.2 (t, J=7.1 Hz, 3 H), 1.8 (td, J=13.8, 8.0 Hz, 1H), 1.9 (m, 1H), 2.3 (m, 2H), 2.5 (t, J=7.9 Hz, 2H), 2.7 (m, 1H), 3.1 (m, 2H), 4.1 (q, J=7.1 Hz, 2H), 7.5 (dd, J=7.9, 4.6 Hz, 1H), 7.8 (d, J=8.8 Hz, 1H), 8.6 (d, J=4.6 Hz, 1H); MS m/z 248 (M+1).

### Example 17

### Ethyl 3-[(7S,8R)-8-({(1R)-1-[4-(methyloxy)phenyl]ethyl}amino)-5,6,7,8-tetrahydro-7-quinolinyl]propanoate (intermediate)

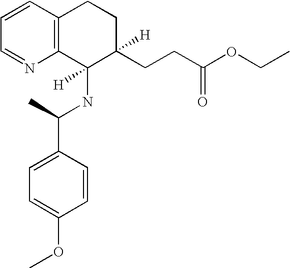

To a solution of ethyl 3-(8-oxo-5,6,7,8-tetrahydro-7-quinolinyl)propanoate (0.21 g, 0.84 mmol) in 10 mL of toluene was added p-toluenesulfonic acid (0.014 g, 0.084 mmol) and (1R)-1-[4-(methyloxy)phenyl]ethanamine (0.25 g, 1.7 mmol). The reaction mixture was heated to reflux with a Dean Stark trap overnight. The mixture was concentrated and the residue taken up in 10 mL of 1,2-dichloroethane. Sodium triacetoxyborohydride (0.53 g, 2.5 mmol) was added and the reaction mixture stirred at room temperature for 4 hours. The mixture was quenched with 10 mL of water and extracted 3 times with 10 mL of dichloromethane. The combined organic layers were concentrated and the residue purified by silica chromatography eluting with acetonitrile to yield 0.19 g (60%) of ethyl 3-[(7S,8R)-8-({(1R)-1-[4-(methyloxy)phenyl]ethyl}amino)-5,6,7,8-tetrahydro-7-quinolinyl]propanoate. ¹H NMR (400 MHz, METHANOL-D4) δ ppm 1.2 (t, J=7.1 Hz, 3H), 1.4 (d, J=6.8 Hz, 3H), 1.4 (m, 1H), 1.7 (m, 2H), 1.8 (m, 3H), 2.1 (m, 1H), 2.8 (m, 1H), 2.9 (m, 1H), 3.7 (d, J=3.7 Hz, 1H), 3.8 (s, 3H), 4.1 (q, J=7.1 Hz, 2H), 4.4 (q, J=6.7 Hz, 1H), 6.9 (d, J=8.8 Hz, 2H), 7.2 (dd, J=7.7, 4.8 Hz, 1H), 7.4 (d, J=8.8 Hz, 2H), 7.5 (d, J=9.1 Hz, 1H), 8.4 (d, J=4.8 Hz, 1H); MS m/z 383 (M+1).

### Example 18

### 3-[(7S,8R)-8-({(1R)-1-[4-(Methyloxy)phenyl]ethyl}amino)-5,6,7,8-tetrahydro-7-quinolinyl]-1-propanol (intermediate)

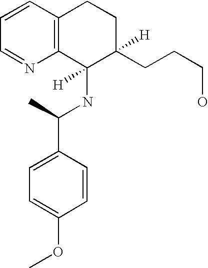

To a 0° C. solution of ethyl 3-[(7S,8R)-8-({(1R)-1-[4-(methyloxy)phenyl]ethyl}amino)-5,6,7,8-tetrahydro-7-quinolinyl]propanoate (0.26 g, 0.68 mmol) in 10 mL of tetrahydrofuran was added dropwise 1 M lithium aluminum hydride (1.36 mL, 1.4 mmol) in tetrahydrofuran. The reaction mixture was allowed to warm to room temperature and stirred for 2 hours. The mixture was sequentially quenched with 0.053 mL of water, 0.053 mL of 1 M aqueous sodium hydroxide, and 0.16 mL of water. A white precipitate was filtered off and the wash concentrated. The residue was purified by silica chromatography eluting with a gradient of 0% to 5% methanol in ethyl acetate to yield 0.11 g (48%) of 3-[(7S,8R)-8-({(1R)-1-[4-(methyloxy)phenyl]ethyl}amino)-5,6,7,8-tetrahydro-7-quinolinyl]-1-propanol. ¹H NMR (400 MHz, METHANOL-D4) δ ppm 1.1 (m, 2H), 1.3 (d, J=6.6 Hz, 3H), 1.4 (m, 1H), 1.5 (m, 1H), 1.6 (m, 1H), 1.7 (m, 1H), 1.8 (m, 1H), 2.7 (m, 1H), 2.9 (m, 1H), 3.4 (m, 2H), 3.6 (d, J=3.8 Hz, 1H), 3.8 (s, 3H), 4.1 (m, 1H), 6.9 (d, J=8.6 Hz, 2H), 7.2 (dd, J=7.7, 4.8 Hz, 1H), 7.4 (d, J=8.8 Hz, 2H), 7.5 (d, J=9.3 Hz, 1H), 8.3 (d, J=4.8 Hz, 1H); MS m/z 341 (M+1).

### Example 19

### (4aS,10bR)-1-{(1R)-1-[4-(Methyloxy)phenyl]ethyl}-1,2,3,4,4a,5,6,10b-octahydro-1,10-phenanthroline (intermediate)

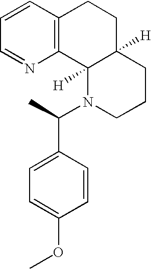

3-[(7S,8R)-8-({(1R)-1-[4-(Methyloxy)phenyl]ethyl}amino)-5,6,7,8-tetrahydro-7-quinolinyl]-1-propanol (0.020 g, 0.059 mmol), N,N-diisopropylethylamine (0.0080 g, 0.065 mmol), methanesulfonyl chloride (0.0074 g, 0.065 mmol), and 4-dimethylaminopyridine (0.0010 g, 0.0082 mmol) were added to 1 mL of dichloromethane and stirred for 2.5 hours. The reaction mixture was quenched with 2 mL of saturated sodium bicarbonate and extracted 3 times with 3 mL of dichloromethane. The combined organic layers were dried over sodium sulfate, concentrated, and the residue purified by silica chromatography eluting with a 0% to 10% gradient of 2M ammonia in methanol and dichloromethane as co-solvent to yield 0.018 g (95%) of (4aS,10bR)-1-{(1R)-1-[4-(methyloxy)phenyl]ethyl}-1,2,3,4,4a,5,6,10b-octahydro-1,10-phenanthroline. ¹H NMR (400 MHz, METHANOL-D4) δ ppm 1.3 (d, J=6.8 Hz, 3H), 1.5 (m, 3H), 1.6 (m, 1H), 1.8 (m, 1H), 2.0 (m, 1 H), 2.1 (m, 1H), 2.2 (m, 1H), 2.6 (m, 2H), 3.1 (m, 1H), 3.8 (m, 3H), 3.9 (s, 1H), 4.1 (m, 1H), 6.8 (d, J=8.8 Hz, 2H), 7.2 (dd, J=7.7, 4.9 Hz, 1H), 7.4 (d, J=8.6 Hz, 2H), 7.5 (d, J=7.7 Hz, 1H), 8.4 (d, J=4.4 Hz, 1H); MS m/z 323 (M+1).

### Example 20

### (4aS,10bR)-1,2,3,4,4a,5,6,10b-Octahydro-1,10-phenanthroline (intermediate)

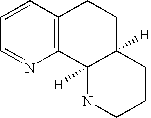

To a solution of (4aS,10bR)-1-{(1R)-1-[4-(methyloxy)phenyl]ethyl}-1,2,3,4,4a,5,6,10b-octahydro-1,10-phenanthroline (0.018 g, 0.056 mmol) in 5 mL of dichloromethane was added 5 mL of trifluoroacetic acid. The reaction mixture was stirred for 30 minutes and concentrated. The residue was dissolved in 5 mL of saturated sodium bicarbonate and extracted 3 times with 5 mL of dichloromethane. The combined organic layers were dried over sodium sulfate and concentrated. The residue purified by silica chromatography eluting with a 0% to 10% gradient of 30% aqueous ammonium hydroxide in acetonitrile to yield 0.0080 g (76%) of (4aS,10bR)-1,2,3,4,4a,5,6,10b-octahydro-1,10-phenanthroline. ¹H NMR (400 MHz, METHANOL-D4) δ ppm 1.5 (m, 1H), 1.7 (m, 3H), 1.8 (m, 1H), 2.1 (m, 2H), 2.9 (m, 4H), 3.8 (d, J=3.8 Hz, 1H), 7.2 (dd, J=7.7, 4.8 Hz, 1H), 7.6 (d, J=7.7 Hz, 1H), 8.4 (d, J=−4.9 Hz, 1H); MS m/z 189 (M+1).

### Example 21

### (4aS,10bR)-1-[(5-Fluoroimidazo[1,2-a]pyridin-2-yl)methyl]-1,2,3,4,4a,5,6,10b-octahydro-1,10-phenanthroline (intermediate)

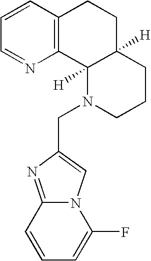

To a solution of (4aS,10bR)-1,2,3,4,4a,5,6,10b-octahydro-1,10-phenanthroline (0.065 g, 0.35 mmol) in 5 mL of acetonitrile was added potassium carbonate (0.23 g, 1.7 mmol), 2-(chloromethyl)-5-fluoroimidazo[1,2-a]pyridine (0.077 g, 0.42 mmol), and 10 mg of potassium iodide. The reaction mixture was stirred for 16 hours at room temperature and quenched with 10 mL of water. The mixture was extracted 3 times with 20 mL of dichloromethane. The combined organic layers were concentrated and the residue purified by silica chromatography eluting with a 0% to 10% gradient of 2M ammonia in methanol and dichloromethane as co-solvent to yield 0.073 g (62%) of (4aS,10bR)-1-[(5-fluoroimidazo[1,2-a]pyridin-2-yl)methyl]-1,2,3,4,4a,5,6,10b-octahydro-1,10-phenanthroline. ¹H NMR (400 MHz, METHANOL-D4) δ ppm 1.5 (m, 1 H), 1.7 (m, 3H), 1.8 (m, 1H), 2.0 (m, 1H), 2.3 (td, J=11.2, 2.9 Hz, 1H), 2.4 (m, J=13.2, 10.2, 7.6, 7.6 Hz, 1H), 2.7 (m, 1H), 2.9 (dt, J=11.8, 4.0 Hz, 1H), 3.1 (ddd, J=17.2, 7.3, 5.5 Hz, 1H), 3.5 (d, J=2.9 Hz, 1H), 3.6 (d, J=14.8 Hz, 1H), 4.0 (d, J=14.8 Hz, 1H), 6.6 (m, 1H), 7.1 (dd, J=7.7, 4.8 Hz, 1H), 7.3 (m, 2H), 7.5 (d, J=9.0 Hz, 1H), 7.6 (s, 1H), 8.3 (d, J=4.8 Hz, 1H); MS m/z 337 (M+1).

### Example 22

### (4aS,10bR)-1-{[5-(4-Methyl-1-piperazinyl)imidazo[1,2-a]pyridin-2-yl]methyl}-1,2,3,4,4a,5,6,10b-octahydro-1,10-phenanthroline

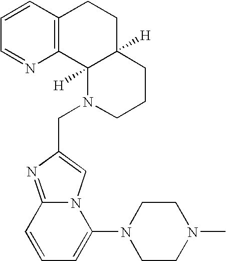

(4aS,10bR)-1-[(5-Fluoroimidazo[1,2-a]pyridin-2-yl)methyl]-1,2,3,4,4a,5,6,10b-octahydro-1,10-phenanthroline (0.073 g, 0.22 mmol) was dissolved in 3 mL of 1-methylpiperazine and heated to 200° C. in a microwave reactor for 20 minutes. The reaction was allowed to cool and diluted with 5 mL of brine. The mixture was extracted 3 times with 5 mL of dichloromethane. The combined organic layers were washed 3 times with 5 mL of water, dried over sodium sulfate, and concentrated. The residue purified by silica chromatography eluting with a 0% to 10% gradient of 30% aqueous ammonium hydroxide in acetonitrile to yield 0.073 g (81%) of (4aS,10bR)-1-{[5-(4-methyl-1-piperazinyl)imidazo[1,2-a]pyridin-2-yl]methyl}-1,2,3,4,4a,5,6,10b-octahydro-1,10-phenanthroline. ¹H NMR (400 MHz, METHANOL-D4) δ ppm 1.7 (m, 5H), 2.1 (m, 1H), 2.4 (m, 5H), 2.8 (m, 5H), 2.9 (m, 1H), 3.1 (m, 5H), 3.6 (d, J=2.4 Hz, 1H), 3.7 (d, J=14.8 Hz, 1H), 4.1 (d, J=14.6 Hz, 1H), 6.4 (d, J=6.0 Hz, 1H), 7.2 (m, 2H), 7.2 (m, 1H), 7.5 (d, J=9.0 Hz, 1H), 7.6 (s, 1H), 8.3 (d, J=6.4 Hz, 1H); MS m/z 417 (M+1).

### Example 23

### 3-[(7R,8S)-8-({(1S)-1-[4-(Methyloxy)phenyl]ethyl}amino)-5,6,7,8-tetrahydro-7-quinolinyl]-1-propanol (intermediate)

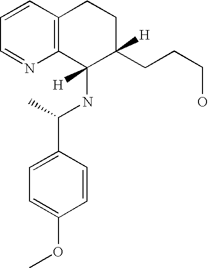

To a solution of ethyl 3-(8-oxo-5,6,7,8-tetrahydro-7-quinolinyl)propanoate (0.32 g, 1.3 mmol) in 25 mL of toluene was added p-toluenesulfonic acid (0.022 g, 0.13 mmol) and (1S)-1-[4-(methyloxy)phenyl]ethanamine (0.39 g, 2.6 mmol). The reaction mixture was refluxed with a Dean Stark trap overnight. The mixture was concentrated and the residue taken up 25 mL of 1,2-dichloroethane. Sodium triacetoxyborohydride (0.82 g, 3.9 mmol) was added and the reaction mixture stirred at room temperature for 4 hours. The mixture was quenched with 25 mL of water and extracted 3 times with 25 mL of dichloromethane. The combined organic layers were concentrated and the residue passed through a short silica gel chromatography column eluting with 5% methanol in ethyl acetate to yield 0.46 g of crude ethyl 3-[(7R,8S)-8-({(1S)-1-[4-(methyloxy)phenyl]ethyl}amino)-5,6,7,8-tetrahydro-7-quinolinyl]propanoate. The material was dissolved in 50 mL of dry tetrahydrofuran before 2M lithium borohydride (0.98 mL, 1.9 mmol) in tetrahydrofuran was added. The reaction mixture was heated to 65° C. for 2 hours. Another portion of 2M lithium borohydride (0.98 mL, 1.9 mmol) in tetrahydrofuran was added and the mixture continued to heat at 65° C. for 16 hours. The reaction mixture was allowed to cool to room temperature and was quenched with 100 mL of water. The mixture was extracted 3 times with 50 mL of dichloromethane and the combined organic layers concentrated. The residue was purified by silica chromatography eluting with a 0% to 10% gradient of methanol in ethyl acetate to yield 0.19 g (43%) of 3-[(7R,8S)-8-({(1S)-1-[4-(methyloxy)phenyl]ethyl}amino)-5,6,7,8-tetrahydro-7-quinolinyl]-1-propanol. ¹H NMR (400 MHz, METHANOL-D4) δ ppm 1.1 (m, 2H), 1.3 (d, J=6.8 Hz, 3H), 1.4 (m, 1H), 1.5 (m, 1H), 1.6 (m, 1H), 1.7 (m, 1H), 1.8 (m, 1H), 2.8 (m, 1H), 2.9 (m, 1H), 3.4 (m, 2H), 3.6 (d, J=3.7 Hz, 1H), 3.8 (s, 3H), 4.1 (q, J=7.0 Hz, 1H), 6.9 (d, J=8.8 Hz, 2H), 7.2 (dd, J=7.7, 4.8 Hz, 1H), 7.4 (d, J=8.6 Hz, 2H), 7.5 (d, J=7.7 Hz, 1H), 8.3 (d, J=6.4 Hz, 1H); MS m/z 341 (M+1).

### Example 24

### (4aR,10bS)-1-{(1S)-1-[4-(Methyloxy)phenyl]ethyl}-1,2,3,4,4a,5,6,10b-octahydro-1,10-phenanthroline (intermediate)

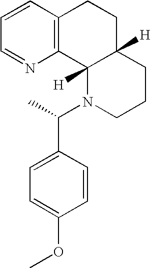

3-[(7R,8S)-8-({(1S)-1-[4-(Methyloxy)phenyl]ethyl}amino)-5,6,7,8-tetrahydro-7-quinolinyl]-1-propanol (0.19 g, 0.56 mmol), N,N-diisopropylethylamine (0.10 g, 0.78 mmol), methanesulfonyl chloride (0.083 g, 0.73 mmol), and 4-dimethylaminopyridine (0.0070 g, 0.056 mmol) were added to 10 mL of dichloromethane and stirred for 16 hours. The reaction mixture was quenched with 20 mL of saturated sodium bicarbonate and extracted 3 times with 30 mL of dichloromethane. The combined organic layers were dried over sodium sulfate, concentrated, and the residue purified by silica chromatography eluting with a 0% to 10% gradient of 2M ammonia in methanol and dichloromethane as co-solvent to yield 0.16 g (91%) of (4aR,10bS)-1-{(1S) 1-[4-(methyloxy)phenyl]ethyl}-1,2,3,4,4a,5,6,10b-octahydro-1,10-phenanthroline. ¹H NMR (400 MHz, METHANOL-D4) δ ppm 1.3 (d, J=6.8 Hz, 3H), 1.5 (m, 3H), 1.6 (d, J=11.5 Hz, 1H), 1.8 (m, 1H), 2.0 (m, 1H), 2.1 (m, 1H), 2.2 (m, 1H), 2.6 (ddd, J=16.1, 7.3, 3.1 Hz, 2H), 3.1 (ddd, J=17.3, 8.5, 8.2 Hz, 1H), 3.8 (s, 3 H), 3.9 (m, 1H), 4.1 (m, 1H), 6.8 (d, J=8.8 Hz, 2H), 7.2 (dd, J=7.7, 4.8 Hz, 1H), 7.4 (d, J=8.8 Hz, 2H), 7.5 (d, J=7.7 Hz, 1H), 8.4 (d, J=4.8 Hz, 1H); MS m/z 323 (M+1).

### Example 25

### (4aR,10bS)-1,2,3,4,4a,5,6,10b-Octahydro-1,10-phenanthroline (intermediate)

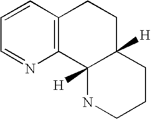

To a solution of (4aR,10bS)-1-{(1S)-1-[4-(methyloxy)phenyl]ethyl}-1,2,3,4,4a,5,6,10b-octahydro-1,10-phenanthroline (0.15 g, 0.45 mmol) in 10 mL of dichloromethane was added 10 mL of trifluoroacetic acid. The reaction mixture was stirred for 1 hour and concentrated. The residue was taken up in 10 mL of saturated sodium bicarbonate and extracted 2 times with 20 mL of dichloromethane. The combined organic layers were dried over sodium sulfate and concentrated. The residue was purified by silica chromatography eluting with a 0% to 10% gradient of 30% aqueous ammonium hydroxide in acetonitrile to yield 0.082 g (97%) of (4aR,10bS)-1,2,3,4,4a,5,6,10b-octahydro-1,10-phenanthroline. ¹H NMR (400 MHz, METHANOL-D4) δ ppm 1.6 (m, 1 H), 1.7 (m, 3H), 1.8 (m, 1H), 2.1 (m, 2H), 2.9 (m, 4H), 3.9 (d, J=3.5 Hz, 1H), 7.2 (dd, J=7.7, 4.8 Hz, 1H), 7.6 (d, J=6.4 Hz, 1H), 8.4 (d, J=5.5 Hz, 1H); MS m/z 189 (M+1).

### Example 26

### (4aR,10bS)-1-[(5-Fluoroimidazo[1,2-a]pyridin-2-yl)methyl]-1,2,3,4,4a,5,6,10b-octahydro-1,10-phenanthroline (intermediate)

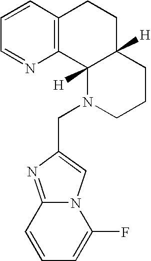

To a solution of (4aR,10bS)-1,2,3,4,4a,5,6,10b-octahydro-1,10-phenanthroline (0.080 g, 0.43 mmol) in 10 mL of acetonitrile was added potassium carbonate (0.29 g, 2.1 mmol), 2-(chloromethyl)-5-fluoroimidazo[1,2-a]pyridine (0.095 g, 0.52 mmol), and 10 mg of potassium iodide. The reaction mixture was stirred for 16 hours at room temperature and quenched with 10 mL of water. The mixture was extracted 3 times with 20 mL of dichloromethane. The combined organic layers were concentrated and the residue purified by silica chromatography eluting with a 0% to 10% gradient of 2M ammonia in methanol and dichloromethane as co-solvent to yield 0.11 g (77%) of (4aR,10bS)-1-[(5-fluoroimidazo[1,2-a]pyridin-2-yl)methyl]-1,2,3,4,4a,5,6,10b-octahydro-1,10-phenanthroline. ¹H NMR (400 MHz, METHANOL-D4) δ ppm 1.5 (m, 1 H), 1.7 (m, 3H), 1.8 (m, 1H), 2.1 (m, 1H), 2.3 (td, J=11.2, 3.0 Hz, 1H), 2.5 (m, 1H), 2.8 (m, 1H), 3.0 (m, 1H), 3.1 (m, 1H), 3.5 (s, 1H), 3.6 (d, J=15.2 Hz, 1H), 4.0 (d, J=14.8 Hz, 1H), 6.6 (m, 1H), 7.2 (dd, J=7.7, 4.9 Hz, 1H), 7.3 (m, 2H), 7.5 (d, J=7.7 Hz, 1H), 7.6 (s, 1H), 8.3 (d, J=4.8 Hz, 1H); MS m/z 337 (M+1).

### Example 27

### (4aR,10bS)-1-{[5-(4-Methyl-1-piperazinyl)imidazo[1,2-a]pyridin-2-yl]methyl}-1,2,3,4,4a,5,6,10b-octahydro-1,10-phenanthroline

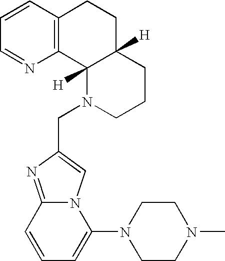

(4aR,10bS)-1-[(5-Fluoroimidazo[1,2-a]pyridin-2-yl)methyl]-1,2,3,4,4a,5,6,10b-octahydro-1,10-phenanthroline (0.11 g, 0.32 mmol) was dissolved in 5 mL 1-methylpiperazine and heated to 200° C. in a microwave reactor for 20 minutes. The reaction was allowed to cool and diluted with 10 mL of brine. The mixture was extracted 3 times with 10 mL of dichloromethane. The combined organic layers were washed with 10 mL of water, dried over sodium sulfate, and concentrated. The residue purified by silica chromatography eluting with a 0% to 10% gradient of 30% aqueous ammonium hydroxide in acetonitrile to yield 0.080 g (68%) of (4aR,10bS)-1-{[5-(4-methyl-1-piperazinyl)imidazo[1,2-a]pyridin-2-yl]methyl}-1,2,3,4,4a,5,6,10b-octahydro-1,10-phenanthroline. ¹H NMR (400 MHz, METHANOL-D4) δ ppm 1.7 (m, 5 H), 2.1 (m, 1H), 2.4 (m, 5H), 2.7 (m, 5H), 3.0 (m, 1H), 3.1 (m, 5H), 3.6 (m, 1H), 3.6 (d, J=14.6 Hz, 1H), 4.0 (d, J=14.5 Hz, 1H), 6.4 (d, J=7.3 Hz, 1H), 7.2 (m, 3H), 7.5 (d, J=9.1 Hz, 1H), 7.6 (s, 1H), 8.3 (d, J=6.4 Hz, 1H); MS m/z 417 (M+1).

Alternatively (4aR,10bS)-1-{[5-(4-methyl-1-piperazinyl)imidazo[1,2-a]pyridin-2-yl]methyl}-1,2,3,4,4a,5,6,10b-octahydro-1,10-phenanthroline can be synthesized in the following manner:

### A) Ethyl 3-(8-oxo-5,6,7,8-tetrahydro-7-quinolinyl)propanoate

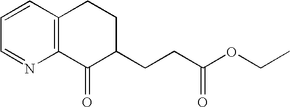

To a solution of 6,7-dihydro-8(5″-quinolinone (11.8 g, 80 mmol) in 400 mL of benzene was added p-toluenesulfonic acid (1.5 g, 8.0 mmol) and pyrrolidine (6.8 g, 96 mmol). The mixture was heated to reflux under a Dean-Stark trap for 4 hours. The reaction mixture was allowed to cool to room temperature and was concentrated under vacuum. The residue was dissolved in 400 mL of absolute ethanol before ethyl acrylate (11.2 g, 112 mmol) was added. The mixture was heated to 78° C. for 6 hours and then stirred overnight at room temperature before 35 mL of water was added. The reaction mixture was heated at 78° C. for 4 hours and then allowed to cool to room temperature. The mixture was concentrated to about 50 mL, diluted with 150 mL of water, and extracted 3 times with 100 mL of dichloromethane. The combined organic layers were concentrated and the residue purified by silica chromatography eluting with acetonitrile to yield 11.5 g (58%) of ethyl 3-(8-oxo-5,6,7,8-tetrahydro-7-quinolinyl)propanoate. ¹H NMR (400 MHz, METHANOL-D4) δ ppm 1.2 (t, J=7.1 Hz, 3H), 1.8 (td, J=14.2, 7.1 Hz, 1H), 2.0 (m, 1H), 2.3 (m, 2H), 2.5 (t, J=7.8 Hz, 2H), 2.7 (m, 1H), 3.1 (m, 2H), 4.1 (q, J=7.1 Hz, 2H), 7.5 (dd, J=7.9, 4.6 Hz, 1H), 7.8 (d, J=8.8 Hz, 1H), 8.6 (d, J=4.6 Hz, 1H); m/z 248 (M+1).

### B) ethyl 3-[(7R,8S)-8-({(1S)-1-[4-(methyloxy)phenyl]ethyl}amino)-5,6,7,8-tetrahydro-7-quinolinyl]propanoate

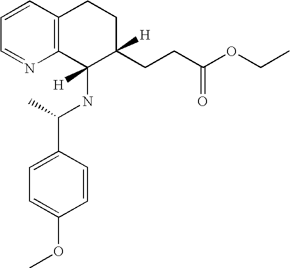

To a solution of ethyl 3-(8-oxo-5,6,7,8-tetrahydro-7-quinolinyl)propanoate (11.5 g, 46.5 mmol) in 350 mL of toluene was added p-toluenesulfonic acid (0.89 g, 4.6 mmol) and (1S)-1-[4-(methyloxy)phenyl]ethanamine (10.5 g, 69.8 mmol). The reaction mixture was refluxed under a Dean Stark trap for 4 hours. The mixture was allowed to cool, concentrated, and the residue dissolved in 350 mL of 1,2-dichloroethane. Sodium triacetoxyborohydride (29.6 g, 140 mmol) was added and the reaction mixture stirred at room temperature for 16 hours. The mixture was diluted with 350 mL of dichloromethane and washed with water. The organic phase was concentrated and the residue purified by silica chromatography eluting with ethyl acetate to yield 10.8 g of ethyl 3-[(7R,8S)-8-({(1S)-1-[4-(methyloxy)phenyl]ethyl}amino)-5,6,7,8-tetrahydro-7-quinolinyl]propanoate. ¹H NMR (400 MHz, METHANOL-D4) δ ppm 1.2 (t, J=7.1 Hz, 3H), 1.4 (m, 1H), 1.4 (d, J=6.8 Hz, 3H), 1.8 (m, 5H), 2.1 (m, 1H), 2.9 (m, 2H), 3.8 (s, 3H), 3.9 (d, J=2.9 Hz, 1H), 4.1 (q, J=7.1 Hz, 2H), 4.5 (q, J=6.5 Hz, 1H), 6.9 (d, J=8.8 Hz, 2H), 7.3 (dd, J=7.7, 4.8 Hz, 1H), 7.5 (d, J=8.6 Hz, 2H), 7.6 (d, J=7.5 Hz, 1H), 8.4 (d, J=4.0 Hz, 1H); m/z 383 (M+1).

### C) 3-[(7R,8S)-8-({(1S)-1-[4-(methyloxy)phenyl]ethyl}amino)-5,6,7,8-tetrahydro-7-quinolinyl]-1-propanol

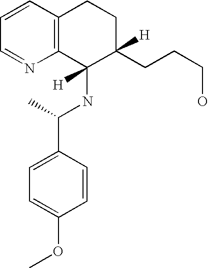

To a 0° C. solution of ethyl 3-[(7R,8S)-8-({(1S)-1-[4-(methyloxy)phenyl]ethyl}amino)-5,6,7,8-tetrahydro-7-quinolinyl]propanoate (10.8 g, 28.2 mmol) in 250 mL of tetrahydrofuran was added slowly dropwise a 1 M solution of lithium aluminum hydride (28.2 mL, 28.2 mmol) in tetrahydrofuran. The mixture was stirred for 1 hour and quenched sequentially with dropwise addition of 0.33 mL of water, 0.33 mL of 1 M sodium hydroxide_{(aq)}, and 0.99 mL of water. The mixture was diluted with 250 mL of tetrahydrofuran and allowed to warm to room temperature. The mixture was stirred for 30 minutes and the solids filtered off. The wash was concentrated and the residue purified by silica chromatography eluting with a 0% to 10% gradient of 2M ammonia in isopropanol with dichloromethane as co-eluent. Appropriate fractions were concentrated to yield 6.1 g (64%) of 3-[(7R,8S)-8-({(1S)-1-[4-(methyloxy)phenyl]ethyl}amino)-5,6,7,8-tetrahydro-7-quinolinyl]-1 propanol. ¹H NMR (400 MHz, METHANOL-D4) δ ppm 1.1 (m, 2H), 1.3 (m, 3H), 1.4 (m, 2H), 1.7 (m, 2 H), 1.8 (m, 1H), 2.8 (m, 2H), 3.4 (m, 2H), 3.6 (d, J=3.7 Hz, 1H), 3.8 (s, 3H), 4.1 (m, 1H), 6.9 (d, J=8.6 Hz, 2H), 7.2 (dd, J=7.7, 4.8 Hz, 1H), 7.4 (m, 2H), 7.5 (d, J=7.5 Hz, 1H), 8.3 (d, J=4.8 Hz, 1H); m/z 341 (M+1).

### D) (4aR,10bS)-1-{(1S)-1-[4-(methoxy)phenyl]ethyl}-1,2,3,4,4a,5,6,10b-octahydro-1,10-phenanthroline

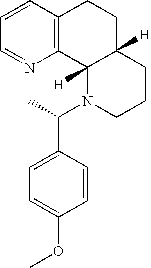

To a solution of 3-[(7R,8S)-8-({(1S)-1-[4-(methyloxy)phenyl]ethyl}amino)-5,6,7,8-tetrahydro-7-quinolinyl]-1-propanol (12.6 g, 37 mmol) and diisopropylethylamine (7.2 g, 55 mmol) in 175 mL of dichloromethane was added methanesulfonyl chloride (5.5 g, 48 mmol) slowly dropwise. 4-Dimethylaminopyridine (0.45 g, 3.7 mmol) was added and the mixture stirred at room temperature for 16 hours. The reaction mixture was diluted with water and extracted 3 times with 100 mL of dichloromethane. The combined organic layers were concentrated and the residue purified by silica chromatography eluting with a 0% to 10% gradient of 2M ammonia in methanol with dichloromethane as co-eluent to yield 11.9 g (99%) of (4aR,10bS)-1-{(1S)-1-[4-(methyloxy)phenyl]ethyl}-1,2,3,4,4a,5,6,10b-octahydro-1,10-phenanthroline. ¹H NMR (400 MHz, METHANOL-D4) δ ppm 1.3 (d, J=6.8 Hz, 3H), 1.5 (m, 3H), 1.6 (m, 1H), 1.8 (m, 1H), 2.0 (m, 1H), 2.1 (m, 1H), 2.2 (m, 1H), 2.6 (ddd, J=16.5, 7.2, 3.2 Hz, 2H), 3.1 (ddd, J=16.8, 8.7, 8.5 Hz, 1H), 3.8 (s, 3H), 3.9 (d, J=3.5 Hz, 1H), 4.1 (q, J=6.7 Hz, 1H), 6.8 (d, J=8.8 Hz, 2H), 7.2 (dd, J=7.6, 4.8 Hz, 1H), 7.4 (d, J=8.6 Hz, 2H), 7.5 (d, J=7.5 Hz, 1H), 8.4 (d, J=4.8 Hz, 1H); m/z 323 (M+1).

### E) (4aR,10bS)-1,2,3,4,4a,5,6,10b-octahydro-1,10-phenanthroline

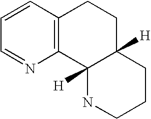

(4aR,10bS)-1-{(1S)-1-[4-(methyloxy)phenyl]ethyl}-1,2,3,4,4a,5,6,10b-octahydro-1,10-phenanthroline (11.9 g, 36 mmol) was stirred in a mixture of 300 mL of dichloromethane and 300 mL of trifluoroacetic acid for 2 hours. The mixture was concentrated under vacuum and the residue carefully quenched with saturated sodium bicarbonate. The mixture was extracted 3 times with 100 mL of dichloromethane and concentrated. The residue was purified by silica chromatography eluting with a 0% to 10% gradient of 30% aqueous ammonium hydroxide in acetonitrile to yield 6.8 g (>99%) of (4aR,10bS)-1,2,3,4,4a,5,6,10b-octahydro-1,10-phenanthroline. ¹H NMR (400 MHz, METHANOL-D4) δ ppm 1.6 (m, 1H), 1.8 (m, 3H), 1.9 (m, 1H), 2.0 (m, 1H), 2.4 (m, 1H), 2.9 (m, 3H), 3.1 (m, 1H), 4.4 (d, J=4.0 Hz, 1H), 7.3 (dd, J=7.7, 4.9 Hz, 1H), 7.6 (d, J=7.9 Hz, 1H), 8.5 (d, J=4.6 Hz, 1H); m/z 189 (M+1).

### F) (4aR,10bS)-1-[(5-fluoroimidazo[1,2-a]pyridin-2-yl)methyl]-1,2,3,4,4a,5,6,10b-octahydro-1,10-phenanthroline

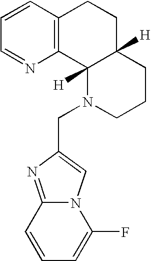

(4aR,10bS)-1,2,3,4,4a,5,6,10b-octahydro-1,10-phenanthroline (6.2 g, 33 mmol), 2-(chloromethyl)-5-fluoroimidazo[1,2-a]pyridine (7.3 g, 40 mmol), potassium carbonate (23 g, 160 mmol), and potassium iodide (0.82 g, 5.0 mmol) in 400 mL of acetonitrile were stirred for 16 hours at room temperature. The solids were filtered off and the wash concentrated. The residue was purified by silica chromatography eluting with a 0% to 3% gradient of 30% aqueous ammonium hydroxide in acetonitrile to yield 6.2 g (56%) of (4aR,10bS)-1-[(5-fluoroimidazo[1,2-a]pyridin-2-yl)methyl]-1,2,3,4,4a,5,6,10b-octahydro-1,10-phenanthroline. ¹H NMR (400 MHz, METHANOL-D4) δ ppm 1.7 (m, 5H), 2.2 (m, 1H), 2.4 (m, 1H), 2.5 (m, 1H), 2.8 (dt, J=17.2, 6.7 Hz, 1H), 3.0 (m, 1H), 3.1 (m, 1H), 3.8 (m, 2H), 4.2 (m, 1H), 6.6 (m, 1H), 7.2 (dd, J=7.5, 4.9 Hz, 1H), 7.3 (m, 2H), 7.5 (d, J=7.5 Hz, 1H), 7.7 (s, 1H), 8.3 (d, J=4.0 Hz, 1H); m/z 337 (M+1).

### G) (4aR,10bS)-1-{[5-(4-methyl-1-piperazinyl)imidazo[1,2-a]pyridin-2-yl]methyl}-1,2,3,4,4a,5,6,10b-octahydro-1,10-phenanthroline

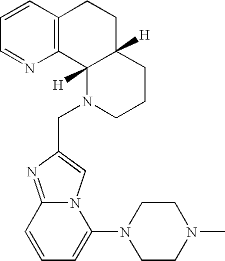

(4aR,10bS)-1-[(5-fluoroimidazo[1,2-a]pyridin-2-yl)methyl]-1,2,3,4,4a,5,6,10b-octahydro-1,10-phenanthroline (6.1 g, 18 mmol) was dissolved in 8 mL of 1-methylpiperazine and 10 mL of dimethyl sulfoxide. The mixture was stirred at 60° C. for 16 hours and then allowed to cool to room temperature. The mixture was quenched with water and extracted 3 times with 100 mL of dichloromethane. The combined organic layers were washed 1 time with brine, dried over sodium sulfate, concentrated, and the residue dissolved in acetonitrile. The solution was cooled to 0° C. Crystals formed and were collected by filtration. The wash was concentrated and the residue again dissolved in acetonitrile. The solution was cooled to 0° C. Crystals formed and were collected by filtration. The first crop and second crop of crystals were combined to yield 4.75 g (63%) of (4aR,10bS)-1-{[5-(4-methyl-1-piperazinyl)imidazo[1,2-a]pyridin-2-yl]methyl}-1,2,3,4,4a,5,6,10b-octahydro-1,10-phenanthroline. ¹H NMR (400 MHz, METHANOL-D4) δ ppm 1.6 (m, 1H), 1.7 (m, 3 H), 1.8 (m, 1H), 2.1 (m, 1H), 2.4 (m, 5H), 2.8 (m, 5H), 3.0 (m, 1H), 3.1 (m, 5H), 3.6 (d, J=3.1 Hz, 1H), 3.6 (d, J=14.6 Hz, 1H), 4.0 (d, J=14.6 Hz, 1H), 6.4 (d, J=7.1 Hz, 1H), 7.2 (m, 2H), 7.2 (m, 1H), 7.5 (d, J=7.3 Hz, 1H), 7.6 (s, 1H), 8.3 (d, J=4.8 Hz, 1H); m/z 417 (M+1).

### Example 28

### 3-(4-Methyl-1-piperazinyl)-2-nitroaniline (intermediate)

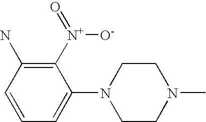

To a solution of 3-chloro-2-nitroaniline (0.65 g, 3.8 mmol) in 3 mL of 1-methylpiperazine was heated to 150° C. in a microwave reactor for 20 minutes. The mix was allowed to cool and was quenched with 10 mL of water. The mixture was extracted 3 times with 10 mL of dichloromethane. The combined organic layers were washed 2 times with 10 mL of water, dried over sodium sulfate, and concentrated. The residue was recrystallized from dichloromethane and hexanes to yield 0.90 g (>99%) of 3-(4-methyl-1-piperazinyl)-2-nitroaniline. Journal of the Chemical Society, Chemical Communications (1980), (6), 281-2.

### Example 29

### Phenylmethyl 3,4,4a,5,6,10b-hexahydro-1,10-phenanthrolin-1(2H)-′H-NMR-ylacetate (intermediate)

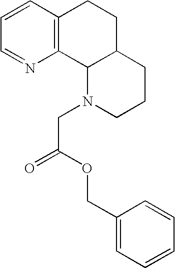

To a solution of 1,2,3,4,4a,5,6,10b-octahydro-1,10-phenanthroline (0.63 g, 3.3 mmol) in 15 mL of acetonitrile was added potassium carbonate (2.3 g, 17 mmol), phenylmethyl bromoacetate (0.83 g, 3.6 mmol), and 10 mg of potassium iodide. The reaction mixture was stirred for 16 hours at room temperature and diluted with 50 mL of dichloromethane. The organic phase was washed with 20 mL of water and then 20 mL of brine. The organic phase was dried over sodium sulfate and concentrated. The residue was purified by silica chromatography eluting with a 0% to 10% gradient of 2M ammonia in methanol and dichloromethane as co-solvent to yield 0.90 g (81%) of phenylmethyl 3,4,4a,5,6,10b-hexahydro-1,10-phenanthrolin-1(2H)-ylacetate. ¹H NMR (400 MHz, METHANOL-D4) δ ppm 1.5 (m, 3H), 1.8 (m, 2H), 1.9 (m, 2H), 2.5-3.1 (m, 4H), 3.5 (m, 2H), 3.7 (d, J=10.6 Hz, 1H), 4.9 (m, 1H), 5.1 (m, 1H), 7.1 (2 dd, J=7.8, 4.7 Hz, 1H), 7.3 (m, 5H), 7.5 (2d, J=7.7 Hz, 1H), 8.2 (2d, J=4.8 Hz, 1H); MS m/z 337 (M+1).

### Example 30

### 3,4,4a,5,6,10b-Hexahydro-1,10-phenanthrolin-1(2H)-ylacetic acid (intermediate)

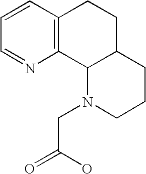

A solution of phenylmethyl 3,4,4a,5,6,10b-hexahydro-1,10-phenanthrolin-1(2H)-ylacetate (0.90 g, 206 mmol) was hydrogenated under 20 psi of hydrogen with 0.19 g of 10% Pd/C in 70 mL of methanol. After 2 hours, the catalyst was filtered off over celite and the wash concentrated. The residue was dried under high vacuum to yield 0.68 g (>99%) of 3,4,4a,5,6,10b-hexahydro-1,10-phenanthrolin-1(2H)-ylacetic acid.

¹H NMR (400 MHz, METHANOL-D4) δ ppm 1.5 (m, 1H), 1.7 (m, 2H), 2.0 (m, 4H), 2.6-3.2 (m, 4H), 3.6 (m, 2H), 3.8-4.0 (m, 1H), 4.4-4.7 (m, 1H), 7.3 (m, 1H), 7.6 (m, 1H), 8.5 (2d, J=4.6 Hz, 1H); MS m/z 247 (M+1).

### Example 31A

### (trans)-1,1-Dimethylethyl 2-(3,4,4a,5,6,10b-hexahydro-1,10-phenanthrolin-1(2H)-ylmethyl)-4-(4-methyl-1-piperazinyl)-1H-benzimidazole-1-carboxylate

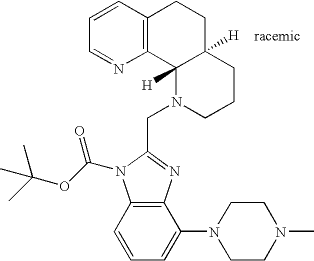

### Example 31B

### (cis)-1,1-Dimethylethyl 2-(3,4,4a,5,6,10b-hexahydro-1,10-phenanthrolin-1(2H)-ylmethyl)-4-(4-methyl-1-piperazinyl)-1H-benzimidazole-1-carboxylate

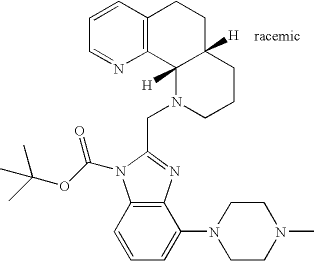

3-(4-Methyl-1-piperazinyl)-2-nitroaniline (0.61 g, 2.6 mmol) in 100 mL of ethanol was hydrogenated under 50 psi of hydrogen with 0.060 g of 10% Pd/C for 1 hour. The catalyst was filtered off over celite and the wash concentrated. The residue was dissolved in 50 mL of acetonitrile before 3,4,4a,5,6,10b-hexahydro-1,10-phenanthrolin-1(2H)-ylacetic acid (0.61 g, 2.5 mmol), N,N-diisopropylethylamine (0.96 g, 7.5 mmol), and bis(2-oxo-1,3-oxazolidin-3-yl)phosphinic chloride (0.95 g, 3.7 mmol) were added sequentially. The reaction mixture was stirred for 2.5 hours before being concentrated. The residue was taken up in 50 mL of dichloromethane and washed with 50 mL of saturated sodium bicarbonate followed by 50 mL of brine. The organic phase was dried over sodium sulfate and concentrated. The residue was dissolved in 50 mL of acetic acid and heated to 70° C. for 1 hour and then at room temperature for 16 hours. The mixture was concentrated, diluted with 100 mL of water, neutralized with solid sodium bicarbonate, and extracted 3 times with 50 mL of dichloromethane. The combined organic layers were concentrated and the residue purified by silica chromatography eluting with a 0% to 10% gradient of 30% aqueous ammonium hydroxide in acetonitrile to yield 0.56 g of 1-{[4-(4-methyl-1-piperazinyl)-1H-benzimidazol-2-yl]methyl}-1,2,3,4,4a,5,6,10b-octahydro-1,10-phenanthroline. To a solution of 1-{[4-(4-methyl-1-piperazinyl)-1H-benzimidazol-2-yl]methyl}-1,2,3,4,4a,5,6,10b-octahydro-1,10-phenanthroline (0.43 g, 1.0 mmol) in 30 mL of dichloromethane was added N,N-dimethyl-4-pyridinamine (0.012 g, 0.10 mmol) and di-tert-butyl-dicarbonate (0.34 g, 1.5 mmol). The reaction mixture was stirred at room temperature for 16 hours. The mixture was filtered and the wash concentrated. The residue was purified by silica chromatography eluting with a 0% to 10% gradient of 30% aqueous ammonium hydroxide in acetonitrile to yield 0.25 g (48%) of (trans)-1,1-dimethylethyl 2-(3,4,4a,5,6,10b-hexahydro-1,10-phenanthrolin-1(2H)-ylmethyl)-4-(4-methyl-1-piperazinyl)-1H-benzimidazole-1-carboxylate and 0.14 g (27%) of (cis)-1,1-dimethylethyl 2-(3,4,4a,5,6,10b-hexahydro-1,10-phenanthrolin-1(2H)-ylmethyl)-4-(4-methyl-1-piperazinyl)-1H-benzimidazole-1-carboxylate. 31A: ¹H NMR (400 MHz, METHANOL-D4) δ ppm 1.5 (m, 3H), 1.6 (s, 9H), 1.9 (m, 1H), 2.0 (m, 2H), 2.1 (m, 1 H), 2.4 (s, 3H), 2.7 (m, 5H), 2.9 (ddd, J=16.8, 11.9, 4.9 Hz, 1H), 3.1 (m, 1H), 3.4 (m, 4H), 3.7 (m, 1H), 3.8 (d, J=11.0 Hz, 1H), 4.3 (m, 2H), 6.7 (d, J=8.2 Hz, 1H), 6.8 (dd, J=7.1, 4.8 Hz, 1H), 7.1 (t, J=8.1 Hz, 1H), 7.3 (d, J=8.4 Hz, 1H), 7.5 (d, J=7.5 Hz, 1H), 7.9 (d, J=4.6 Hz, 1H); MS m/z 517 (M+1). 31B: ¹H NMR (400 MHz, METHANOL-D4) δ ppm 1.4 (m, 2H), 1.5 (m, 1H), 1.6 (s, 9H), 1.7 (m, 1H), 2.0 (m, 1 H), 2.3 (m, 1H), 2.4 (s, 3H), 2.6 (m, 2H), 2.7 (m, 4H), 2.7 (m, 1H), 2.9 (m, 1H), 3.0 (m, 1H), 3.4 (m, 4H), 3.6 (m, 1H), 3.8 (d, J=14.8 Hz, 1H), 4.5 (d, J=15.0 Hz, 1H), (d, J=7.3 Hz, 1H), 7.1 (dd, J=7.7, 4.8 Hz, 1H), 7.1 (t, J=8.1 Hz, 1H), 7.4 (m, 2H), 8.2 (m, 1H); MS m/z 517 (M+1).

### Example 32

### (trans)-1-{[4-(4-Methyl-1-piperazinyl)-1H-benzimidazol-2-yl]methyl}-1,2,3,4,4a,5,6,10b-octahydro-1,10-phenanthroline trifluoroacetate

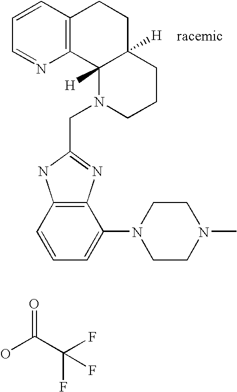

(trans)-1,1-Dimethylethyl 2-(3,4,4a,5,6,10b-hexahydro-1,10-phenanthrolin-1(2H)-ylmethyl)-4-(4-methyl-1-piperazinyl)-1H-benzimidazole-1-carboxylate (0.25 g, 0.48 mmol) was dissolved in 5 mL of dichloromethane and then treated with 5 mL of trifluoroacetic acid. The reaction mixture was stirred at room temperature for 2 hours and then concentrated to dryness. The residue was dried under vacuum to yield 0.32 g (99%) of (trans) 1-{[4-(4-methyl-1-piperazinyl)-1H-benzimidazol-2-yl]methyl}-1,2,3,4,4a,5,6,10b-octahydro-1,10-phenanthroline trifluoroacetate. ¹H NMR (400 MHz, METHANOL-D4) δ ppm 1.6 (td, J=12.6, 4.3 Hz, 1H), 1.7 (m, 2H), 2.1 (m, 2H), 2.3 (m, 2H), 3.1 (m, 5H), 3.2 (t, J=12.5 Hz, 2H), 3.3 (m, 2H), 3.5 (t, J=11.9 Hz, 2 H), 3.7 (m, 2H), 3.9 (t, J=11.1 Hz, 2H), 4.1 (d, J=15.7 Hz, 1H), 4.5 (d, J=11.3 Hz, 1 H), 4.7 (d, J=15.7 Hz, 1H), 7.1 (m, 1H), 7.4 (m, 2H), 7.6 (dd, J=8.0, 5.4 Hz, 1H), 8.1 (d, J=7.9 Hz, 1H), 8.6 (d, J=5.3 Hz, 1H); MS m/z 417 (M+1).

### Example 33

### (cis)-1-{[4-(4-Methyl-1-piperazinyl)-1H-benzimidazol-2-yl]methyl}-1,2,3,4,4a,5,6,10b-octahydro-1,10-phenanthroline

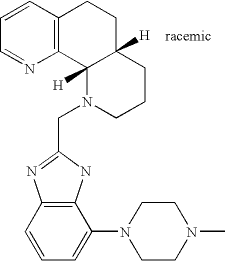

(cis)-1,1-Dimethylethyl 2-(3,4,4a,5,6,10b-hexahydro-1,10-phenanthrolin-1(2H)-ylmethyl)-4-(4-methyl-1-piperazinyl)-1H-benzimidazole-1-carboxylate (0.14 g, 0.28 mmol) was dissolved in 10 mL of dichloromethane and then treated with 10 mL of trifluoroacetic acid. The reaction mixture was stirred at room temperature for 2 hours and then concentrated to dryness. The residue was taken up in 30 mL of saturated sodium bicarbonate and extracted 3 times with 30 mL of dichloromethane. The combined organic layers were washed with 30 mL of saturated sodium chloride and dried over sodium sulfate before being concentrated. The residue was purified by standard reverse phase HPLC methods to yield 0.46 g (80%) of (cis)-1-{[4-(4-methyl-1-piperazinyl)-1H-benzimidazol-2-yl]methyl}-1,2,3,4,4a,5,6,10b-octahydro-1,10-phenanthroline. ¹H NMR (400 MHz, METHANOL-D4) δ ppm 1.5 (m, 2H), 1.8 (d, J=13.2 Hz, 1H), 2.0 (m, 3H), 2.6 (m, 2H), 2.8 (d, J=13.7 Hz, 1H), 3.0 (s, 5H), 3.2 (m, 2H), 3.5 (m, 2H), 3.7 (m, 2H), 3.8 (d, J=13.4 Hz, 2H), 4.5 (d, J=4.6 Hz, 1H), 4.8 (m, 2H), 7.2 (m, 1H), 7.5 (m, 2H), 7.9 (dd, J=7.7, 5.9 Hz, 1H), 8.3 (d, J=7.3 Hz, 1H), 8.8 (d, J=5.5 Hz, 1H); MS m/z 417 (M+1).

### Example 34

### (trans) 5-[2-(3,4,4a,5,6,10b-hexahydro-1,10-phenanthrolin-1(2H)-ylmethyl)-1H-benzimidazol-1-yl]pentanenitrile (intermediate)

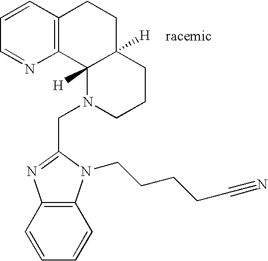

To a solution of (trans) 1-(1H-benzimidazol-2-ylmethyl)-1,2,3,4,4a,5,6,10b-octahydro-1,10-phenanthroline (0.050 g, 0.16 mmol) in 5 mL of N,N-dimethylformamide was added potassium carbonate (0.10 g, 0.72 mmol), 5-bromopentanenitrile (0.039 g, 0.24 mmol), and potassium iodide (10 mg). The reaction mixture was heated to 80° C. in a sealed tube for 16 hours. After being allowed to cool, the mixture was diluted with 10 mL of water and extracted 3 times with 10 mL of dichloromethane. The combined organic layers were concentrated and the residue purified by silica chromatography eluting with a 0% to 10% gradient of 30% aqueous ammonium hydroxide in acetonitrile to yield 0.034 g (53%) of (trans) 5-[2-(3,4,4a,5,6,10b-hexahydro-1,10-phenanthrolin-1(2H)-ylmethyl)-1H-benzimidazol-1-yl]pentanenitrile.

¹H NMR (400 MHz, METHANOL-D4) d ppm 1.4 (m, 3H), 1.6 (m, 1H), 1.7 (m, 2H), 1.9 (m, 1H), 2.0 (m, 3H), 2.2 (m, 2H), 2.6 (m, 2H), 2.9 (m, 4H) 3.7 (d, J=13.0 Hz, 1H) 3.9 (m, 1H), 4.2 (d, J=12.8 Hz, 1H), 5.0 (m, 1H), 7.3 (m, 3H), 7.6 (m, 3H), 8.5 (d, J=3.8 Hz, 1H); MS m/z 400 (M+1).

### Example 35

### (trans)-5-[2-(3,4,4a,5,6,10b-hexahydro-1,10-phenanthrolin-1(2H)-ylmethyl)-1H-benzimidazol-1-yl]-1-pentanamine

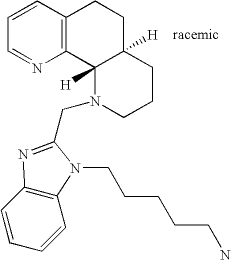

(trans) 5-[2-(3,4,4a,5,6,10b-hexahydro-1,10-phenanthrolin-1(2H)-ylmethyl)-1H-benzimidazol-1-yl]pentanenitrile (0.020 g, 0.050 mmol) was dissolved in a 7M solution of NH₃ in methanol (10 mL). Raney Nickel (˜20 mg) was added and the mixture hydrogenated under 40 psi H₂ for 16 hours. The catalyst was filtered off over celite and the wash concentrated. The residue was dissolved in dichloromethane (10 mL) and filtered through celite. The wash was concentrated and the residue dried under vacuum to yield 0.012 g (66%) of (trans)-5-[2-(3,4,4a,5,6,10b-hexahydro-1,10-phenanthrolin-1(2H)-ylmethyl)-1H-benzimidazol-1-yl]-1-pentanamine. 1H NMR (400 MHz, METHANOL-D4) δ ppm 1.4 (m, 7H), 1.8 (m, 2H), 1.9 (m, 1H), 2.0 (dd, J=12.6, 2.2 Hz, 1H), 2.2 (m, 2H), 2.5 (m, 2H), 2.8 (m, 3 H), 2.9 (m, 1H), 3.6 (d, J=13.0 Hz, 1H), 3.9 (d, J=10.8 Hz, 1H), 4.2 (d, J=13.0 Hz, 1 H), 4.6 (m, 1H), 5.0 (m, 1H), 7.2 (m, 3H), 7.5 (m, 3H), 8.4 (d, J=4.8 Hz, 1H); MS m/z 404 (M+1).

### Example 36

### (trans)-4-[2-(3,4,4a,5,6,10b-hexahydro-1,10-phenanthrolin-1(2H)-ylmethyl)-1H-benzimidazol-1-yl]butanenitrile (intermediate)

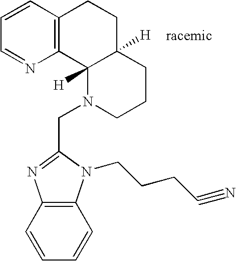

To a solution of (trans)-1-(1H-benzimidazol-2-ylmethyl)-1,2,3,4,4a,5,6,10b-octahydro-1,10-phenanthroline (0.050 g, 0.16 mmol) in 5 mL of N,N-dimethylformamide was added potassium carbonate (0.10 g, 0.72 mmol), 4-bromobutanenitrile (0.035 g, 0.24 mmol), and potassium iodide (10 mg). The reaction mixture was heated to 80° C. in a sealed tube for 16 hours. After being allowed to cool the mixture was diluted with 10 mL of water and extracted 3 times with 10 mL of dichloromethane. The combined organic layers were concentrated and the residue purified by silica chromatography eluting with a 0% to 10% gradient of 30% aqueous ammonium hydroxide in acetonitrile to yield 0.030 g (48%) of (trans)-4-[2-(3,4,4a,5,6,10b-hexahydro-1,10-phenanthrolin-1(2H)-ylmethyl)-1H-benzimidazol-1-yl]butanenitrile. ¹H NMR (400 MHz, CHLOROFORM-D) δ ppm 1.4 (m, 2H), 1.6 (m, 1H), 1.9 (m, 1H), 2.0 (m, 1H), 2.2 (m, 4H), 2.6 (m, 2H), 2.8 (m, 3H), 2.9 (m, 1H), 3.7 (d, J=13.2 Hz, 1H), 3.9 (d, J=10.6 Hz, 1H), 4.2 (d, J=10.7 Hz, 1H), 4.9 (m, 2H), 7.2 (dd, J=7.5, 4.8 Hz, 1H), 7.2 (t, J=7.7 Hz, 1H), 7.3 (t, J=7.2 Hz, 1H), 7.5 (m, 3H), 8.4 (d, J=6.2 Hz, 1H); MS m/z 386 (M+1).

### Example 37

### (trans) 4-[2-(3,4,4a,5,6,10b-hexahydro-1,10-phenanthrolin-1(2H)-ylmethyl)-1H-benzimidazol-1-yl]-1-butanamine

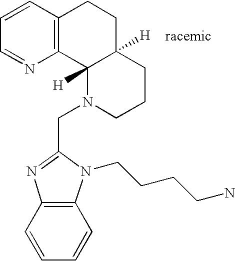

(trans)-4-[2-(3,4,4a,5,6,10b-hexahydro-1,10-phenanthrolin-1(2H)-ylmethyl)-1H-benzimidazol-1-yl]butanenitrile (0.030 g, 0.050 mmol) was dissolved in a 7M solution of NH₃ in methanol (10 mL). Raney Nickel (˜20 mg) was added and the mixture hydrogenated under 40 psi H₂ for 16 hours. The catalyst was filtered off over celite and the wash concentrated. The residue was dissolved in dichloromethane (10 mL) and filtered through celite. The wash was concentrated and the residue dried under vacuum to yield 0.010 g (33%) of (trans) 4-[2-(3,4,4a,5,6,10b-hexahydro-1,10-phenanthrolin-1(2H)-ylmethyl)-1H-benzimidazol-1-yl]-1-butanamine. ¹H NMR (400 MHz, METHANOL-D4) δ ppm 1.4 (m, 2H), 1.5 (m, 3H), 1.8 (m, 3H), 2.0 (m, 1H), 2.2 (m, 2H), 2.7 (t, J=7.6 Hz, 2H), 2.8 (m, 3H), 3.0 (m, 1H), 3.7 (d, J=13.0 Hz, 1H), 3.9 (d, J=11.2 Hz, 1H), 4.2 (d, J=13.0 Hz, 1H), 4.7 (m, 1H), 4.9 (m, 1H), 7.2 (m, 3 H), 7.5 (m, 3H), 8.5 (d, J=4.8 Hz, 1H); MS m/z 390 (M+1).

### Example 38

### (cis) 4-[2-(3,4,4a,5,6,10b-hexahydro-1,10-phenanthrolin-1(2H)-ylmethyl)-1H-benzimidazol-1-yl]-1-butanamine

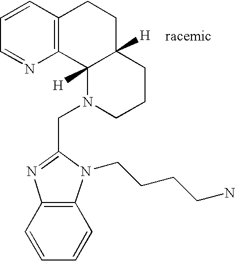

(cis) 1-(1H-Benzimidazol-2-ylmethyl)-1,2,3,4,4a,5,6,10b-octahydro-1,10-phenanthroline (0.045 g, 0.14 mmol), 2-(4-bromobutyl)-1H-isoindole-1,3(2H)-dione (0.059 g, 0.21 mmol), potassium iodide (5 mg), and N,N-diisopropylethylamine (0.050 g, 0.31 mmol) in 3 mL of dimethylformamide were heated to 80° C. for 16 hours. The mixture was allowed to cool to room temperature and hydrazine (0.4 mL, 14.0 mmol) was added. The mixture was stirred at room temperature overnight. The mixture was quenched with water and extracted 3 times with ethyl ether. The ether layers were combined, dried over sodium sulfate, and concentrated. The residue was purified by silica chromatography eluting with a 0% to 10% gradient of 30% aqueous ammonium hydroxide in acetonitrile to yield 0.010 g (18%) of (cis) 4-[2-(3,4,4a,5,6,10b-hexahydro-1,10-phenanthrolin-1(2H)-ylmethyl)-1H-benzimidazol-1-yl]-1-butanamine.

¹H NMR (400 MHz, METHANOL-D4) δ ppm 1.2 (m, 2H), 1.4 (m, 3H), 1.7 (m, 1H), 1.8 (m, 3H), 2.1 (m, 1H), 2.3 (m, 1H), 2.5 (t, J=7.4 Hz, 2H), 2.6 (m, 1H), 2.8 (m, 1 H), 2.9 (m, 1H), 3.1 (m, 1H), 3.5 (d, J=2.7 Hz, 1H), 3.5 (d, J=13.9 Hz, 1H), 3.9 (m, 1H), 4.1 (m, 1H), 4.2 (d, J=13.9 Hz, 1H), 7.2 (m, 2H), 7.3 (m, 1H), 7.4 (d, J=8.6 Hz, 1H), 7.6 (m, 1H), 7.7 (d, J=7.9 Hz, 1H), 8.4 (d, J=6.4 Hz, 1H); MS m/z 390 (M+1).

### Example 39

### (cis) 5-[2-(3,4,4a,5,6,10b-hexahydro-1,10-phenanthrolin-1(2H)-ylmethyl)-1H-benzimidazol-1-yl]-1-pentanamine

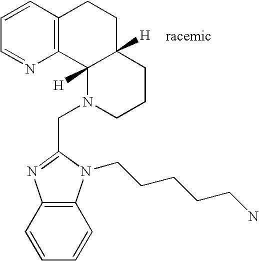

(cis) 1-(1H-Benzimidazol-2-ylmethyl)-1,2,3,4,4a,5,6,10b-octahydro-1,10-phenanthroline (0.045 g, 0.14 mmol), 2-(5-bromopentyl)-1H-isoindole-1,3(2H)-dione (0.062 g, 0.21 mmol), potassium iodide (5 mg), and N,N-diisopropylethylamine (0.050 g, 0.31 mmol) in 3 mL of dimethylformamide were heated to 80° C. for 16 hours. The mixture was allowed to cool to room temperature and hydrazine (0.4 mL, 14.0 mmol) was added. The mixture stirred at room temperature overnight. The mixture was quenched with water and extracted 3 times with ethyl ether. The ether layers were combined, dried over sodium sulfate, and concentrated. The residue was purified by silica chromatography eluting with a 0% to 10% gradient of 30% aqueous ammonium hydroxide in acetonitrile to yield 0.010 g (18%) of (cis)-5-[2-(3,4,4a,5,6,10b-hexahydro-1,10-phenanthrolin-1(2H)-ylmethyl)-1H-benzimidazol-1-yl]-1-pentanamine. ¹H NMR (400 MHz, METHANOL-D4) δ ppm 1.0 (m, 2H), 1.3 (m, 3 H), 1.4 (s, 2H), 1.6 (m, 1H), 1.7 (m, 2H), 2.1 (m, 1H), 2.3 (m, 2H), 2.5 (m, 3H), 2.8 (m, 1H), 2.9 (m, 1H), 3.1 (m, 1H), 3.4 (s, 1H), 3.5 (d, J=13.9 Hz, 1H), 3.9 (m, 1H), 4.1 (m, 1H), 4.2 (d, J=13.9 Hz, 1H), 7.2 (m, 2H), 7.3 (m, 2H), 7.5 (d, J=7.1 Hz, 1 H), 7.7 (d, J=7.9 Hz, 1H), 8.4 (s, 1H); MS m/z 404 (M+1).

### Example 40

### (trans) 3-[2-(3,4,4a,5,6,10b-hexahydro-1,10-phenanthrolin-1(2H)-ylmethyl)-1H-benzimidazol-1-yl]-N,N-dimethyl-1-propanamine

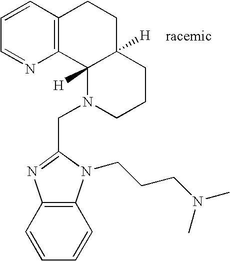

To a solution of (trans) 1-(1H-benzimidazol-2-ylmethyl)-1,2,3,4,4a,5,6,10b-octahydro-1,10-phenanthroline (0.060 g, 0.19 mmol) in 5 mL of N,N-dimethylformamide was added potassium carbonate (0.10 g, 0.72 mmol), (3-chloropropyl)dimethylamine hydrochloride (0.033 g, 0.21 mmol), and potassium iodide (5 mg). The reaction mixture was heated to 80° C. in a sealed tube for 16 hours. After being allowed to cool, the mixture was diluted with 10 mL of water and extracted 3 times with 10 mL of dichloromethane. The combined organic layers were concentrated and the residue purified by silica chromatography eluting with a 0% to 10% gradient of 30% aqueous ammonium hydroxide in acetonitrile to yield 0.048 g (63%) of (trans) 3-[2-(3,4,4a,5,6,10b-hexahydro-1,10-phenanthrolin-1(2H)-ylmethyl)-1H-benzimidazol-1-yl]-N,N-dimethyl-1-propanamine. ¹H NMR (400 MHz, METHANOL-D4) δ ppm 1.4 (m, 2H), 1.5 (m, 1H), 1.9 (m, 1H), 2.0 (m, 3H), 2.2 (m, 8H), 2.3 (m, 2H), 2.8 (m, 3H), 3.0 (m, 1H), 3.7 (d, J=12.8 Hz, 1H), 3.9 (d, J=11.0 Hz, 1H), 4.2 (d, J=13.0 Hz, 1H), 4.7 (ddd, J=14.1, 7.0, 6.9 Hz, 1H), 5.0 (dt, J=14.5, 7.3 Hz, 1H), 7.2 (m, 3H), 7.5 (m, 3H), 8.5 (d, J=4.6 Hz, 1H); MS m/z 404 (M+1).

### Example 41

### (cis) 3-[2-(3,4,4a,5,6,10b-hexahydro-1,10-phenanthrolin-1(2H)-ylmethyl)-1H-benzimidazol-1-yl]-N,N-dimethyl-1-propanamine

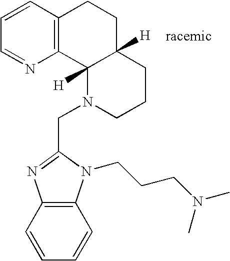

To a solution of (cis) 1-(1H-benzimidazol-2-ylmethyl)-1,2,3,4,4a,5,6,10b-octahydro-1,10-phenanthroline (0.054 g, 0.17 mmol) in 5 mL of N,N-dimethylformamide was added potassium carbonate (0.094 g, 0.68 mmol), (3-chloropropyl)dimethylamine hydrochloride (0.030 g, 0.19 mmol), and potassium iodide (5 mg). The reaction mixture was heated to 80° C. in a sealed tube for 16 hours. After being allowed to cool, the mixture was diluted with 10 mL of water and extracted 3 times with 10 mL of dichloromethane. The combined organic layers were concentrated and the residue purified by silica chromatography eluting with a 0% to 10% gradient of 30% aqueous ammonium hydroxide in acetonitrile to yield 0.036 g (52%) of (cis) 3-[2-(3,4,4a,5,6,10b-hexahydro-1,10-phenanthrolin-1(2H)-ylmethyl)-1H-benzimidazol-1-yl]-N,N-dimethyl-1-propanamine. ¹H NMR (400 MHz, METHANOL-D4) δ ppm 1.1 (m, 1H), 1.5 (m, 1H), 1.6 (m, 3H), 1.8 (m, 3H), 1.9 (m, 1H), 2.0 (m, 1H), 2.1 (s, 6H), 2.3 (t, J=11.4 Hz, 1H), 2.6 (m, 1H), 2.8 (d, J=11.9 Hz, 1H), 2.9 (m, 1H), 3.1 (m, 1 H), 3.5 (d, J=2.9 Hz, 1H), 3.5 (d, J=14.1 Hz, 1H), 4.0 (m, 1H), 4.2 (m, 2H), 7.2 (m, 2H), 7.3 (dd, J=7.7, 4.9 Hz, 1H), 7.4 (d, J=8.1 Hz, 1H), 7.5 (d, J=6.8 Hz, 1H), 7.7 (d, J=7.1 Hz, 1H), 8.4 (d, 1H); MS m/z 404 (M+1).

### Example 42

### (trans) 1-({1-[(1-methyl-3-piperidinyl)methyl]-1H-benzimidazol-2-yl}methyl)-1,2,3,4,4a,5,6,10b-octahydro-1,10-phenanthroline

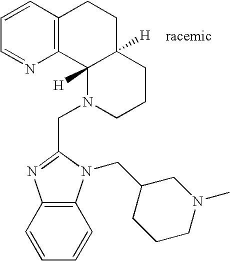

To a solution of (trans) 1-(1H-benzimidazol-2-ylmethyl)-1,2,3,4,4a,5,6,10b-octahydro-1,10-phenanthroline (0.027 g, 0.19 mmol) in 5 mL of N,N-dimethylformamide was added potassium carbonate (0.047 g, 0.34 mmol), 3-(chloromethyl)-1-methylpiperidine hydrochloride (0.023 g, 0.13 mmol), and potassium iodide (2 mg). The reaction mixture was heated to 80° C. in a sealed tube for 16 hours. The mixture was then transferred to a microwave reactor and heated at 120° C. for 60 minutes. After being allowed to cool, the mixture was diluted with 10 mL of water and extracted 3 times with 10 mL of dichloromethane. The combined organic layers were concentrated and the residue purified by silica chromatography eluting with a 0% to 10% gradient of 30% aqueous ammonium hydroxide in acetonitrile to yield 0.015 g (39%) of (trans) 1-({1-[(1-methyl-3-piperidinyl)methyl]-1H-benzimidazol-2-yl}methyl)-1,2,3,4,4a,5,6,10b-octahydro-1,10-phenanthroline. ¹H NMR (400 MHz, METHANOL-D4) δ ppm 1.4 (m, 2H), 1.5 (m, 2H), 1.7 (m, 1H), 1.8 (m, 1H), 2.0 (m, 5H), 2.3 (m, 5H), 2.8 (m, 3H), 3.0 (m, 2H), 3.4 (m, 1H), 3.7 (d, J=13.0 Hz, 1H), 3.9 (m, 1H), 4.2 (m, 1H), 4.4 (m, 1H), 4.6 (m, 1H), 4.9 (m, 1H), 7.3 (s, 3H), 7.5 (m, 3H), 8.5 (s, 1 H); MS m/z 430 (M+1).

### Example 43

### (cis) 1-({1-[3-(1-piperidinyl)propyl]-1H-benzimidazol-2-yl}methyl)-1,2,3,4,4a,5,6,10b-octahydro-1,10-phenanthroline

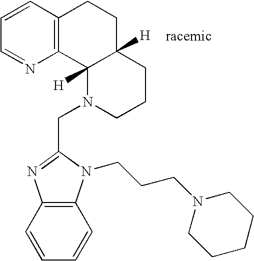

To a solution of (cis) 1-(1H-benzimidazol-2-ylmethyl)-1,2,3,4,4a,5,6,10b-octahydro-1,10-phenanthroline (0.032 g, 0.10 mmol) in 5 mL of N,N-dimethylformamide was added potassium carbonate (0.14 g, 1.0 mmol), 1-(3-chloropropyl)piperidine hydrochloride (0.050 g, 0.25 mmol), and potassium iodide (10 mg). The reaction mixture was heated to 80° C. in a sealed tube for 16 hours. After being allowed to cool, the mixture was diluted with 10 mL of water and extracted 3 times with 10 mL of dichloromethane. The combined organic layers were concentrated and the residue purified by silica chromatography eluting with a 0% to 10% gradient of 30% aqueous ammonium hydroxide in acetonitrile to yield 0.028 g (63%) of (cis) 1-({1-[3-(1-piperidinyl)propyl]-1H-benzimidazol-2-yl}methyl)-1,2,3,4,4a,5,6,10b-octahydro-1,10-phenanthroline. ¹H NMR (400 MHz, METHANOL-D4) δ ppm 1.5 (m, 6H), 1.6 (m, 4 H), 1.8 (m, 2H), 1.9 (m, 2H), 2.1 (m, 1H), 2.3 (m, 5H), 2.5 (m, 2H), 2.8 (d, J=11.7 Hz, 1H), 2.9 (m, 1H), 3.1 (ddd, J=17.5, 7.2, 2.7 Hz, 1H), 3.4 (m, 1H), 3.6 (m, 1H), 4.0 (ddd, J=14.3, 7.1, 7.0 Hz, 1H), 4.2 (m, 2H), 7.2 (m, 2H), 7.3 (m, 1H), 7.4 (d, J=7.0 Hz, 1H), 7.5 (d, J=7.3 Hz, 1H), 7.7 (d, J=7.5 Hz, 1H), 8.4 (d, J=4.8 Hz, 1H); MS m/z 444 (M+1).

### Example 44

### (cis) 1-({1-[3-(4-morpholinyl)propyl]-1H-benzimidazol-2-yl}methyl)-1,2,3,4,4a,5,6,10b-octahydro-1,10-phenanthroline

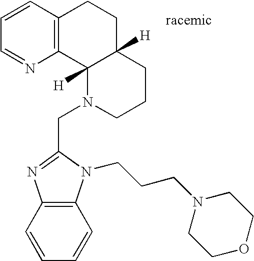

To a solution of (cis) 1-(1H-benzimidazol-2-ylmethyl)-1,2,3,4,4a,5,6,10b-octahydro-1,10-phenanthroline (0.032 g, 0.10 mmol) in 5 mL of N,N-dimethylformamide was added potassium carbonate (0.14 g, 1.0 mmol), 4-(3-chloropropyl)morpholine hydrochloride (0.050 g, 0.25 mmol), and potassium iodide (10 mg). The reaction mixture was heated to 80° C. in a sealed tube for 16 hours. After being allowed to cool, the mixture was diluted with 10 mL of water and extracted 3 times with 10 mL of dichloromethane. The combined organic layers were concentrated and the residue purified by silica chromatography eluting with a 0% to 10% gradient of 30% aqueous ammonium hydroxide in acetonitrile to yield 0.025 g (56%) of (cis) 1-({1-[3-(4-morpholinyl)propyl]-1H-benzimidazol-2-yl}methyl)-1,2,3,4,4a,5,6,10b-octahydro-1,10-phenanthroline. ¹H NMR (400 MHz, METHANOL-D4) δ ppm 1.5 (m, 2H), 1.7 (m, 4 H), 1.9 (t, J=7.2 Hz, 2H), 2.1 (m, 1H), 2.2 (m, 2H), 2.3 (m, 1H), 2.4 (m, 1H), 2.4 (m, 1H), 2.6 (m, 1H), 2.8 (d, J=12.1 Hz, 1H), 2.9 (m, 1H), 3.1 (m, 1H), 3.5 (m, 5 H), 3.7 (m, 2H), 4.0 (m, 1H), 4.2 (m, 2H), 7.2 (m, 2H), 7.3 (dd, J=7.9, 4.8 Hz, 1H), 7.4 (d, J=6.8 Hz, 1H), 7.6 (m, 1H), 7.7 (d, J=9.0 Hz, 1H), 8.4 (d, J=6.4 Hz, 1H); MS m/z 446 (M+1).

### Example 45

### (cis) 1-{[1-(2-pyridinylmethyl)-1H-benzimidazol-2-yl]methyl}-1,2,3,4,4a,5,6,10b-octahydro-1,10-phenanthroline

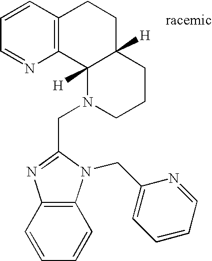

To a solution of (cis) 1-(1H-benzimidazol-2-ylmethyl)-1,2,3,4,4a,5,6,10b-octahydro-1,10-phenanthroline (0.032 g, 0.10 mmol) in 5 mL of N,N-dimethylformamide was added potassium carbonate (0.14 g, 1.0 mmol), 2-(bromomethyl)pyridine hydrobromide (0.063 g, 0.25 mmol), and potassium iodide (10 mg). The reaction mixture was heated to 80° C. in a sealed tube for 16 hours. After being allowed to cool, the mixture was diluted with 10 mL of water and extracted 3 times with 10 mL of dichloromethane. The combined organic layers were concentrated and the residue purified by silica chromatography eluting with a 0% to 10% gradient of 30% aqueous ammonium hydroxide in acetonitrile to yield 0.010 g (24%) of (cis) 1-{[1-(2-pyridinylmethyl)-1H-benzimidazol-2-yl]methyl}-1,2,3,4,4a,5,6,10b-octahydro-1,10-phenanthroline. ¹H NMR (400 MHz, METHANOL-D4) δ ppm 1.4 (m, 2H), 1.6 (m, 2 H), 1.7 (m, 1H), 1.9 (m, 1H), 2.1 (m, 1H), 2.3 (m, 1H), 2.8 (m, 1H), 2.9 (m, 2H), 3.4 (m, 1H), 3.5 (d, J=13.9 Hz, 1H), 4.3 (d, J=14.1 Hz, 1H), 5.1 (d, J=17.4 Hz, 1H), 5.4 (d, J=17.4 Hz, 1H), 6.5 (d, J=7.9 Hz, 1H), 7.1 (m, 2H), 7.2 (m, 3H), 7.5 (d, J=7.7 Hz, 1H), 7.6 (m, 2H), 8.2 (d, J=3.1 Hz, 1H), 8.4 (d, J=4.9 Hz, 1H); MS m/z 410 (M+1).

### Example 46

### (trans) 1-[(1-{[1-(phenylmethyl)-1H-imidazol-2-yl]methyl}-1H-benzimidazol-2-yl)methyl]-1,2,3,4,4a,5,6,10b-octahydro-1,10-phenanthroline

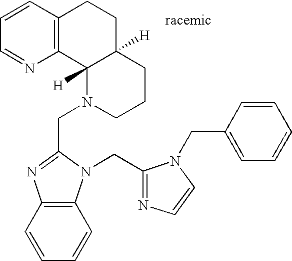

To a solution of (trans) 1-(1H-benzimidazol-2-ylmethyl)-1,2,3,4,4a,5,6,10b-octahydro-1,10-phenanthroline (0.038 g, 0.12 mmol) in 5 mL of N,N-dimethylformamide was added potassium carbonate (0.17 g, 1.2 mmol), 2-(chloromethyl)-1-(phenylmethyl)-1H-imidazole hydrochloride (0.044 g, 0.18 mmol), and potassium iodide (5 mg). The reaction mixture was heated to 80° C. in a sealed tube for 16 hours. After being allowed to cool, the mixture was diluted with 10 mL of water and extracted 3 times with 10 mL of dichloromethane. The combined organic layers were concentrated and the residue purified by silica chromatography eluting with a 0% to 10% gradient of 30% aqueous ammonium hydroxide in acetonitrile to yield 0.043 g (73%) of (trans) 1-[(1-{[1-(phenylmethyl)-1H-imidazol-2-yl]methyl}-1H-benzimidazol-2-yl)methyl]-1,2,3,4,4a,5,6,10b-octahydro-1,10-phenanthroline. ¹H NMR (400 MHz, METHANOL-D4) δ ppm 1.3 (m, 2H), 1.5 (m, 1H), 1.8 (m, 1H), 2.0 (m, 1H), 2.1 (m, 1H), 2.7 (m, 1H), 2.8 (m, 2H), 2.9 (m, 1H), 3.7 (d, J=13.6 Hz, 1H), 3.8 (d, J=11.0 Hz, 1H), 4.2 (d, J=13.4 Hz, 1H), 5.2 (d, J=15.9 Hz, 1H), 5.3 (s, 1H), 5.4 (d, J=15.9 Hz, 1H), 6.3 (m, 2H), 6.9 (m, 2H), 7.0 (dd, J=7.8, 1.7 Hz, 2H), 7.1 (m, 2H), 7.2 (m, 4H), 7.3 (m, 1H), 7.5 (d, J=7.7 Hz, 1H), 7.5 (d, J=8.1 Hz, 1H), 8.0 (d, J=4.6 Hz, 1H); MS m/z 489 (M+1).

### Example 47

### Phenylmethyl (4aR,10bS)-3,4,4a,5,6,10b-hexahydro-1,10-phenanthrolin-1(2H)-ylacetate (intermediate)

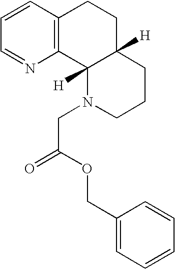

To a solution of (4aR,10bS)-1,2,3,4,4a,5,6,10b-octahydro-1,10-phenanthroline (0.73 g, 3.9 mmol) in 20 mL of acetonitrile was added potassium carbonate (2.7 g, 19 mmol), phenylmethyl bromoacetate (0.98 g, 4.3 mmol), and 10 mg of potassium iodide. The reaction mixture was stirred for 16 hours at room temperature and diluted with 50 mL of dichloromethane. The organic phase was washed with 20 mL of water and then 20 mL of brine. The organic phase was dried over sodium sulfate and concentrated. The residue was purified by silica chromatography eluting with a 0% to 10% gradient of 2M ammonia in methanol and dichloromethane as co-solvent to yield 0.88 g (67%) of phenylmethyl (4aR,10bS)-3,4,4a,5,6,10b-hexahydro-1,10 phenanthrolin-1(2H)-ylacetate. ¹H NMR (300 MHz, METHANOL-D4) δ ppm 1.6 (m, 2 H), 1.8 (m, 2H), 2.0 (m, 2H), 2.6 (m, 1H), 2.8 (m, 2H), 3.0 (m, 1H), 3.1 (m, 1H), 3.1 (d, J=17.3 Hz, 1H), 3.6 (m, 2H), 5.0 (d, J=3.8 Hz, 2H), 7.2 (dd, J=7.7, 4.8 Hz, 1 H), 7.4 (m, 5H), 7.6 (d, J=7.7 Hz, 1H), 8.2 (dd, J=5.3, 1.3 Hz, 1H); MS m/z 337 (M+1).

### Example 48

### (4aR,10bS)-1-{[4-(4-methyl-1-piperazinyl)-1H-benzimidazol-2-yl]methyl}-1,2,3,4,4a,5,6,10b-octahydro-1,10-phenanthroline

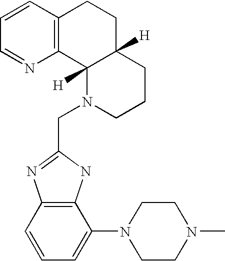

A mixture of phenylmethyl (4aR,10bS)-3,4,4a,5,6,10b-hexahydro-1,10-phenanthrolin-1(2H)-ylacetate (0.884 g, 2.6 mmol) and 10% palladium on carbon (10 mg) in 25 mL of methanol were hydrogenated under 50 psi of hydrogen for 2 hours. The catalyst was filtered off over celite and the wash concentrated. The residue was dissolved in 50 mL of acetonitrile. 3-(4-methyl-1-piperazinyl)-1,2-benzenediamine (0.53 g, 2.6 mmol) and N,N-diisopropylethylamine (1.0 g, 7.8 mmol) were added followed by portionwise addition of bis(2-oxo-3-oxazolidinyl)phosphinic chloride (0.99 g, 3.9 mmol). The mixture was stirred for 3 hours and concentrated. The residue was dissolved in dichloromethane and washed with saturated sodium bicarbonate. The organic phase was washed with brine, dried over sodium sulfate, and concentrated. The residue was dissolved in 50 mL of acetic acid and heated to 70° C. for 1 hour, and then stirred at room temperature overnight. The mixture was concentrated and the residue diluted with saturated sodium bicarbonate. The aqueous mixture was extracted 3 times with dichloromethane. The organic layers were combined and concentrated. The residue was purified by silica chromatography eluting with a 0% to 10% gradient of 30% aqueous ammonium hydroxide in acetonitrile to yield 0.61 (56%) of (4aR,10bS)-1-{[4-(4-methyl-1-piperazinyl)-1H-benzimidazol-2-yl]methyl}-1,2,3,4,4a,5,6,10b-octahydro-1,10-phenanthroline. ¹H NMR (400 MHz, METHANOL-D4) δ ppm 1.5 (m, 2H), 1.8 (m, 1H), 2.0 (m, 3H), 2.6 (m, 2H), 2.8 (d, J=14.6 Hz, 1 H), 3.0 (m, 5H), 3.2 (s, 2H), 3.5 (t, J=12.0 Hz, 2H), 3.7 (m, 4H), 4.5 (d, J=4.9 Hz, 1 H), 4.8 (m, 2H), 7.2 (m, 2H), 7.5 (m, 2H), 7.9 (dd, J=8.0, 5.8 Hz, 1H), 8.3 (d, J=7.9 Hz, 1H), 8.8 (d, J=5.9 Hz, 1H); MS m/z 417 (M+1).

### Example 49

### (4aR,10bS)-1-{[1-(triphenylmethyl)-1H-imidazol-4-yl]methyl}-1,2,3,4,4a,5,6,10b-octahydro-1,10-phenanthroline (intermediate)

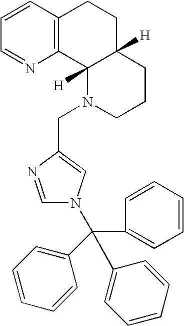

To a solution of (4aR,10bS)-1,2,3,4,4a,5,6,10b-octahydro-1,10-phenanthroline (0.25 g, 1.3 mmol), 1-(triphenylmethyl)-1H-imidazole-4-carbaldehyde (similar methods as described in J. Org. Chem. 2002, 67, 620-624; 0.89 g, 2.6 mmol), and acetic acid (0.078 g, 1.3 mmol) in 5 mL of 1,2-dichloroethane was added sodium triacetoxyborohydride (0.82 g, 3.9 mmol). The mixture was stirred at room temperature for 16 hours. The mixture was diluted with water and extracted 3 times with 10 mL of dichloromethane. The organic phase was concentrated and the residue purified by silica chromatography eluting with a 0% to 5% gradient of 2 M ammonia in methanol with dichloromethane as co-eluent to yield 0.27 g (41%) of (4aR,10bS)-1-{[1-(triphenylmethyl)-1H-imidazol-4-yl]methyl}-1,2,3,4,4a,5,6,10b-octahydro-1,10-phenanthroline. ¹H NMR (400 MHz, METHANOL-D4) δ ppm 1.6 (m, 5H), 2.0 (m, 1H), 2.3 (m, 2H), 2.7 (m, 1H), 2.9 (m, 1H), 3.0 (m, 1H), 3.4 (m, 2H), 3.8 (m, 1H), 6.8 (s, 1H), 7.1 (m, 6H), 7.2 (dd, J=7.7, 4.6 Hz, 1H), 7.3 (s, 1H), 7.3 (m, 9H), 7.5 (d, J=8.1 Hz, 1H), 8.2 (d, J=4.4 Hz, 1H); MS m/z 511 (M+1).

### Example 50

### (4aR,10bS)-1-(1H-imidazol-4-ylmethyl)-1,2,3,4,4a,5,6,10b-octahydro-1,10-phenanthroline (intermediate)

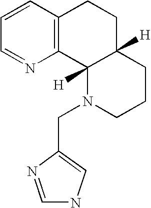

(4aR,10bS)-1-{[1-(triphenylmethyl)-1H-imidazol-4-yl]methyl}-1,2,3,4,4a,5,6,10b-octahydro-1,10-phenanthroline (0.27 g, 0.54 mmol) was dissolved in a 2 mL of dichloromethane and 2 mL of trifluoroacetic acid. The mixture was stirred for 2 hours and concentrated. The residue was dissolved in saturated sodium bicarbonate and extracted 3 times with 10 mL of dichloromethane. The organic phase was concentrated and the residue purified by silica chromatography eluting with a 0% to 10% gradient of 2 M ammonia in methanol with dichloromethane as co-eluent to yield 0.090 g (62%) of (4aR,10bS)-1-(1H-imidazol-4-ylmethyl)-1,2,3,4,4a,5,6,10b-octahydro-1,10-phenanthroline. ¹H NMR (400 MHz, METHANOL-D4) δ ppm 1.6 (m, 1H), 1.7 (m, 3H), 1.7 (m, 1H), 2.1 (m, 1H), 2.3 (m, 1H), 2.4 (m, 1H), 2.8 (m, 2H), 3.1 (m, 1H), 3.5 (d, J=3.1 Hz, 1H), 3.6 (m, 1H), 3.7 (m, 1H), 6.8 (s, 1H), 7.3 (dd, J=7.7, 4.9 Hz, 1H), 7.6 (d, J=6.2 Hz, 2H), 8.4 (d, J=5.1 Hz, 1H); MS m/z 269 (M+1).

### Example 51a

### (3-{4-[(4aR,10bS)-3,4,4a,5,6,10b-hexahydro-1,10-phenanthrolin-1(2H)-ylmethyl]-1H-imidazol-1-yl}propyl)dimethylamine and;

### Example 51b

### (3-{5-[(4aR,10bS)-3,4,4a,5,6,10b-hexahydro-1,10-phenanthrolin-1(2H)-ylmethyl]-1H-imidazol-1-yl}propyl)dimethylamine

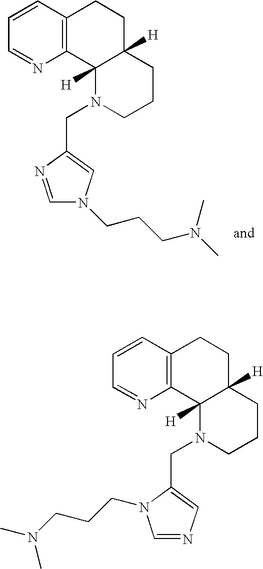

A mixture of (4aR,10bS)-1-(1H-imidazol-4-ylmethyl)-1,2,3,4,4a,5,6,10b-octahydro-1,10-phenanthroline (0.050 g, 0.19 mmol), 3-(dimethylamino)propyl chloride hydrochloride (0.027 g, 0.22 mmol), potassium carbonate (0.26 g, 1.9 mmol), and potassium iodide (7.7 mg, 0.047 mmol) in 5 mL of acetonitrile was heated to 75° C. in a sealed tube for 4 hours. The mixture was allowed to cool and the solids filtered off. The wash was concentrated and the residue purified by silica chromatography eluting with a 0% to 10% gradient of 30% aqueous ammonium hydroxide in acetonitrile. Appropriate fractions were concentrated to give 12 mg (18%) of (3-{4-[(4aR,10bS)-3,4,4a,5,6,10b-hexahydro-1,10-phenanthrolin-1(2H)-ylmethyl]-1H-imidazol-1-yl}propyl)dimethylamine and 6 mg (9%) of (3-{5-[(4aR,10bS)-3,4,4a,5,6,10b-hexahydro-1,10-phenanthrolin-1(2H)-ylmethyl]-1H-imidazol-1-yl}propyl)dimethylamine. 51a: ¹H NMR (400 MHz, METHANOL-D4) δ ppm 1.6 (m, 1 H), 1.7 (m, 4H), 1.8 (m, 1H), 1.9 (qd, J=7.3, 7.0 Hz, 2H), 2.1 (m, 1H), 2.3 (m, 8H), 2.4 (m, 1H), 2.8 (dt, J=16.9, 7.2 Hz, 1H), 2.9 (td, J=8.1, 4.0 Hz, 1H), 3.1 (dt, J=17.0, 6.8 Hz, 1H), 3.4 (d, J=12.8 Hz, 1H), 3.5 (s, 1H), 3.7 (d, J=13.7 Hz, 1H), 4.0 (t, J=7.0 Hz, 2H), 7.0 (s, 1H), 7.2 (dd, J=7.7, 4.8 Hz, 1H), 7.5 (s, 1H), 7.6 (d, J=7.9 Hz, 1H), 8.3 (d, J=5.7 Hz, 1H); MS m/z 377 (M+Na⁺). 51b: ¹H NMR (400 MHz, METHANOL-D4) δ ppm 1.5 (m, 3H), 1.6 (m, 1H), 1.8 (m, 3H), 2.1 (m, 3H), 2.2 (m, 1H), 2.2 (s, 6H), 2.5 (m, 1H), 2.9 (m, 2H), 3.1 (m, 1H), 3.2 (d, J=14.1 Hz, 1H), 3.4 (d, J=2.6 Hz, 1H), 3.8 (m, 3H), 6.7 (s, 1H), 7.3 (dd, J=7.8, 4.9 Hz, 1H), 7.5 (s, 1H), 7.7 (d, J=7.7 Hz, 1H), 8.4 (d, J=3.1 Hz, 1H); MS m/z 354 (M+1).

### Example 52

### (cis) 1-{[5-(4-morpholinyl)imidazo[1,2-a]pyridin-2-yl]methyl}-1,2,3,4,4a,5,6,10b-octahydro-1,10-phenanthroline

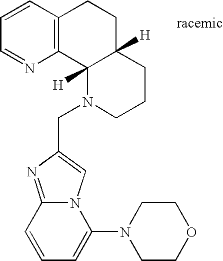

(cis) 1-[(5-Fluoroimidazo[1,2-a]pyridin-2-yl)methyl]-1,2,3,4,4a,5,6,10b-octahydro-1,10-phenanthroline (0.036 g, 0.11 mmol) was dissolved in 0.5 mL morpholine and heated to 200° C. in a microwave reactor for 20 minutes. The reaction was allowed to cool and diluted with 5 mL of brine. The mixture was extracted 3 times with 5 mL of dichloromethane. The combined organic layers were washed 3 times with 5 mL of water, dried over sodium sulfate, and concentrated. The residue purified by silica chromatography eluting with a 0% to 10% gradient of 30% aqueous ammonium hydroxide in acetonitrile to yield 0.017 g (38%) of (cis) 1-{[5-(4-morpholinyl)imidazo[1,2-a]pyridin-2-yl]methyl}-1,2,3,4,4a,5,6,10b-octahydro-1,10-phenanthroline. ¹H NMR (400 MHz, METHANOL-D4) δ ppm 1.7 (m, 6H), 2.1 (m, 1 H), 2.4 (m, 2H), 2.8 (m, 1H), 2.9 (m, 1H), 3.1 (m, 4H), 3.6 (d, J=3.1 Hz, 1H), 3.6 (d, J=14.5 Hz, 1H), 3.9 (m, 4H), 4.0 (d, J=14.3 Hz, 1H), 6.4 (d, J=8.2 Hz, 1H), 7.2 (m, 2H), 7.2 (m, 1H), 7.5 (d, J=9.1 Hz, 1H), 7.6 (s, 1H), 8.3 (d, J=4.9 Hz, 1H); MS m/z 404 (M+1).

### Example 53

### (4aR,10bR)-1-{[5-(4-methyl-1-piperazinyl)imidazo[1,2-a]pyridin-2-yl]methyl}-1,2,3,4,4a,5,6,10b-octahydro-1,10-phenanthroline

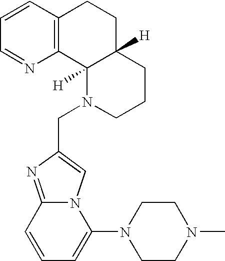

(4aR,10bR)-1-[(5-Fluoroimidazo[1,2-a]pyridin-2-yl)methyl]-1,2,3,4,4a,5,6,10b-octahydro-1,10-phenanthroline (1.8 g, 0.32 mmol) was dissolved in 5 mL 1-methylpiperazine and heated to 200° C. in a microwave reactor for 20 minutes. The reaction was allowed to cool and diluted with 10 mL of brine. The mixture was extracted 3 times with 10 mL of dichloromethane. The combined organic layers were washed with 10 mL of water, dried over sodium sulfate, and concentrated. The residue was slurried in 50 mL of acetonitrile. The resulting solid, (4aR,10bR)-1-{[5-(4-methyl-1-piperazinyl)imidazo[1,2-a]pyridin-2-yl]methyl}-1,2,3,4,4a,5,6,10b-octahydro-1,10-phenanthroline (1.4 g), was collected by filtration. The filtrate was concentrated and the residue purified by silica chromatography eluting with a 0% to 10% gradient of 30% aqueous ammonium hydroxide in acetonitrile to yield 165 mg (7%) of (4aR,10bR)-1-{[5-(4-methyl-1-piperazinyl)imidazo[1,2-a]pyridin-2-yl]methyl}-1,2,3,4,4a,5,6,10b-octahydro-1,10-phenanthroline. ¹H NMR (400 MHz, METHANOL-D4) δ ppm 1.4 (m, 2H), 1.5 (qd, J=12.5, 5.3 Hz, 1H), 1.8 (m, 1H), 2.0 (m, 1H), 2.0 (m, 1H), 2.1 (m, 1H), 2.4 (s, 3H), 2.8 (m, 5H), 2.9 (m, 2H), 3.2 (m, 5H), 3.5 (d, J=14.1 Hz, 1H), 3.9 (d, J=11.0 Hz, 1H), 4.0 (d, J=14.3 Hz, 1H), 6.4 (d, J=7.1 Hz, 1 H), 7.2 (m, 3H), 7.5 (d, J=7.7 Hz, 1H), 7.9 (s, 1H), 8.4 (d, J=6.0 Hz, 1H); MS m/z 417 (M+1).

### Example 54

### [2-[(4aR,10bS)-3,4,4a,5,6,10b-hexahydro-1,10-phenanthrolin-1(2H)-ylmethyl]-5-(4-methyl-1-piperazinyl)imidazo[1,2-a]pyridin-3-yl]methanol

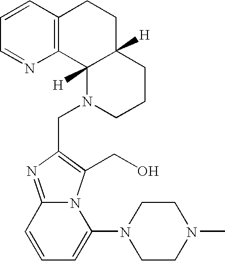

A solution of (4aR,10bS)-1-{[5-(4-methyl-1-piperazinyl)imidazo[1,2-a]pyridin-2-yl]methyl}-1,2,3,4,4a,5,6,10b-octahydro-1,10-phenanthroline (0.082 g, 0.20 mmol) and acetic acid (0.25 mL, 0.20 mmol) in 1 mL of 37% aqueous formaldehyde was heated to 50° C. for 16 hours. The mixture was allowed to cool to room temperature, was made basic with 10 mL of saturated sodium bicarbonate, and was extracted 3 times with 5 mL of dichloromethane. The organic layers were combined and concentrated. The residue was purified by silica chromatography eluting with a 0% to 10% gradient of 30% aqueous ammonium hydroxide in acetonitrile to yield 70 mg (80%) of [2-[(4aR,10bS)-3,4,4a,5,6,10b-hexahydro-1,10-phenanthrolin-1(2H)-ylmethyl]-5-(4-methyl-1-piperazinyl)imidazo[1,2-a]pyridin-3-yl]methanol. ¹H NMR (400 MHz, METHANOL-D4) δ ppm 1.6 (m, 2H), 1.7 (m, 3H), 2.2 (m, 1H), 2.3 (m, 1 H), 2.4 (s, 3H), 2.5 (m, 3H), 2.7 (m, 1H), 2.8 (dt, J=17.3, 6.4 Hz, 1H), 2.9 (m, 4H), 3.1 (m, 1H), 3.3 (m, 2H), 3.6 (d, J=3.5 Hz, 1H), 3.8 (d, J=13.5 Hz, 1H), 4.2 (d, J=13.5 Hz, 1H), 4.9 (d, J=13.2 Hz, 1H), 5.2 (d, J=13.2 Hz, 1H), 6.7 (d, J=6.2 Hz, 1 H), 7.2 (m, 1H), 7.3 (m, 2H), 7.6 (d, J=7.7 Hz, 1H), 8.3 (d, J=4.8 Hz, 1H); MS m/z 429 (M+1).

### Example 55

### N-ethyl-N-{[2-[(4aR,10bS)-3,4,4a,5,6,10b-hexahydro-1,10-phenanthrolin-1(2H)-ylmethyl]-5-(4-methyl-1-piperazinyl)imidazo[1,2-a]pyridin-3-yl]methyl}ethanamine

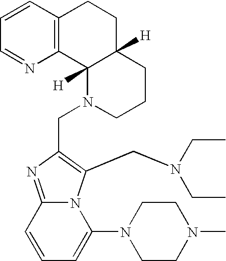

(4aR,10bS)-1-{[5-(4-methyl-1-piperazinyl)imidazo[1,2-a]pyridin-2-yl]methyl}-1,2,3,4,4a,5,6,10b-octahydro-1,10-phenanthroline (0.050 g, 0.12 mmol), diethylamine (0.12 mL, 1.2 mmol), 0.1 mL of acetic acid, and 0.10 mL (1.2 mmol) of a 37% formaldehyde solution in water were heated to 50° C. for 2 hours. The mixture was allowed to cool and was quenched with 10 mL of saturated sodium bicarbonate. The mixture was extracted three times with 5 mL of dichloromethane and the combined organic layers concentrated. The residue was purified by silica chromatography eluting with a 0% to 10% gradient of 30% aqueous ammonium hydroxide in acetonitrile to yield 45 mg (75%) of N-ethyl-N-{[2-[(4aR,10bS)-3,4,4a,5,6,10b-hexahydro-1,10-phenanthrolin-1(2H)-ylmethyl]-5-(4-methyl-1-piperazinyl)imidazo[1,2-a]pyridin-3-yl]methyl}ethanamine. ¹H NMR (400 MHz, METHANOL-D4) δ ppm 0.8 (m, 6H), 1.5 (m, 1H), 1.6 (m, 1H), 1.8 (m, 3H), 2.0 (m, 1H), 2.2 (m, 3H), 2.4 (s, 5 H), 2.5 (m, 3H), 2.7 (m, 1H), 2.9 (m, 4H), 3.0 (m, 1H), 3.1 (m, 3H), 3.4 (m, 2H), 3.9 (m, 1H), 4.0 (d, J=13.7 Hz, 2H), 6.7 (m, 1H), 7.3 (m, 3H), 7.6 (d, J=7.7 Hz, 1 H), 8.3 (d, J=4.2 Hz, 1H); m/z 524 (M+Na⁺).

### Example 56

### (4aR,10bS)-1-({5-(4-methyl-1-piperazinyl)-3-[(4-methyl-1-piperazinyl)methyl]imidazo[1,2-a]pyridin-2-yl}methyl)-1,2,3,4,4a,5,6,10b-octahydro-1,10-phenanthroline

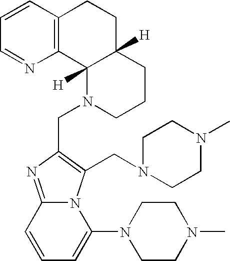

(4aR,10bS)-1-{[5-(4-methyl-1-piperazinyl)imidazo[1,2-a]pyridin-2-yl]methyl}-1,2,3,4,4a,5,6,10b-octahydro-1,10-phenanthroline (0.050 g, 0.12 mmol), 1-methylpiperazine (0.13 mL, 1.2 mmol), 0.1 mL of acetic acid, and 0.10 mL (1.2 mmol) of a 37% formaldehyde solution in water were heated to 50° C. for 2 hours. The mixture was allowed to cool and was quenched with 10 mL of saturated sodium bicarbonate. The mixture was extracted three times with 5 mL of dichloromethane and the combined organic layers concentrated. The residue was purified by silica chromatography eluting with a 0% to 10% gradient of 30% aqueous ammonium hydroxide in acetonitrile to yield 55 mg (87%) of (4aR,10bS)-1-({5-(4-methyl-1-piperazinyl)-3-[(4-methyl-1-piperazinyl)methyl]imidazo[1,2-a]pyridin-2-yl}methyl)-1,2,3,4,4a,5,6,10b-octahydro-1,10-phenanthroline. 1H NMR (400 MHz, METHANOL-D4) δ ppm 1.5 (m, 1H), 1.6 (m, 1H), 1.8 (m, 3H), 2.0 (m, 2H), 2.2 (m, 9H), 2.4 (m, 4H), 2.5 (m, 4H), 2.7 (m, 1H), 2.9 (m, 4H), 3.1 (m, 2H), 3.2 (m, 2H), 3.4 (d, J=13.4 Hz, 1H), 3.4 (s, 1H), 3.8 (d, J=13.5 Hz, 1H), 4.0 (m, 2H), 6.7 (ddd, J=8.7, 5.4, 5.1 Hz, 1H), 7.3 (m, 3H), 7.6 (d, J=7.7 Hz, 1H), 8.3 (d, J=4.8 Hz, 1H); m/z 551 (M+Na⁺).

### Example 57

### (4aR,10bS)-1-{[5-(4-methyl-1-piperazinyl)-3-(1-piperidinylmethyl)imidazo[1,2-a]pyridin-2-yl]methyl}-1,2,3,4,4a,5,6,10b-octahydro-1,10-phenanthroline

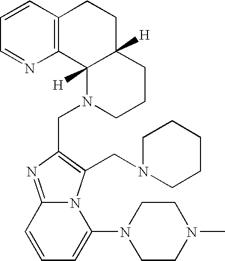

(4aR,10bS)-1-{[5-(4-methyl-1-piperazinyl)imidazo[1,2-a]pyridin-2-yl]methyl}-1,2,3,4,4a,5,6,10b-octahydro-1,10-phenanthroline (0.050 g, 0.12 mmol), piperidine (0.12 mL, 1.2 mmol), 0.1 mL of acetic acid, and 0.10 mL (1.2 mmol) of a 37% formaldehyde solution in water were heated to 50° C. for 2 hours. The mixture was allowed to cool and was quenched with 10 mL of saturated sodium bicarbonate. The mixture was extracted three times with 5 mL of dichloromethane and the combined organic layers concentrated. The residue was purified by silica chromatography eluting with a 0% to 10% gradient of 30% aqueous ammonium hydroxide in acetonitrile to yield 60 mg (97%) of (4aR,10bS)-1-{[5-(4-methyl-1-piperazinyl)-3-(1-piperidinylmethyl)imidazo[1,2-a]pyridin-2-yl]methyl}-1,2,3,4,4a,5,6,10b-octahydro-1,10-phenanthroline. ¹H NMR (400 MHz, METHANOL-D4) δ ppm 1.3 (m, 6H), 1.4 (m, 1H), 1.6 (m, 1H), 1.8 (m, 4H), 1.9 (m, 1H), 2.0 (m, 3H), 2.2 (m, 1H), 2.4 (s, 3 H), 2.4 (m, 1H), 2.5 (m, 1H), 2.6 (m, 1H), 2.7 (m, 1H), 2.9 (m, 4H), 3.0 (m, 2H), 3.2 (m, 2H), 3.3 (m, 1H), 3.4 (s, 1H), 3.7 (d, J=13.4 Hz, 1H), 4.0 (m, 2H), 6.7 (dd, J=5.5, 2.9 Hz, 1H), 7.2 (d, J=5.5 Hz, 2H), 7.3 (dd, J=7.7, 4.9 Hz, 1H), 7.7 (d, J=7.7 Hz, 1H), 8.3 (d, J=4.8 Hz, 1H); m/z 514 (M+1).

### Example 58

### (4aR,10bS)-1-{[5-(4-methyl-1-piperazinyl)-3-(1-pyrrolidinylmethyl)imidazo[1,2-a]pyridin-2-yl]methyl}-1,2,3,4,4a,5,6,10b-octahydro-1,10-phenanthroline

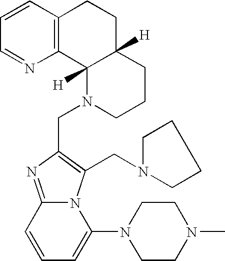

(4aR,10bS)-1-{[5-(4-methyl-1-piperazinyl)imidazo[1,2-a]pyridin-2-yl]methyl}-1,2,3,4,4a,5,6,10b-octahydro-1,10-phenanthroline (0.050 g, 0.12 mmol), pyrrolidine (0.10 mL, 1.2 mmol), 0.1 mL of acetic acid, and 0.10 mL (1.2 mmol) of a 37% formaldehyde solution in water were heated to 50° C. for 2 hours. The mixture was allowed to cool and was quenched with 10 mL of saturated sodium bicarbonate. The mixture was extracted three times with 5 mL of dichloromethane and the combined organic layers concentrated. The residue was purified by silica chromatography eluting with a 0% to 10% gradient of 30% aqueous ammonium hydroxide in acetonitrile to yield 55 mg (92%) of (4aR,10bS)-1-{[5-(4-methyl-1-piperazinyl)-3-(1-pyrrolidinylmethyl)imidazo[1,2-a]pyridin-2-yl]methyl}-1,2,3,4,4a,5,6,10b-octahydro-1,10-phenanthroline. ¹H NMR (400 MHz, METHANOL-D4) δ ppm 1.5 (m, 1H), 1.5 (m, 4H), 1.6 (m, 1H), 1.8 (m, 3H), 2.0 (m, 3H), 2.2 (m, 3H), 2.4 (s, 3H), 2.5 (m, 2 H), 2.6 (m, 1H), 2.7 (m, 1H), 2.9 (m, 4H), 3.0 (m, 1H), 3.1 (m, 1H), 3.2 (m, 2H), 3.4 (m, 1H), 3.4 (m, 1H), 3.9 (d, J=13.0 Hz, 1H), 4.1 (d, J=13.7 Hz, 1H), 4.3 (d, J=13.7 Hz, 1H), 6.7 (dd, J=5.1, 3.1 Hz, 1H), 7.3 (m, 3H), 7.6 (d, J=7.5 Hz, 1H), 8.3 (d, J=4.8 Hz, 1H); m/z 522 (M+Na⁺).

### Example 59

### (4aR,10bS)-1-{[5-(4-methyl-1-piperazinyl)-3-(4-morpholinylmethyl)imidazo[1,2-a]pyridin-2-yl]methyl}-1,2,3,4,4a,5,6,10b-octahydro-1,10-phenanthroline

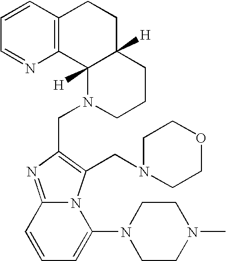

(4aR,10bS)-1-{[5-(4-methyl-1-piperazinyl)imidazo[1,2-a]pyridin-2-yl]methyl}-1,2,3,4,4a,5,6,10b-octahydro-1,10-phenanthroline (0.050 g, 0.12 mmol), morpholine (0.10 mL, 1.2 mmol), 0.1 mL of acetic acid, and 0.10 mL (1.2 mmol) of a 37% formaldehyde solution in water were heated to 50° C. for 2 hours. The mixture was allowed to cool and was quenched with 10 mL of saturated sodium bicarbonate. The mixture was extracted three times with 5 mL of dichloromethane and the combined organic layers concentrated. The residue was purified by silica chromatography eluting with a 0% to 10% gradient of 30% aqueous ammonium hydroxide in acetonitrile to yield 59 mg (95%) of (4aR,10bS)-1-{[5-(4-methyl-1-piperazinyl)-3-(4-morpholinylmethyl)imidazo[1,2-a]pyridin-2-yl]methyl}-1,2,3,4,4a,5,6,10b-octahydro-1,10-phenanthroline. ¹H NMR (400 MHz, METHANOL-D4) δ ppm 1.5 (m, 1H), 1.6 (m, 1H), 1.8 (m, 3H), 1.9 (m, 1H), 2.0 (m, 4H), 2.1 (m, 1H), 2.4 (m, 4H), 2.5 (m, 1 H), 2.6 (m, 1H), 2.8 (m, 1H), 2.9 (m, 6H), 3.1 (m, 1H), 3.2 (m, 2H), 3.3 (m, 2H), 3.7 (m, 2H), 4.0 (m, 2H), 4.8 (m, 1H), 5.5 (d, J=7.3 Hz, 1H), 6.7 (m, 1H), 7.3 (m, 3 H), 7.6 (t, J=8.5 Hz, 1H), 8.4 (dd, J=9.5, 4.8 Hz, 1H); m/z 516 (M+1).

### Example 60

### 1,1-dimethylethyl (3R)-3-{[(2-nitrophenyl)amino]methyl}-1-piperidinecarboxylate (intermediate)

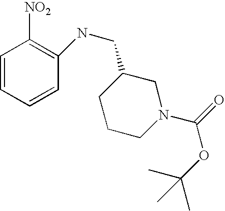

A mixture of 1,1-dimethylethyl (3R)-3-(aminomethyl)-1-piperidinecarboxylate (3.50 g, 16.3 mmol, Ennova MedChem Group, Inc.), 1-fluoro-2-nitrobenzene (3.46 g, 24.5 mmol, Avocado Research Chemicals Ltd.), and K₂CO₃ (11.3 g, 81.5 mmol) in 40 mL of anhydrous acetonitrile was heated to reflux with stirring. After 5 hours the mixture was cooled to RT and filtered through a medium fritted funnel to remove solids. The filter cake was rinsed with an additional 40 mL portion of acetonitrile and the filtrate concentrated to dryness at reduced pressure. The crude oil was subjected to flash chromatography (silica gel, gradient elution of hexane to 6:4 hexane/EtOAc) to afford 1,1-dimethylethyl (3R)-3-{[(2-nitrophenyl)amino]methyl}-1-piperidinecarboxylate as a viscous, yellow oil in quantitative yield. ¹H NMR (DMSO-d₆): δ 8.19 (br s, 1H), 8.05 (d, 1H), 7.52 (t, 1H), 7.06 (d, 1H), 6.68 (t, 1H), 3.93-3.58 (m, 2H), 3.29-3.20 (m, 2H), 2.89-2.48 (m, 2H), 1.85-1.68 (m, 2H), 1.60 (m, 1H), 1.50-1.10 (br s, 11H). MS m/z 358 (M+Na).

### Example 61

### 1,1-Dimethylethyl (3R)-3-{[(2-aminophenyl)amino]methyl}-1-piperidinecarboxylate (intermediate)

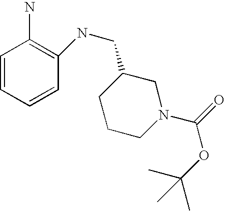

A solution of 1,1-dimethylethyl (3R)-3-{[(2-nitrophenyl)amino]methyl}-1-piperidinecarboxylate (5.00 g, 14.9 mmol) in 150 mL of EtOH was subjected to balloon hydrogenation in the presence of 0.50 g of 10% Pd on carbon. After 18 hours the reaction vessel was purged with nitrogen, catalyst removed by filtration through celite, and the filtrate concentrated to dryness at reduced pressure to afford 4.38 g (96%) of 1,1-dimethylethyl (3R)-3-{[(2-aminophenyl)amino]methyl}-1-piperidinecarboxylate as a viscous brown oil. ¹H NMR (DMSO-d₆): δ 6.53-6.30 (m, 4H), 4.51-4.29 (m, 3H), 4.05-3.60 (m, 2H), 2.93-2.40 (m, 4H), 1.81 (m, 1H), 1.69 (m, 1H), 1.59 (m, 1H), 1.43-1.05 (m, 11H). MS m/z 306 (M+H).

### Example 62

### 1,1-Dimethylethyl (3R)-3-{[2-(chloromethyl)-1H-benzimidazol-1-yl]methyl}-1-piperidinecarboxylate (intermediate)

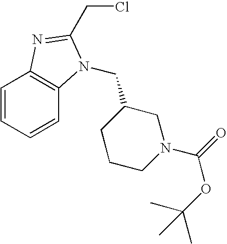

A solution of 1,1-dimethylethyl (3R)-3-{[(2-aminophenyl)amino]methyl}-1-piperidinecarboxylate (4.20 g, 13.8 mmol), 2-chloro-1,1,1-trimethoxyethane (6.40 g, 41.4 mmol, Aldrich), and p-toluenesulfonic acid (0.26 g, 1.4 mmol) in 70 mL of dichloromethane was stirred at RT. After 18 hours the solution was diluted with 100 mL of dichloromethane, washed twice with saturated aqueous NaHCO₃, dried over Na₂SO₄, and concentrated to dryness at reduced pressure. The crude product was purified by flash chromatography (silica gel, gradient elution of hexane to 6:4 hexane/EtOAc) to afford 4.71 g (94%) of 1,1-dimethylethyl (3R)-3-{([2-(chloromethyl)-1H-benzimidazol-1-yl]methyl}-1-piperidinecarboxylate as a light tan foam. ¹H NMR (DMSO-d₆): δ 7.67-7.58 (m, 2H), 7.28 (t, 1H), 7.23 (t, 1H), 5.06 (s, 2H), 4.28-4.13 (m, 2H), 3.79 (d, 1H), 3.72-3.38 (m, 1H), 2.80-2.58 (m, 2H), 2.05 (m, 1H), 1.72-1.54 (m, 2H), 1.50-0.97 (m, 11H). MS m/z 364 (M+H).

### Example 63

### 1,1-dimethylethyl (3R)-3-({2-[(4aR,10bS)-3,4,4a,5,6,10b-hexahydro-1,10-phenanthrolin-1(2H)-ylmethyl]-1H-benzimidazol-1-yl}methyl)-1-piperidinecarboxylate (intermediate)

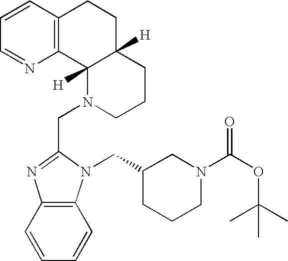

A mixture of (4aR,10bS)-1,2,3,4,4a,5,6,10b-octahydro-1,10-phenanthroline (0.12 g, 0.66 mmol), 1,1-dimethylethyl (3R)-3-{[2-(chloromethyl)-1H-benzimidazol-1-yl]methyl}-1-piperidinecarboxylate (0.24 g, 0.66 mmol), potassium carbonate (0.45 g, 3.3 mmol) and potassium iodide (0.010 g, 0.066 mmol) in 10 mL of acetonitrile was stirred for 5 hours at room temperature. The mixture was diluted with brine, extracted 3 times with 10 mL of dichloromethane, and concentrated. The residue was purified by silica chromatography eluting with a 0% to 10% gradient of 2M ammoniated methanol in dichloromethane to yield 102 mg (30%) of 1,1-dimethylethyl (3R)-3-({2-[(4aR,10bS)-3,4,4a,5,6,10b-hexahydro-1,10-phenanthrolin-1(2H)-ylmethyl]-1H-benzimidazol-1-yl}methyl)-1-piperidinecarboxylate. ¹H NMR (400 MHz, METHANOL-D4) δ ppm 1.3 (m, 17H), 1.7 (m, 1H), 1.8 (m, 2H), 2.1 (m, 1H), 2.3 (m, 2H), 2.6 (m, 2H), 2.8 (m, 1H), 3.0 (m, 1H), 3.2 (m, 1H), 3.4 (d, J=1.8 Hz, 1H), 3.6 (d, J=13.0 Hz, 1H), 3.9 (m, 2H), 4.2 (m, 1H), 4.3 (d, J=14.3 Hz, 1H), 7.2 (m, 2 H), 7.4 (dd, J=7.8, 4.7 Hz, 1H), 7.4 (d, J=7.5 Hz, 1H), 7.6 (d, J=7.3 Hz, 1H), 7.7 (d, J=7.0 Hz, 1H), 8.4 (d, J=5.3 Hz, 1H); m/z 516 (M+1).

### Example 64

### (4aR,10bS)-1-({1-[(3S)-3-piperidinylmethyl]-1H-benzimidazol-2-yl}methyl)-1,2,3,4,4a,5,6,10b-octahydro-1,10-phenanthroline

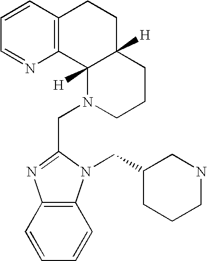

To a solution of (3R)-3-({2-[(4aR,10bS)-3,4,4a,5,6,10b-hexahydro-1,10-phenanthrolin-1(2H)-ylmethyl]-1H-benzimidazol-1-yl}methyl)-1-piperidinecarboxylate (0.10 g, 0.19 mmol) in 3 mL of dichloromethane was added 3 mL of trifluoroacetic acid. The mixture was stirred for 3 hours and concentrated. The residue was slurried in 20 mL of saturated sodium bicarbonate and extracted 3 times with 10 mL of dichloromethane. The combined organic layers were dried over sodium sulfate and concentrated to yield 70 mg (87%) of (4aR,10bS)-1-({1-[(3S)-3-piperidinylmethyl]-1H-benzimidazol-2-yl}methyl)-1,2,3,4,4a,5,6,10b-octahydro-1,10-phenanthroline. ¹H NMR (400 MHz, METHANOL-D4) δ ppm 0.3 (m, 1H), 1.1 (m, 2 H), 1.3 (m, 1H), 1.4 (m, 2H), 1.6 (m, 2H), 1.8 (m, 3H), 2.1 (m, 2H), 2.3 (m, 2H), 2.7 (m, 2H), 2.8 (m, 1H), 2.8 (m, 1H), 3.0 (m, 1H), 3.1 (m, 1H), 3.4 (d, J=2.7 Hz, 1 H), 3.5 (d, J=13.9 Hz, 1H), 3.9 (dd, J=14.4, 7.1 Hz, 1H), 4.1 (dd, J=14.5, 8.6 Hz, 1 H), 4.2 (d, J=13.9 Hz, 1H), 7.2 (m, 2H), 7.4 (m, 2H), 7.5 (ddd, J=7.5, 1.1, 0.7 Hz, 1 H), 7.7 (d, J=8.6 Hz, 1H), 8.4 (dd, J=4.8, 1.6 Hz, 1H); m/z 416 (M+1).

### Example 65

### (4aR,10bS)-1-[(1-{[(3S)-1-(1-methylethyl)-3-piperidinyl]methyl}-1H-benzimidazol-2-yl)methyl]-1,2,3,4,4a,5,6,10b-octahydro-1,10-phenanthroline

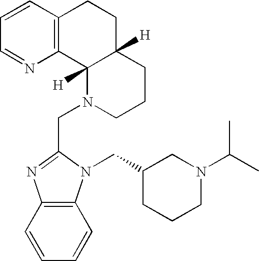

A mixture of (4aR,10bS)-1-({1-[(3S)-3-piperidinylmethyl]-1H-benzimidazol-2-yl}methyl)-1,2,3,4,4a,5,6,10b-octahydro-1,10-phenanthroline (0.030 g, 0.072 mmol), acetone (0.013 g, 0.22 mmol), acetic acid (0.0040 g, 0.072 mmol), and sodium triacetoxyborohydride (0.045 g, 0.22 mmol) in 1 mL of 1,2-dichloroethane was stirred for 16 hours at room temperature. The mixture was quenched with 10 mL saturated sodium bicarbonate, extracted 3 times with 10 mL of dichloromethane, and the combined organic layers concentrated. The residue was purified by silica chromatography eluting with a 0% to 10% gradient of 30% aqueous ammonium hydroxide in acetonitrile to yield 9 mg (27%) of (4aR,10bS)-1-[(1-{[(3S)-1-(1-methylethyl)-3-piperidinyl]methyl}-1H-benzimidazol-2-yl)methyl]-1,2,3,4,4a,5,6,10b-octahydro-1,10-phenanthroline. ¹H NMR (400 MHz, METHANOL-D4) δ ppm 0.1 (m, 1H), 1.0 (m, 1H), 1.0 (d, J=6.6 Hz, 6H), 1.2 (m, 1H), 1.5 (m, 2H), 1.8 (m, 6H), 2.0 (m, 1H), 2.1 (m, 1H), 2.3 (m, 1H), 2.7 (m, 3H), 2.8 (d, J=11.7 Hz, 2H), 3.0 (ddd, J=17.7, 10.2, 7.4 Hz, 1H), 3.1 (m, 1H), 3.4 (d, J=2.6 Hz, 1H), 3.5 (d, J=14.1 Hz, 1 H), 4.0 (dd, J=14.4, 6.5 Hz, 1H), 4.2 (dd, J=14.5, 8.8 Hz, 1H), 4.3 (d, J=13.9 Hz, 1 H), 7.2 (m, 2H), 7.4 (dd, J=7.9, 4.8 Hz, 1H), 7.4 (d, J=8.1 Hz, 1H), 7.6 (m, 1H), 7.7 (d, J=7.7 Hz, 1H), 8.4 (d, J=4.6 Hz, 1H); m/z 458 (M+1).

### Example 66

### (4aR,10bS)-1-[(1-{[(3S)-1-methyl-3-piperidinyl]methyl}-1H-benzimidazol-2-yl)methyl]-1,2,3,4,4a,5,6,10b-octahydro-1,10-phenanthroline

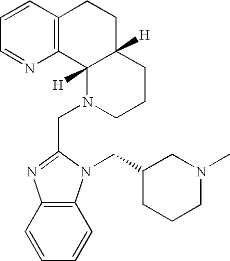

A mixture of (4aR,10bS)-1-({1-[(3S)-3-piperidinylmethyl]-1H-benzimidazol-2-yl}methyl)-1,2,3,4,4a,5,6,10b-octahydro-1,10-phenanthroline (0.030 g, 0.072 mmol), a 37% solution of formaldehyde (0.0066 g, 0.22 mmol) in water, acetic acid (0.0040 g, 0.072 mmol), and sodium triacetoxyborohydride (0.045 g, 0.22 mmol) in 1 mL of 1,2-dichloroethane was stirred for 16 hours at room temperature. The mixture was quenched with 10 mL saturated sodium bicarbonate, extracted 3 times with 10 mL of dichloromethane, and the combined organic layers concentrated. The residue was purified by silica chromatography eluting with a 0% to 10% gradient of 30% aqueous ammonium hydroxide in acetonitrile to yield 26 mg (84%) of (4aR,10bS)-1-[(1-{[(3S)-1-methyl-3-piperidinyl]methyl}-1H-benzimidazol-2-yl)methyl]-1,2,3,4,4a,5,6,10b-octahydro-1,10-phenanthroline. ¹H NMR (400 MHz, METHANOL-D4) δ ppm 0.1 (qd, J=12.3, 3.6 Hz, 1H), 1.0 (d, J=13.2 Hz, 1H), 1.2 (m, 1H), 1.5 (m, 3H), 1.6 (m, 1H), 1.8 (m, 5H), 2.1 (m, 1H), 2.2 (s, 3H), 2.3 (m, 1H), 2.5 (d, J=11.0 Hz, 1H), 2.6 (m, 1 H), 2.7 (m, 1H), 2.8 (d, J=11.9 Hz, 1H), 3.0 (ddd, J=17.7, 10.2, 7.6 Hz, 1H), 3.1 (m, 1H), 3.4 (d, J=2.4 Hz, 1H), 3.5 (d, J=13.9 Hz, 1H), 3.9 (dd, J=14.5, 7.0 Hz, 1H), 4.2 (dd, J=14.4, 8.7 Hz, 1H), 4.3 (d, J=13.9 Hz, 1H), 7.2 (m, 2H), 7.4 (dd, J=7.7, 4.8 Hz, 1H), 7.4 (d, J=7.0 Hz, 1H), 7.6 (d, J=6.4 Hz, 1H), 7.7 (d, J=7.7 Hz, 1H), 8.4 (d, J=5.5 Hz, 1H); m/z 430 (M+1).

### Example 67

### 1-{2-[(4aR,10bS)-3,4,4a,5,6,10b-hexahydro-1,10-phenanthrolin-1(2′-ylmethyl]imidazo[1,2-a]pyridin-5-yl}-N N-dimethyl-4-piperidinamine

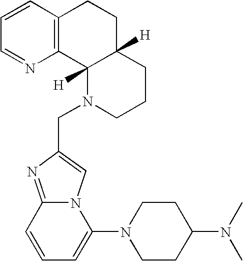

To a solution of (4aR,10bS)-1-[(5-fluoroimidazo[1,2-a]pyridin-2-yl)methyl]-1,2,3,4,4a,5,6,10b-octahydro-1,10-phenanthroline (0.030 g, 0.089 mmol) in 2 mL of dimethyl sulfoxide was added potassium carbonate (0.12 g, 0.87 mmol) and N,N-dimethyl-4-piperidinamine dihydrochloride (0.066 g, 0.33 mmol). The mixture was heated at 60° C. for 16 hours and then allowed to cool to room temperature. The mixture was quenched with water and extracted 3 times with 10 mL of dichloromethane. The combined organic layers were concentrated and the residue purified by silica chromatography eluting with a 0% to 10% gradient of 30% aqueous ammonium hydroxide in acetonitrile to yield 39 mg (98%) of 1-{2-[(4aR,10bS)-3,4,4a,5,6,10b-hexahydro-1,10-phenanthrolin-1(2H)-ylmethyl]imidazo[1,2-a]pyridin-5-yl}-N,N-dimethyl-4-piperidinamine. ¹H NMR (400 MHz, METHANOL-D4) δ ppm 1.7 (m, 7H), 2.1 (m, 3H), 2.4 (m, 9H), 2.8 (m, 3H), 3.0 (m, 1H), 3.1 (m, 1H), 3.5 (m, 2 H), 3.7 (m, 2H), 4.1 (d, J=15.0 Hz, 1H), 6.4 (d, J=7.1 Hz, 1H), 7.2 (m, 2H), 7.2 (m, 1H), 7.5 (d, J=7.7 Hz, 1H), 7.6 (s, 1H), 8.3 (d, J=4.9 Hz, 1H); m/z 445 (M+1).

### Example 68

### 2-[(4aR,10bS)-3,4,4a,5,6,10b-hexahydro-1,10-phenanthrolin-1(2H)-ylmethyl]-N-(1-methyl-4-piperidinyl)imidazo[1,2-a]pyridin-5-amine

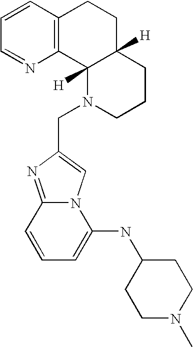

To a solution of (4aR,10bS)-1-[(5-fluoroimidazo[1,2-a]pyridin-2-yl)methyl]-1,2,3,4,4a,5,6,10b-octahydro-1,10-phenanthroline (0.030 g, 0.089 mmol) in 2 mL of dimethyl sulfoxide was added 1-methyl-4-piperidinamine (0.038 g, 0.33 mmol). The mixture was heated at 60° C. for 16 hours and then allowed to cool to room temperature. The mixture was quenched with water and extracted 3 times with 10 mL of dichloromethane. The combined organic layers were concentrated and the residue purified by silica chromatography eluting with a 0% to 10% gradient of 30% aqueous ammonium hydroxide in acetonitrile to yield 28 mg (73%) of 2-[(4aR,10bS)-3,4,4a,5,6,10b-hexahydro-1,10-phenanthrolin-1(2H)-ylmethyl]-N-(1-methyl-4-piperidinyl)imidazo[1,2-a]pyridin-5-amine. ¹H NMR (400 MHz, METHANOL-D4) δ ppm 1.6 (m, 1H), 1.7 (m, 5H), 1.9 (m, 1H), 2.1 (m, 3H), 2.3 (m, 3H), 2.4 (s, 3H), 2.5 (m, 1H), 2.8 (m, 1H), 2.9 (d, J=13.0 Hz, 2H), 3.0 (m, 1H), 3.2 (m, 1H), 3.5 (m, 2H), 3.6 (m, 1H), 3.9 (d, J=14.1 Hz, 1H), 6.0 (d, J=7.7 Hz, 1H), 6.8 (d, J=8.8 Hz, 1 H), 7.2 (m, 2H), 7.6 (d, J=7.7 Hz, 1H), 7.7 (s, 1H), 8.3 (d, J=4.8 Hz, 1H); m/z 431 (M+1).

### Example 69

### 1-{2-[(4aR,10bS)-3,4,4a,5,6,10b-hexahydro-1,10-phenanthrolin-1(2H)-ylmethyl]imidazo[1,2-a]pyridin-5-yl}-4-piperidinol

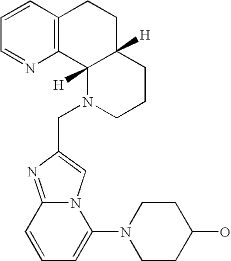

To a solution of (4aR,10bS)-1-[(5-fluoroimidazo[1,2-a]pyridin-2-yl)methyl]-1,2,3,4,4a,5,6,10b-octahydro-1,10-phenanthroline (0.030 g, 0.089 mmol) in 2 mL of dimethyl sulfoxide was added 4-piperidinol (0.033 g, 0.33 mmol). The mixture was heated at 60° C. for 16 hours and then allowed to cool to room temperature. The mixture was quenched with water and extracted 3 times with 10 mL of dichloromethane. The combined organic layers were concentrated and the residue purified by silica chromatography eluting with a 0% to 10% gradient of 30% aqueous ammonium hydroxide in acetonitrile to yield 28 mg (73%) of 1-{2-[(4aR,10bS)-3,4,4a,5,6,10b-hexahydro-1,10-phenanthrolin-1(2H)-ylmethyl]imidazo[1,2-a]pyridin-5-yl}-4-piperidinol. ¹H NMR (400 MHz, METHANOL-D4) δ ppm 1.7 (m, 3H), 1.8 (m, 4 H), 2.1 (m, 2H), 2.2 (m, 1H), 2.4 (m, 1H), 2.5 (m, 1H), 2.8 (m, 1H), 2.9 (m, 2H), 3.0 (m, 1H), 3.1 (ddd, J=17.2, 7.1, 7.0 Hz, 1H), 3.3 (m, 2H), 3.7 (m, 2H), 3.9 (m, 1 H), 4.1 (d, J=14.3 Hz, 1H), 6.4 (d, J=7.3 Hz, 1H), 7.2 (m, 2H), 7.2 (m, 1H), 7.5 (d, J=7.1 Hz, 1H), 7.6 (s, 1H), 8.3 (d, J=3.8 Hz, 1H); m/z 418 (M+1).

### Example 70

### N-{2-[(4aR,10bS)-3,4,4a,5,6,10b-hexahydro-1,10-phenanthrolin-1(2H)-ylmethyl]imidazo[1,2-a]pyridin-5-yl}-N,N′,N′-trimethyl-1,2-ethanediamine

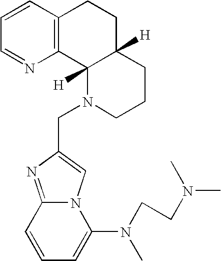

To a solution of (4aR,10bS)-1-[(5-fluoroimidazo[1,2-a]pyridin-2-yl)methyl]-1,2,3,4,4a,5,6,10b-octahydro-1,10-phenanthroline (0.050 g, 0.15 mmol) in 2 mL of dimethyl sulfoxide was added N,N,N′-trimethyl-1,2-ethanediamine (0.30 g). The mixture was heated at 60° C. for 16 hours and then allowed to cool to room temperature. The mixture was quenched with water and extracted 3 times with 10 mL of dichloromethane, and washed with brine. The organic layer was concentrated and the residue purified by silica chromatography eluting with a 0% to 10% gradient of 30% aqueous ammonium hydroxide in acetonitrile to yield 20 mg (32%) of N-{2-[(4aR,10bS)-3,4,4a,5,6,10b-hexahydro-1,10-phenanthrolin-1(2H)-ylmethyl]imidazo[1,2-a]pyridin-5-yl}-N,N′,N′-trimethyl-1,2-ethanediamine. ¹H NMR (400 MHz, METHANOL-D4) δ ppm 1.6 (m, 1H), 1.7 (m, 3H), 1.8 (m, 1H), 2.1 (m, 1 H), 2.2 (s, 6H), 2.3 (t, J=9.4 Hz, 1H), 2.4 (m, 1H), 2.6 (m, 2H), 2.8 (m, 1H), 2.9 (s, 3H), 3.0 (m, 1H), 3.1 (m, 1H), 3.2 (m, 2H), 3.6 (m, 2H), 4.0 (d, J=14.6 Hz, 1H), 6.5 (d, J=7.3 Hz, 1H), 7.1 (d, J=9.0 Hz, 1H), 7.2 (dd, J=7.7, 4.8 Hz, 1H), 7.2 (m, 1 H), 7.5 (d, J=7.7 Hz, 1H), 7.7 (s, 1H), 8.3 (d, J==3.1 Hz, 1H); m/z 419 (M+1).

### Example 71

### (4aR,10bS)-1-{[5-(1,4′-bipiperidin-1′-yl)imidazo[1,2-a]pyridin-2-yl]methyl}-1,2,3,4,4a,5,6,10b-octahydro-1,0-phenanthroline

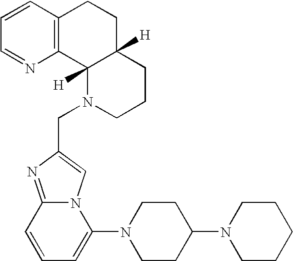

To a solution of (4aR,10bS)-1-[(5-fluoroimidazo[1,2-a]pyridin-2-yl)methyl]-1,2,3,4,4a,5,6,10b-octahydro-1,10-phenanthroline (0.050 g, 0.15 mmol) in 2 mL of dimethyl sulfoxide was added 1,4′-bipiperidine (0.30 g). The mixture was heated at 60° C. for 16 hours and then allowed to cool to room temperature. The mixture was quenched with water and extracted 3 times with 10 mL of dichloromethane, and washed with brine. The organic layer was concentrated and the residue purified by silica chromatography eluting with a 0% to 10% gradient of 30% aqueous ammonium hydroxide in acetonitrile to yield 36 mg (49%) of (4aR,10bS)-1-{[5-(1,4′-bipiperidin-1′-yl)imidazo[1,2-a]pyridin-2-yl]methyl}-1,2,3,4,4a,5,6,10b-octahydro-1,10-phenanthroline. ¹H NMR (400 MHz, METHANOL-D4) δ ppm 1.5 (m, 2H), 1.7 (m, 7 H), 1.8 (m, 3H), 2.1 (m, 3H), 2.4 (m, 2H), 2.5 (m, 1H), 2.7 (m, 8H), 2.9 (m, 1H), 3.1 (dt, J=17.1, 6.7 Hz, 1H), 3.5 (m, 2H), 3.6 (d, J=3.1 Hz, 1H), 3.6 (d, J=14.8 Hz, 1 H), 4.0 (d, J=14.6 Hz, 1H), 6.4 (d, J=7.1 Hz, 1H), 7.2 (m, 3H), 7.5 (d, J=7.7 Hz, 1 H), 7.5 (s, 1H), 8.3 (d, J=3.8 Hz, 1H); m/z 485 (M+1).

### Example 72

### (4aR,10bS)-1-({5-[4-(1-pyrrolidinyl)-1-piperidinyl]imidazo[1,2-a]pyridin-2-yl}methyl)-1,2,3,4,4a,5,6,10b-octahydro-1,10-phenanthroline

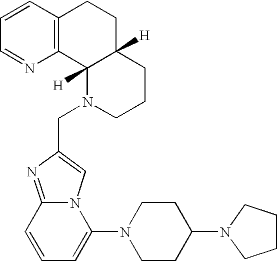

To a solution of (4aR,10bS)-1-[(5-fluoroimidazo[1,2-a]pyridin-2-yl)methyl]-1,2,3,4,4a,5,6,10b-octahydro-1,10-phenanthroline (0.050 g, 0.15 mmol) in 2 mL of dimethyl sulfoxide was added 4-(1-pyrrolidinyl)piperidine (0.30 g). The mixture was heated at 60° C. for 16 hours and then allowed to cool to room temperature. The mixture was quenched with water and extracted 3 times with 10 mL of dichloromethane, and then washed with brine. The organic layer was concentrated and the residue purified by silica chromatography eluting with a 0% to 10% gradient of 30% aqueous ammonium hydroxide in acetonitrile to yield 37 mg (52%) of (4aR,10bS)-1-({5-[4-(1-pyrrolidinyl)-1-piperidinyl]imidazo[1,2-a]pyridin-2-yl}methyl)-1,2,3,4,4a,5,6,10b-octahydro-1,10-phenanthroline. ¹H NMR (400 MHz, METHANOL-D4) δ ppm 1.6 (m, 1H), 1.7 (m, 2H), 1.8 (m, 3H), 1.9 (s, 4H), 2.1 (m, 3H), 2.4 (m, 3 H), 2.7 (m, 8H), 3.0 (m, 1H), 3.1 (m, 1H), 3.4 (m, 2H), 3.6 (d, J=1.8 Hz, 1H), 3.6 (d, J=15.0 Hz, 1H), 4.0 (d, J=14.6 Hz, 1H), 6.4 (d, J=7.3 Hz, 1H), 7.2 (m, 3H), 7.5 (d, J=7.9 Hz, 1H), 7.5 (s, 1H), 8.3 (d, J=4.8 Hz, 1H); m/z 471 (M+1).

### Example 73

### (4aR,10bS)-1-{[5-(4-ethyl-1-piperazinyl)imidazo[1,2-a]pyridin-2-yl]methyl}-1,2,3,4,4a,5,6,10b-octahydro-1,10-phenanthroline

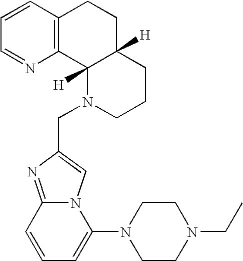

To a solution of (4aR,10bS)-1-[(5-fluoroimidazo[1,2-a]pyridin-2-yl)methyl]-1,2,3,4,4a,5,6,10b-octahydro-1,10-phenanthroline (0.050 g, 0.15 mmol) in 2 mL of dimethyl sulfoxide was added 1-ethylpiperazine (0.30 g). The mixture was heated at 60° C. for 16 hours and then allowed to cool to room temperature. The mixture was quenched with water and extracted 3 times with 10 mL of dichloromethane, and then washed with brine. The organic layer was concentrated and the residue purified by silica chromatography eluting with a 0% to 10% gradient of 30% aqueous ammonium hydroxide in acetonitrile to yield 30 mg (46%) of (4aR,10bS)-1-{[5-(4-ethyl-1-piperazinyl)imidazo[1,2-a]pyridin-2-yl]methyl}-1,2,3,4,4a,5,6,10b-octahydro-1,10-phenanthroline. ¹H NMR (400 MHz, METHANOL-D4) δ ppm 1.2 (t, J=7.2 Hz, 3H), 1.7 (m, 5H), 2.1 (m, 1H), 2.4 (m, 2H), 2.6 (q, J=7.1 Hz, 2H), 2.7 (m, 5H), 2.9 (m, 1 H), 3.1 (m, 5H), 3.6 (s, 1H), 3.6 (d, J=14.7 Hz, 1H), 4.0 (d, J=14.7 Hz, 1H), 6.4 (d, J=7.1 Hz, 1H), 7.2 (m, 3H), 7.5 (d, J=7.7 Hz, 1H), 7.6 (s, 1H), 8.3 (d, J=4.8 Hz, 1 H); m/z 431 (M+1).

### Example 74

### 2-[(4aR,10bS)-3,4,4a,5,6,10b-hexahydro-1,10-phenanthrolin-1(2H)-ylmethyl]-N-methyl-N-(1-methyl-4-piperidinyl)imidazo[1,2-a]pyridin-5-amine

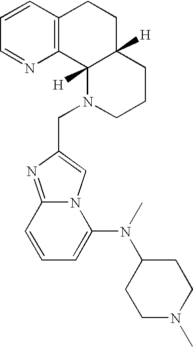

To a solution of (4aR,10bS)-1-[(5-fluoroimidazo[1,2-a]pyridin-2-yl)methyl]-1,2,3,4,4a,5,6,10b-octahydro-1,10-phenanthroline (0.050 g, 0.15 mmol) in 2 mL of dimethyl sulfoxide was added N,1-dimethyl-4-piperidinamine (0.30 g). The mixture was heated at 60° C. for 16 hours and then allowed to cool to room temperature. The mixture was quenched with water and extracted 3 times with 10 mL of dichloromethane, and then washed with brine. The organic layer was concentrated and the residue purified by silica chromatography eluting with a 0% to 10% gradient of 30% aqueous ammonium hydroxide in acetonitrile to yield 17 mg (25%) of 2-[(4aR,10bS)-3,4,4a,5,6,10b-hexahydro-1,10-phenanthrolin-1(2H)-ylmethyl]-N-methyl-N-(1-methyl-4-piperidinyl)imidazo[1,2-a]pyridin-5-amine. ¹H NMR (400 MHz, METHANOL-D4) δ ppm 1.7 (m, 3H), 1.8 (m, 3H), 1.9 (m, 2H), 2.1 (m, 2H), 2.2 (m, 1H), 2.3 (s, 3H), 2.4 (m, 1H), 2.5 (m, 1H), 2.8 (m, 4H), 2.9 (m, 4H), 3.1 (m, 2H), 3.8 (m, 2H), 4.1 (d, J=15.7 Hz, 1H), 6.5 (d, J=7.1 Hz, 1H), 7.2 (m, 3H), 7.6 (d, J=7.7 Hz, 1H), 7.7 (s, 1H), 8.4 (d, J=4.8 Hz, 1H); m/z 445 (M+1).

### Example 75

### 2-[(4aR,10bS)-3,4,4a,5,6,10b-hexahydro-1,10-phenanthrolin-1(2H)-ylmethyl]-N-methyl-N-(1-methyl-3-pyrrolidinyl)imidazo[1,2-a]pyridin-5-amine

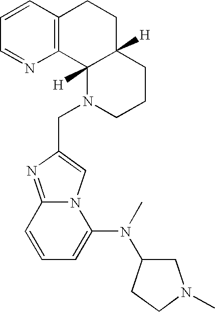

To a solution of (4aR,10bS)-1-[(5-fluoroimidazo[1,2-a]pyridin-2-yl)methyl]-1,2,3,4,4a,5,6,10b-octahydro-1,10-phenanthroline (0.050 g, 0.15 mmol) in 2 mL of dimethyl sulfoxide was added N,1-dimethyl-3-pyrrolidinamine (0.30 g). The mixture was heated at 60° C. for 16 hours and then allowed to cool to room temperature. The mixture was quenched with water and extracted 3 times with 10 mL of dichloromethane, and then washed with brine. The organic layer was concentrated and the residue purified by silica chromatography eluting with a 0% to 10% gradient of 30% aqueous ammonium hydroxide in acetonitrile to yield 11 mg (17%) of 2-[(4aR,10bS)-3,4,4a,5,6,10b-hexahydro-1,10-phenanthrolin-1(2H)-ylmethyl]-N-methyl-N-(1-methyl-3-pyrrolidinyl)imidazo[1,2-a]pyridin-5-amine. ¹H NMR (400 MHz, METHANOL-D4) δ ppm 1.7 (m, 5H), 1.9 (m, 2H), 2.2 (m, 2H), 2.4 (m, 4H), 2.7 (m, 3H), 2.8 (m, 3H), 2.8 (m, 3H), 3.1 (dt, J=17.0, 6.9 Hz, 1H), 3.7 (m, 2H), 4.0 (m, 1 H), 4.1 (m, 1H), 6.5 (d, J=7.1 Hz, 1H), 7.2 (m, 3H), 7.5 (d, J=7.3 Hz, 1H), 7.7 (s, 1 H), 8.3 (d, J=4.8 Hz, 1H); m/z 431 (M+1).

### Example 76a

### (4aR,10bS)-1-{[5-(4,4′-bipiperidin-1-yl)imidazo[1,2-a]pyridin-2-yl]methyl}-1,2,3,4,4a,5,6,10b-octahydro-1,10-phenanthroline and;

### Example 76b

### (4aR,10bS)-1-{[5-(1′-{2-[(6aS,10aR)-5,6,6a,7,8,9,10,10a-octahydrobenzo[h]quinolin-10-ylmethyl]imidazo[1,2-a]pyridin-5-yl}-4,4′-bipiperidin-1-yl)imidazo[1,2-a]pyridin-2-yl]methyl}-1,2,3,4,4a,5,6,10b-octahydro-1,10-phenanthroline

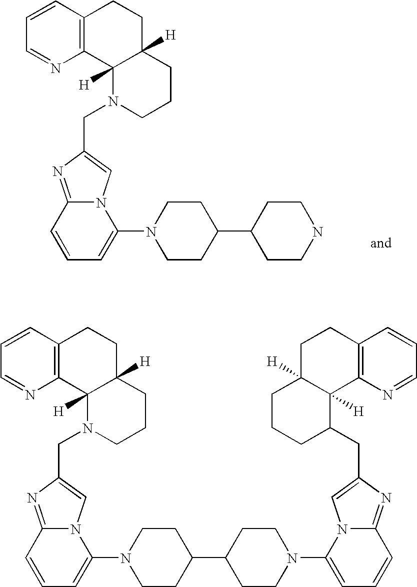

To a solution of (4aR,10bS)-1-[(5-fluoroimidazo[1,2-a]pyridin-2-yl)methyl]-1,2,3,4,4a,5,6,10b-octahydro-1,10-phenanthroline (0.050 g, 0.15 mmol) in 2 mL of dimethyl sulfoxide was added 4,4′-bipiperidine dihydrochloride (0.30 g). The mixture was heated at 60° C. for 16 hours and then allowed to cool to room temperature. The mixture was quenched with water and extracted 3 times with 10 mL of dichloromethane, and then washed with brine. The organic layer was concentrated and the residue purified by silica chromatography eluting with a 0% to 10% gradient of 30% aqueous ammonium hydroxide in acetonitrile to yield 29 mg (40%) of (4aR,10bS)-1-{[5-(4,4′-bipiperidin-1-yl)imidazo[1,2-a]pyridin-2-yl]methyl}-1,2,3,4,4a,5,6,10b-octahydro-1,10-phenanthroline and 27 mg (22%) of (4aR,10bS)-1-{[5-(1′-{2-[(6aS,10aR)-5,6,6a,7,8,9,10,10a-octahydrobenzo[h]quinolin-10-ylmethyl]imidazo[1,2-a]pyridin-5-yl}-4,4′-bipiperidin-1-yl)imidazo[1,2-a]pyridin-2-yl]methyl}-1,2,3,4,4a,5,6,10b-octahydro-1,10-phenanthroline.

76a: ¹H NMR (400 MHz, METHANOL-D4) δ ppm 1.4 (m, 4H), 1.6 (m, 2H), 1.7 (m, 4 H), 1.8 (m, 4H), 1.9 (m, 2H), 2.1 (m, 1H), 2.4 (m, 2H), 2.7 (m, 3H), 2.8 (m, 1H), 3.0 (m, 1H), 3.1 (m, 3H), 3.4 (m, 2H), 3.6 (m, 2H), 4.0 (m, 1H), 6.3 (d, J=7.0 Hz, 1 H), 7.1 (d, J=8.8 Hz, 1H), 7.2 (m, 2H), 7.5 (m, 2H), 8.3 (d, J=6.2 Hz, 1H); m/z 485 (M+1).

76b: ¹H NMR (400 MHz, METHANOL-D4) δ ppm 1.8 (m, 16H), 2.0 (m, 4H), 2.2 (m, 2H), 2.4 (m, 4H), 2.8 (m, 6H), 3.0 (m, 2H), 3.1 (m, 2H), 3.5 (m, 4H), 3.7 (m, 4H), 4.1 (m, 2H), 6.4 (d, J=7.1 Hz, 2H), 7.2 (m, 4H), 7.3 (m, 2H), 7.5 (d, J=7.9 Hz, 2H), 7.6 (s, 2H), 8.4 (d, J=4.4 Hz, 2H); m/z 801 (M+1).

### Example 77

### (4aR,10bS)-1-({5-[(3R,5S)-3,5-dimethyl-1-piperazinyl]imidazo[1,2-a]pyridin-2-yl}methyl)-1,2,3,4,4a,5,6,10b-octahydro-1,10-phenanthroline

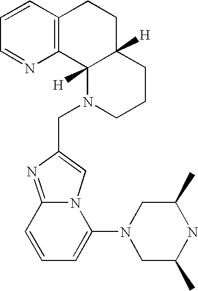

To a solution of (4aR,10bS)-1-[(5-fluoroimidazo[1,2-a]pyridin-2-yl)methyl]-1,2,3,4,4a,5,6,10b-octahydro-1,10-phenanthroline (0.050 g, 0.15 mmol) in 2 mL of dimethyl sulfoxide was added (2R,6S)-2,6-dimethylpiperazine (0.30 g). The mixture was heated at 60° C. for 16 hours and then allowed to cool to room temperature. The mixture was quenched with water and extracted 3 times with 10 mL of dichloromethane, and then washed with brine. The organic layer was concentrated and the residue purified by silica chromatography eluting with a 0% to 10% gradient of 30% aqueous ammonium hydroxide in acetonitrile to yield 36 mg (56%) of (4aR,10bS)-1-({5-[(3R,5S)-3,5-dimethyl-1-piperazinyl]imidazo[1,2-a]pyridin-2-yl}methyl)-1,2,3,4,4a,5,6,10b-octahydro-1,10-phenanthroline. ¹H NMR (400 MHz, METHANOL-D4) δ ppm 1.1 (d, J=6.4 Hz, 3H), 1.2 (d, J=6.4 Hz, 3H), 1.7 (m, 6H), 2.2 (m, 1H), 2.4 (q, J=10.4 Hz, 3H), 2.5 (m, 1H), 2.8 (ddd, J=17.0, 7.2, 7.0 Hz, 1H), 2.9 (m, 1H), 3.1 (m, 3H), 3.3 (d, J=2.2 Hz, 1H), 3.3 (m, 1H), 3.7 (m, 2H), 4.1 (d, J=14.8 Hz, 1H), 6.4 (d, J=6.2 Hz, 1H), 7.2 (m, 2H), 7.2 (m, 1H), 7.5 (d, J=7.7 Hz, 1 H), 7.6 (s, 1H), 8.3 (d, J=4.8 Hz, 1H); m/z 431 (M+1).

### Example 78

### (4aR,10bS)-1-{[5-(4-thiomorpholinyl)imidazo[1,2-a]pyridin-2-yl]methyl}-1,2,3,4,4a,5,6,10b-octahydro-1,10-phenanthroline

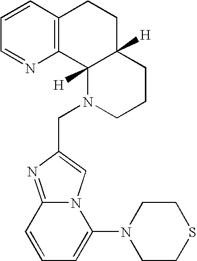

To a solution of (4aR,10bS)-1-[(5-fluoroimidazo[1,2-a]pyridin-2-yl)methyl]-1,2,3,4,4a,5,6,10b-octahydro-1,10-phenanthroline (0.050 g, 0.15 mmol) in 2 mL of dimethyl sulfoxide was added thiomorpholine (0.30 g). The mixture was heated at 60° C. for 16 hours and then allowed to cool to room temperature. The mixture was quenched with water and extracted 3 times with 10 mL of dichloromethane, and then washed with brine. The organic layer was concentrated and the residue purified by silica chromatography eluting with a 0% to 10% gradient of 30% aqueous ammonium hydroxide in acetonitrile to yield 45 mg (71%) of (4aR,10bS)-1-{[5-(4-thiomorpholinyl)imidazo[1,2-a]pyridin-2-yl]methyl}-1,2,3,4,4a,5,6,10b-octahydro-1,10-phenanthroline. ¹H NMR (400 MHz, METHANOL-D4) δ ppm 1.7 (m, 5H), 2.1 (m, 1 H), 2.4 (m, 2H), 2.6 (m, 4H), 2.8 (m, 1H), 2.9 (m, 1H), 3.0 (m, 4H), 3.1 (m, 1H), 3.6 (m, 2H), 4.0 (d, J=14.7 Hz, 1H), 6.4 (d, J=7.1 Hz, 1H), 7.2 (m, 2H), 7.2 (m, 1 H), 7.5 (d, J=8.2 Hz, 1H), 7.6 (s, 1H), 8.3 (m, 1H); m/z 420 (M+1).

### Example 79

### (4aR,10bS)-1-({5-[(2R,5S)-2,5-dimethyl-1-piperazinyl]imidazo[1,2-a]pyridin-2-yl}methyl)-1,2,3,4,4a,5,6,10b-octahydro-1,10-phenanthroline

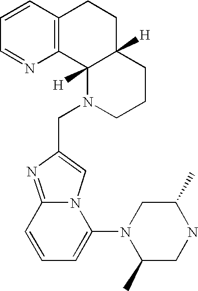

To a solution of (4aR,10bS)-1-[(5-fluoroimidazo[1,2-a]pyridin-2-yl)methyl]-1,2,3,4,4a,5,6,10b-octahydro-1,10-phenanthroline (0.050 g, 0.15 mmol) in 2 mL of dimethyl sulfoxide was added (2R,5S)-2,5-dimethylpiperazine (0.30 g). The mixture was heated at 60° C. for 16 hours and then allowed to cool to room temperature. The mixture was quenched with water and extracted 3 times with 10 mL of dichloromethane, and then washed with brine. The organic layer was concentrated and the residue purified by silica chromatography eluting with a 0% to 10% gradient of 30% aqueous ammonium hydroxide in acetonitrile to yield 21 mg (32%) of (4aR,10bS)-1-({5-[(2R,5S)-2,5-dimethyl-1-piperazinyl]imidazo[1,2-a]pyridin-2-yl}methyl)-1,2,3,4,4a,5,6,10b-octahydro-1,10-phenanthroline. ¹H NMR (400 MHz, METHANOL-D4) δ ppm 0.9 (dd, J=15.1, 5.9 Hz, 3H), 1.1 (dd, J=36.1, 6.3 Hz, 3H), 1.7 (m, 5H), 2.1 (m, 1H), 2.3 (m, 1H), 2.4 (m, 2H), 2.8 (m, 2H), 3.0 (m, 1H), 3.1 (m, 5H), 3.7 (m, 2H), 4.1 (t, J=14.3 Hz, 1H), 6.6 (d, J=6.8 Hz, 1H), 7.2 (m, 1H), 7.3 (m, 2H), 7.5 (d, J=7.7 Hz, 1H), 7.7 (s, 1H), 8.3 (dd, J=12.4, 4.8 Hz, 1H); m/z 431 (M+1).

### Example 80

### (4aR,10bS)-1-({5-[4-(2-pyridinyl)-1-piperazinyl]imidazo[1,2-a]pyridin-2-yl}methyl)-1,2,3,4,4a,5,6,10b-octahydro-1,10-phenanthroline

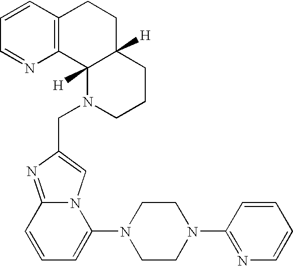

To a solution of (4aR,10bS)-1-[(5-fluoroimidazo[1,2-a]pyridin-2-yl)methyl]-1,2,3,4,4a,5,6,10b-octahydro-1,10-phenanthroline (0.050 g, 0.15 mmol) in 2 mL of dimethyl sulfoxide was added 1-(2-pyridinyl)piperazine (0.30 g). The mixture was heated at 60° C. for 16 hours and then allowed to cool to room temperature. The mixture was quenched with water and extracted 3 times with 10 mL of dichloromethane, and then washed with brine. The organic layer was concentrated and the residue purified by silica chromatography eluting with a 0% to 10% gradient of 30% aqueous ammonium hydroxide in acetonitrile to yield 24 mg (33%) of (4aR,10bS)-1-({5-[4-(2-pyridinyl)-1-piperazinyl]imidazo[1,2-a]pyridin-2-yl}methyl)-1,2,3,4,4a,5,6,10b-octahydro-1,10-phenanthroline. ¹H NMR (400 MHz, METHANOL-D4) δ ppm 1.7 (m, 4H), 2.2 (m, 1H), 2.4 (m, 2H), 2.8 (m, 1H), 3.0 (m, 1H), 3.1 (m, 1H), 3.2 (m, 3H), 3.7 (m, 8H), 4.1 (m, 1H), 6.5 (d, J=7.5 Hz, 1H), 6.7 (dd, J=7.0, 4.9 Hz, 1H), 6.9 (d, J=8.6 Hz, 1H), 7.2 (m, 2H), 7.3 (m, 1H), 7.5 (d, J=7.7 Hz, 1H), 7.6 (m, 1H), 7.7 (s, 1H), 8.1 (d, J=7.0 Hz, 1H), 8.3 (d, J=3.5 Hz, 1H); m/z 480 (M+1).

### Example 81

### N-{2-[(4aR,10bS)-3,4,4a,5,6,10b-hexahydro-1,10-phenanthrolin-1(2H)-ylmethyl]imidazo[1,2-a]pyridin-5-yl}-N,N′-dimethyl-1,3-propanediamine

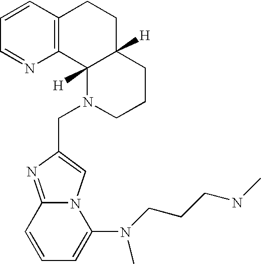

To a solution of (4aR,10bS)-1-[(5-fluoroimidazo[1,2-a]pyridin-2-yl)methyl]-1,2,3,4,4a,5,6,10b-octahydro-1,10-phenanthroline (0.050 g, 0.15 mmol) in 2 mL of dimethyl sulfoxide was added N,N′-dimthyl-1,3-propanediamine (0.30 g). The mixture was heated at 60° C. for 16 hours and then allowed to cool to room temperature. The mixture was quenched with water and extracted 3 times with 10 mL of dichloromethane, and then washed with brine. The organic layer was concentrated and the residue purified by silica chromatography eluting with a 0% to 10% gradient of 30% aqueous ammonium hydroxide in acetonitrile to yield 44 mg (70%) of N-{2-[(4aR,10bS)-3,4,4a,5,6,10b-hexahydro-1,10-phenanthrolin-1(2H)-ylmethyl]imidazo[1,2-a]pyridin-5-yl}-N,N′-dimethyl-1,3-propanediamine. ¹H NMR (400 MHz, METHANOL-D4) δ ppm 1.6 (m, 1H), 1.7 (m, 3H), 1.8 (m, 3H), 2.1 (m, 1H), 2.3 (m, 4H), 2.4 (m, 1H), 2.6 (t, J=7.3 Hz, 2H), 2.8 (t, J=7.6 Hz, 4H), 3.0 (m, 1H), 3.1 (m, 3H), 3.6 (m, 2H), 4.0 (d, J=14.5 Hz, 1H), 6.4 (d, J=7.1 Hz, 1H), 7.1 (d, J=9.0 Hz, 1H), 7.2 (m, 2H), 7.5 (d, J=7.3 Hz, 1H), 7.6 (s, 1H), 8.3 (d, J=4.8 Hz, 1 H); m/z 419 (M+1).

### Example 82

### (4aR,10bS)-1-({5-[4-(1-methylethyl)-1-piperazinyl]imidazo[1,2-a]pyridin-2-yl}methyl)-1,2,3,4,4a,56,10b-octahydro-1,10-phenanthroline

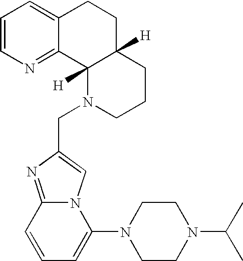

Dissolved (4aR,10bS)-1-[(5-fluoroimidazo[1,2-a]pyridin-2-yl)methyl]-1,2,3,4,4a,5,6,10b-octahydro-1,10-phenanthroline (0.05 g, 0.149 mmol) in 1-(1-methylethyl)piperazine (1 mL) and heated in a microwave reactor at 100° C. for one hour, then at 200° C. for 30 additional minutes. Diluted reaction mixture with saturated aqueous sodium chloride and extracted three times with dichloromethane. Organics were dried over sodium sulfate and concentrated. Residue was purified by reverse phase chromatography on a 0-100% gradient of acetonitrile in water (0.1% trifluoroacetic acid) to recover 0.0592 g (89%) of (4aR,10bS)-1-({5-[4-(1-methylethyl)-1-piperazinyl]imidazo[1,2-a]pyridin-2-yl}methyl)-1,2,3,4,4a,5,6,10b-octahydro-1,10-phenanthroline as the trifluoroacetic acid salt. ¹H NMR (400 MHz, DMSO-D4) δ 1.25-1.44 (m, 7H), 1.71 (m, 2H), 1.97 (m, 3H), 2.85 (m, 4H), 3.15-3.47 (m, 5H), 3.67 (m, 5H), 4.66 (m, 2H), 4.91 (d, 1H), 6.81 (m, 1H), 7.54 (m, 3H), 7.83 (d, 1H) 8.24 (s, 1H), 8.61 (d, 1H); MS m/z 445 (M+1).

### Example 83

### 1-{2-[(4aR,10bS)-3,4,4a,5,6,10b-hexahydro-1,10-phenanthrolin-1(2H)-ylmethyl]imidazo[1,2-a]pyridin-5-yl}-N,N-dimethyl-3-pyrrolidinamine

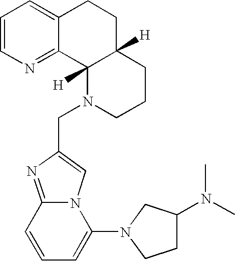

Dissolved (4aR,10bS)-1-[(5-fluoroimidazo[1,2-a]pyridin-2-yl)methyl]-1,2,3,4,4a,5,6,10b-octahydro-1,10-phenanthroline (0.05 g, 0.149 mmol) in N,N-dimethyl-3-pyrrolidinamine (1 mL) and heated in a microwave reactor at 100° C. for one hour. Diluted reaction mixture with saturated aqueous sodium chloride and extracted three times with dichloromethane. Organics were dried over sodium sulfate and concentrated. Residue was purified by reverse phase chromatography on a 0-100% gradient of acetonitrile in water (0.1% trifluoroacetic acid) to recover 0.060 g (95%) of 1-{2-[(4aR,10bS)-3,4,4a,5,6,10b-hexahydro-1,10-phenanthrolin-1(2″-ylmethyl]imidazo[1,2-a]pyridin-5-yl}-N,N-dimethyl-3-pyrrolidinamine as the trifluoroacetic acid salt. ¹H NMR (300 MHz, DMSO-D4) δ 1.40 (m, 1H), 1.68 (t, 2H), 1.97 (m, 3H), 2.32 (m, 1H), 2.65-3.00 (m, 11H), 3.15 (m, 1H), 3.51 (m, 1H), 3.77 (m, 3H), 4.13 (m, 1H), 4.62 (m, 2H), 4.91 (d, 1H), 6.76 (d, 1H), 7.46 (d, 1H), 7.58 (m, 1H), 7.68 (m, 1H), 7.90 (d, 1H), 8.40 (s, 1H), 8.63 (d, 1H); MS m/z 431 (M+1).

### Example 84

### 1,1-dimethylethyl [(1-{2-[(4aR,10bS)-3,4,4a,5,6,10b-hexahydro-1,10-phenanthrolin-1(2H)-ylmethyl]imidazo[1,2-a]pyridin-5-yl}-4-piperidinyl)methyl]carbamate (intermediate)

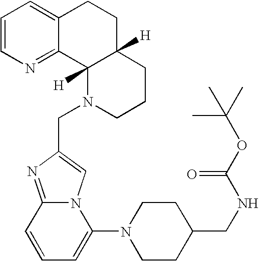

Dissolved (4aR,10bS)-1-[(5-fluoroimidazo[1,2-a]pyridin-2-yl)methyl]-1,2,3,4,4a,5,6,10b-octahydro-1,10-phenanthroline (0.03 g, 0.089 mmol) and 1,1-dimethylethyl (4-piperidinylmethyl)carbamate (0.45 g, 2.10 mmol) in 1-methyl-2-pyrrolidinone (0.75 mL) and heated in a microwave reactor at 150° C. for 30 minutes. Diluted reaction mixture with water and extracted three times with ethyl acetate. Organics were dried over sodium sulfate and concentrated. Residue was purified by reverse phase chromatography on a 0-100% gradient of acetonitrile in water (0.1% trifluoroacetic acid) to recover 0.056 g (97%) of 1,1-dimethylethyl [(1-{2-[(4aR,10bS)-3,4,4a,5,6,10b-hexahydro-1,10-phenanthrolin-1(2H)-ylmethyl]imidazo[1,2-a]pyridin-5-yl}-4-piperidinyl)methyl]carbamate as the trifluoroacetic acid salt. ¹H NMR (300 MHz, DMSO-D4) δ 1.42 (m, 10H), 1.71 (m, 3H), 1.82-2.10 (m, 6H), 2.72-3.03 (m, 8H), 3.22 (m, 1H), 3.44 (m, 3H), 4.67 (m, 2H), 4.93 (d, 1H), 6.71 (d, 1H), 7.01 (t, 1H), 7.52 (m, 3H), 7.81 (m, 1H), 8.08 (s, 1H), 8.59 (d, 1H); MS m/z 531 (M+1).

### Example 85

### [(1-{2-[(4aR,10bS)-3,4,4a,5,6,10b-hexahydro-1,10-phenanthrolin-1(2H)-ylmethyl]imidazo[1,2-a]pyridin-5-yl}-4-piperidinyl)methyl]amine

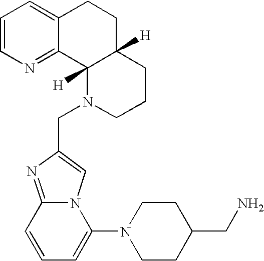

1,1-dimethylethyl [(1-{2-[(4aR,10bS)-3,4,4a,5,6,10b-hexahydro-1,10-phenanthrolin-1(2H)-ylmethyl]imidazo[1,2-a]pyridin-5-yl}-4-piperidinyl)methyl]carbamate (0.0527 g, 0.099 mmol) was dissolved in dichloromethane (4 mL) and trifluoroacetic acid (2 mL) was added. The reaction mixture was stirred for 30 minutes and concentrated to afford 0.039 g (92%) of [(1-{2-[(4aR,10bS)-3,4,4a,5,6,10b-hexahydro-1,10-phenanthrolin-1(2H)-ylmethyl]imidazo[1,2-a]pyridin-5-yl}-4-piperidinyl)methyl]amine as the trifluoroacetic acid salt. ¹H NMR (400 MHz, DMSO-D4) δ 0.84 (m, 1H), 1.17-1.37 (m, 4H), 1.44 (m, 2H), 1.63 (m, 2H), 1.75-2.03 (m, 5H), 2.78 (m, 6H), 3.41 (t, 2H), 3.93 (m, 1H), 4.11 (m, 1H), 4.59 (m, 2H), 4.86 (d, 1H), 6.66 (d, 1H), 7.44 (m, 1H), 7.50 (t, 1H), 7.67 (m, 1H), 7.76 (d, 1H), 8.00 (s, 1H), 8.53 (d, 1H); MS m/z 431 (M+1).

### Example 86

### [(1-{2-[(4aR,10bS)-3,4,4a,5,6,10b-hexahydro-1,10-phenanthrolin-1(2H)-ylmethyl]imidazo[1,2-a]pyridin-5-yl}-4-piperidinyl)methyl]dimethylamine

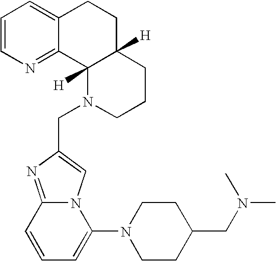

To a solution of [(1-{2-[(4aR,10bS)-3,4,4a,5,6,10b-hexahydro-1,10-phenanthrolin-1(2H)-ylmethyl]imidazo[1,2-a]pyridin-5-yl}-4-piperidinyl)methyl]amine (0.034 g, 0.078 mmol) in 1,2-dichloroethane (1 mL) was added 37% aqueous formaldehyde (0.037 mL, 0.467 mmol) and acetic acid (0.009 mL, 0.156 mmol). Reaction stirred for 30 minutes, then sodium triacetoxyborohydride (0.033 g, 0.156 mmol) was added and stirred for an additional two hours. Mixture was diluted with dichloromethane and stirred vigorously with 10% aqueous sodium carbonate overnight. The layers were separated and washed with water. Dried organics over sodium sulfate and concentrated. Residue was purified by reverse phase chromatography on a 0-100% gradient of acetonitrile in water (0.1% trifluoroacetic acid) to recover 0.006 g (17%) of [(1-{2-[(4aR,10bS)-3,4,4a,5,6,10b-hexahydro-1,10-phenanthrolin-1(2″-ylmethyl]imidazo[1,2-a]pyridin-5-yl}-4-piperidinyl)methyl]dimethylamine as the trifluoroacetic acid salt. ¹H NMR (300 MHz, METHANOL-D4) δ 1.46-1.89 (m, 6H), 2.08 (m, 6H), 2.76 (m, 2H), 3.04 (m, 9H), 3.23 (d, 2H), 3.61 (m, 3H), 4.53 (d, 1H), 4.72 (s, 2H), 6.96 (d, 1H), 7.61 (d, 1H), 7.73 (m, 1H), 7.85 (m, 1H), 8.12 (d, 1H), 8.17 (s, 1H), 8.73 (d, 1H); MS m/z 459 (M+1).

### Example 87

### (4aR,10bS)-1-{[5-(hexahydro-1H-1,4-diazepin-1-yl)imidazo[1,2-a]pyridin-2-yl]methyl}-1,2,3,4,4a,5,6,10b-octahydro-1,10-phenanthroline

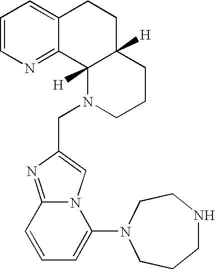

Combined (4aR,10bS)-1-[(5-fluoroimidazo[1,2-a]pyridin-2-yl)methyl]-1,2,3,4,4a,5,6,10b-octahydro-1,10-phenanthroline (0.05 g, 0.149 mmol) and hexahydro-1H-1,4-diazepine (1.0 g, 9.98 mmol) as solids and heated in a microwave reactor at 150° C. for one hour. Added 1-methyl-2-pyrrolidinone (1 mL) and reacted an additional 30 minutes at 150° C. Diluted reaction mixture with water and extracted three times with dichloromethane. Organics were dried over sodium sulfate and concentrated. Residue was purified by reverse phase chromatography on a 0-100% gradient of acetonitrile in water (0.1% trifluoroacetic acid) to recover 0.057 g (93%) of (4aR,10bS)-1-{[5-(hexahydro-1H-1,4-diazepin-1-yl)imidazo[1,2-a]pyridin-2-yl]methyl}-1,2,3,4,4a,5,6,10b-octahydro-1,10-phenanthroline as the trifluoroacetic acid salt. ¹H NMR (300 MHz, DMSO-D4) δ 1.40 (m, 1H), 1.72 (m, 2H), 1.97 (m, 3H), 2.16 (m, 2H), 2.72-2.95 (m, 4H), 3.23 (m, 1H), 3.42 (m, 7H), 3.58 (m, 2H), 4.64 (m, 2H), 4.93 (d, 1H), 6.77 (d, 1H), 7.48 (m, 3H), 7.78 (d, 1H), 8.09 (s, 1H), 8.58 (d, 1H); MS m/z 417 (M+1).

### Example 88

### 1,1-dimethylethyl (1-{2-[(4aR,10bS)-3,4,4a,5,6,10b-hexahydro-1,10-phenanthrolin-1(2H)-ylmethyl]imidazo[1,2-a]pyridin-5-yl}-4-piperidinyl)carbamate (intermediate)

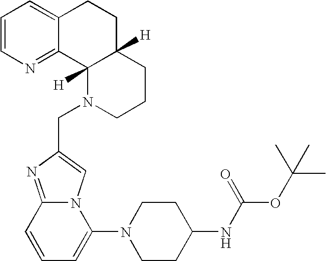

Dissolved (4aR,10bS)-1-[(5-fluoroimidazo[1,2-a]pyridin-2-yl)methyl]-1,2,3,4,4a,5,6,10b-octahydro-1,10-phenanthroline (0.04 g, 0.119 mmol) in 1-methyl-2-pyrrolidinone (0.5 mL) and added 1,1-dimethylethyl 4-piperidinylcarbamate (0.216 g, 1.08 mmol). The reaction was heated in a microwave reactor at 150° C. for 30 minutes. Diluted reaction mixture with water and extracted three times with ethyl acetate. Organics were dried over sodium sulfate and concentrated. Residue was purified by reverse phase chromatography on a 0-100% gradient of acetonitrile in water (0.1% trifluoroacetic acid) to recover 0.051 g (84%) of 1,1-dimethylethyl (1-{2-[(4aR,10bS)-3,4,4a,5,6,10b-hexahydro-1,10-phenanthrolin-1(2H)-ylmethyl]imidazo[1,2-a]pyridin-5-yl}-4-piperidinyl)carbamate as the trifluoroacetic acid salt. ¹H NMR (300 MHz, DMSO-D4) δ 1.43 (m, 10H), 1.71 (m, 4H), 1.97 (m, 5H), 2.74-2.97 (m, 5H), 3.22 (m, 1H), 3.36-3.60 (m, 4H), 4.67 (m, 2H), 4.93 (d, 1H), 6.75 (d, 1H), 7.12 (d, 1H), 7.52 (m, 2H), 7.61 (t, 1H), 7.83 (d, 1H), 8.09 (s, 1H), 8.61 (d, 1H); MS m/z 417 (M+1).

### Example 89

### 1-{2-[(4aR,10bS)-3,4,4a,5,6,10b-hexahydro-1,10-phenanthrolin-1(2H)-ylmethyl]imidazo[1,2-a]pyridin-5-yl}-4-piperidinamine

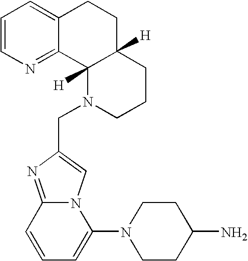

1,1-dimethylethyl (1-{2-[(4aR,10bS)-3,4,4a,5,6,10b-hexahydro-1,10-phenanthrolin-1(2H)-ylmethyl]imidazo[1,2-a]pyridin-5-yl}-4-piperidinyl)carbamate (0.0517 g, 0.100 mmol) was dissolved in dichloromethane (4 mL) and trifluoroacetic acid (2 mL) was added. The reaction mixture was stirred for two hours and concentrated to afford 0.040 g (96%) of 1-{2-[(4aR,10bS)-3,4,4a,5,6,10b-hexahydro-1,10-phenanthrolin-1(2H)-ylmethyl]imidazo[1,2-a]pyridin-5-yl}-4-piperidinamine as the trifluoroacetic acid salt. ¹H NMR (300 MHz, DMSO-D4) δ 0.90 (m, 2H), 1.23-1.45 (m, 2H), 1.66-2.17 (m, 10H), 2.74-2.98 (m, 5H), 3.36 (m, 1H), 3.50 (t, 2H), 4.66 (m, 2H), 4.95 (d, 1H), 6.63 (d, 1H), 7.44 (m, 2H), 7.75 (m, 2H), 8.00 (s, 1H), 8.57 (d, 1H); MS m/z 417 (M+1).

### Example 90

### N′-{2-[(4aR,10bS)-3,4,4a,5,6,10b-hexahydro-1,10-phenanthrolin-1(2H)-ylmethyl]imidazo[1,2-a]pyridin-5-yl}-N,N-dimethyl-1,2-ethanediamine

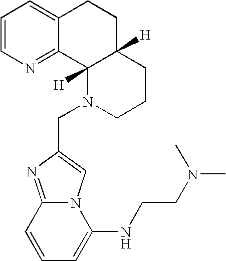

Dissolved (4aR,10bS)-1-[(5-fluoroimidazo[1,2-a]pyridin-2-yl)methyl]-1,2,3,4,4a,5,6,10b-octahydro-1,10-phenanthroline (0.05 g, 0.149 mmol) in N,N-dimethyl-1,2-ethanediamine (1.0 mL, 9.1 mmol). The reaction was heated in a microwave reactor at 100° C. for one hour. Diluted reaction mixture with water and extracted three times with dichloromethane. Organics were dried over sodium sulfate and concentrated. Residue was purified by reverse phase chromatography on a 0-100% gradient of acetonitrile in water (0.1% trifluoroacetic acid) to recover 0.029 g (48%) of N-{2-[(4aR,10bS)-3,4,4a,5,6,10b-hexahydro-1,10-phenanthrolin-1(2H)-ylmethyl]imidazo[1,2-a]pyridin-5-yl}-N,N-dimethyl-1,2-ethanediamine as the trifluoroacetic acid salt. ¹H NMR (400 MHz, DMSO-D4) δ 1.39 (m, 1H), 1.65 (m, 2H), 1.93 (m, 3H), 2.62 (m, 1H), 2.77-2.94 (m, 9H), 3.01 (m, 1H), 3.38 (m, 2H), 3.73 (m, 2H), 4.47 (s, 1H), 4.56 (d, 1H), 4.76 (d, 1H), 6.44 (d, 1H), 7.14 (d, 1H), 7.53 (t, 1H), 7.63 (m, 2H), 7.86 (d, 1H), 8.22 (s, 1H), 8.59 (d, 1H); MS m/z 405 (M+1).

### Example 91

### 1,1-dimethylethyl [2-(4-{2-[(4aR,10bS)-3,4,4a,5,6,10b-hexahydro-1,10-phenanthrolin-1(2H)-ylmethyl]imidazo[1,2-a]pyridin-5-yl}-1-piperazinyl)ethyl]carbamate (intermediate)

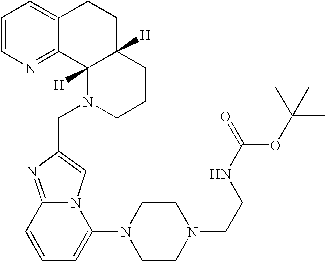

Dissolved (4aR,10bS)-1-[(5-fluoroimidazo[1,2-a]pyridin-2-yl)methyl]-1,2,3,4,4a,5,6,10b-octahydro-1,10-phenanthroline (0.03 g, 0.089 mmol) in 1-methyl-2-pyrrolidinone (1.0 mL) and added 1,1-dimethylethyl [2-(1-piperazinyl)ethyl]carbamate (1.0 mL, 4.36 mmol). The reaction was heated in a microwave reactor at 150° C. for 30 minutes. Diluted reaction mixture with water and extracted three times with ethyl acetate. Organics were dried over sodium sulfate and concentrated. Residue was purified by reverse phase chromatography on a 0-100% gradient of acetonitrile in water (0.1% trifluoroacetic acid) to recover 0.020 g (40%) of 1,1-dimethylethyl [2-(4-{2-[(4aR,10bS)-3,4,4a,5,6,10b-hexahydro-1,10-phenanthrolin-1(2″-ylmethyl]imidazo[1,2-a]pyridin-5-yl}-1-piperazinyl)ethyl]carbamate as the trifluoroacetic acid salt. ¹H NMR (300 MHz, DMSO-D4) δ 1.39-1.48 (m, 10H), 1.72 (d, 2H), 1.84-2.12 (m, 3H), 2.72-2.97 (m, 4H), 3.09-3.47 (m, 11H), 3.57-3.84 (m, 2H), 4.67 (m, 2H), 4.93 (d, 1H), 6.73 (d, 1H), 7.46 (m, 3H), 7.77 (m, 2H), 8.09 (s, 1H), 8.58 (d, 1H); MS m/z 546 (M+1).

### Example 92

### [2-(4-{2-[(4aR,10bS)-3,4,4a,5,6,10b-hexahydro-1,10-Phenanthrolin-1(2H)-ylmethyl]imidazo[1,2-a]pyridin-5-yl}-1-piperazinyl)ethyl]amine

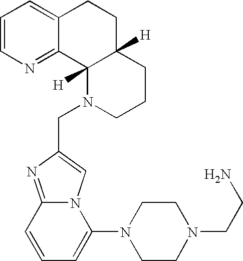

1,1-dimethylethyl [2-(4-{2-[(4aR,10bS)-3,4,4a,5,6,10b-hexahydro-1,10-phenanthrolin-1(2H)-ylmethyl]imidazo[1,2-a]pyridin-5-yl}-1-piperazinyl)ethyl]carbamate (0.014 g, 0.026 mmol) was dissolved in dichloromethane (1 mL) and trifluoroacetic acid (1 mL) was added. The reaction mixture was stirred for 30 minutes and concentrated to afford 0.010 g (87%) of [2-(4-{2-[(4aR,10bS)-3,4,4a,5,6,10b-hexahydro-1,10-phenanthrolin-1(2H)-ylmethyl]imidazo[1,2-a]pyridin-5-yl}-1-piperazinyl)ethyl]amine as the trifluoroacetic acid salt. ¹H NMR (300 MHz, DMSO-D4) δ 0.89 (m, 1H), 1.34 (m, 2H), 1.72 (d, 1H), 1.97 (m, 2H), 2.72-3.15 (m, 12H), 3.16-3.36 (m, 5H), 4.22 (m, 2H), 4.66 (m, 2H), 4.95 (d, 1H), 6.67 (d, 1H), 7.46 (m, 2H), 7.76 (m, 2H), 8.08 (s, 1H), 8.58 (d, 1H); MS m/z 446 (M+1).

### Example 93

### (4aR,10bS)-1-{[5-(4-methylhexahydro-1H-1,4-diazepin-1-yl)imidazo[1,2-a]pyridin-2-yl]methyl}-1,2,3,4,4a,5,6,10b-octahydro-1,10-phenanthroline

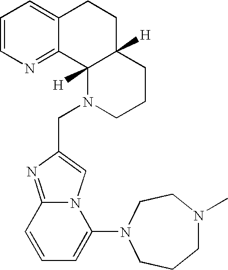

Dissolved (4aR,10bS)-1-[(5-fluoroimidazo[1,2-a]pyridin-2-yl)methyl]-1,2,3,4,4a,5,6,10b-octahydro-1,10-phenanthroline (0.05 g, 0.149 mmol) in 1-methyl-2-pyrrolidinone (1.0 mL) and added 1-methylhexahydro-1H-1,4-diazepine (1.0 mL, 8.04 mmol). The reaction was heated in a microwave reactor at 150° C. for 30 minutes. Diluted reaction mixture with water and extracted three times with ethyl acetate. Organics were dried over sodium sulfate and concentrated. Residue was purified by reverse phase chromatography on a 0-100% gradient of acetonitrile in water (0.1% trifluoroacetic acid) to recover 0.032 g (50%) of (4aR,10bS)-1-{[5-(4-methylhexahydro-1H-1,4-diazepin-1-yl)imidazo[1,2-a]pyridin-2-yl]methyl}-1,2,3,4,4a,5,6,10b-octahydro-1,10-phenanthroline as the trifluoroacetic acid salt. ¹H NMR (300 MHz, DMSO-D4) δ 1.41 (m, 1H), 1.71 (d, 2H), 1.97 (m, 3H), 2.22 (m, 2H), 2.69-2.99 (m, 7H), 3.20 (d, 1H), 3.44 (m, 4H), 3.66 (m, 4H), 4.66 (m, 2H), 4.92 (d, 1H), 6.79 (d, 1H), 7.52 (m, 3H), 7.81 (d, 1H), 8.11 (s, 1H), 8.60 (d, 1H); MS m/z 431 (M+1).

### Example 94

### 1,1-dimethylethyl (1-{2-[(4aR,10bS)-3,4,4a,5,6,10b-hexahydro-1,10-phenanthrolin-1(2H)-ylmethyl]imidazo[1,2-a]pyridin-5-yl}-3-pyrrolidinyl)methylcarbamate (intermediate)

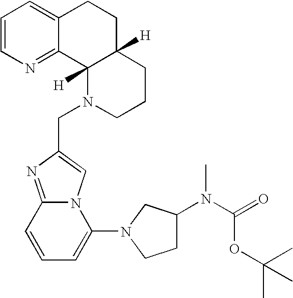

Dissolved 1-[(5-fluoroimidazo[1,2-a]pyridin-2-yl)methyl]-1,2,3,4,4a,5,6,10b-octahydro-1,10-phenanthroline (0.05 g, 0.149 mmol) in 1-methyl-2-pyrrolidinone (1.0 mL) and added 1,1-dimethylethyl methyl(3-pyrrolidinyl)carbamate (1.0 mL, 5.09 mmol). The reaction was heated in a microwave reactor at 150° C. for 30 minutes. Diluted reaction mixture with ethyl acetate and extracted three times with water, then back extracted combined aqueous layers with ethyl acetate. Organics were dried over sodium sulfate and concentrated. Residue was purified by reverse phase chromatography on a 0-100% gradient of acetonitrile in water (0.1% trifluoroacetic acid) to recover 0.031 g (41%) of 1,1-dimethylethyl (1-{2-[(4aR,10bS)-3,4,4a,5,6,10b-hexahydro-1,10-phenanthrolin-1(2″-ylmethyl]imidazo[1,2-a]pyridin-5-yl}-3-pyrrolidinyl)methylcarbamate as the trifluoroacetic acid salt. ¹H NMR (300 MHz, DMSO-D4) δ 1.40 (m, 10H), 1.65 (m, 2H), 1.94 (m, 3H), 2.14 (m, 2H), 2.64-2.89 (m, 8H), 3.11 (m, 1H), 3.48 (m, 4H), 4.57 (m, 2H), 4.79 (m, 1H), 6.56 (d, 1H), 7.29 (d, 1H), 7.46 (m, 1H), 7.53 (t, 1H), 7.79 (d, 1H), 8.27 (s, 1H), 8.55 (d, 1H); MS m/z 517 (M+1).

### Example 95

### 1-{2-[(4aR,10bS)-3,4,4a,5,6,10b-hexahydro-1,10-phenanthrolin-1(2H)-ylmethyl]imidazo[1,2-a]pyridin-5-yl}-N-methyl-3-pyrrolidinamine

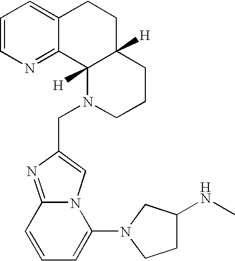

1,1-dimethylethyl (1-{2-[(4aR,10bS)-3,4,4a,5,6,10b-hexahydro-1,10-phenanthrolin-1(2H)-ylmethyl]imidazo[1,2-a]pyridin-5-yl}-3-pyrrolidinyl)methylcarbamate (0.034 g, 0.066 mmol) was dissolved in dichloromethane (2 mL) and trifluoroacetic acid (2 mL) was added. The reaction mixture was stirred for one hour and concentrated. Residue was purified by reverse phase chromatography on a 0-100% gradient of acetonitrile in water (0.1% trifluoroacetic acid) to recover 0.022 g (80%) of 1-{2-[(4aR,10bS)-3,4,4a,5,6,10b-hexahydro-1,10-phenanthrolin-1(2H)-ylmethyl]imidazo[1,2-a]pyridin-5-yl}-N-methyl-3-pyrrolidinamine as the trifluoroacetic acid salt. ¹H NMR (400 MHz, METHANOL-D4) δ 1.48 (m, 2H), 1.78 (m, 1H), 2.02 (m, 3H), 2.31 (m, 1H), 2.52-2.71 (m, 3H), 2.81 (s, 3H), 2.87-3.08 (m, 3H), 3.39 (m, 1H), 3.72 (m, 2H), 3.87 (m, 1H), 4.03 (m, 1H), 4.44 (s, 1H), 4.62 (s, 2H), 6.85 (d, 1H), 7.49 (d, 1H), 7.69 (t, 1H), 7.77 (t, 1H), 8.09 (d, 1H), 8.37 (s, 1H), 8.66 (d, 1H); MS m/z 417 (M+1).

### Example 96

### 1,1-dimethylethyl (1-{2-[(4aR,10bS)-3,4,4a,5,6,10b-hexahydro-1,10-phenanthrolin-1(2H)-ylmethyl]imidazo[1,2-a]pyridin-5-yl}-3-azetidinyl)carbamate (intermediate)

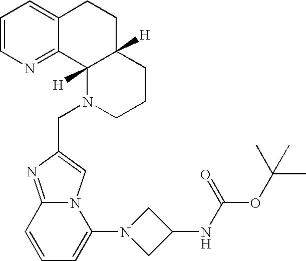

Dissolved (4aR,10bS)-1-[(5-fluoroimidazo[1,2-a]pyridin-2-yl)methyl]-1,2,3,4,4a,5,6,10b-octahydro-1,10-phenanthroline (0.05 g, 0.149 mmol) in 1-methyl-2-pyrrolidinone (1.0 mL) and added 1,1-dimethylethyl 3-azetidinylcarbamate (0.25 g, 1.45 mmol). The reaction was heated in a microwave reactor at 150° C. for 30 minutes. Diluted reaction mixture with water and extracted three times with ethyl acetate. Organics were dried over sodium sulfate and concentrated. Residue was purified by reverse phase chromatography on a 0-100% gradient of acetonitrile in water (0.1% trifluoroacetic acid) to recover 0.031 g (42%) of 1,1-dimethylethyl (1-{2-[(4aR,10bS)-3,4,4a,5,6,10b-hexahydro-1,10-phenanthrolin-1(2H)-ylmethyl]imidazo[1,2-a]pyridin-5-yl}-3-azetidinyl)carbamate as the trifluoroacetic acid salt. ¹H NMR (300 MHz, METHANOL-D4) δ 1.51 (m, 12H), 1.86 (m, 1H), 2.08 (m, 4H), 2.71 (m, 2H), 3.05 (m, 3H), 4.24 (m, 2H), 4.48 (m, 1H), 4.66 (m, 3H), 6.49 (d, 1H), 7.32 (d, 1H), 7.80 (m, 2H), 8.19 (m, 2H), 8.73 (d, 1H); MS m/z 489 (M+1).

### Example 97

### 1-{2-[(4aR,10bS)-3,4,4a,5,6,10b-hexahydro-1,10-phenanthrolin-1(2H)-ylmethyl]imidazo[1,2-a]pyridin-5-yl}-3-azetidinamine

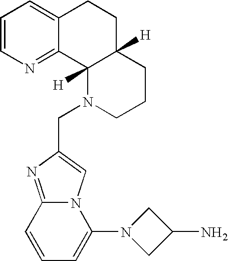

1,1-dimethylethyl (1-{2-[(4aR,10bS)-3,4,4a,5,6,10b-hexahydro-1,10-phenanthrolin-1(2H)-ylmethyl]imidazo[1,2-a]pyridin-5-yl}-3-azetidinyl)carbamate (0.026 g, 0.052 mmol) was dissolved in dichloromethane (2 mL) and trifluoroacetic acid (2 mL) was added. The reaction mixture was stirred for 90 minutes and concentrated to afford 0.0209 (100%) of 1-{2-[(4aR,10bS)-3,4,4a,5,6,10b-hexahydro-1,10-phenanthrolin-1(2H)-ylmethyl]imidazo[1,2-a]pyridin-5-yl}-3-azetidinamine as the trifluoroacetic acid salt. ¹H NMR (300 MHz, DMSO-D4) δ 0.90 (m, 1H), 1.34 (m, 3H), 1.70 (m, 2H), 1.97 (m, 3H), 2.67-2.99 (m, 5H), 3.16 (m, 1H), 4.25 (m, 2H), 4.48-4.69 (m, 3H), 4.86 (d, 1H), 6.41 (d, 1H), 7.31 (d, 1H), 7.57 (m, 2H), 7.86 (m, 1H), 8.16 (s, 1H), 8.61 (d, 1H); MS m/z 389 (M+1).

### Example 98

### 1-{2-[(4aR,10bS)-3,4,4a,5,6,10b-hexahydro-1,10-phenanthrolin-1(2H)-ylmethyl]imidazo[1,2-a]pyridin-5-yl}-N,N-dimethyl-3-azetidinamine

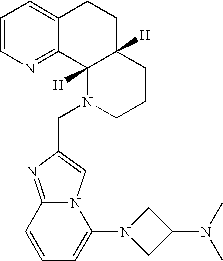

To a solution of 1-{2-[(4aR,10bS)-3,4,4a,5,6,10b-hexahydro-1,10-phenanthrolin-1(2″-ylmethyl]imidazo[1,2-a]pyridin-5-yl}-3-azetidinamine (0.020 g, 0.052 mmol) in 1,2-dichloroethane (2 mL) was added 37% aqueous formaldehyde (0.012 mL, 0.155 mmol), acetic acid (0.006 mL, 0.104 mmol) and sodium triacetoxyborohydride (0.022 g, 0.104 mmol) was added and stirred at room temperature overnight. Mixture was diluted with dichloromethane and stirred vigorously with 10% aqueous sodium carbonate for 30 minutes. The layers were separated and washed with water and saturated aqueous sodium chloride. Organics were dried over sodium sulfate and concentrated. Residue was purified by reverse phase chromatography on a 0-100% gradient of acetonitrile in water (0.1% trifluoroacetic acid) to recover 0.007 g (33%) of 1-{2-[(4aR,10bS)-3,4,4a,5,6,10b-hexahydro-1,10-phenanthrolin-1(2″-ylmethyl]imidazo[1,2-a]pyridin-5-yl}-N,N-dimethyl-3-azetidinamine as the trifluoroacetic acid salt. ¹H NMR (400 MHz, METHANOL-D4) δ 1.48 (m, 2H), 1.79 (m, 1H), 2.01 (m, 3H), 2.64 (m, 2H), 2.97 (m, 10H), 4.33 (m, 1H), 4.43 (m, 1H), 4.58 (m, 3H), 4.66 (m, 2H), 6.54 (d, 1H), 7.38 (d, 1H), 7.72 (t, 1H), 7.79 (t, 1H), 8.12 (m, 2H), 8.68 (d, 1H); MS m/z 417 (M+1).

### Example 99

### (4aR,10bS)-1-[(5-{4-[2-(methyloxy)ethyl]-1-piperazinyl}imidazo[1,2-a]pyridin-2-yl)methyl]-1,2,3,4,4a,5,6,10b-octahydro-1,10-phenanthroline

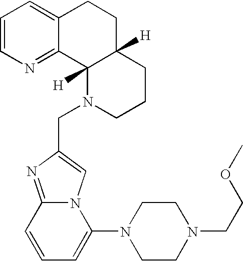

Dissolved (4aR,10bS)-1-[(5-fluoroimidazo[1,2-a]pyridin-2-yl)methyl]-1,2,3,4,4a,5,6,10b-octahydro-1,10-phenanthroline (0.05 g, 0.149 mmol) in 1-[2-(methyloxy)ethyl]piperazine (1.0 mL, 6.93 mmol). The reaction was heated in a microwave reactor at 85° C. for 45 minutes. Diluted reaction mixture with water and extracted three times with dichloromethane. Organics were dried over sodium sulfate and concentrated. Residue was purified by reverse phase chromatography on a 0-100% gradient of acetonitrile in water (0.1% trifluoroacetic acid) to recover 0.027 g (40%) of (4aR,10bS)-1-[(5-{4-[2-(methyloxy)ethyl]-1-piperazinyl}imidazo[1,2-a]pyridin-2-yl)methyl]-1,2,3,4,4a,5,6,10b-octahydro-1,10-phenanthroline as the trifluoroacetic acid salt. ¹H NMR (400 MHz, METHANOL-D4) δ 1.49 (m, 2H), 1.80 (m, 1H), 2.02 (m, 3H), 2.68 (m, 2H), 2.97 (m, 3H), 3.33-3.46 (m, 5H), 3.52 (m, 3H), 3.67 (m, 4H), 3.79 (m, 3H), 4.46 (m, 1H), 4.63 (m, 2H), 7.03 (d, 1H), 7.69 (m, 2H), 7.84 (t, 1H), 8.11 (d, 1H), 8.27 (s, 1H), 8.69 (d, 1H); MS m/z 461 (M+1).

### Example 100

### (3S)-1-{2-[(4aR,10bS)-3,4,4a,5,6,10b-hexahydro-1,10-phenanthrolin-1(2H)-ylmethyl]imidazo[1,2-a]pyridin-5-yl}-N,N-dimethyl-3-pyrrolidinamine

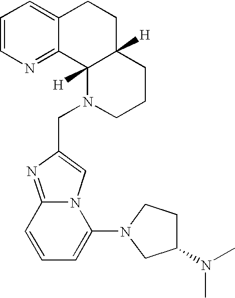

Dissolved (4aR,10bS)-1-[(5-fluoroimidazo[1,2-a]pyridin-2-yl)methyl]-1,2,3,4,4a,5,6,10b-octahydro-1,10-phenanthroline (0.05 g, 0.149 mmol) and (3S)—N,N-dimethyl-3-pyrrolidinamine (0.4 mL, 3.15 mmol) in 1-methyl-2-pyrrolidinone (0.5 mL). The reaction was heated in a microwave reactor at 85° C. for two hours. Diluted reaction mixture with water and extracted three times with dichloromethane. Organics were dried over sodium sulfate and concentrated. Residue was purified by reverse phase chromatography on a 0-100% gradient of acetonitrile in water (0.1% trifluoroacetic acid) to recover 0.027 g (43%) of (3S)-1-{2-[(4aR,10bS)-3,4,4a,5,6,10b-hexahydro-1,10-phenanthrolin-1(2H)-ylmethyl]imidazo[1,2-a]pyridin-5-yl}-N,N-dimethyl-3-pyrrolidinamine as the trifluoroacetic acid salt. ¹H NMR (400 MHz, METHANOL-D4) δ 1.43 (s, 6H), 1.47 (m, 1H), 1.80 (m, 3H), 2.12 (m, 1H), 2.61 (m, 2H), 2.82-3.04 (m, 3H), 3.11 (t, 2H), 3.34 (s, 3H), 3.56 (t, 2H), 4.41 (d, 1H), 4.54 (m, 2H), 6.54 (d, 1H), 7.15 (d, 1H), 7.79 (m, 2H), 8.19 (m, 1H), 8.32 (s, 1H), 8.70 (d, 1H); MS m/z 431 (M+1).

### Example 101

### (4aR,10bS)-1-[(5-{4-[2-oxo-2-(1-pyrrolidinyl)ethyl-1-piperazinyl}imidazol-1,2-a]pyridin-2-yl)methyl]-1,2,3,4,4a,5,6,10b-octahydro-1,10-phenanthroline

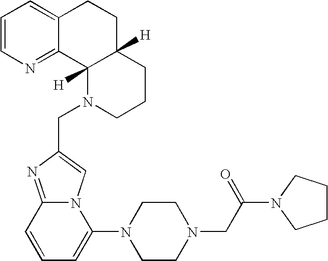

Dissolved (4aR,10bS)-1-[(5-fluoroimidazo[1,2-a]pyridin-2-yl)methyl]-1,2,3,4,4a,5,6,10b-octahydro-1,10-phenanthroline (0.075 g, 0.223 mmol) and 1-[2-oxo-2-(1-pyrrolidinyl)ethyl]piperazine (1.0 g, 6.06 mmol) in 1-methyl-2-pyrrolidinone (1.0 mL). The reaction was heated in a microwave reactor at 85° C. for 45 minutes, then heat was increased to 100° C. and reacted an additional 90 minutes. Diluted reaction mixture with water and extracted three times with dichloromethane. Organics were dried over sodium sulfate and concentrated. Residue was purified by reverse phase chromatography on a 0-100% gradient of acetonitrile in water (0.1% trifluoroacetic acid) to recover 0.034 g (30%) of (4aR,10bS)-1-[(5-{4-[2-oxo-2-(1-pyrrolidinyl)ethyl]-1-piperazinyl}imidazo[1,2-a]pyridin-2-yl)methyl]-1,2,3,4,4a,5,6,10b-octahydro-1,10-phenanthroline as the trifluoroacetic acid salt. ¹H NMR (400 MHz, METHANOL-D4) δ 1.48 (m, 2H), 1.79 (m, 1H), 1.94 (m, 2H), 2.64 (m, 2H), 2.99 (m, 3H), 3.49 (m, 9H), 3.59 (m, 4H), 3.74 (m, 4H), 4.32 (s, 2H), 4.44 (d, 1H), 4.61 (s, 2H), 7.08 (d, 1H), 7.74 (m, 2H), 7.89 (t, 1H), 8.15 (d, 1H), 8.31 (s, 1H), 8.70 (d, 1H); MS m/z 514 (M+1).

### Example 102

### 2-(4-{2-[(4aR,10bS)-3,4,4a,5,6,10b-hexahydro-1,10-phenanthrolin-1(2H)-ylmethyl]imidazo[1,2-a]pyridin-5-yl}-1-piperazinyl)-N,N-dimethylacetamide

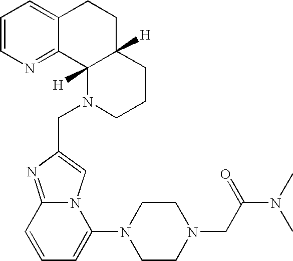

Dissolved (4aR,10bS)-1-[(5-fluoroimidazo[1,2-a]pyridin-2-yl)methyl]-1,2,3,4,4a,5,6,10b-octahydro-1,10-phenanthroline (0.05 g, 0.149 mmol) and N,N-dimethyl-2-(1-piperazinyl)acetamide (1.0 g, 5.84 mmol) in 1-methyl-2-pyrrolidinone (1.0 mL). The reaction was heated in a microwave reactor at 85° C. for 45 minutes. Diluted reaction mixture with water and extracted three times with dichloromethane. Organics were dried over sodium sulfate and concentrated. Residue was purified by reverse phase chromatography on a 0-100% gradient of acetonitrile in water (0.1% trifluoroacetic acid) to recover 0.028 g (38%) of 2-(4-{2-[(4aR,10bS)-3,4,4a,5,6,10b-hexahydro-1,10-phenanthrolin-1(2H)-ylmethyl]imidazo[1,2-a]pyridin-5-yl}-1-piperazinyl)-N,N-dimethylacetamide as the trifluoroacetic acid salt. ¹H NMR (400 MHz, METHANOL-D4) δ 1.49 (m, 2H), 1.80 (m, 2H), 2.02 (m, 4H), 2.69 (m, 2H), 2.96 (m, 3H), 3.01 (s, 3H), 3.03 (s, 3H), 3.57 (m, 3H), 3.73 (m, 3H), 4.41 (s, 2H), 4.46 (d, 1H), 4.64 (s, 2H), 7.03 (d, 1H), 7.69 (m, 2H), 7.82 (t, 1H), 8.09 (d, 1H), 8.27 (s, 1H), 8.67 (d, 1H); MS m/z 488 (M+1).

### Example 103

### 2-(4-{2-[(4aR,10bS)-3,4,4a,5,6,10b-hexahydro-1,10-phenanthrolin-1(2H)-ylmethyl]imidazo[1,2-a]pyridin-5-yl}-1-piperazinyl)ethanol

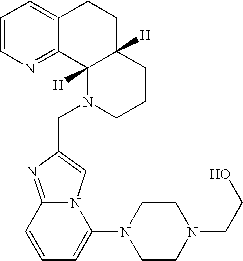

Dissolved (4aR,10bS)-1-[(5-fluoroimidazo[1,2-a]pyridin-2-yl)methyl]-1,2,3,4,4a,5,6,10b-octahydro-1,10-phenanthroline (0.075 g, 0.223 mmol) and 2-(1-piperazinyl)ethanol (0.2 mL, 1.63 mmol) in 1-methyl-2-pyrrolidinone (1.0 mL). The reaction was heated in a microwave reactor at 85° C. for one hour, then at 100° C. for an additional 30 minutes. Diluted reaction mixture with water and extracted three times with dichloromethane. Organics were dried over sodium sulfate and concentrated. Residue was purified by reverse phase chromatography on a 0-100% gradient of acetonitrile in water (0.1% trifluoroacetic acid) to recover 0.022 g (22%) of 2-(4-{2-[(4aR,10bS)-3,4,4a,5,6,10b-hexahydro-1,10-phenanthrolin-1(2H)-ylmethyl]imidazo[1,2-a]pyridin-5-yl}-1-piperazinyl)ethanol as the trifluoroacetic acid salt. ¹H NMR (400 MHz, METHANOL-D4) δ 1.74 (m, 4H), 2.17 (m, 1H), 2.45 (m, 2H), 2.71 (t, 2H), 2.85 (m, 5H), 3.01 (m, 1H), 3.17 (m, 5H), 3.35 (m, 2H), 3.69 (m, 2H), 3.80 (t, 2H), 4.09 (d, 1H), 6.45 (d, 1H), 7.24 (m, 3H), 7.56 (d, 1H), 7.61 (s, 1H), 8.38 (d, 1H); MS m/z 447 (M+1).

### Example 104

### 2-[(4aR,10bS)-3,4,4a,5,6,10b-hexahydro-1,10-phenanthrolin-1(2H)-ylmethyl]-N-[2-(1-methyl-2-pyrrolidinyl)ethyl]imidazo[1,2-a]pyridin-5-amine

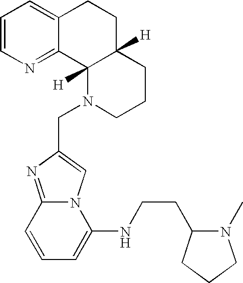

Dissolved (4aR,10bS)-1-[(5-fluoroimidazo[1,2-a]pyridin-2-yl)methyl]-1,2,3,4,4a,5,6,10b-octahydro-1,10-phenanthroline (0.075 g, 0.223 mmol) and [2-(1-methyl-2-pyrrolidinyl)ethyl]amine (0.2 mL, 1.38 mmol) in 1-methyl-2-pyrrolidinone (0.5 mL). The reaction was heated in a microwave reactor at 85° C. for one hour. Diluted reaction mixture with water and extracted three times with dichloromethane. Organics were dried over sodium sulfate and concentrated. Residue was purified by reverse phase chromatography on a 0-100% gradient of acetonitrile in water (0.1% trifluoroacetic acid) to recover 0.019 g (19%) of 2-[(4aR,10bS)-3,4,4a,5,6,10b-hexahydro-1,10-phenanthrolin-1(2″-ylmethyl]-N-[2-(1-methyl-2-pyrrolidinyl)ethyl]imidazo[1,2-a]pyridin-5-amine as the trifluoroacetic acid salt. ¹H NMR (300 MHz, METHANOL-D4) δ 1.50-1.91 (m, 10H), 2.03-2.33 (m, 6H), 2.39 (s, 3H), 2.53 (m, 1H), 2.80 (m, 1H), 3.01-3.26 (m, 3H), 3.37 (m, 3H), 3.56 (m, 1H), 3.93 (d, 1H), 5.93 (d, 1H), 6.82 (d, 1H), 7.22 (m, 2H), 7.56 (m, 2H), 8.35 (d, 1H); MS m/z 445 (M+1).

### Example 105

### (3R)-1-{2-[(4aR,10bS)-3,4,4a,5,6,10b-hexahydro-1,10-phenanthrolin-1(2H)-ylmethyl]imidazo[1,2-a]pyridin-5-yl}-N,N-dimethyl-3-pyrrolidinamine

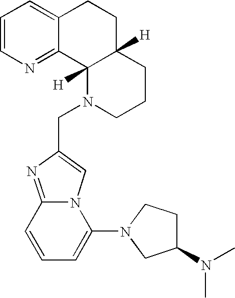

Dissolved (4aR,10bS)-1-[(5-fluoroimidazo[1,2-a]pyridin-2-yl)methyl]-1,2,3,4,4a,5,6,10b-octahydro-1,10-phenanthroline (0.05 g, 0.149 mmol) in (3R)—N,N-dimethyl-3-pyrrolidinamine (1.0 mL, 7.88 mmol). The reaction was heated in a microwave reactor at 85° C. for two hours. Reaction mixture was purified without workup by reverse phase chromatography on a 0-60% gradient of acetonitrile in water (0.1% trifluoroacetic acid). Material was converted to the free base by stirring with MP-carbonate resin in methanol until pH was neutral. Recovered 0.007 g (10%) of (3R)-1-{2-[(4aR,10bS)-3,4,4a,5,6,10b-hexahydro-1,10-phenanthrolin-1(2H)-ylmethyl]imidazo[1,2-a]pyridin-5-yl}-N,N-dimethyl-3-pyrrolidinamine. ¹H NMR (300 MHz, METHANOL-D4) δ 1.52 (m, 2H), 1.85 (m, 1H), 2.05 (m, 1H), 2.46 (m, 1H), 2.69 (m, 3H), 3.01 (m, 2H), 3.08 (s, 6H), 3.37 (m, 3H), 3.60 (q, 1H), 3.82-4.00 (m, 3H), 4.19 (m, 1H), 4.50 (d, 1H), 4.65 (m, 2H), 6.99 (d, 1H), 7.60 (d, 1H), 7.84 (m, 1H), 7.93 (t, 1H), 8.26 (d, 1H), 8.47 (s, 1H), 8.78 (d, 1H); MS m/z 431 (M+1).

### Example 106

### (4aR,10bS)-1-({5-[(8aS)-hexahydropyrrolo[1,2-a]pyrazin-2(1H)-yl]imidazo[1,2-a]pyridin-2-yl}methyl)-1,2,3,4,4a,5,6,10b-octahydro-1,10-phenanthroline

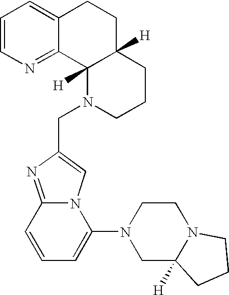

Dissolved (4aR,10bS)-1-[(5-fluoroimidazo[1,2-a]pyridin-2-yl)methyl]-1,2,3,4,4a,5,6,10b-octahydro-1,10-phenanthroline (0.05 g, 0.149 mmol) and (8aS)-octahydropyrrolo[1,2-a]pyrazine (0.1 mL, 0.792 mmol). The reaction was heated in a microwave reactor at 100° C. for one hour. Diluted reaction mixture with dichloromethane and washed three times with water. Extracted combined aqueous layers with dichloromethane twice. Organics were dried over sodium sulfate and concentrated. Residue was purified by silica gel chromatography on a 0-20% gradient of ammonium hydroxide in acetic acid to recover 0.013 g (19%) of (4aR,10bS)-1-({5-[(8aS)-hexahydropyrrolo[1,2-a]pyrazin-2(1H)-yl]imidazo[1,2-a]pyridin-2-yl}methyl)-1,2,3,4,4a,5,6,10b-octahydro-1,10-phenanthroline. ¹H NMR (300 MHz, METHANOL-D4) δ 1.55 (m, 1H), 1.62-1.83 (m, 5H), 1.93 (m, 3H), 2.18 (m, 1H), 2.36-2.52 (m, 4H), 2.65 (m, 2H), 2.82 (m, 1H), 3.00 (m, 2H), 3.11-3.30 (m, 3H), 3.50 (t, 2H), 3.70 (d, 2H), 4.10 (d, 1H), 6.49 (d, 1H), 7.18-7.33 (m, 3H), 7.56 (d, 1H), 7.64 (s, 1H), 8.39 (d, 1H); MS m/z 443 (M+1).

### Example 107

### (3S)-1-{2-[(4aR,10bS)-3,4,4a,5,6,10b-hexahydro-1,10-phenanthrolin-1(2H)-ylmethyl]imidazo[1,2-a]pyridin-5-yl}-3-pyrrolidinamine

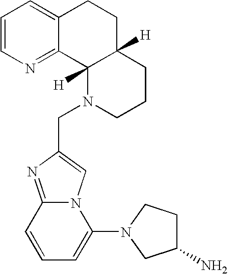

Dissolved (4aR,10bS)-1-[(5-fluoroimidazo[1,2-a]pyridin-2-yl)methyl]-1,2,3,4,4a,5,6,10b-octahydro-1,10-phenanthroline (0.110 g, 0.327 mmol) and 1,1-dimethylethyl (3S)-3-pyrrolidinylcarbamate (0.2 mL, 1.07 mmol) in absolute ethanol (2.0 mL). The reaction was heated in a microwave reactor at 100° C. for four hours. Added trifluoroacetic acid (1 mL) to reaction and concentrated. Residue was purified by reverse phase chromatography on a 0-60% gradient of acetonitrile in water (0.1% trifluoroacetic acid). Treated the recovered material with trifluoroacetic acid (5 mL) in dichloromethane (5 mL) for 30 minutes and concentrated. Residue was further purified by reverse phase chromatography on a 0-60% gradient of acetonitrile in water (0.1% trifluoroacetic acid) to afford 0.042 g (26%) of (3S)-1-{2-[(4aR,10bS)-3,4,4a,5,6,10b-hexahydro-1,10-phenanthrolin-1(2H)-ylmethyl]imidazo[1,2-a]pyridin-5-yl}-3-pyrrolidinamine of the trifluoroacetic acid salt. ¹H NMR (300 MHz, METHANOL-D4) δ 1.48 (m, 2H), 1.79 (m, 1H), 1.93 (m, 3H), 2.21 (m, 1H), 2.51-2.68 (m, 3H), 2.86-3.05 (m, 3H), 3.46 (m, 1H), 3.61 (dd, 1H), 3.80 (dd, 1H), 3.90 (m, 1H), 4.12 (m, 1H), 4.46 (d, 1H), 4.61 (d, 2H), 6.83 (d, 1H), 7.48 (d, 1H), 7.71 (dd, 1H), 7.79 (t, 1H), 8.11 (d, 1H), 8.36 (s, 1H), 8.67 (d, 1H); MS m/z 403 (M+1).

### Example 108

### 2-(4-{2-[(4aR,10bS)-3,4,4a,5,6,10b-hexahydro-1,10-phenanthrolin-1(2H)-ylmethyl]imidazo[1,2-a]pyridin-5-yl}hexahydro-1H-1,4-diazepin-1-yl)ethanol

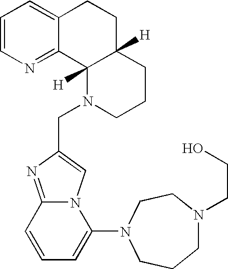

Dissolved (4aR,10bS)-1-[(5-fluoroimidazo[1,2-a]pyridin-2-yl)methyl]-1,2,3,4,4a,5,6,10b-octahydro-1,10-phenanthroline (0.105 g, 0.312 mmol) and 2-(hexahydro-1H-1,4-diazepin-1-yl)ethanol (0.1 mL, 0.70 mmol) in absolute ethanol (2.0 mL). The reaction was heated in a microwave reactor at 85° C. for two hours. Reaction mixture was purified without workup by reverse phase chromatography on a 0-20% gradient of acetonitrile in water (0.1% trifluoroacetic acid) to afford 0.086 g (60%) of 2-(4-{2-[(4aR,10bS)-3,4,4a,5,6,10b-hexahydro-1,10-phenanthrolin-1(2H)-ylmethyl]imidazo[1,2-a]pyridin-5-yl}hexahydro-1H-1,4-diazepin-1-yl)ethanol as the trifluoroacetic acid salt. ¹H NMR (400 MHz, METHANOL-D4) δ 1.45 (m, 2H), 1.79 (m, 1H), 2.02 (m, 3H), 2.36 (m, 2H), 2.64 (m, 2H), 2.09-3.09 (m, 3H), 3.42 (t, 2H), 3.52 (m, 2H), 3.57-3.89 (m, 6H), 3.94 (m, 2H), 4.44 (m, 2H), 4.61 (m, 2H), 7.09 (d, 1H), 7.65 (d, 1H), 7.76 (dd, 1H), 7.89 (t, 1H), 8.17 (d, 1H), 8.25 (s, 1H), 8.71 (d, 1H); MS m/z 461 (M+1).

### Example 109

### 2-[(4aR,10bS)-3,4,4a,5,6,10b-hexahydro-1,10-phenanthrolin-1(2H)-ylmethyl]-N-3-pyrrolidinylimidazo[1,2-a]pyridin-5-amine

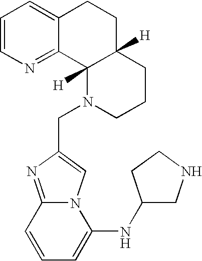

Dissolved (4aR,10bS)-1-[(5-fluoroimidazo[1,2-a]pyridin-2-yl)methyl]-1,2,3,4,4a,5,6,10b-octahydro-1,10-phenanthroline (0.10 g, 0.211 mmol) and 1,1-dimethylethyl 3-amino-1-pyrrolidinecarboxylate (0.2 mL, 1.07 mmol) in absolute ethanol (2.0 mL). The reaction was heated in a microwave reactor at 85° C. for 20 minutes, then at 100° C. for an additional 30 minutes. Added trifluoroacetic acid (1 mL) to reaction and concentrated. Residue was purified by reverse phase chromatography on a 0-60% gradient of acetonitrile in water (0.1% trifluoroacetic acid). Treated the recovered material with trifluoroacetic acid (5 mL) in dichloromethane (5 mL) for 30 minutes and concentrated. Residue was further purified by reverse phase chromatography on a 0-60% gradient of acetonitrile in water (0.1% trifluoroacetic acid) to afford 0.028 g (26%) of 2-[(4aR,10bS)-3,4,4a,5,6,10b-hexahydro-1,10-phenanthrolin-1(2H)-ylmethyl]-N-3-pyrrolidinylimidazo[1,2-a]pyridin-5-amine as the trifluoroacetic acid salt. ¹H NMR (300 MHz, METHANOL-D4) δ 1.46 (m, 2H), 1.79 (m, 1H), 2.02 (m, 3H), 2.29 (m, 1H), 2.47-2.68 (m, 3H), 2.86-3.11 (m, 3H), 3.42-3.73 (m, 5H), 4.38-4.63 (m, 5H), 6.59 (d, 1H), 7.24 (d, 1H), 7.77 (t, 1H), 7.83 (t, 1H), 8.19 (d, 1H), 8.43 (d, 1H), 8.69 (d, 1H); MS m/z 403 (M+1).

### Example 110

### (3R)-1-{2-[(4aR,10bS)-3,4,4a,5,6,10b-hexahydro-1,10-phenanthrolin-1(2H)-ylmethyl]imidazo[1,2-a]pyridin-5-yl}-3-pyrrolidinamine

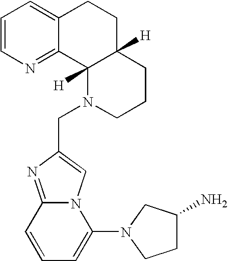

Dissolved (4aR,10bS)-1-[(5-fluoroimidazo[1,2-a]pyridin-2-yl)methyl]-1,2,3,4,4a,5,6,10b-octahydro-1,10-phenanthroline (0.11 g, 0.327 mmol) and 1,1-dimethylethyl (3R)-3-pyrrolidinylcarbamate (0.2 mL, 1.07 mmol) in absolute ethanol (2.0 mL). The reaction was heated in a microwave reactor at 85° C. for 20 minutes, then at 100° C. for an additional four hours. Added trifluoroacetic acid (1 mL) to reaction and concentrated. Residue was purified by reverse phase chromatography on a 0-60% gradient of acetonitrile in water (0.1% trifluoroacetic acid). Treated the recovered material with trifluoroacetic acid (5 mL) in dichloromethane (5 mL) for 30 minutes and concentrated. Residue was further purified by reverse phase chromatography on a 0-60% gradient of acetonitrile in water (0.1% trifluoroacetic acid) to afford 0.067 g (51%) of (3R)-1-{2-[(4aR,10bS)-3,4,4a,5,6,10b-hexahydro-1,10-phenanthrolin-1(2H)-ylmethyl]imidazo[1,2-a]pyridin-5-yl}-3-pyrrolidinamine as the trifluoroacetic acid salt. ¹H NMR (300 MHz, METHANOL-D4) δ 1.54 (m, 2H), 1.86 (m, 1H), 2.08 (m, 3H), 2.29 (m, 1H), 2.58-2.80 (m, 3H), 2.94-3.17 (m, 3H), 3.51 (m, 1H), 3.66 (m, 1H), 3.87 (m, 1H), 3.96 (m, 1H), 4.19 (m, 1H), 4.52 (d, 1H), 4.68 (s, 2H), 6.89 (d, 1H), 7.54 (d, 1H), 7.76 (dd, 1H), 7.84 (t, 1H), 8.16 (d, 1H), 8.44 (s, 1H), 8.73 (d, 1H); MS m/z 403 (M+1).

### Example 111

### (4aR,10bS)-1-({5-[(2S)-2-methyl-1-pyrrolidinyl]imidazo[1,2-a]pyridin-2-yl}methyl)-1,2,3,4,4a,5,6,10b-octahydro-1,10-phenanthroline

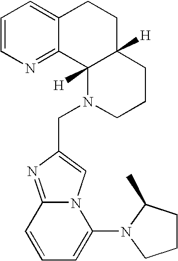

Dissolved (4aR,10bS)-1-[(5-fluoroimidazo[1,2-a]pyridin-2-yl)methyl]-1,2,3,4,4a,5,6,10b-octahydro-1,10-phenanthroline (0.10 g, 0.297 mmol) and (2S)-2-methylpyrrolidine (0.1 mL, 1.18 mmol) in absolute ethanol (1.0 mL). The reaction was heated in a microwave reactor at 100° C. for two hours. Concentrated to half the volume. Residue was purified by reverse phase chromatography on a 0-60% gradient of acetonitrile in water (0.1% trifluoroacetic acid) afford 0.020 g (17%) of (4aR,10bS)-1-({5-[(2S)-2-methyl-1-pyrrolidinyl]imidazo[1,2-a]pyridin-2-yl}methyl)-1,2,3,4,4a,5,6,10b-octahydro-1,10-phenanthroline as the trifluoroacetic acid salt. ¹H NMR (300 MHz, METHANOL-D4) δ 1.20 (m, 3H), 1.47 (m, 2H), 1.79 (m, 2H), 1.88-2.08 (m, 4H), 2.13 (m, 1H), 2.38 (m, 1H), 2.64 (m, 2H), 2.87-3.08 (m, 3H), 3.36 (m, 1H), 3.99 (m, 1H), 4.11 (m, 1H), 4.48 (d, 1H), 4.60 (q, 2H), 6.82 (d, 1H), 7.41 (d, 1H), 7.77 (dd, 1H), 7.84 (t, 1H), 8.18 (d, 1H), 8.27 (s, 1H), 8.73 (d, 1H); MS m/z 402 (M+1).

### Example 112

### 2-[(4aR,10bS)-3,4,4a,5,6,10b-hexahydro-1,10-phenanthrolin-1(2H)-ylmethyl]-N-[(3S)-3-piperidinyl]imidazo[1,2-a]pyridin-5-amine

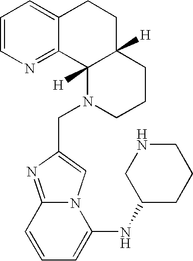

Dissolved (4aR,10bS)-1-[(5-fluoroimidazo[1,2-a]pyridin-2-yl)methyl]-1,2,3,4,4a,5,6,10b-octahydro-1,10-phenanthroline (0.11 g, 0.31 mmol) and 1,1-dimethylethyl (2S)-2-amino-1-piperidinecarboxylate (0.14 mL, 0.67 mmol) in absolute ethanol (1.0 mL). The reaction was heated in a microwave reactor at 85° C. for four hours, then at 150° C. for two hours. Concentrated to half the volume. Residue was purified by reverse phase chromatography on a 0-60% gradient of acetonitrile in water (0.1% trifluoroacetic acid) to afford 1,1-dimethylethyl (2S)-2-({2-[(4aR,10bS)-3,4,4a,5,6,10b-hexahydro-1,10-phenanthrolin-1(2H)-ylmethyl]imidazo[1,2-a]pyridin-5-yl}amino)-1-piperidinecarboxylate which lost the protecting group upon standing while the fractions were evaporating over 48 hours to afford 0.039 g (30%) of 2-[(4aR,10bS)-3,4,4a,5,6,10b-hexahydro-1,10-phenanthrolin-1(2H)-ylmethyl]-N-[(3S)-3-piperidinyl]imidazo[1,2-a]pyridin-5-amine as the trifluoroacetic acid salt. ¹H NMR (400 MHz, METHANOL-D4) δ 1.47 (m, 1H), 1.82 (d, 1H), 1.88-2.18 (m, 6H), 2.25 (m, 1H), 2.63 (m, 2H), 2.87-3.19 (m, 6H), 3.38 (d, 1H), 3.61 (m, 1H), 4.11 (m, 1H), 4.43 (d, 1H), 4.56 (q, 2H), 6.66 (d, 1H), 7.22 (d, 1H), 7.78 (dd, 1H), 7.84 (t, 1H), 8.19 (d, 1H), 8.46 (s, 1H), 8.72 (d, 1H); MS m/z 417 (M+1).

### Example 113

### 2-[(4aR,10bS)-3,4,4a,5,6,10b-hexahydro-1,10-phenanthrolin-1(2H)-ylmethyl]-N-[(3R)-3-piperidinylmethyl]imidazo[1,2-a]pyridin-5-amine

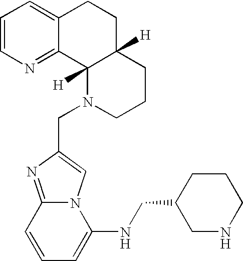

Dissolved (4aR,10bS)-1-[(5-fluoroimidazo[1,2-a]pyridin-2-yl)methyl]-1,2,3,4,4a,5,6,10b-octahydro-1,10-phenanthroline (0.11 g, 0.312 mmol) and 1,1-dimethylethyl (3S)-3-(aminomethyl)-1-piperidinecarboxylate (0.1 mL, 0.47 mmol) in absolute ethanol (1.0 mL). The reaction was heated in a microwave reactor at 85° C. for 20 minutes, then at 100° C. for an additional four hours. Concentrated to half the volume. Residue was purified by reverse phase chromatography on a 0-60% gradient of acetonitrile in water (0.1% trifluoroacetic acid) afford 1,1-dimethylethyl (3S)-3-[({2-[(4aR,10bS)-3,4,4a,5,6,10b-hexahydro-1,10-phenanthrolin-1(2H)-ylmethyl]imidazo[1,2-a]pyridin-5-yl}amino)methyl]-1-piperidinecarboxylate as the trifluoroacetic acid salt. Residue was dissolved in dichloromethane (2 mL) and trifluoroacetic acid (2 mL) was added. Reaction was stirred at room temperature for one hour then concentrated to afford 0.082 g (74%) of 2-[(4aR,10bS)-3,4,4a,5,6,10b-hexahydro-1,10-phenanthrolin-1(2H)-ylmethyl]-N-[(3R)-3-piperidinylmethyl]imidazo[1,2-a]pyridin-5-amine as the trifluoroacetic acid salt with no further purification. ¹H NMR (400 MHz, METHANOL-D4) δ 1.46 (m, 3H), 1.78 (m, 2H), 1.98-2.10 (m, 5H), 2.30 (m, 1H), 2.63 (m, 2H), 2.79-3.10 (m, 5H), 3.34-3.57 (m, 6H), 4.43 (d, 1H), 4.54 (t, 2H), 6.58 (d, 1H), 7.17 (d, 1H), 7.83 (m, 2H), 8.23 (d, 1H), 8.32 (s, 1H), 8.73 (d, 1H); MS m/z 431 (M+1).

### Example 114

### 6,7,8,9-tetrahydro-5H-cyclohepta[b]pyridin-9-ol (intermediate)

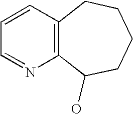

To a solution of 6,7,8,9-tetrahydro-5H-cyclohepta[b]pyridine (26.32 g, 179 mmol) in 100 mL of acetic acid was added 30% aqueous hydrogen peroxide (36 mL) and the mixture heated at 70° C. for 16 hours. The reaction mixture was concentrated and the residue dissolved in chloroform. Solid sodium carbonate (100 g) was added and the mixture stirred for 2 hours. The solids were filtered off and the wash concentrated. The residue was dissolved in acetic anhydride (400 mL) and the mixture heated at 90° C. for 48 hours. The mixture was concentrated, the residue dissolved in water (500 mL), and potassium carbonate (50 g) was added carefully portionwise. Methanol (20 mL) was added and the mixture heated to 70° C. for 16 hours. The mixture was allowed to cool and extracted 3 times with 150 mL of dichloromethane. The organic layers were combined and concentrated. The residue was purified by silica chromatography eluting with a 1% to 2% gradient of 2M ammonia/methanol in dichloromethane. Appropriate fractions were concentrated to give 6.97 g (24%) of 6,7,8,9-tetrahydro-5H-cyclohepta[b]pyridin-9-ol. ¹H NMR (400 MHz, CHLOROFORM-D) δ ppm 1.2 (m, 1H), 1.4 (m, 1H), 1.8 (m, 1H), 2.0 (m, 1H), 2.1 (m, 1H), 2.2 (m, 1H), 2.7 (m, 2H), 4.8 (d, J=11.2 Hz, 1H), 5.9 (s, 1H), 7.1 (dd, J=7.5, 4.9 Hz, 1H), 7.4 (d, J=7.3 Hz, 1H), 8.4 (d, J=4.8 Hz, 1H); MS m/z 164 (M+1)

### Example 115

### 5,6,7,8-tetrahydro-9H-cyclohepta[b]pyridin-9-one (intermediate)

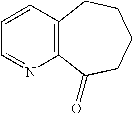

To a −78° C. solution of 2M oxalyl chloride in dichloromethane (23 mL, 46 mmol) in dichloromethane (150 mL) was added a solution of dimethyl sulfoxide (7.1 mL, 100 mmol) in dichloromethane (20 mL). The mixture was stirred for 10 minutes and a solution of 6,7,8,9-tetrahydro-5H-cyclohepta[b]pyridin-9-ol (6.9 g, 42 mmol) in 30 mL of dichloromethane added dropwise. The mixture was stirred for 30 minutes and triethylamine (21 g, 210 mmol) added dropwise. The mixture was allowed to warm to room temperature and stirred for 1 hour. The mixture was washed with water, dried over magnesium sulfate, and concentrated. A solid formed while concentrating and was filtered off. The wash was concentrated and the residue purified by silica chromatography eluting with ethyl acetate. Appropriate fractions were concentrated to yield 5.12 g (74%) of 5,6,7,8-tetrahydro-9H-cyclohepta[b]pyridin-9-one. ¹H NMR (400 MHz, CHLOROFORM-D) δ ppm 1.9 (m, 4H), 2.8 (m, 2H), 2.9 (m, 2H), 7.3 (m, 1H), 7.6 (d, 1H), 1.9 (d, 1H); MS m/z 162 (M+1).

### Example 116

### ethyl 3-(9-oxo-6,7,8,9-tetrahydro-5H-cyclohepta[b]pyridin-8-yl)propanoate (intermediate)

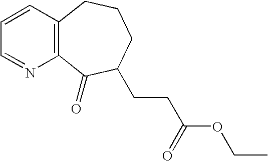

5,6,7,8-tetrahydro-9H-cyclohepta[b]pyridin-9-one (1.51 g, 9.4 mmol), p-toluenesulfonic acid monohydrate (0.16 g, 0.94 mmol), and pyrrolidine (1.3 g, 19 mmol) in 50 mL of benzene were refluxed with a Dean-Stark trap for 4 hours. The mixture was concentrated and the residue dissolved in 35 mL of absolute ethanol. Ethyl acrylate (1.3 g, 13 mmol) was added dropwise and the mixture heated to reflux for 3 hours. Water (10 mL) was added and the mixture continued to heat at 78° C. for 2 hours. The mixture was concentrated to 10 mL and then diluted with 50 mL of brine. The mixture was extracted 3 times with 50 mL of dichloromethane. The organic layers were combined, concentrated, and the residue purified by silica chromatography eluting with acetonitrile. Appropriate fractions were concentrated to yield 1.5 g (60%) of ethyl 3-(9-oxo-6,7,8,9-tetrahydro-5H-cyclohepta[b]pyridin-8-yl)propanoate. ¹H NMR (400 MHz, METHANOL-D4) δ ppm 1.2 (t, J=7.1 Hz, 3H), 1.6 (m, 1H), 1.7 (m, 1H), 1.8 (m, 1H), 2.1 (m, 3H), 2.4 (m, 2H), 3.0 (m, 3H), 4.1 (q, J=7.1 Hz, 2H), 7.4 (dd, J=7.9, 4.8 Hz, 1H), 7.8 (d, J=8.6 Hz, 1H), 8.5 (d, J=4.8 Hz, 1H); MS m/z 262 (M+1).

### Example 117

### 3-[9-({(1R)-1-[4-(methyloxy)phenyl]ethyl}amino)-6,7,8,9-tetrahydro-5H-cyclohepta[b]pyridin-8-yl]-1-propanol (intermediate)

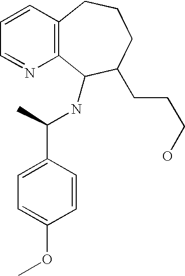

To a solution of ethyl 3-(9-oxo-6,7,8,9-tetrahydro-5H-cyclohepta[b]pyridin-8-yl)propanoate (1.5 g, 5.7 mmol) in 50 mL of toluene was added p-toluenesulfonic acid monohydrate (0.097 g, 0.057 mmol) and (1R)-1-[4-(methyloxy)phenyl]ethanamine (1.7 g, 11 mmol). The reaction mixture was refluxed with a Dean Stark trap overnight. The mixture was concentrated and the residue taken up in 50 mL of 1,2-dichloroethane. Sodium triacetoxyborohydride (3.6 g, 17 mmol) was added and the mix stirred at room temperature for 4 hours. The mixture was quenched with water, extracted 3 times with dichloromethane, concentrated, and the residue partially purified by silica chromatography eluting with acetonitrile. Appropriate fractions were concentrated and the residue dissolved in 50 mL of tetrahydrofuran. A 2M lithium borohydride solution (2.6 mL, 5.3 mmol) in tetrahydrofuran was added and the mixture heated to 65° C. for 2 hours. A second portion of 2M lithium borohydride solution (2.6 mL, 5.3 mmol) in tetrahydrofuran was added and the mixture heated to 65° C. for 16 hours. The mixture was allowed to cool to room temperature, quenched with 50 mL of water, extracted 3 times with 25 mL of dichloromethane, and concentrated. The residue was purified by silica chromatography eluting with a 0% to 10% gradient of methanol in ethyl acetate to yield 0.79 g (39%) of 3-[9-({(1R)-1-[4-(methyloxy)phenyl]ethyl}amino)-6,7,8,9-tetrahydro-5H-cyclohepta[b]pyridin-8-yl]-1-propanol. ¹H NMR (400 MHz, METHANOL-D4) δ ppm 1.3 (d, J=6.6 Hz, 3H), 1.5 (m, 4H), 1.7 (m, 2H), 2.0 (bs, 2 H), 2.2 (m, 1H), 2.5 (m, 2H), 3.4 (m, 2H), 3.6 (q, J=6.6 Hz, 1H), 3.7 (s, 3H), 4.0 (d, J=5.5 Hz, 1H), 6.7 (d, J=8.8 Hz, 2H), 7.0 (m, 2H), 7.1 (m, 1H), 7.4 (d, J=9.1 Hz, 1 H), 8.1 (d, J=6.4 Hz, 1H); MS m/z 355 (M+1).

### Example 118a

### (4aR,11bS)-1-{(1R)-1-[4-(methyloxy)phenyl]ethyl}-2,3,4,4a,5,6,7,11b-octahydro-1H-pyrido[3′,2′:6,7]cyclohepta[1,2-b]pyridine (intermediate)

### Example 118b

### (4aS,11bS)-1-{(1R)-1-[4-(methyloxy)phenyl]ethyl}-2,3,4,4a,5,6,7,11b-octahydro-1H-pyrido[3′,2′:6,7]cyclohepta[1,2-b]pyridine (intermediate)

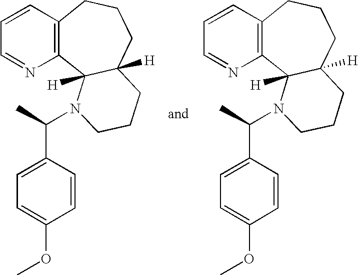

To a solution of 3-[9-({(1R)-1-[4-(methyloxy)phenyl]ethyl}amino)-6,7,8,9-tetrahydro-5H-cyclohepta[b]pyridin-8-yl]-1-propanol (0.56 g, 1.6 mmol) in 50 mL of dichloromethane was added N,N-diisopropylethylamine (0.28 g, 2.2 mmol), methanesulfonyl chloride (0.16 mL, 2.1 mmol), and N,N-dimethyl-4-pyridinamine (0.019 g, 0.16 mmol). The mixture was stirred for 16 hours at room temperature. The mixture was quenched with saturated sodium bicarbonate and extracted 3 times with 25 mL of dichloromethane. The dichloromethane layers were combined and concentrated. Silica chromatography eluting with a 0% to 10% gradient of 2M ammonia in methanol and dichloromethane as co-solvent yielded 0.075 g (14%) of (4aR,11bS)-1-{(1R)-1-[4-(methyloxy)phenyl]ethyl}-2,3,4,4a,5,6,7,11b-octahydro-1H-pyrido[3′,2′:6,7]cyclohepta[1,2-b]pyridine and 0.32 g (58%) of (4aS,11bS)-1-{(1R)-1-[4-(methyloxy)phenyl]ethyl}-2,3,4,4a,5,6,7,11b-octahydro-1H-pyrido[3′,2′:6,7]cyclohepta[1,2-b]pyridine. 118a (cis): MS m/z 337 (M+1), Rₜ=1.90 min. 118b (trans): ¹H NMR (400 MHz, METHANOL-D4) δ ppm 1.1 (d, J=7.0 Hz, 3 H), 1.4 (m, 2H), 1.5 (m, 4H), 1.6 (m, 2H), 1.9 (m, 1H), 2.4 (td, J=10.9, 3.4 Hz, 1H), 2.7 (dt, J=14.6, 5.5 Hz, 1H), 2.8 (m, 1H), 3.0 (dt, J=14.5, 7.2 Hz, 1H), 3.8 (s, 3H), 3.9 (d, J=9.3 Hz, 1H), 4.2 (q, J=7.0 Hz, 1H), 6.8 (d, J=8.8 Hz, 2H), 7.1 (dd, J=7.6, 4.8 Hz, 1H), 7.4 (d, J=8.6 Hz, 2H), 7.5 (d, J=7.5 Hz, 1H), 8.5 (d, J=6.4 Hz, 1H); MS m/z 337 (M+1), Rₜ=1.82 min.

### Example 119

### (4aS,11bS)-2,3,4,4a,5,6,7,11b-octahydro-1H-pyrido[3′,2′:6,7]cyclohepta[1,2-b]pyridine (intermediate)

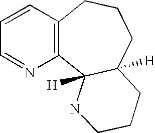

To a solution of (4aS,11bS)-1-{(1R)-1-[4-(methyloxy)phenyl]ethyl}-2,3,4,4a,5,6,7,11b-octahydro-1H-pyrido[3′,2′:6,7]cyclohepta[1,2-b]pyridine (0.31 g, 0.94 mmol) in 10 mL of dichloromethane was added 10 mL of trifluoroacetic acid. The mixture was stirred at room temperature for 1 hour and was concentrated. The residue was treated with saturated sodium bicarbonate and extracted 3 times with 15 mL of dichloromethane. The organic layers were combined, concentrated, and the residue purified by silica chromatography eluting with a 0% to 10% gradient of 30% aqueous ammonium hydroxide in acetonitrile to yield 0.19 (>99%) of (4aS,11bS)-2,3,4,4a,5,6,7,11b-octahydro-1H-pyrido[3′,2′:6,7]cyclohepta[1,2-b]pyridine. ¹H NMR (400 MHz, METHANOL-D4) δ ppm 1.3 (m, 1H), 1.4 (m, 1H), 1.6 (m, 2H), 1.8 (m, 1H), 1.9 (m, 4H), 2.8 (m, 3H), 3.3 (m, 1H), 4.1 (d, J=10.1 Hz, 1H), 7.2 (m, 1H), 7.5 (d, J=7.7 Hz, 1H), 8.4 (d, J=4.8 Hz, 1H); MS m/z 203 (M+1).

### Example 120

### (4aR,11bS)-2,3,4,4a,5,6,7,11b-octahydro-1H-pyrido[3′,2′:6,7]cyclohepta[1,2-b]pyridine (intermediate)

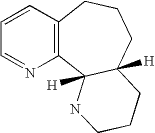

To a solution of (4aR,11bS)-1-{(1R)-1-[4-(methyloxy)phenyl]ethyl}-2,3,4,4a,5,6,7,11b-octahydro-1H-pyrido[3′,2′:6,7]cyclohepta[1,2-b]pyridine (0.075 g, 0.22 mmol) in 5 mL of dichloromethane was added 10 mL of trifluoroacetic acid. The mixture was stirred at room temperature for 1 hour and was concentrated. The residue was treated with saturated sodium bicarbonate and extracted 3 times with 10 mL of dichloromethane. The organic layers were combined, concentrated, and the residue purified by silica chromatography eluting with a 0% to 10% gradient of 30% aqueous ammonium hydroxide in acetonitrile to yield 0.017 (40%) of (4aR,11bS)-2,3,4,4a,5,6,7,11b-octahydro-1H-pyrido[3′,2′:6,7]cyclohepta[1,2-b]pyridine. ¹H NMR (400 MHz, METHANOL-D4) δ ppm 1.5 (m, 3H), 1.7 (m, 2H), 1.9 (m, 2H), 2.0 (m, 1H), 2.2 (m, 1H), 2.7 (m, 1H), 2.9 (m, 1H), 3.1 (m, 1H), 3.4 (m, 1H), 4.3 (d, J=3.3 Hz, 1H), 7.2 (dd, J=7.6, 4.8 Hz, 1H), 7.5 (d, J=9.3 Hz, 1H), 8.3 (d, J=6.6 Hz, 1H); MS m/z 203 (M+1).

### Example 121

### (4aS,11bS)-1-[(5-fluoroimidazo[1,2-a]pyridin-2-yl)methyl]-2,3,4,4a,5,6,7,11b-octahydro-1H-pyrido[3′,2′:6,7]cyclohepta[1,2-b]pyridine (intermediate)

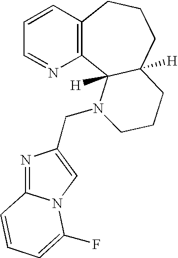

To a solution of (4aS,11bS)-2,3,4,4a,5,6,7,11b-octahydro-1H-pyrido[3′,2′:6,7]cyclohepta[1,2-b]pyridine (0.21 g, 1.0 mmol) in 10 mL of acetonitrile was added potassium carbonate (0.69 g, 5.0 mmol), 2-(chloromethyl)-5-fluoroimidazo[1,2-a]pyridine (0.23 g, 1.2 mmol), and 10 mg of potassium iodide. The reaction mixture was stirred for 16 hours at room temperature and quenched with 50 mL of water. The mixture was extracted 3 times with 25 mL of dichloromethane. The combined organic layers were concentrated and the residue purified by silica chromatography eluting with a 0% to 10% gradient of 2M ammonia in methanol and dichloromethane as co-solvent to yield 0.18 g (51%) of (4aS,11bS)-1-[(5-fluoroimidazo[1,2-a]pyridin-2-yl)methyl]-2,3,4,4a,5,6,7,11b-octahydro-1H-pyrido[3′,2′:6,7]cyclohepta[1,2-b]pyridine. ¹H NMR (400 MHz, METHANOL-D4) δ ppm 1.3 (m, 1H), 1.4 (m, 2H), 1.6 (m, 2H), 1.7 (m, 3H), 1.9 (m, 1H), 2.4 (m, 1H), 2.7 (m, 1H), 3.0 (m, 1H), 3.2 (m, 1H), 3.6 (m, 1H), 3.7 (m, 1H), 3.9 (d, J=14.5 Hz, 1 H), 6.7 (td, J=5.2, 3.2 Hz, 1H), 7.2 (dd, J=7.6, 4.8 Hz, 1H), 7.4 (m, 2H), 7.5 (d, J=8.8 Hz, 1H), 7.6 (s, 1H), 8.5 (d, J=4.8 Hz, 1H); MS m/z 351 (M+1).

### Example 122

### (4aR,11bS)-1-[(5-fluoroimidazo[1,2-a]pyridin-2-yl)methyl]-2,3,4,4a,5,6,7,11b-octahydro-1H-pyrido[3′,2′:6,7]cyclohepta[1,2-b]pyridine (intermediate)

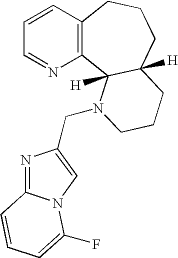

To a solution of ((4aR,11bS)-2,3,4,4a,5,6,7,11b-octahydro-1H-pyrido[3′,2′:6,7]cyclohepta[1,2-b]pyridine (0.017 g, 0.084 mmol) in 3 mL of acetonitrile was added potassium carbonate (0.058 g, 0.42 mmol), 2-(chloromethyl)-5-fluoroimidazo[1,2-a]pyridine (0.017 g, 0.093 mmol), and 5 mg of potassium iodide. The reaction mixture was stirred for 16 hours at room temperature and quenched with 10 mL of water. The mixture was extracted 3 times with 10 mL of dichloromethane. The combined organic layers were concentrated and the residue purified by silica chromatography eluting with a 0% to 10% gradient of 2M ammonia in methanol and dichloromethane as co-solvent to yield 0.015 g (50%) of (4aR,11bS)-1-[(5-fluoroimidazo[1,2-a]pyridin-2-yl)methyl]-2,3,4,4a,5,6,7,11b-octahydro-1H-pyrido[3′,2′:6,7]cyclohepta[1,2-b]pyridine. ¹H NMR (400 MHz, METHANOL-D4) δ ppm 1.4 (m, 1H), 1.5 (m, 1H), 1.6 (m, 2H), 1.8 (m, 1H), 2.0 (m, 3H), 2.3 (m, 1H), 2.5 (dd, J=13.5, 6.6 Hz, 1H), 2.6 (m, 1H), 3.3 (m, 2H), 3.4 (m, 2 H), 3.8 (m, 1H), 6.6 (m, 1H), 7.2 (dd, J=7.7, 4.9 Hz, 1H), 7.3 (m, 2H) 7.4 (d, J=8.6 Hz, 1H) 7.5 (s, 1H) 8.3 (d, J=6.0 Hz, 1H); MS m/z 351 (M+1).

### Example 123

### (4aS,11bS)-1-{[5-(4-methyl-1-piperazinyl)imidazo[1,2-a]pyridin-2-yl]methyl}-2,3,4,4a,5,6,7,11b-octahydro-1H-pyrido[3′,2′:6,7]cyclohepta[1,2-b]pyridine

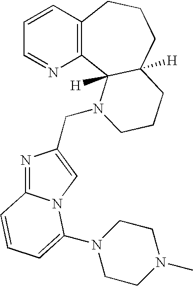

(4aS,11bS)-1-[(5-Fluoroimidazo[1,2-a]pyridin-2-yl)methyl]-2,3,4,4a,5,6,7,11b-octahydro-1H-pyrido[3′,2′:6,7]cyclohepta[1,2-b]pyridine (0.045 g, 0.13 mmol) was dissolved in 4 mL 1-methylpiperazine and heated to 200° C. in a microwave reactor for 20 minutes. The reaction was allowed to cool and diluted with 5 mL of brine. The mixture was extracted 3 times with 5 mL of dichloromethane. The combined organic layers were washed 3 times with 5 mL of water, dried over sodium sulfate, and concentrated. The residue purified by silica chromatography eluting with a 0% to 10% gradient of 30% aqueous ammonium hydroxide in acetonitrile to yield 0.024 g (43%) of (4aS,11bS)-1-{[5-(4-methyl-1-piperazinyl)imidazo[1,2-a]pyridin-2-yl]methyl}-2,3,4,4a,5,6,7,11b-octahydro-1H-pyrido[3′,2′:6,7]cyclohepta[1,2-b]pyridine. ¹H NMR (400 MHz, METHANOL-D4) δ ppm 1.3 (m, 2H), 1.4 (m, 2H), 1.6 (m, 4H), 1.8 (m, 1 H), 2.4 (s, 3H), 2.5 (m, 1H), 2.7 (m, 5H), 2.9 (m, 1H), 3.1 (m, 4H), 3.2 (d, J=11.5 Hz, 1H), 3.4 (d, J=9.9 Hz, 1H), 3.9 (s, 2H), 6.5 (d, J=7.0 Hz, 1H), 7.2 (m, 4H), 7.5 (d, J=9.0 Hz, 1H), 8.5 (d, J=4.8 Hz, 1H); MS m/z 431 (M+1).

### Example 124

### (4aR,11bS)-1-{[5-(4-methyl-1-piperazinyl)imidazo[1,2-a]pyridin-2-yl]methyl}-2,3,4,4a,5,6,7,11b-octahydro-1H-pyrido[3′,2′:6,7]cyclohepta[1,2-b]pyridine

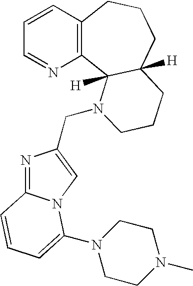

(4aR,11bS)-1-[(5-Fluoroimidazo[1,2-a]pyridin-2-yl)methyl]-2,3,4,4a,5,6,7,11b-octahydro-1H-pyrido[3′,2′:6,7]cyclohepta[1,2-b]pyridine (0.015 g, 0.043 mmol) was dissolved in 3 mL 1-methylpiperazine and heated to 200° C. in a microwave reactor for 20 minutes. The reaction was allowed to cool and diluted with 5 mL of brine. The mixture was extracted 3 times with 5 mL of dichloromethane. The combined organic layers were washed 3 times with 5 mL of water, dried over sodium sulfate, and concentrated. The residue purified by silica chromatography eluting with a 0% to 10% gradient of 30% aqueous ammonium hydroxide in acetonitrile to yield 0.011 g (60%) of (4aR,11bS)-1-{[5-(4-methyl-1-piperazinyl)imidazo[1,2-a]pyridin-2-yl]methyl}-2,3,4,4a,5,6,7,11b-octahydro-1H-pyrido[3′,2′:6,7]cyclohepta[1,2-b]pyridine. ¹H NMR (400 MHz, METHANOL-D4) δ ppm 1.4 (m, 1H), 1.5 (d, J=16.1 Hz, 1H), 1.6 (m, 2H), 1.7 (m, 1H), 2.0 (m, 3H), 2.3 (m, 1H), 2.4 (s, 3H), 2.5 (dd, J=12.9, 6.0 Hz, 1H), 2.7 (m, 5H), 3.2 (m, 4H), 3.3 (m, 2H), 3.4 (m, 2H), 3.8 (m, 1H), 6.4 (d, J=7.1 Hz, 1 H), 7.2 (m, 2H), 7.3 (m, 2H), 7.4 (d, J=7.5 Hz, 1H), 8.3 (d, J=6.4 Hz, 1H); MS m/z 431 (M+1).

### Example 125

### (trans) 1-{[5-(4-methylhexahydro-1H-1,4-diazepin-1-yl)imidazo[1,2-a]pyridin-2-yl]methyl}-2,3,4,4a,5,6,7,11b-octahydro-1H-pyrido[3′,2′:6,7]cyclohepta[1,2-b]pyridine

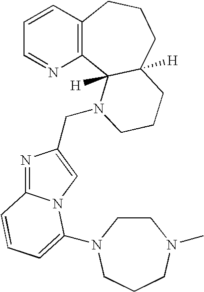

(4aS,11bS)-1-[(5-Fluoroimidazo[1,2-a]pyridin-2-yl)methyl]-2,3,4,4a,5,6,7,11b-octahydro-1H-pyrido[3′,2′:6,7]cyclohepta[1,2-b]pyridine (0.030 g, 0.086 mmol) was dissolved in 4 mL 1-methylpiperazine and heated to 150° C. in a microwave reactor for 20 minutes. The reaction was not complete by TLC analysis. The reaction mixture was heated to 150° C. in a microwave reactor for an additional 20 minutes. The reaction was allowed to cool and diluted with 5 mL of brine. The mixture was extracted 3 times with 5 mL of dichloromethane. The combined organic layers were washed 3 times with 5 mL of water, dried over sodium sulfate, and concentrated. The residue purified by silica chromatography eluting with a 0% to 10% gradient of 30% aqueous ammonium hydroxide in acetonitrile to yield 12 mg (31%) of (trans) 1-{[5-(4-methylhexahydro-1H-1,4-diazepin-1-yl)imidazo[1,2-a]pyridin-2-yl]methyl}-2,3,4,4a,5,6,7,11b-octahydro-1H-pyrido[3′,2′:6,7]cyclohepta[1,2-b]pyridine. ¹H NMR (400 MHz, METHANOL-D4) δ ppm 1.3 (m, 2H), 1.5 (m, 2H), 1.7 (m, 4H), 1.8 (m, 1 H), 2.0 (m, 2H), 2.4 (s, 3H), 2.5 (m, 1H), 2.7 (m, 1H), 2.8 (m, 5H), 3.2 (d, J=11.9 Hz, 1H), 3.4 (m, 4H), 3.5 (d, J=9.7 Hz, 1H), 3.9 (m, 2H), 6.5 (d, J=7.3 Hz, 1H), 7.2 (m, 4H), 7.5 (d, J=7.5 Hz, 1H), 8.5 (d, J=6.4 Hz, 1H); MS m/z 445 (M+1).

### Example 126

### (8-oxo-5,6,7,8-tetrahydro-7-quinolinyl)acetonitrile (intermediate)

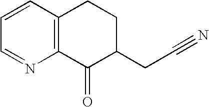

To a −78° C. solution of diisopropylamine (0.76 g, 7.5 mmol) in 20 mL of tetrahydrofuran was added 1.6 M n-butyllithium (4.7 mL, 7.5 mmol) dropwise. The mixture was allowed to warm to 0° C. and stirred for 30 minutes before being cool to −78° C. A solution of 6,7-dihydro-8(5H)-quinolinone (1.0 g, 6.8 mmol) in 10 mL of tetrahydrofuran was added quickly dropwise. Mixture stirred for 3 h, then bromoacetonitrile (8.2 mmol) added. The mixture was allowed to warm to 4° and stirred overnight. The mixture quenched with water and allowed to warm to room temperature. The mixture was extracted 3 times with 20 mL of dichloromethane. The combined organic layers washed with brine, dried over sodium sulfate, and concentrated. The residue was purified by silica chromatography eluting with acetonitrile to yield 550 mg (43%) of (8-oxo-5,6,7,8-tetrahydro-7-quinolinyl)acetonitrile. ¹H NMR (400 MHz, METHANOL-D4) δ ppm 2.1 (m, 1H), 2.4 (m, 1H), 2.9 (d, J=6.8 Hz, 1H), 3.0 (d, J=5.1 Hz, 1H), 3.1 (m, 1H), 3.2 (d, J=11.3 Hz, 1H), 3.3 (m, 1H), 7.6 (dd, J=7.9, 4.6 Hz, 1H), 7.9 (d, J=7.9 Hz, 1H), 8.6 (d, J=4.6 Hz, 1H); MS m/z 187 (M+1).

### Example 127

### 2,3,3a,4,5,9b-hexahydro-1H-pyrrolo[3,2-h]quinoline (intermediate)

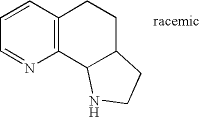

(8-Oxo-5,6,7,8-tetrahydro-7-quinolinyl)acetonitrile (0.40 g, 2.2 mmol) was hydrogenated for 3 hours under 50 psi of hydrogen, at 70° C., with raney nickel (0.040 g) as catalyst. The reaction was allowed to cool to room temperature and the catalyst carefully filtered off over celite. The filtrate was concentrated and the residue dried under vacuum to yield 344 mg (89%) of 2,3,3a,4,5,9b-hexahydro-1H-pyrrolo[3,2-h]quinoline. ¹H NMR (400 MHz, METHANOL-D4) δ ppm 1.6 (m, 2H), 1.8 (m, 2H), 2.2 (m, 1H), 2.7 (m, 3H), 3.0 (m, 1H), 4.1 (m, 1H), 7.2 (m, 1H), 7.6 (m, 1 H), 8.4 (m, 1H); MS m/z 175 (M+1).

### Example 128a

### (trans) 1,1-dimethylethyl 2-(2,3,3a,4,5,9b-hexahydro-1H-pyrrolo[3,2-h]quinolin-1-ylmethyl)-1H-benzimidazole-1-carboxylate (intermediate)

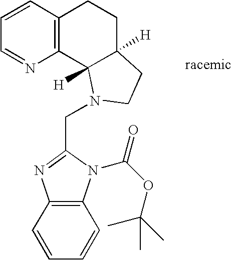

### Example 128b

### (trans) 1,1-dimethylethyl 2-{[2-(2,3,3a,4,5,9b-hexahydro-1H-pyrrolo[3,2-h]quinolin-1-ylmethyl)-1H-benzimidazol-1-yl]methyl}-1H-benzimidazole-1-carboxylate (intermediate)

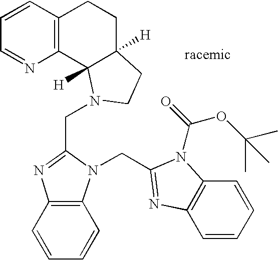

2,3,3a,4,5,9b-Hexahydro-1H-pyrrolo[3,2-h]quinoline (0.34 g, 1.9 mmol), 1,1-dimethylethyl 2-(chloromethyl)-1H-benzimidazole-1-carboxylate (0.67 g, 2.5 mmol), potassium carbonate (1.3 g, 9.8 mmol) and potassium iodide (0.032 g, 0.19 mmol) were stirred at room temperature in 5 mL of acetonitrile for 16 hours. The mixture was quenched with 15 mL of water and extracted with 15 mL of dichloromethane. The organic phase was concentrated and the residue purified by silica chromatography eluting with a 0% to 10% gradient of 2M ammonia in methanol with dichloromethane as co-solvent to yield 360 mg (46%) of (trans) 1,1-dimethylethyl 2-(2,3,3a,4,5,9b-hexahydro-1H-pyrrolo[3,2-h]quinolin-1-ylmethyl)-1H-benzimidazole-1-carboxylate and 56 mg (5%) of (trans) 1,1-dimethylethyl 2-{[2-(2,3,3a,4,5,9b-hexahydro-1H-pyrrolo[3,2-h]quinolin-1-ylmethyl)-1H-benzimidazol-1-yl]methyl}-1H-benzimidazole-1-carboxylate. 128a; ¹H NMR (400 MHz, METHANOL-D4) δ ppm 1.6 (s, 9H), 1.6 (m, 1H), 1.7 (m, 2H), 1.8 (m, 1H), 2.2 (m, 1H), 2.5 (m, 1H), 2.6 (m, 1 H), 2.7 (m, 1H), 2.9 (m, 1H), 3.0 (m, 1H), 3.6 (d, J=8.4 Hz, 1H), 3.9 (d, J=14.8 Hz, 1H), 7.2 (dd, J=7.5, 4.9 Hz, 1H), 7.3 (m, 2H), 7.6 (m, 2H), 7.8 (d, J=7.7 Hz, 1H), 8.3 (d, J=6.2 Hz, 1H); MS m/z 427 (M+Na⁺). 128b; ¹H NMR (400 MHz, METHANOL-D4) δ ppm 1.6 (m, 2H), 1.8 (s, 9H), 2.1 (m, 1H), 2.4 (m, 1H), 2.5 (m, 1H), 2.6 (m, 2 H), 2.9 (t, J=7.4 Hz, 2H), 3.4 (d, J=8.4 Hz, 1H), 3.7 (d, J=13.7 Hz, 1H), 4.8 (d, J=13.9 Hz, 1H), 5.6 (d, J=18.8 Hz, 1H), 5.9 (d, J=18.8 Hz, 1H), 6.7 (m, 1H), 7.2 (m, 3H), 7.4 (m, 4H), 7.5 (d, J=4.8 Hz, 2H), 7.7 (d, J=8.6 Hz, 1H), 7.9 (d, J=8.1 Hz, 1 H); MS m/z 535 (M+1).

### Example 129

### (trans) 1-(1H-benzimidazol-2-ylmethyl)-2,3,3a,4,5,9b-hexahydro-1H-pyrrolo[3,2-h]quinoline (intermediate)

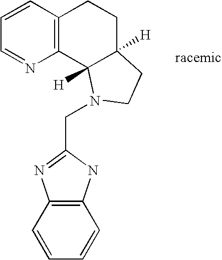

(trans) 1,1-Dimethylethyl 2-(2,3,3a,4,5,9b-hexahydro-1H-pyrrolo[3,2-h]quinolin-1-ylmethyl)-1H-benzimidazole-1-carboxylate (0.34 g, 0.84 mmol) was dissolved in 5 mL of dichloromethane and then 5 mL of trifluoroacetic acid was added. The mixture was stirred for 2 hours and concentrated. The residue was slurried in 15 mL of saturated sodium bicarbonate and extracted with 15 mL of dichloromethane. The organic phase was washed with brine, dried over sodium sulfate, and concentrated to yield 225 mg (88%) of (trans) 1-(1H-benzimidazol-2-ylmethyl)-2,3,3a,4,5,9b-hexahydro-1H-pyrrolo[3,2-h]quinoline. ¹H NMR (400 MHz, METHANOL-D4) δ ppm 1.7 (m, 2H), 1.9 (m, 1H), 2.1 (m, 1H), 2.5 (td, J=9.5, 7.3 Hz, 1H), 2.7 (m, 2H), 3.0 (m, 2H), 3.6 (d, J=8.2 Hz, 1H), 3.8 (d, J=14.6 Hz, 1H), 4.4 (d, J=14.5 Hz, 1H), 7.2 (m, 2H), 7.3 (m, 1H), 7.5 (dd, J=5.9, 3.2 Hz, 2H), 7.6 (d, J=7.5 Hz, 1H), 8.4 (d, J=6.6 Hz, 1H); MS m/z 305 (M+1).

### Example 130

### (trans) 4-[2-(2,3,3a,4,5,9b-hexahydro-1H-pyrrolo[3,2-h]quinolin-1-ylmethyl)-1H-benzimidazol-1-yl]-1-butanamine

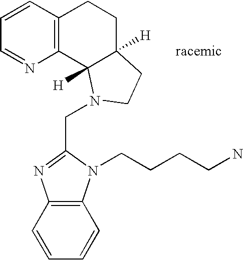

(trans) 1-(1H-benzimidazol-2-ylmethyl)-2,3,3a,4,5,9b-hexahydro-1H-pyrrolo[3,2-h]quinoline (0.032 g, 0.11 mmol), potassium carbonate (0.075 g, 0.55 mmol), 4-bromobutanenitrile (0.024 g, 0.16 mmol), and potassium iodide (5 mg) in 3 mL of N,N-dimethylformamide were heated to 80° C. in a sealed tube for 16 hours. The mixture was allowed to cool to room temperature and quenched with 5 mL of water. The mixture was extracted 3 times with 5 mL of dichloromethane and the combined organic layers concentrated. The residue was dissolved in 20 mL of a 2M ammonia solution in methanol and hydrogenated under 40 psi of hydrogen for 18 hours, with 30 mg of raney nickel as catalyst. The catalyst was filtered off over celite and the filtrate concentrated. The residue was purified by silica chromatography eluting with a 0% to 10% gradient of aqueous ammonia in acetonitrile to yield 29 mg (70%) of (trans) 4-[2-(2,3,3a,4,5,9b-hexahydro-1H-pyrrolo[3,2-h]quinolin-1-ylmethyl)-1H-benzimidazol-1-yl]-1-butanamine. ¹H NMR (400 MHz, METHANOL-D4) δ ppm 1.1 (m, 2H), 1.4 (m, 2H), 1.7 (m, 2H), 1.8 (m, 1H), 2.1 (m, 1H), 2.4 (m, 1H), 2.5 (t, J=7.4 Hz, 2H), 2.7 (m, 1H), 2.8 (m, 2H), 2.9 (m, 1H), 3.6 (d, J=8.6 Hz, 1H), 3.6 (d, J=13.6 Hz, 1H), 3.7 (m, 1H), 3.8 (m, 1H), 4.7 (d, J=13.6 Hz, 1H), 7.2 (m, 2H), 7.4 (m, 2H), 7.6 (m, 1H), 7.7 (d, J=7.9 Hz, 1H), 8.4 (d, J=6.4 Hz, 1H); MS m/z 376 (M+1).

### Example 131

### (trans) 1-{[1-(1H-benzimidazol-2-ylmethyl)-1H-benzimidazol-2-yl]methyl}-2,3,3a,4,5,9b-hexahydro-1H-pyrrolo[3,2-h]quinoline

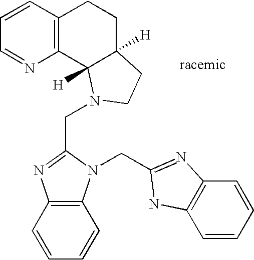

(trans) 1,1-Dimethylethyl 2-{[2-(2,3,3a,4,5,9b-hexahydro-1H-pyrrolo[3,2-h]quinolin-1-ylmethyl)-1H-benzimidazol-1-yl]methyl}-1H-benzimidazole-1-carboxylate was dissolved in 2.5 mL of dichloromethane and 2.5 mL of trifluoroacetic acid was added. The mixture was stirred at room temperature for 2 hours and then concentrated. The residue was slurried in 25 mL of saturated sodium bicarbonate and extracted 3 times with 15 mL of dichloromethane. The combined organic layers were washed with brine, dried over sodium sulfate, and concentrated. The residue was purified by silica chromatography eluting with a 0% to 10% gradient of 2M ammonia in methanol with dichloromethane as co-solvent to yield 18 mg (44%) of (trans) 1-{[1-(1H-benzimidazol-2-ylmethyl)-1H-benzimidazol-2-yl]methyl}-2,3,3a,4,5,9b-hexahydro-1H-pyrrolo[3,2-h]quinoline. ¹H NMR (400 MHz, METHANOL-D4) δ ppm 1.6 (m, 3H), 2.1 (m, 1H), 2.5 (m, 3H), 2.7 (m, 1H), 3.0 (m, 1H), 3.6 (d, J=8.8 Hz, 1H), 3.8 (d, J=13.7 Hz, 1H), 5.0 (d, J=13.9 Hz, 1H), 5.2 (d, J=17.2 Hz, 1H), 5.4 (d, J=17.0 Hz, 1 H), 6.9 (dd, J=7.7, 4.9 Hz, 1H), 7.2 (m, 5H), 7.4 (d, J=7.9 Hz, 3H), 7.6 (d, J=8.1 Hz, 1H), 8.0 (d, J=4.8 Hz, 1H); MS m/z 435 (M+1).

### Example 132

### (trans) 3-[2-(2,3,3a,4,5,9b-hexahydro-1H-pyrrolo[3,2-h]quinolin-1-ylmethyl)-1H-benzimidazol-1-yl]-N,N-dimethyl-1-propanamine

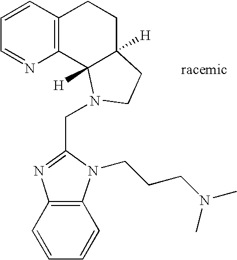

(trans) 1-(1H-benzimidazol-2-ylmethyl)-2,3,3a,4,5,9b-hexahydro-1H-pyrrolo[3,2-h]quinoline (0.032 g, 0.11 mmol), potassium carbonate (0.075 g, 0.55 mmol), (3-chloropropyl)dimethylamine (0.025 g, 0.16 mmol), and potassium iodide (5 mg) in 3 mL of N,N-dimethylformamide were heated to 80° C. in a sealed tube for 16 hours. The mixture was allowed to cool to room temperature and quenched with 5 mL of water. The mixture was extracted 3 times with 5 mL of dichloromethane and the combined organic layers concentrated. The residue was purified by silica chromatography eluting with a 0% to 10% gradient of aqueous ammonia in acetonitrile to yield 30 mg (70%) of (trans) 3-[2-(2,3,3a,4,5,9b-hexahydro-1H-pyrrolo[3,2-h]quinolin-1-ylmethyl)-1H-benzimidazol-1-yl]-N,N-dimethyl-1-propanamine. ¹H NMR (400 MHz, METHANOL-D4) δ ppm 1.6 (m, 4H), 1.9 (m, 3H), 2.1 (m, 7H), 2.4 (td, J=9.7, 7.0 Hz, 1H), 2.7 (m, 3H), 2.9 (m, 1H), 3.6 (d, J=8.6 Hz, 1H), 3.6 (d, J=13.6 Hz, 1H), 3.7 (m, 1H), 3.9 (m, 1H), 4.7 (d, J=13.6 Hz, 1H), 7.2 (m, 2H), 7.3 (dd, J=7.6, 4.9 Hz, 1H), 7.4 (d, J=7.1 Hz, 1H), 7.6 (d, J=7.0 Hz, 1H), 7.7 (d, J=7.5 Hz, 1H), 8.4 (d, J=4.8 Hz, 1H); MS m/z 390 (M+1).

### Example 133

### (trans) 1-({1-[3-(1-piperidinyl)propyl]-1H-benzimidazol-2-yl}methyl)-2,3,3a,4,5,9b-hexahydro-1H-pyrrolo[3,2-h]quinoline

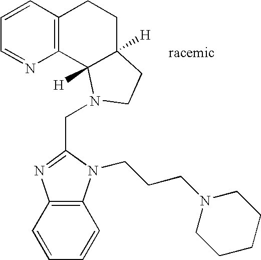

(trans) 1-(1H-benzimidazol-2-ylmethyl)-2,3,3a,4,5,9b-hexahydro-1H-pyrrolo[3,2-h]quinoline (0.032 g, 0.11 mmol), potassium carbonate (0.075 g, 0.55 mmol), 1-(3-chloropropyl)piperidine hydrochloride (0.025 g, 0.16 mmol), and potassium iodide (5 mg) in 3 mL of N,N-dimethylformamide were heated to 80° C. in a sealed tube for 16 hours. The mixture was allowed to cool to room temperature and quenched with 5 mL of water. The mixture was extracted 3 times with 5 mL of dichloromethane and the combined organic layers concentrated. The residue was purified by silica chromatography eluting with a 0% to 10% gradient of aqueous ammonia in acetonitrile to yield 33 mg (70%) of (trans) 1-({1-[3-(1-piperidinyl)propyl]-1H-benzimidazol-2-yl}methyl)-2,3,3a,4,5,9b-hexahydro-1H-pyrrolo[3,2-h]quinoline. ¹H NMR (400 MHz, METHANOL-D4) δ ppm 1.4 (d, J=5.1 Hz, 2H), 1.5 (m, 5H), 1.6 (m, 3H), 1.8 (m, 3H), 2.1 (m, 1H), 2.2 (m, 3H), 2.4 (m, 2H), 2.7 (m, 3H), 2.9 (m, 1H), 3.6 (d, J=8.6 Hz, 1H), 3.6 (d, J=13.5 Hz, 1H), 3.7 (m, 1H), 3.9 (dt, J=14.3, 7.2 Hz, 1 H), 4.7 (d, J=13.5 Hz, 1H), 7.2 (m, 2H), 7.3 (m, 1H), 7.4 (d, J=6.4 Hz, 1H), 7.6 (m, 1H), 7.7 (d, J=7.7 Hz, 1H), 8.4 (d, J=4.8 Hz, 1H); MS m/z 430 (M+1).

### Example 134

### (4aR,10bS)-1-{[1-methyl-7-(4-methyl-1-piperazinyl)-1H-benzimidazol-2-yl]methyl}-1,2,3,4,4a,5,6,10b-octahydro-1,10-phenanthroline

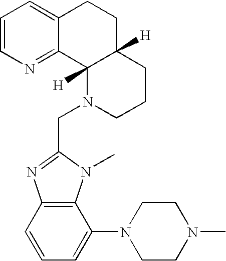

A) 3-Chloro 2-nitroaniline (25 g, 144.9 mmol) and 1-boc-piperazine (68.0 g, 365.3 mmol) were dissolved in N,N-diisopropylethylamine (75.7 mL, 434.7 mmol) and the reaction mixture was heated for 48 h at 135° C. The reaction was cooled, concentrated and loaded onto alumina before passing through a plug of silica eluting with 10% EtOAc in hexanes. Recovered 39.5 g (71% yield) of tert-butyl 4-(3-amino-2-nitrophenyl)-1-piperazinecarboxylate as a dark yellow solid: ¹H-NMR (DMSO-d₆) δ 7.10 (t, 1H), 6.55 (d, 1H), 6.38 (d, 1H), 5.85 (br s, 2H), 3.34-3.32 (m, 4H), 2.79-2.77 (m, 4H), 1.38 (s, 9H).

B) Tert-butyl 4-(3-amino-2-nitrophenyl)-1-piperazinecarboxylate (39.5 g, 122.6 mmol) and palladium on carbon (10% w/w, catalytic) were dissolved in ethanol (500 mL) and the reaction was stirred under a hydrogen atmosphere for 48 h. The reaction was filtered through celite, washing with EtOH then concentrated to provide tert-butyl 4-(2,3-diaminophenyl)-1-piperazinecarboxylate (35.7 g, 100%) as a brown solid: ¹H-NMR (DMSO-d₆) δ 6.39-6.32 (m, 2H), 6.30-6.27 (m, 1H), 4.44 (br s, 2H), 4.18 (br s, 2H), 3.48-3.41 (m, 4H), 2.70-2.62 (m, 4H), 9.28 (s, 9H).

C) Tert-butyl 4-(2,3-diaminophenyl)-1-piperazinecarboxylate (39.73 g, 135 mmol), acetoxyacetic acid (16.04 g, 135.8 mmol), bis(2-oxo-3-oxazolidinyl)phosphinic chloride (51.89 g, 203.8 mmol), and N,N-diisopropylethylamine (35.5 mL, 203.8 mmol) were dissolved in acetonitrile (350 mL) and the reaction was stirred for 12 h at room temperature. The reaction was concentrated to 50 mL and diluted with ethyl acetate (200 mL). The organic phase was washed with water (200 mL) and the aqueous phase was extracted with ethyl acetate four times (100 mL). The organic extracts were combined, dried over sodium sulfate, filtered and concentrated to a dark oil. The crude amide was dissolved in acetic acid (350 mL), heated at 70° C. for 2 h, cooled and concentrated. The crude material was diluted with dichloromethane (200 mL) and saturated sodium bicarbonate (200 mL). The aqueous phase was extracted with dichloromethane (100 mL) and the organic extracts were combined, dried over sodium sulfate, filtered and concentrated to a red oily foam with no further purification (41.86 g, 82%): ¹H-NMR (DMSO-d₆) δ 12.44 (s, 1H), 7.07-6.98 (m, 2H), 6.49 (d, 1H), 5.20 (s, 2H), 3.55-3.36 (m, 8H), 2.09 (s, 3H), 1.40 (s, 9H).

D) Tert-butyl 4-{2-[(acetyloxy)methyl]-1-methyl-1H-benzimidazol-7-yl}-1-piperazinecarboxylate A slurry of tert-butyl 4-{2-[(acetyloxy)methyl]-1H-benzimidazol-4-yl}-1-piperazinecarboxylate (19.49 g, 52.0 mmol), cesium carbonate (25.41 g, 78.0 mmol) and iodomethane (3.97 mL, 62.0 mmol) in N,N-dimethylformamide (550 mL) was stirred at room temperature for 3 h. Concentrated solvent to near dryness. Diluted with ethyl acetate and water and the phases separated. The organic phase was dried over sodium sulfate, filtered, concentrated and purified on silica (0% to 100% ethyl acetate/hexanes gradient) to provide the products as red solids. Isolated the desired isomer, tert-butyl 4-{2-[(acetyloxy)methyl]-1-methyl-1H-benzimidazol-7-yl}-1-piperazinecarboxylate (11.94 g, 48%). ¹H-NMR (CDCl₃) δ 7.55 (d, 1H), 7.19 (t, 1H), 7.02 (d, 1H), 5.36 (s, 2H), 4.19-4.10 (m, 5H), 3.14-3.05 (m, 4H), 2.90-2.83 (m, 2H), 2.14 (s, 3H), 1.48 (s, 9H). MS m/z 389 (M+1).

E) To a solution of tert-butyl 4-{2-[(acetyloxy)methyl]-1-methyl-1H-benzimidazol-7-yl}-1-piperazinecarboxylate (7.01 g, 18.0 mmol) in methanol (185 mL) was added cesium carbonate (catalytic). The reaction was stirred at room temperature for 1 h, concentrated and purified by passing through a plug of silica rinsing with 4 L of 5% 2M NH₃ in methanol/dichloromethane, followed by 3 L of 10% 2M NH₃ in methanol/dichloromethane, to provide tert-butyl 4-[2-(hydroxymethyl)-1-methyl-1H-benzimidazol-7-yl]-1-piperazinecarboxylate (5.6 g, 90%) as a light tan foam: ¹H-NMR (DMSO-d₆): δ 7.30 (d, 1H), 7.05 (t, 1H), 6.94 (d, 1H), 5.49 (t, 1H), 4.66 (d, 2H), 4.10 (s, 3H), 4.02-3.92 (m, 2H), 3.14-3.05 (m, 4H), 2.74-2.67 (m, 2H), 1.41 (s, 9H). MS m/z 347 (M+1).

F) Tert-butyl 4-[2-(hydroxymethyl)-1-methyl-1H-benzimidazol-7-yl]-1-piperazinecarboxylate (9.59 g, 28.0 mmol) was heated in 1.0 L of acetonitrile to 60° C. until all solids had dissolved. Added manganese dioxide (85% w/w, 28.3 g, 280 mmol) to warm solution and stirred for 24 h at room temperature. The reaction was filtered through celite. The celite pad was rinsed with an additional portion of hot acetonitrile (5 L). The filtrate was concentrated to provide tert-butyl 4-(2-formyl-1-methyl-1H-benzimidazol-7-yl)-1-piperazinecarboxylate (6.59, 69%) as a yellow oil: ¹H-NMR (CDCl₃): δ10.11 (s, 1H), 7.67 (d, 1H), 7.29 (t, 1H), 7.11 (d, 1H), 4.51 (s, 3H), 4.20-4.12 (m, 2H), 3.18-3.10 (m, 4H), 2.90-2.83 (m, 2H), 1.50 (s, 9H). MS m/z 345 (M+1).

G) (4aR,10bS)-1-{[1-methyl-7-(4-methyl-1-piperazinyl)-1H-benzimidazol-2-yl]methyl}-1,2,3,4,4a,5,6,10b-octahydro-1,10-phenanthroline: Dissolved (0.065 g, 0.346 mmol) (4aR,10bS)-1,2,3,4,4a,5,6,10b-octahydro-1,10-phenanthroline in 2 mL of anhydrous 1,2-dichloroethane. Added 1,1-dimethylethyl 4-(2-formyl-1-methyl-1H-benzimidazol-7-yl)-1-piperazinecarboxylate (0.119 g, 0.346 mmol) and acetic acid (0.029 mL, 0.518 mmol) and let stir for 15 minutes. Added Na(OAc)₃BH and let stir at room temperature overnight. Quenched reaction with 10% aqueous sodium carbonate, separated layers and washed twice with dichloromethane. Dried organics over sodium sulfate, filtered, and concentrated. Residue was purified by reverse phase chromatography eluting with a 0-50% gradient of acetonitrile (0.1% trifluoroacetic acid) in water to yield 0.054 g of 1,1-dimethylethyl 4-{2-[(4aR,10bS)-3,4,4a,5,6,10b-hexahydro-1,10-phenanthrolin-1(2H)-ylmethyl]-1-methyl-1H-benzimidazol-7-yl}-1-piperazinecarboxylate. This was dissolved in 2 mL of dichloromethane and 2 mL of trifluoroacetic acid was added. Reaction was concentrated after 30 minutes and residue was dissolved in 5 mL of anhydrous methanol. MP-Carbonate resin was added, and stirred until pH was neutral. Filtered resin and concentrated to afford 0.035 g of (4aR,10bS)-1-{[1-methyl-7-(1-piperazinyl)-1H-benzimidazol-2-yl]methyl}-1,2,3,4,4a,5,6,10b-octahydro-1,10-phenanthroline. Dissolved product in 5.0 mL of 1,2-dichloroethane and added 37% aqueous formaldehyde (0.013 mL, 0.168 mmol), acetic acid (0.010 mL, 0.168 mmol), and Na(OAc)₃BH (0.036 g, 0.168 mmol) and let stir three days. Quenched reaction with 10% aqueous sodium carbonate, separated layers and washed twice with dichloromethane. Dried organics over sodium sulfate, filtered, and concentrated. Residue was purified by reverse phase chromatography eluting with a 0-100% gradient of acetonitrile (0.1% trifluoroacetic acid) in water to afford (4aR,10bS)-1-{[1-methyl-7-(4-methyl-1-piperazinyl)-1H-benzimidazol-2-yl]methyl}-1,2,3,4,4a,5,6,10b-octahydro-1,10-phenanthroline. ¹H NMR (300 MHz, METHANOL-D4) δ 1.51 (m, 2H), 1.88 (m, 1H), 2.12 (m, 3H), 2.58-2.79 (m, 2H), 2.89 (m, 1H), 3.08 (m, 5H), 3.38 (m, 4H), 3.53 (m, 4H), 3.74 (m, 2H), 4.44 (s, 3H), 4.55 (m, 1H), 7.53 (m, 1H), 7.63 (t, 1H), 7.75 (m, 1H), 8.00 (m, 1H), 8.44 (d, 1H), 8.90 (d, 1H); MS m/z 431 (M+1).

### Biological Section

### Fusion Assay

### Plasmid Generation

The complete coding sequences of HIV-1 tat (GenBank Accession No. X07861) and rev (GenBank Accession No. M34378) were cloned into pcDNA3.1 expression vectors containing G418 and hygromycin resistance genes, respectively. The complete coding sequence of the HIV-1 (HXB2 strain) gp160 envelope gene (nucleotide bases 6225-8795 of GenBank Accession No. K03455) was cloned into plasmid pCRII-TOPO. The three HIV genes were additionally inserted into the baculovirus shuttle vector, pFastBacMam1, under the transcriptional control of the CMV promoter. A construction of the pHIV-I LTR containing mutated NFkB sequences linked to the luciferase reporter gene was prepared by digesting pcDNA3.1, containing the G418 resistance gene, with Nru I and Bam HI to remove the CMV promoter. LTR-luc was then cloned into the Nru I/Bam HI sites of the plasmid vector. Plasmid preparations were performed after the plasmids were amplified in Escherichia coli strain DH5-alpha. The fidelity of the inserted sequences was confirmed by double-strand nucleotide sequencing using an ABI Prism Model 377 automated sequencer.

### BacMam Baculovirus Generation

Recombinant BacMam baculoviruses were constructed from pFastBacMam shuttle plasmids by using the bacterial cell-based Bac-to-Bac system. Viruses were propagated in Sf9 (Spodoptera frugiperda) cells cultured in Hink's TNM-FH Insect media supplemented with 10% (v/v) fetal bovine serum and 0.1% (v/v) pluronic F-68 according to established protocols.

### Cell Culture

Human osteosarcoma (HOS) cells that naturally express human CXCR4 were transfected with human CCR5, human CD4 and the pHIV-LTR-luciferase plasmid using FuGENE 6 transfection reagent. Single cells were isolated and grown under selection condition in order to generate a stable HOS (hCXCR4/hCCR5/hCD4/pHIV-LTR-luciferase) clonal cell line. The cells were maintained in Dulbeccos modified Eagles media supplemented with 10% fetal calf serum (FCS), G418 (400 ug/ml), puromycin (1 ug/ml), mycophenolic acid (40 ug/ml), xanthine (250 ug/ml) and hypoxanthine (13.5 ug/ml) to maintain a selection pressure for cells expressing the LTR-luciferase, hCCR5 and hCD4, respectively. Human embryonic kidney (HEK-293) cells stably transfected to express the human macrophage scavenging receptor (Class A, type 1; GenBank Accession No. D90187), were maintained in DMEM/F-12 media (1:1) supplemented with 10% FCS and 1.5 ug/ml puromycin. The expression of this receptor by the HEK-293 cells enhances their ability to stick to tissue culture treated plasticware.

### Transduction of HEK-293 Cells

HEK-293 cells were harvested using enzyme-free cell dissociation buffer. The cells were resuspended in DMEM/F-12 media supplemented with 10% FCS and 1.5 ug/ml and counted. Tranductions were performed by direct addition of BacMam baculovirus containing insect cell media to cells. The cells were simultaneously transduced with BacMam baculovirus expressing HIV-1 tat, HIV-1 rev and HIV-1 gp160 (from the HXB2 HIV strain). Routinely an MOI of 10 of each virus was added to the media containing the cells. 2 mM butyric acid was also added to the cells at this stage to increase protein expression in transduced cells. The cells were subsequently mixed and seeded into a flask at 30 million cells per T225. The cells were incubated at 37° C., 5% CO₂, 95% humidity for 24 h to allow for protein expression.

### Cell/Cell Fusion Assay Format

HEK and HOS cells were harvested in DMEM/F-12 media containing 2% FCS and DMEM media containing 2% FCS, respectively, with no selection agents added. Compounds were plated as 1 ul spots in 100% DMSO on a 96-well CulturPlate plates. HOS cells (50 ul) were added first to the wells, followed immediately by the HEK cells (50 ul). The final concentration of each cell type was 20,000 cells per well. Following these additions, the cells were returned to a tissue culture incubator (37° C.; 5% CO₂/95% air) for an additional 24 h.

### Measurement of Luciferase Production

Following the 24 h incubation, total cellular luciferase activity was measured using the LucLite Plus assay kit (Packard, Meridien, Conn.). In brief, 100 ul of this reagent was added to each well. The plates were sealed and mixed. The plates were dark adapted for approximately 10 min prior to the luminescence being read on a Packard TopCount.

### Functional Assay

### Cell Culture

Human embryonic kidney (HEK-293) cells were maintained and harvested as described above. Cells were plated in 96-well, black clear bottom, poly-lysine coated plates at a concentration of 40,000 cells per well in a final volume of 100 ul containing human CXCR4BacMam (MOI=25) and Gqi5 BacMam (MOI=12.5). The cells were incubated at 37° C., 5% CO₂, 95% humidity for 24 h to allow for protein expression.

### Functional FLIPR Assay

After the required incubation time the cells were washed once with 50 ul of fresh serum-free DMEM/F12 media containing probenicid. 50 ul of dye solution was then added to the cells (Calcium Plus Assay Kit Dye; Molecular Devices) was dissolved in 200 ml of the above probenicid/BSA containing media and incubated for 1 h. Cell plates were transferred to a Fluorometric Imaging Plate Reader (FLIPR). Upon addition the effect of the compounds on the change in [Ca²⁺]ᵢ was examined to determine if the compounds were agonists or antagonists (ability to block SDF-1 alpha activity) at the CXCR4 receptor. IC₅₀ values are determined and pK_{b} values are calculated using the Leff and Dougall equation: K_{B}=IC₅₀/((2+([agonist]/EC₅₀̂b) ̂1/b−1) Where IC₅₀ is that defined by the antagonist concentration-response curve [agonist] is the EC₈₀ concentration of agonist used EC₅₀ is that defined by the agonist concentration-response curve b is the slope of the agonist concentration-response curve.

### HOS HIV-1 Infectivity Assay

### HIV Virus Preparation

Compounds were profiled against two HIV-1 viruses, the M-tropic (CCR5 utilizing) Ba-L strain and the T-tropic (CXCR4 utilizing) IIIB strain. Both viruses were propagated in human peripheral blood lymphocytes. Compounds were tested for there ability to block infection of the HOS cell line (expressing hCXCR4/hCCR5/hCD4/pHIV-LTR-luciferase) by either HIV-1 Ba-L or HIV-1, IIIB. Compound cytotoxicity was also examined in the absence of virus addition.

### HOS HIV-1 Infectivity Assay Format

HOS cells (expressing hCXCR4/hCCR5/hCD4/pHIV-LTR-luciferase) were harvested and diluted in Dulbeccos modified Eagles media supplemented with 2% FCS and non-essential amino acid to a concentration of 60,000 cells/ml. The cells were plated into 96-well plates (100 ul per well) and the plates were placed in a tissue culture incubator (37° C.; 5% CO₂/95% air) for a period of 24 h.

Subsequently, 50 ul of the desired drug solution (4 times the final concentration) was added to each well and the plates were returned to the tissue culture incubator (37° C.; 5% CO₂/95% air) for 1 h. Following this incubation 50 ul of diluted virus was added to each well (approximately 2 million RLU per well of virus). The plates were returned to the tissue culture incubator (37° C.; 5% CO₂/95% air) and were incubated for a further 96 h.

Following this incubation the endpoint for the virally infected cultures was quantified following addition of Steady-Glo Luciferase assay system reagent (Promega, Madison, Wis.). Cell viability or non-infected cultures was measured using a CellTiter-Glo luminescent cell viability assay system (Promega, Madison, Wis.). All luminescent readouts are performed on a Topcount luminescence detector (Packard, Meridien, Conn.).

Alternatively the HOS assay was conducted as outlined below:

### HOS HIV-1 Infectivity Assay

### HIV Virus Preparation

Compounds were profiled against two HIV-1 viruses, the M-tropic (CCR5 utilizing) Ba-L strain and the T-tropic (CXCR4 utilizing) IIIB strain. Ba-L was propagated in either peripheral blood lymphocytes or SupT1/CCR5+/CXCR4+ cells. IIIB was propagated in peripheral blood lymphocytes. Compounds were tested for their ability to block infection of the HOS cell line (expressing hCXCR4/hCCR5/hCD4/pHIV-LTR-luciferase) by either HIV-1 Ba-L or HIV-1, IIIB. Compound cytotoxicity was also examined in the absence of virus addition.

### HOS HIV-1 Infectivity Assay Format

HOS cells (expressing hCXCR4/hCCR5/hCD4/pHIV-LTR-luciferase) were harvested and diluted in Dulbeccos modified Eagles media supplemented with 2% FCS to a concentration of 120,000 cells/ml. The cells were plated into 96-well plates (50 μl per well) and the plates were placed in a tissue culture incubator (370° C.; 5% CO₂/95% air) for a period of 24 h.

Subsequently, 50 μl of the desired drug solution (2 times the final concentration) was added to each well and the plates were returned to the tissue culture incubator (370° C.; 5% CO₂/95% air) for 1 h. Following this incubation, 60 μl of diluted virus (4 times the final concentration) was added to 60 μl of 2× the final desired concentration of the compound and 100 μl of this compound/virus mix was added to each well (approximately 2 million RLU per well of virus). The plates were returned to the tissue culture incubator (370° C.; 5% CO₂/95% air) and were incubated for a further 96 h.

Following this incubation the endpoint for the virally infected cultures was quantified following addition of Steady-Glo Luciferase assay system reagent (Promega, Madison, Wis.). Cell viability or non-infected cultures was measured using a CellTiter-Glo luminescent cell viability assay system (Promega, Madison, Wis.). All luminescent readouts are performed on a Topcount luminescence detector (Packard, Meridien, Conn.).

TABLE 1

| | Functional | | | |
|---|---|---|---|---|
| | assay | Fusion assay | Cytotox | |
| Example | (pIC50) | (pIC50) | (pIC50) | HOS (3B) (μM) |
| 6 | 6.9 | 5.95 | 4.5 | 0.47 |
| 7 | 6.36 | 5.5 | <4.0 | 1.0 |
| 8 | 6.9 | 6.31 | <4 | 0.22 |
| 9 | 7.71 | 7.05 | 4.54 | 0.073 |
| 10A | 6.74 | 6.38 | <4 | 0.33 |
| 10B | 7.03 | 6.70 | <4 | 0.14 |
| 11A | 6.07 | 5.9 | <4 | 0.53 |
| 11B | 7.98 | 7.6 | <4 | 0.035 |
| 12 | 5.98 | 5.1 | <4 | 0.77 |
| 13 | 8.12 | 7.5 | <4 | 0.05 |
| 15A | 7.39 | 7.11 | <4 | 0.033 |
| 15B | 8.47 | 8.26 | <4 | 0.004 |
| 22 | 7.5 | 6.8 | <4 | 0.1 |
| 27 | 8.9 | 8.9 | <5 | 0.004 |
| 32 | 6.32 | 6.7 | <5 | 0.26 |
| 33 | 8.62 | 9.4 | 4.43 | 0.0035 |

Compounds of the present invention demonstrate desired potency. Moreover, compounds of the present invention are believed to provide a desired pharmacokinetic profile. Also, compounds of the present invention are believed to provide a desired secondary biological profile.

Although specific pIC₅₀ values were generated for certain of the present compounds, these values should be considered exemplary. Those skilled in the art will appreciate the variability in performing and recording data using the biological activity assays that are herein described.

Test compounds were employed in free or salt form.

All research complied with the principles of laboratory animal care (NIH publication No. 85-23, revised 1985) and GlaxoSmithKline policy on animal use.

TABLE 2

| Example | Activity Level* |
|---|---|
| 35 | B |
| 37 | A |
| 38 | B |
| 39 | B |
| 40 | B |
| 41 | A |
| 42 | B |
| 43 | A |
| 44 | B |
| 45 | C |
| 46 | C |
| 48 | A |
| 50 | C |
| 51a | C |
| 51b | C |
| 52 | C |
| 53 | A |
| 54 | A |
| 55 | A |
| 56 | A |
| 57 | A |
| 58 | A |
| 59 | A |
| 64 | A |
| 65 | A |
| 66 | A |
| 67 | A |
| 68 | A |
| 69 | B |
| 70 | A |
| 71 | A |
| 72 | A |
| 73 | A |
| 74 | A |
| 75 | A |
| 76a | B |
| 76b | C |
| 77 | A |
| 78 | B |
| 79 | A |
| 80 | B |
| 81 | A |
| 82 | A |
| 83 | A |
| 85 | A |
| 86 | A |
| 87 | A |
| 89 | A |
| 90 | B |
| 92 | A |
| 93 | A |
| 95 | A |
| 97 | A |
| 98 | B |
| 99 | A |
| 100 | A |
| 101 | A |
| 102 | A |
| 103 | A |
| 104 | A |
| 105 | A |
| 106 | A |
| 107 | A |
| 108 | A |
| 109 | B |
| 110 | A |
| 111 | B |
| 112 | A |
| 113 | A |
| 123 | A |
| 124 | A |
| 125 | B |
| 130 | B |
| 131 | C |
| 132 | B |
| 133 | B |
| 134 | A |

As used in the above table, “A” indicates an activity level of less than 100 nM in the HIV infectivity assay. “B” indicates an activity level of between 100 nM to 500 nM in the HIV infectivity assay. “C” indicates an activity level of between 500 nM and 10 μM in the HIV infectivity assay.

Compounds of the present invention demonstrate anti-HIV activity in the range of IC₅₀ of about 1 nM to about 50 μM. In one aspect of the invention, compounds of the present invention have anti-HIV activity in the range of up to about 100 nM. In another aspect of the invention, compounds of the present invention have anti-HIV activity in the range of from about 100 nM to about 500 nM. In another aspect of the invention, compounds of the present invention have anti-HIV activity in the range of from about 500 nM to 10 μM. In another aspect of the invention, compounds have anti-HIV activity in the range of from about 10 μM to about 50 μM. Moreover, compounds of the present invention are believed to provide a desired pharmacokinetic profile. Also, compounds of the present invention are believed to provide a desired selectivity, such as specificity between toxicity and activity.

Although specific embodiments of the present invention are herein illustrated and described in detail, the invention is not limited thereto. The above detailed descriptions are provided as exemplary of the present invention and should not be construed as constituting any limitation of the invention. Modifications will be obvious to those skilled in the art, and all modifications that do not depart from the spirit of the invention are intended to be included with the scope of the appended claims.

What is claimed is:

1. A compound of formula (I) comprising: 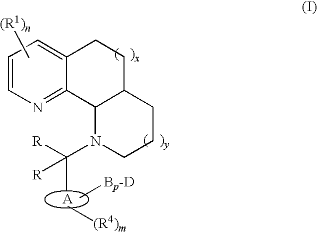
or a salt, solvate, or physiologically functional derivative thereof, wherein
x and y are each independently 0, 1, or 2;
each R independently is H, alkyl, alkenyl, alkynyl, haloalkyl, cycloalkyl, —R^{a}Ay, R^{a}OR¹⁰, —R^{a}NR⁶R⁷, —R^{a}C(O)R¹⁰, —C(O)R¹⁰, —CO₂R¹⁰, —R^{a}CO₂R¹⁰ or —R^{a}S(O)_{q}R¹⁰;
n is 0, 1, 2, or 3;
each R¹ independently is halogen, haloalkyl, alkyl, alkenyl, alkynyl, cycloalkyl, cycloalkenyl, -Ay, —NHAy, -Het, —NHHet, —OR¹⁰, —OAy, —OHet, —R^{a}OR¹⁰, —NR⁶R⁷, —R^{a}NR⁶R⁷, —R^{a}C(O)R¹⁰, —C(O)R¹⁰, —CO₂R¹⁰, —R^{a}CO₂R¹⁰, —C(O)NR⁶R⁷, —C(O)Ay, —C(O)Het, —S(O)₂NR⁶R⁷, —R^{a}S(O)₂NR⁶R⁷, —S(O)_{q}R¹⁰, —S(O)_{q}Ay, —S(O)_{q}Het, —R^{a}S(O)_{q}R¹⁰, cyano, nitro, or azido; 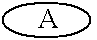
is heteroaryl;
each R⁴ independently is halogen, haloalkyl, alkyl, alkenyl, alkynyl, cycloalkyl, cycloalkenyl, -Ay, —NHAy, -Het, —NHHet, —OR¹⁰, —OAy, —OHet, —R^{a}OR¹⁰, —NR⁶R⁷, —R^{a}NR⁶R⁷, —R^{a}C(O)R¹⁰, —C(O)R¹⁰, —CO₂R¹⁰, —R^{a}CO₂R¹⁰, —C(O)NR⁶R⁷, —C(O)Ay, —C(O)Het, —S(O)₂NR⁶R⁷, —R^{a}S(O)₂NR⁶R⁷, —S(O)_{q}R¹⁰, —S(O)_{q}Ay, —S(O)_{q}Het, cyano, nitro, or azido;
m is 0, 1, or 2;
p is 0 or 1;
B is —NR¹⁰—, —O—, —C(O)NR¹⁰—, —NR¹⁰C(O)—, —C(O)—, —C(O)O—, —NR¹⁰C(O)N(R¹⁰)—, —S(O)_{q}—, —S(O)_{q}NR¹⁰, or —NR¹⁰S(O)_{q}—;
D is —N(R¹⁰)₂, —R^{a}N(R¹⁰)₂, -AyN(R¹⁰)₂, —R^{a}AyN(R¹⁰)₂, -AyR^{a}N(R¹⁰)₂, —R^{a}AyR^{a}N(R¹⁰)₂, -Het, —R^{a}Het, -HetN(R¹⁰)₂, —R^{a}HetN(R¹⁰)₂, -HetR^{a}N(R¹⁰)₂, —R^{a}HetR^{a}N(R¹⁰)₂, -HetR^{a}Ay, or -HetR^{a}Het;
each R^{a} independently is alkylene, cycloalkylene, alkenylene, cycloalkenylene, or alkynylene, where each may be further substituted with alkyl, hydroxyl, or oxo;
each R¹⁰ independently is H, alkyl, cycloalkyl, alkenyl, alkynyl, cycloalkenyl, —R^{a}cycloalkyl, —R^{a}OH, —R^{a}NR⁸R⁹, or R^{a}Het;
each of R⁶ and R⁷ independently are selected from H, alkyl, alkenyl, alkynyl, cycloalkyl, cycloalkenyl, —R^{a}cycloalkyl, —R^{a}OH, —R^{a}OR¹⁰, —R^{a}NR⁸R⁹, -Ay, -Het, —R^{a}Ay, —R^{a}Het, or —S(O)_{q}R¹⁰;
each of R⁸ and R⁹ independently are selected from H or alkyl;
each q independently is 0, 1, or 2;
each Ay independently represents an unsubstituted or substituted aryl group; and
each Het independently represents an unsubstituted or substituted 4-, 5-, or 6-membered heterocyclyl or heteroaryl group.

2. The compound of claim 1 wherein x and y are both 1.

3. The compound of claim 1 wherein x is 1 and y is 2.

4. The compound of claim 1 wherein x is 1 and y is 0.

5. The compound of claim 1 wherein x is 0 and y is 0.

6. The compound of claim 1 wherein x is 0 and y is 1.

7. The compound of claim 1 wherein x is 0 and y is 2.

8. The compound of claim 1 wherein x is 2 and y is 0.

9. The compound of claim 1 wherein x is 2 and y is 1.

10. The compound of claim 1 wherein x is 2 and y is 2.

11. The compound of claim 1 wherein R is hydrogen or an alkyl.

12. The compound of claim 11 herein R is hydrogen.

13. The compound of claim 1 wherein n is 0.

14. The compound of claim 1 wherein n is 1 and R¹ is halogen, haloalkyl, alkyl, OR¹⁰, NR⁶R⁷, CO₂R¹⁰, CONR⁶R⁷ or cyano.

15. The compound of claim 1 wherein heteroaryl A is benzimidazole.

16. The compound of claim 1 wherein heteroaryl A is imidazole.

17. The compound of claim 1 wherein heteroaryl A is imidazopyridine.

18. The compound of claim 1 wherein m is 0.

19. The compound of claim 1 wherein m is 1 or 2.

20. The compound of claim 19 wherein m is 1.

21. The compound of claim 19 wherein R⁴ is one or more of halogen, haloalkyl, alkyl, OR¹⁰, NR⁶R⁷, CO₂R¹⁰, CONR⁶R⁷ or cyano.

22. The compound of claim 1 wherein p is 0 and D is —R^{a}N(R¹⁰)₂, AyR^{a}N(R¹⁰)₂, —R^{a}AyR^{a}N(R¹⁰)₂, -Het, —R^{a}Het, -HetN(R¹⁰)₂, —R^{a}HetN(R¹⁰)₂, or —HetR^{a}N(R¹⁰)₂.

23. The compound of claim 22 wherein X is —R^{a}N(R¹⁰)₂, -Het, —R^{a}Het, -HetN(R¹⁰)₂, —R^{a}HetN(R¹⁰)₂, or -HetR^{a}N(R¹⁰)₂.

24. The compound of claim 22 wherein D is R^{a}N(R¹⁰)₂, -Het, —R^{a}Het, or -HetN(R¹⁰)₂.

25. The compound of claim 22 wherein D is Het.

26. The compound of claim 1 wherein p is 1; B is —N(R¹⁰)—, —O—, —S—, —CONR¹⁰—, —NR¹⁰CO—, or —S(O)_{q}NR¹⁰—; and D is —R^{a}N(R¹⁰)₂, -AyR^{a}N(R¹⁰)₂, —R^{a}AyR^{a}N(R¹⁰)₂, -Het, —R^{a}Het, -HetN(R¹⁰)₂, —R^{a}HetN(R¹⁰)₂, or -HetR^{a}N(R¹⁰)₂.

27. The compound of claim 26 wherein B is —N(R¹⁰)—, —O—, —CONR¹⁰—, —NR¹⁰CO— and D is —R^{a}N(R¹⁰)₂, -Het, —R^{a}Het, or -HetN(R¹⁰)₂.

28. The compound of claim 26 wherein B is NR¹⁰ and D is Het.

29. The compound of claim 1 wherein Het is a substituted or unsubstituted piperidine, piperazine, azetidine, pyrrolidine, imidazole, pyridine.

30. The compound of claim 1 wherein p is 0 and D is -Het.

31. The compound of claim 1 wherein heteroaryl A is imidazole or benzimidazole and substituent —Bₚ-D is attached via imidazole nitrogen as shown by formula 1-A and 1-B:

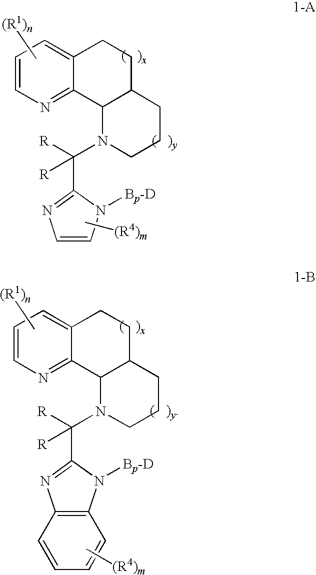

32. The compound of claim 1 wherein heteroaryl A is benzimidazole or imidazopyridine and substituent —Bₚ-D is attached via carbon as shown by formula 1-C and 1-D:

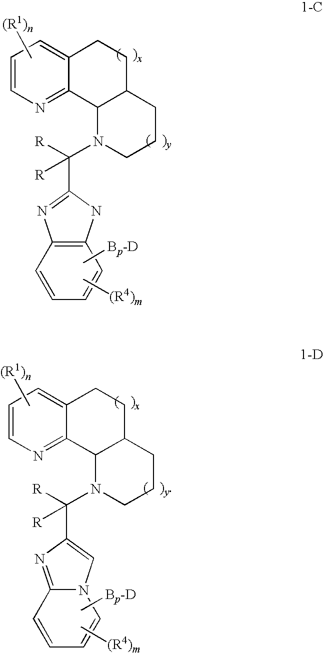

33. The compound of claim 31 wherein heteroaryl A is imidazole as depicted in formula 1-A, and wherein:
n is 0;
x is 1;
y is 1;
R is H;
p is 0;
D is —R^{a}N(R¹⁰)₂, -Het, —R^{a}Het, or -HetN(R¹⁰)₂, where R¹⁰ is H or C₁-C₈ alkyl; and
m is 0.

34. The compound of claim 31 wherein heteroaryl A is benzimidazole as depicted in formula 1-B, and wherein:
n is 0;
x is 1;
y is 1;
R is H;
p is 0;
D is —R^{a}N(R¹⁰)₂, -Het, —R^{a}Het, or -HetN(R¹⁰)₂, where R¹⁰ is H or C₁-C₈ alkyl; and
m is 0.

35. The compound of claim 32 wherein heteroaryl A is benzimidazole as depicted in formula 1-C, and wherein:
n is 0;
x is 1;
y is 1;
R is H;
p is 0;
D is —R^{a}N(R¹⁰)₂, -Het, —R^{a}Het, or -HetN(R¹⁰)₂, where R¹⁰ is H or C₁-C₈ alkyl;
and m is 0.

36. The compound of claim 32 wherein heteroaryl A is imidazopyridine as depicted in formula 1-D, and wherein:
n is 0;
x is 1;
y is 1;
R is H;
p is 0;
D is —R^{a}N(R¹⁰)₂, -Het, —R^{a}Het, or -HetN(R¹⁰)₂, where R¹⁰ is H or C₁-C₈ alkyl;
and m is 0.

37. A compound selected from:
(trans)-3-[2-(3,4,4a,5,6,10b-Hexahydro-1,10-phenanthrolin-1(2H)-ylmethyl)-1H-benzimidazol-1-yl]-1-propanamine;
(cis)-3-[2-(3,4,4a,5,6,10b-Hexahydro-1,10-phenanthrolin-1(2H)-ylmethyl)-1H-benzimidazol-1-yl]-1-propanamine;
(3-{2-[(4aR,10bR)-3,4,4a,5,6,10b-Hexahydro-1,10-phenanthrolin-1(2H)-ylmethyl]-1H-benzimidazol-1-yl}propyl)dimethylamine;
(3-{2-[(4aS,10bS)-3,4,4a,5,6,10b-Hexahydro-1,10-phenanthrolin-1 (2H)-ylmethyl]-1H-benzimidazol-1-yl}propyl)dimethylamine;
(3-{2-[(4aS,10bR)-3,4,4a,5,6,10b-Hexahydro-1,10-phenanthrolin-1(2H)-ylmethyl]-1H-benzimidazol-1-yl}propyl)dimethylamine;
(3-{2-[(4aR,10bS)-3,4,4a,5,6,10b-Hexahydro-1,10-phenanthrolin-1(2H)-ylmethyl]-1H-benzimidazol-1-yl}propyl)dimethylamine;
(cis)-1-{[1-(4-Pyridinylmethyl)-1H-benzimidazol-2-yl]methyl}-1,2,3,4,4a,5,6,10b-octahydro-1,10-phenanthroline;
(cis)-1-({1-[(1-Methyl-3-piperidinyl)methyl]-1H-benzimidazol-2-yl}methyl)-1,2,3,4,4a,5,6,10b-octahydro-1,10-phenanthroline;
(trans)-1-{[5-(4-Methyl-1-piperazinyl)imidazo[1,2-a]pyridin-2-yl]methyl}-1,2,3,4,4a,5,6,10b-octahydro-1,10-phenanthroline;
(cis)-1-{[5-(4-Methyl-1-piperazinyl)imidazo[1,2-a]pyridin-2-yl]methyl}-1,2,3,4,4a,5,6,10b-octahydro-1,10-phenanthroline;
(4aS,10bR)-1-{[5-(4-Methyl-1-piperazinyl)imidazo[1,2-a]pyridin-2-yl]methyl}-1,2,3,4,4a,5,6,10b-octahydro-1,10-phenanthroline;
(4aR,10bS)-1-{[5-(4-Methyl-1-piperazinyl)imidazo[1,2-a]pyridin-2-yl]methyl}-1,2,3,4,4a,5,6,10b-octahydro-1,10-phenanthroline;
(trans)-1,1-Dimethylethyl 2-(3,4,4a,5,6,10b-hexahydro-1,10-phenanthrolin-1(2H)-ylmethyl)-4-(4-methyl-1-piperazinyl)-1H-benzimidazole-1-carboxylate;
(cis)-1,1-Dimethylethyl 2-(3,4,4a,5,6,10b-hexahydro-1,10-phenanthrolin-1(2H)-ylmethyl)-4-(4-methyl-1-piperazinyl)-1H-benzimidazole-1-carboxylate;
(trans)-1-{[4-(4-Methyl-1-piperazinyl)-1H-benzimidazol-2-yl]methyl}-1,2,3,4,4a,5,6,10b-octahydro-1,10-phenanthroline trifluoroacetate;
(cis)-1-{[4-(4-Methyl-1-piperazinyl)-1H-benzimidazol-2-yl]methyl}-1,2,3,4,4a,5,6,10b-octahydro-1,10-phenanthroline;
(trans)-5-[2-(3,4,4a,5,6,10b-hexahydro-1,10-phenanthrolin-1(2H)-ylmethyl)-1H-benzimidazol-1-yl]-1-pentanamine;
(trans) 4-[2-(3,4,4a,5,6,10b-hexahydro-1,10-phenanthrolin-1(2H)-ylmethyl)-1H-benzimidazol-1-yl]-1-butanamine;
(cis) 4-[2-(3,4,4a,5,6,10b-hexahydro-1,10-phenanthrolin-1(2H)-ylmethyl)-1H-benzimidazol-1-yl]-1-butanamine;
(cis) 5-[2-(3,4,4a,5,6,10b-hexahydro-1,10-phenanthrolin-1(2H)-ylmethyl)-1H-benzimidazol-1-yl]-1-pentanamine;
(trans) 3-[2-(3,4,4a,5,6,10b-hexahydro-1,10-phenanthrolin-1(2H)-ylmethyl)-1H-benzimidazol-1-yl]-N,N-dimethyl-1-propanamine;
(cis) 3-[2-(3,4,4a,5,6,10b-hexahydro-1,10-phenanthrolin-1(2H)-ylmethyl)-1H-benzimidazol-1-yl]-N,N-dimethyl-1-propanamine;
(trans) 1-({1-[(1-methyl-3-piperidinyl)methyl]-1H-benzimidazol-2-yl}methyl)-1,2,3,4,4a,5,6,10b-octahydro-1,10-phenanthroline;
(cis) 1-({1-[3-(1-piperidinyl)propyl]-1H-benzimidazol-2-yl}methyl)-1,2,3,4,4a,5,6,10b-octahydro-1,10-phenanthroline;
(cis) 1-({1-[3-(4-morpholinyl)propyl]-1H-benzimidazol-2-yl}methyl)-1,2,3,4,4a,5,6,10b-octahydro-1,10-phenanthroline;
(cis) 1-{[1-(2-pyridinylmethyl)-1H-benzimidazol-2-yl]methyl}-1,2,3,4,4a,5,6,10b-octahydro-1,10-phenanthroline;
(trans) 1-[(1-{[1-(phenylmethyl)-1H-imidazol-2-yl]methyl}-1H-benzimidazol-2-yl)methyl]-1,2,3,4,4a,5,6,10b-octahydro-1,10-phenanthroline;
(4aR,10bS)-1-{[4-(4-methyl-1-piperazinyl)-1H-benzimidazol-2-yl]methyl}-1,2,3,4,4a,5,6,10b-octahydro-1,10-phenanthroline;
(3-{4-[(4aR,10bS)-3,4,4a,5,6,10b-hexahydro-1,10-phenanthrolin-1(2H)-ylmethyl]-1H-imidazol-1-yl}propyl)dimethylamine;
(3-{5-[(4aR,10bS)-3,4,4a,5,6,10b-hexahydro-1,10-phenanthrolin-1(2H)-ylmethyl]-1H-imidazol-1-yl}propyl)dimethylamine;
(cis) 1-{[5-(4-morpholinyl)imidazo[1,2-a]pyridin-2-yl]methyl}-1,2,3,4,4a,5,6,10b-octahydro-1,10-phenanthroline;
(4aR,10bR)-1-{[5-(4-methyl-1-piperazinyl)imidazo[1,2-a]pyridin-2-yl]methyl}-1,2,3,4,4a,5,6,10b-octahydro-1,10-phenanthroline;
[2-[(4aR,10bS)-3,4,4a,5,6,10b-hexahydro-1,10-phenanthrolin-1(2H)-ylmethyl]-5-(4-methyl-1-piperazinyl)imidazo[1,2-a]pyridin-3-yl]methanol;
N-ethyl-N-{[2-[(4aR,10bS)-3,4,4a,5,6,10b-hexahydro-1,10-phenanthrolin-1(2H)-ylmethyl]-5-(4-methyl-1-piperazinyl)imidazo[1,2-a]pyridin-3-yl]methyl}ethanamine;
(4aR,10bS)-1-({5-(4-methyl-1-piperazinyl)-3-[(4-methyl-1-piperazinyl)methyl]imidazo[1,2-a]pyridin-2-yl}methyl)-1,2,3,4,4a,5,6,10b-octahydro-1,10-phenanthroline;
(4aR,10bS)-1-{[5-(4-methyl-1-piperazinyl)-3-(1-piperidinylmethyl)imidazo[1,2-a]pyridin-2-yl]methyl}-1,2,3,4,4a,5,6,10b-octahydro-1,10-phenanthroline;
(4aR,10bS)-1-{[5-(4-methyl-1-piperazinyl)-3-(1-pyrrolidinylmethyl)imidazo[1,2-a]pyridin-2-yl]methyl}-1,2,3,4,4a,5,6,10b-octahydro-1,10-phenanthroline;
(4aR,10bS)-1-{[5-(4-methyl-1-piperazinyl)-3-(4-morpholinylmethyl)imidazo[1,2-a]pyridin-2-yl]methyl}-1,2,3,4,4a,5,6,10b-octahydro-1,10-phenanthroline;
(4aR,10bS)-1-({1-[(3S)-3-piperidinylmethyl]-1H-benzimidazol-2-yl}methyl)-1,2,3,4,4a,5,6,10b-octahydro-1,10-phenanthroline;
(4aR,10bS)-1-[(1-{[(3S)-1-(1-methylethyl)-3-piperidinyl]methyl}-1H-benzimidazol-2-yl)methyl]-1,2,3,4,4a,5,6,10b-octahydro-1,10-phenanthroline;
(4aR,10bS)-1-[(1-{[(3S)-1-methyl-3-piperidinyl]methyl}-1H-benzimidazol-2-yl)methyl]-1,2,3,4,4a,5,6,10b-octahydro-1,10-phenanthroline;
1-{2-[(4aR,10bS)-3,4,4a,5,6,10b-hexahydro-1,10-phenanthrolin-1(2H)-ylmethyl]imidazo[1,2-a]pyridin-5-yl}-N,N-dimethyl-4-piperidinamine;
2-[(4aR,10bS)-3,4,4a,5,6,10b-hexahydro-1,10-phenanthrolin-1(2H)-ylmethyl]-N-(1-methyl-4-piperidinyl)imidazo[1,2-a]pyridin-5-amine;
1-{2-[(4aR,10bS)-3,4,4a,5,6,10b-hexahydro-1,10-phenanthrolin-1(2H)-ylmethyl]imidazo[1,2-a]pyridin-5-yl}-4-piperidinol;
N-{2-[(4aR,10bS)-3,4,4a,5,6,10b-hexahydro-1,10-phenanthrolin-1(2H)-ylmethyl]imidazo[1,2-a]pyridin-5-yl}-N,N′,N′-trimethyl-1,2-ethanediamine;
(4aR,10bS)-1-{[5-(1,4′-bipiperidin-1′-yl)imidazo[1,2-a]pyridin-2-yl]methyl}-1,2,3,4,4a,5,6,10b-octahydro-1,10-phenanthroline;
(4aR,10bS)-1-({5-[4-(1-pyrrolidinyl)-1-piperidinyl]imidazo[1,2-a]pyridin-2-yl}methyl)-1,2,3,4,4a,5,6,10b-octahydro-1,10-phenanthroline;
(4aR,10bS)-1-{[5-(4-ethyl-1-piperazinyl)imidazo[1,2-a]pyridin-2-yl]methyl}-1,2,3,4,4a,5,6,10b-octahydro-1,10-phenanthroline;
2-[(4aR,10bS)-3,4,4a,5,6,10b-hexahydro-1,10-phenanthrolin-1(2H)-ylmethyl]-N-methyl-N-(1-methyl-4-piperidinyl)imidazo[1,2-a]pyridin-5-amine;
2-[(4aR,10bS)-3,4,4a,5,6,10b-hexahydro-1,10-phenanthrolin-1(2H)-ylmethyl]-N-methyl-N-(1-methyl-3-pyrrolidinyl)imidazo[1,2-a]pyridin-5-amine;
(4aR,10bS)-1-{[5-(4,4′-bipiperidin-1-yl)imidazo[1,2-a]pyridin-2-yl]methyl}-1,2,3,4,4a,5,6,10b-octahydro-1,10-phenanthroline;
(4aR,10bS)-1-{[5-(1′-{2-[(6aS,10aR)-5,6,6a,7,8,9,10,10a-octahydrobenzo[h]quinolin-10-ylmethyl]imidazo[1,2-a]pyridin-5-yl}-4,4′-bipiperidin-1-yl)imidazo[1,2-a]pyridin-2-yl]methyl}-1,2,3,4,4a,5,6,10b-octahydro-1,10-phenanthroline;
(4aR,10bS)-1-({5-[(3R,5S)-3,5-dimethyl-1-piperazinyl]imidazo[1,2-a]pyridin-2-yl}methyl)-1,2,3,4,4a,5,6,10b-octahydro-1,10-phenanthroline;
(4aR,10bS)-1-{[5-(4-thiomorpholinyl)imidazo[1,2-a]pyridin-2-yl]methyl}-1,2,3,4,4a,5,6,10b-octahydro-1,10-phenanthroline
(4aR,10bS)-1-({5-[(2R,5S)-2,5-dimethyl-1-piperazinyl]imidazo[1,2-a]pyridin-2-yl}methyl)-1,2,3,4,4a,5,6,10b-octahydro-1,10-phenanthroline
(4aR,10bS)-1-({5-[4-(2-pyridinyl)-1-piperazinyl]imidazo[1,2-a]pyridin-2-yl}methyl)-1,2,3,4,4a,5,6,10b-octahydro-1,10-phenanthroline
N-{2-[(4aR,10bS)-3,4,4a,5,6,10b-hexahydro-1,10-phenanthrolin-1(2H)-ylmethyl]imidazo[1,2-a]pyridin-5-yl}-N,N′-dimethyl-1,3-propanediamine
(4aR,10bS)-1-({5-[4-(1-methylethyl)-1-piperazinyl]imidazo[1,2-a]pyridin-2-yl}methyl)-1,2,3,4,4a,5,6,10b-octahydro-1,10-phenanthroline
1-{2-[(4aR,10bS)-3,4,4a,5,6,10b-hexahydro-1,10-phenanthrolin-1(2H)-ylmethyl]imidazo[1,2-a]pyridin-5-yl}-N,N-dimethyl-3-pyrrolidinamine
[(1-{2-[(4aR,10bS)-3,4,4a,5,6,10b-hexahydro-1,10-phenanthrolin-1(2H)-ylmethyl]imidazo[1,2-a]pyridin-5-yl}-4-piperidinyl)methyl]amine
[(1-{2-[(4aR,10bS)-3,4,4a,5,6,10b-hexahydro-1,10-phenanthrolin-1(2H)-ylmethyl]imidazo[1,2-a]pyridin-5-yl}-4-piperidinyl)methyl]dimethylamine
(4aR,10bS)-1-{[5-(hexahydro-1H-1,4-diazepin-1-yl)imidazo[1,2-a]pyridin-2-yl]methyl}-1,2,3,4,4a,5,6,10b-octahydro-1,10-phenanthroline
1-{2-[(4aR,10bS)-3,4,4a,5,6,10b-hexahydro-1,10-phenanthrolin-1(2H)-ylmethyl]imidazo[1,2-a]pyridin-5-yl}-4-piperidinamine
N′-{2-[(4aR,10bS)-3,4,4a,5,6,10b-hexahydro-1,10-phenanthrolin-1(2H)-ylmethyl]imidazo[1,2-a]pyridin-5-yl}-N,N-dimethyl-1,2-ethanediamine
[2-(4-{2-[(4aR,10bS)-3,4,4a,5,6,10b-hexahydro-1,10-phenanthrolin-1(2H)-ylmethyl]imidazo[1,2-a]pyridin-5-yl}-1-piperazinyl)ethyl]amine
(4aR,10bS)-1-{[5-(4-methylhexahydro-1H-1,4-diazepin-1-yl)imidazo[1,2-a]pyridin-2-yl]methyl}-1,2,3,4,4a,5,6,10b-octahydro-1,10-phenanthroline
1-{2-[(4aR,10bS)-3,4,4a,5,6,10b-hexahydro-1,10-phenanthrolin-1(2H)-ylmethyl]imidazo[1,2-a]pyridin-5-yl}-N-methyl-3-pyrrolidinamine
1-{2-[(4aR,10bS)-3,4,4a,5,6,10b-hexahydro-1,10-phenanthrolin-1(2H)-ylmethyl]imidazo[1,2-a]pyridin-5-yl}-3-azetidinamine
1-{2-[(4aR,10bS)-3,4,4a,5,6,10b-hexahydro-1,10-phenanthrolin-1(2H)-ylmethyl]imidazo[1,2-a]pyridin-5-yl}-N,N-dimethyl-3-azetidinamine
(4aR,10bS)-1-[(5-{4-[2-(methyloxy)ethyl]-1-piperazinyl}imidazo[1,2-a]pyridin-2-yl)methyl]-1,2,3,4,4a,5,6,10b-octahydro-1,1-phenanthroline
(3S)-1-{2-[(4aR,10bS)-3,4,4a,5,6,10b-hexahydro-1,10-phenanthrolin-1(2H)-ylmethyl]imidazo[1,2-a]pyridin-5-yl}-N,N-dimethyl-3-pyrrolidinamine
(4aR,10bS)-1-[(5-{4-[2-oxo-2-(1-pyrrolidinyl)ethyl]-1-piperazinyl}imidazo[1,2-a]pyridin-2-yl)methyl]-1,2,3,4,4a,5,6,10b-octahydro-1,10-phenanthroline
2-(4-{2-[(4aR,10bS)-3,4,4a,5,6,10b-hexahydro-1,10-phenanthrolin-1(2H)-ylmethyl]imidazo[1,2-a]pyridin-5-yl}-1-piperazinyl)-N,N-dimethylacetamide
2-(4-{2-[(4aR,10bS)-3,4,4a,5,6,10b-hexahydro-1,10-phenanthrolin-1(2H)-ylmethyl]imidazo[1,2-a]pyridin-5-yl}-1-piperazinyl)ethanol
2-[(4aR,10bS)-3,4,4a,5,6,10b-hexahydro-1,10-phenanthrolin-1(2H)-ylmethyl]-N-[2-(1-methyl-2-pyrrolidinyl)ethyl]imidazo[1,2-a]pyridin-5-amine
(3R)-1-{2-[(4aR,10bS)-3,4,4a,5,6,10b-hexahydro-1,10-phenanthrolin-1(2H)-ylmethyl]imidazo[1,2-a]pyridin-5-yl}-N,N-dimethyl-3-pyrrolidinamine
(4aR,10bS)-1-({5-[(8aS)-hexahydropyrrolo[1,2-a]pyrazin-2(1H)-yl]imidazo[1,2-a]pyridin-2-yl}methyl)-1,2,3,4,4a,5,6,10b-octahydro-1,10-phenanthroline
(3S)-1-{2-[(4aR,10bS)-3,4,4a,5,6,10b-hexahydro-1,10-phenanthrolin-1(2H)-ylmethyl]imidazo[1,2-a]pyridin-5-yl}-3-pyrrolidinamine
2-(4-{2-[(4aR,10bS)-3,4,4a,5,6,10b-hexahydro-1,10-phenanthrolin-1(2H)-ylmethyl]imidazo[1,2-a]pyridin-5-yl}hexahydro-1H-1,4-diazepin-1-yl)ethanol
2-[(4aR,10bS)-3,4,4a,5,6,10b-hexahydro-1,10-phenanthrolin-1(2H)-ylmethyl]-N-3-pyrrolidinylimidazo[1,2-a]pyridin-5-amine
(3R)-1-{2-[(4aR,10bS)-3,4,4a,5,6,10b-hexahydro-1,10-phenanthrolin-1(2H)-ylmethyl]imidazo[1,2-a]pyridin-5-yl}-3-pyrrolidinamine
(4aR,10bS)-1-({5-[(2S)-2-methyl-1-pyrrolidinyl]imidazo[1,2-a]pyridin-2-yl}methyl)-1,2,3,4,4a,5,6,10b-octahydro-1,0-phenanthroline
2-[(4aR,10bS)-3,4,4a,5,6,10b-hexahydro-1,10-phenanthrolin-1(2H)-ylmethyl]-N-[(3S)-3-piperidinyl]imidazo[1,2-a]pyridin-5-amine
2-[(4aR,10bS)-3,4,4a,5,6,10b-hexahydro-1,10-phenanthrolin-1(2H)-ylmethyl]-N-[(3R)-3-piperidinylmethyl]imidazo[1,2-a]pyridin-5-amine
(4aS,11bS)-1-{[5-(4-methyl-1-piperazinyl)imidazo[1,2-a]pyridin-2-yl]methyl}-2,3,4,4a,5,6,7,11b-octahydro-1H-pyrido[3′,2′:6,7]cyclohepta[1,2-b]pyridine
(4aR,11bS)-1-{[5-(4-methyl-1-piperazinyl)imidazo[1,2-a]pyridin-2-yl]methyl}-2,3,4,4a,5,6,7,11b-octahydro-1H-pyrido[3′,2′:6,7]cyclohepta[1,2-b]pyridine
(trans) 1-{[5-(4-methylhexahydro-1H-1,4-diazepin-1-yl)imidazo[1,2-a]pyridin-2-yl]methyl}-2,3,4,4a,5,6,7,11b-octahydro-1H-pyrido[3′,2′:6,7]cyclohepta[1,2-b]pyridine
(trans) 4-[2-(2,3,3a,4,5,9b-hexahydro-1H-pyrrolo[3,2-h]quinolin-1-ylmethyl)-1H-benzimidazol-1-yl]-1-butanamine
(trans) 1-{[1-(1H-benzimidazol-2-ylmethyl)-1H-benzimidazol-2-yl]methyl}-2,3,3a,4,5,9b-hexahydro-1H-pyrrolo[3,2-h]quinoline
(trans) 3-[2-(2,3,3a,4,5,9b-hexahydro-1H-pyrrolo[3,2-h]quinolin-1-ylmethyl)-1H-benzimidazol-1-yl]-N,N-dimethyl-1-propanamine;
(trans) 1-({1-[3-(1-piperidinyl)propyl]-1H-benzimidazol-2-yl}methyl)-2,3,3a,4,5,9b-hexahydro-1H-pyrrolo[3,2-h]quinoline; and
(4aR,10bS)-1-{[1-methyl-7-(4-methyl-1-piperazinyl)-1H-benzimidazol-2-yl]methyl}-1,2,3,4,4a,5,6,10b-octahydro-1,10-phenanthroline;
or salts, solvates, or physiologically functional derivatives thereof.

38. A compound selected from;
(trans) 4-[2-(3,4,4a,5,6,10b-hexahydro-1,10-phenanthrolin-1(2H)-ylmethyl)-1H-benzimidazol-1-yl]-1-butanamine
(cis) 3-[2-(3,4,4a,5,6,10b-hexahydro-1,10-phenanthrolin-1(2H)-ylmethyl)-1H-benzimidazol-1-yl]-N,N-dimethyl-1-propanamine
(cis) 1-({1-[3-(1-piperidinyl)propyl]-1H-benzimidazol-2-yl}methyl)-1,2,3,4,4a,5,6,10b-octahydro-1,10-phenanthroline
(4aR,10bS)-1-{[4-(4-methyl-1-piperazinyl)-1H-benzimidazol-2-yl]methyl}-1,2,3,4,4a,5,6,10b-octahydro-1,10-phenanthroline
(4aR,10bR)-1-{[5-(4-methyl-1-piperazinyl)imidazo[1,2-a]pyridin-2-yl]methyl}-1,2,3,4,4a,5,6,10b-octahydro-1,10-phenanthroline
[2-[(4aR,10bS)-3,4,4a,5,6,10b-hexahydro-1,10-phenanthrolin-1(2H)-ylmethyl]-5-(4-methyl-1-piperazinyl)imidazo[1,2-a]pyridin-3-yl]methanol
N-ethyl-N-{[2-[(4aR,10bS)-3,4,4a,5,6,10b-hexahydro-1,10-phenanthrolin-1(2H)-ylmethyl]-5-(4-methyl-1-piperazinyl)imidazo[1,2-a]pyridin-3-yl]methyl}ethanamine
(4aR,10bS)-1-({5-(4-methyl-1-piperazinyl)-3-[(4-methyl-1-piperazinyl)methyl]imidazo[1,2-a]pyridin-2-yl}methyl)-1,2,3,4,4a,5,6,10b-octahydro-1,10-phenanthroline
(4aR,10bS)-1-{[5-(4-methyl-1-piperazinyl)-3-(1-piperidinylmethyl)imidazo[1,2-a]pyridin-2-yl]methyl}-1,2,3,4,4a,5,6,10b-octahydro-1,10-phenanthroline
(4aR,10bS)-1-{[5-(4-methyl-1-piperazinyl)-3-(1-pyrrolidinylmethyl)imidazo[1,2-a]pyridin-2-yl]methyl}-1,2,3,4,4a,5,6,10b-octahydro-1,10-phenanthroline
(4aR,10bS)-1-{[5-(4-methyl-1-piperazinyl)-3-(4-morpholinylmethyl)imidazo[1,2-a]pyridin-2-yl]methyl}-1,2,3,4,4a,5,6,10b-octahydro-1,10-phenanthroline
(4aR,10bS)-1-({1-[(3S)-3-piperidinylmethyl]-1H-benzimidazol-2-yl}methyl)-1,2,3,4,4a,5,6,10b-octahydro-1,10-phenanthroline
(4aR,10bS)-1-[(1-{[(3S)-1-(1-methylethyl)-3-piperidinyl]methyl}-1H-benzimidazol-2-yl)methyl]-1,2,3,4,4a,5,6,10b-octahydro-1,10-phenanthroline
(4aR,10bS)-1-[(1-{[(3S)-1-methyl-3-piperidinyl]methyl}-1H-benzimidazol-2-yl)methyl]-1,2,3,4,4a,5,6,10b-octahydro-1,10-phenanthroline
1-{2-[(4aR,10bS)-3,4,4a,5,6,10b-hexahydro-1,10-phenanthrolin-1(2H)-ylmethyl]imidazo[1,2-a]pyridin-5-yl}-N,N-dimethyl-4-piperidinamine
2-[(4aR,10bS)-3,4,4a,5,6,10b-hexahydro-1,10-phenanthrolin-1(2H)-ylmethyl]-N-(1-methyl-4-piperidinyl)imidazo[1,2-a]pyridin-5-amine
N-{2-[(4aR,10bS)-3,4,4a,5,6,10b-hexahydro-1,10-phenanthrolin-1(2H)-ylmethyl]imidazo[1,2-a]pyridin-5-yl}-N,N′,N′-trimethyl-1,2-ethanediamine
(4aR,10bS)-1-{[5-(1,4′-bipiperidin-1′-yl)imidazo[1,2-a]pyridin-2-yl]methyl}-1,2,3,4,4a,5,6,10b-octahydro-1,10-phenanthroline
(4aR,10bS)-1-({5-[4-(1-pyrrolidinyl)-1-piperidinyl]imidazo[1,2-a]pyridin-2-yl}methyl)-1,2,3,4,4a,5,6,10b-octahydro-1,10-phenanthroline
(4aR,10bS)-1-{[5-(4-ethyl-1-piperazinyl)imidazo[1,2-a]pyridin-2-yl]methyl}-1,2,3,4,4a,5,6,10b-octahydro-1,10-phenanthroline
2-[(4aR,10bS)-3,4,4a,5,6,10b-hexahydro-1,10-phenanthrolin-1(2H)-ylmethyl]-N-methyl-N-(1-methyl-4-piperidinyl)imidazo[1,2-a]pyridin-5-amine
2-[(4aR,10bS)-3,4,4a,5,6,10b-hexahydro-1,10-phenanthrolin-1(2H)-ylmethyl]-N-methyl-N-(1-methyl-3-pyrrolidinyl)imidazo[1,2-a]pyridin-5-amine
(4aR,10bS)-1-({5-[(3R,5S)-3,5-dimethyl-1-piperazinyl]imidazo[1,2-a]pyridin-2-yl}methyl)-1,2,3,4,4a,5,6,10b-octahydro-1,10-phenanthroline
(4aR,10bS)-1-({5-[(2R,5S)-2,5-dimethyl-1-piperazinyl]imidazo[1,2-a]pyridin-2-yl}methyl)-1,2,3,4,4a,5,6,10b-octahydro-1,10-phenanthroline
N-{2-[(4aR,10bS)-3,4,4a,5,6,10b-hexahydro-1,10-phenanthrolin-1(2H)-ylmethyl]imidazo[1,2-a]pyridin-5-yl}-N,N′-dimethyl-1,3-propanediamine
(4aR,10bS)-1-({5-[4-(1-methylethyl)-1-piperazinyl]imidazo[1,2-a]pyridin-2-yl}methyl)-1,2,3,4,4a,5,6,10b-octahydro-1,10-phenanthroline
1-{2-[(4aR,10bS)-3,4,4a,5,6,10b-hexahydro-1,10-phenanthrolin-1(2H)-ylmethyl]imidazo[1,2-a]pyridin-5-yl}-N,N-dimethyl-3-pyrrolidinamine
[(1-{2-[(4aR,10bS)-3,4,4a,5,6,10b-hexahydro-1,10-phenanthrolin-1(2H)-ylmethyl]imidazo[1,2-a]pyridin-5-yl}-4-piperidinyl)methyl]amine
[(1-{2-[(4aR,10bS)-3,4,4a,5,6,10b-hexahydro-1,10-phenanthrolin-1(2H)-ylmethyl]imidazo[1,2-a]pyridin-5-yl}-4-piperidinyl)methyl]dimethylamine
(4aR,10bS)-1-{[5-(hexahydro-1H-1,4-diazepin-1-yl)imidazo[1,2-a]pyridin-2-yl]methyl}-1,2,3,4,4a,5,6,10b-octahydro-1,10-phenanthroline
1-{2-[(4aR,10bS)-3,4,4a,5,6,10b-hexahydro-1,10-phenanthrolin-1(2H)-ylmethyl]imidazo[1,2-a]pyridin-5-yl}-4-piperidinamine
[2-(4-{2-[(4aR,10bS)-3,4,4a,5,6,10b-hexahydro-1,10-phenanthrolin-1(2H)-ylmethyl]imidazo[1,2-a]pyridin-5-yl}-1-piperazinyl)ethyl]amine
(4aR,10bS)-1-{[5-(4-methylhexahydro-1H-1,4-diazepin-1-yl)imidazo[1,2-a]pyridin-2-yl]methyl}-1,2,3,4,4a,5,6,10b-octahydro-1,10-phenanthroline
1-{2-[(4aR,10bS)-3,4,4a,5,6,10b-hexahydro-1,10-phenanthrolin-1(2H)-ylmethyl]imidazo[1,2-a]pyridin-5-yl}-N-methyl-3-pyrrolidinamine
1-{2-[(4aR,10bS)-3,4,4a,5,6,10b-hexahydro-1,10-phenanthrolin-1(2H)-ylmethyl]imidazo[1,2-a]pyridin-5-yl}-3-azetidinamine
(4aR,10bS)-1-[(5-{4-[2-(methyloxy)ethyl]-1-piperazinyl}imidazo[1,2-a]pyridin-2-yl)methyl]-1,2,3,4,4a,5,6,10b-octahydro-1,10-phenanthroline
(3S)-1-{2-[(4aR,10bS)-3,4,4a,5,6,10b-hexahydro-1,10-phenanthrolin-1(2H)-ylmethyl]imidazo[1,2-a]pyridin-5-yl}-N,N-dimethyl-3-pyrrolidinamine
(4aR,10bS)-1-[(5-{4-[2-oxo-2-(1-pyrrolidinyl)ethyl]-1-piperazinyl}imidazo[1,2-a]pyridin-2-yl)methyl]-1,2,3,4,4a,5,6,10b-octahydro-1,10-phenanthroline
2-(4-{2-[(4aR,10bS)-3,4,4a,5,6,10b-hexahydro-1,10-phenanthrolin-1(2H)-ylmethyl]imidazo[1,2-a]pyridin-5-yl}-1-piperazinyl)-N,N-dimethylacetamide
2-(4-{2-[(4aR,10bS)-3,4,4a,5,6,10b-hexahydro-1,10-phenanthrolin-1(2H)-ylmethyl]imidazo[1,2-a]pyridin-5-yl}-1-piperazinyl)ethanol
2-[(4aR,10bS)-3,4,4a,5,6,10b-hexahydro-1,10-phenanthrolin-1(2H)-ylmethyl]-N-[2-(1-methyl-2-pyrrolidinyl)ethyl]imidazo[1,2-a]pyridin-5-amine
(3R)-1-{2-[(4aR,10bS)-3,4,4a,5,6,10b-hexahydro-1,10-phenanthrolin-1(2H)-ylmethyl]imidazo[1,2-a]pyridin-5-yl}-N,N-dimethyl-3-pyrrolidinamine
(4aR,10bS)-1-({5-[(8aS)-hexahydropyrrolo[1,2-a]pyrazin-2(1H)-yl]imidazo[1,2-a]pyridin-2-yl}methyl)-1,2,3,4,4a,5,6,10b-octahydro-1,10-phenanthroline
(3S)-1-{2-[(4aR,10bS)-3,4,4a,5,6,10b-hexahydro-1,10-phenanthrolin-1(2H)-ylmethyl]imidazo[1,2-a]pyridin-5-yl}-3-pyrrolidinamine
2-(4-{2-[(4aR,10bS)-3,4,4a,5,6,10b-hexahydro-1,10-phenanthrolin-1(2H)-ylmethyl]imidazo[1,2-a]pyridin-5-yl}hexahydro-1H-1,4-diazepin-1-yl)ethanol
(3R)-1-{2-[(4aR,10bS)-3,4,4a,5,6,10b-hexahydro-1,10-phenanthrolin-1(2H)-ylmethyl]imidazo[1,2-a]pyridin-5-yl}-3-pyrrolidinamine
2-[(4aR,10bS)-3,4,4a,5,6,10b-hexahydro-1,10-phenanthrolin-1(2H)-ylmethyl]-N-[(3S)-3-piperidinyl]imidazo[1,2-a]pyridin-5-amine
2-[(4aR,10bS)-3,4,4a,5,6,10b-hexahydro-1,10-phenanthrolin-1(2H)-ylmethyl]-N-[(3R)-3-piperidinylmethyl]imidazo[1,2-a]pyridin-5-amine
(4aS,11bS)-1-{[5-(4-methyl-1-piperazinyl)imidazo[1,2-a]pyridin-2-yl]methyl}-2,3,4,4a,5,6,7,11b-octahydro-1H-pyrido[3′,2′:6,7]cyclohepta[1,2-b]pyridine
(4aR,11bS)-1-{[5-(4-methyl-1-piperazinyl)imidazo[1,2-a]pyridin-2-yl]methyl}-2,3,4,4a,5,6,7,11b-octahydro-1H-pyrido[3′,2′:6,7]cyclohepta[1,2-b]pyridine;
and
(4aR,10bS)-1-{[1-methyl-7-(4-methyl-1-piperazinyl)-1H-benzimidazol-2-yl]methyl}-1,2,3,4,4a,5,6,10b-octahydro-1,10-phenanthroline;
or salts, solvates, or physiologically functional derivatives thereof.

39. A compound selected from:
(cis)-1-({1-[(1-Methyl-3-piperidinyl)methyl]-1H-benzimidazol-2-yl}methyl)-1,2,3,4,4^{a},5,6,10b-octahydro-1,10-phenanthroline
(trans)-1-{[5-(4-Methyl-1-piperazinyl)imidazo[1,2-a]pyridin-2-yl]methyl}-1,2,3,4,4a,5,6,10b-octahydro-1,10-phenanthroline
(cis)-1-{[5-(4-Methyl-1-piperazinyl)imidazo[1,2-a]pyridin-2-yl]methyl}-1,2,3,4,4^{a},5,6,10b-octahydro-1,10-phenanthroline
(4aR,10bS)-1-{[5-(4-Methyl-1-piperazinyl)imidazo[1,2-a]pyridin-2-yl]methyl}-1,2,3,4,4^{a},5,6,10b-octahydro-1,10-phenanthroline
(trans)-1-{[4-(4-Methyl-1-piperazinyl)-1H-benzimidazol-2-yl]methyl}-1,2,3,4,4^{a},5,6,10b-octahydro-1,10-phenanthroline trifluoroacetate
(cis)-1-{[4-(4-Methyl-1-piperazinyl)-1H-benzimidazol-2-yl]methyl}-1,2,3,4,4^{a},5,6,10b-octahydro-1,10-phenanthroline
(4aR,10bS)-1-{[4-(4-methyl-1-piperazinyl)-1H-benzimidazol-2-yl]methyl}-1,2,3,4,4^{a},5,6,10b-octahydro-1,10-phenanthroline
(4aR,10bR)-1-{[5-(4-methyl-1-piperazinyl)imidazo[1,2-a]pyridin-2-yl]methyl}-1,2,3,4,4a,5,6,10b-octahydro-1,10-phenanthroline
[2-[(4aR,10bS)-3,4,4^{a},5,6,10b-hexahydro-1,10-phenanthrolin-1(2H)-ylmethyl]-5-(4-methyl-1-piperazinyl)imidazo[1,2-a]pyridin-3-yl]methanol
(4aR,10bS)-1-({1-[(3S)-3-piperidinylmethyl]-1H-benzimidazol-2-yl}methyl)-1,2,3,4,4^{a},5,6,10b-octahydro-1,10-phenanthroline
(4aR,10bS)-1-[(1-{[(3S)-1-(1-methylethyl)-3-piperidinyl]methyl}-1H-benzimidazol-2-yl)methyl]-1,2,3,4,4^{a},5,6,10b-octahydro-1,10-phenanthroline
(4aR,10bS)-1-[(1-{[(3S)-1-methyl-3-piperidinyl]methyl}-1H-benzimidazol-2-yl)methyl]-1,2,3,4,4^{a},5,6,10b-octahydro-1,10-phenanthroline
1-{2-[(4aR,10bS)-3,4,4^{a},5,6,10b-hexahydro-1,10-phenanthrolin-1(2H)-ylmethyl]imidazo[1,2-a]pyridin-5-yl}-N,N-dimethyl-4-piperidinamine
2-[(4aR,10bS)-3,4,4^{a},5,6,10b-hexahydro-1,10-phenanthrolin-1(2H)-ylmethyl]-N-(1-methyl-4-piperidinyl)imidazo[1,2-a]pyridin-5-amine
N-{2-[(4aR,10bS)-3,4,4^{a},5,6,10b-hexahydro-1,10-phenanthrolin-1(2H)-ylmethyl]imidazo[1,2-a]pyridin-5-yl}-N,N′,N′-trimethyl-1,2-ethanediamine
(4aR,10bS)-1-{[5-(4-ethyl-1-piperazinyl)imidazo[1,2-a]pyridin-2-yl]methyl}-1,2,3,4,4^{a},5,6,10b-octahydro-1,10-phenanthroline
2-[(4aR,10bS)-3,4,4^{a},5,6,10b-hexahydro-1,10-phenanthrolin-1(2H)-ylmethyl]-N-methyl-N-(1-methyl-4-piperidinyl)imidazo[1,2-a]pyridin-5-amine
2-[(4aR,10bS)-3,4,4^{a},5,6,10b-hexahydro-1,10-phenanthrolin-1(2H)-ylmethyl]-N-methyl-N-(1-methyl-3-pyrrolidinyl)imidazo[1,2-a]pyridin-5-amine
(4aR,10bS)-1-({5-[(3R,5S)-3,5-dimethyl-1-piperazinyl]imidazo[1,2-a]pyridin-2-yl}methyl)-1,2,3,4,4^{a},5,6,10b-octahydro-1,10-phenanthroline
(4aR,10bS)-1-({5-[(2R,5S)-2,5-dimethyl-1-piperazinyl]imidazo[1,2-a]pyridin-2-yl}methyl)-1,2,3,4,4^{a},5,6,10b-octahydro-1,10-phenanthroline
(4aR,10bS)-1-({5-[4-(1-methylethyl)-1-piperazinyl]imidazo[1,2-a]pyridin-2-yl}methyl)-1,2,3,4,4^{a},5,6,10b-octahydro-1,10-phenanthroline
1-{2-[(4aR,10bS)-3,4,4^{a},5,6,10b-hexahydro-1,10-phenanthrolin-1(2H)-ylmethyl]imidazo[1,2-a]pyridin-5-yl}-N,N-dimethyl-3-pyrrolidinamine
[(1-{2-[(4aR,10bS)-3,4,4^{a},5,6,10b-hexahydro-1,10-phenanthrolin-1(2H)-ylmethyl]imidazo[1,2-a]pyridin-5-yl}-4-piperidinyl)methyl]amine
[(1-{2-[(4aR,10bS)-3,4,4^{a},5,6,10b-hexahydro-1,10-phenanthrolin-1(2H)-ylmethyl]imidazo[1,2-a]pyridin-5-yl}-4-piperidinyl)methyl]dimethylamine
1-{2-[(4aR,10bS)-3,4,4^{a},5,6,10b-hexahydro-1,10-phenanthrolin-1(2H)-ylmethyl]imidazo[1,2-a]pyridin-5-yl}-4-piperidinamine
[2-(4-{2-[(4aR,10bS)-3,4,4^{a},5,6,10b-hexahydro-1,10-phenanthrolin-1(2H)-ylmethyl]imidazo[1,2-a]pyridin-5-yl}-1-piperazinyl)ethyl]amine
(4aR,10bS)-1-{[5-(4-methylhexahydro-1H-1,4-diazepin-1-yl)imidazo[1,2-a]pyridin-2-yl]methyl}-1,2,3,4,4^{a},5,6,10b-octahydro-1,10-phenanthroline
1-{2-[(4aR,10bS)-3,4,4^{a},5,6,10b-hexahydro-1,10-phenanthrolin-1(2H)-ylmethyl]imidazo[1,2-a]pyridin-5-yl}-N-methyl-3-pyrrolidinamine
1-{2-[(4aR,10bS)-3,4,4^{a},5,6,10b-hexahydro-1,10-phenanthrolin-1(2H)-ylmethyl]imidazo[1,2-a]pyridin-5-yl}-3-azetidinamine
(3S)-1-{2-[(4aR,10bS)-3,4,4^{a},5,6,10b-hexahydro-1,10-phenanthrolin-1(2H)-ylmethyl]imidazo[1,2-a]pyridin-5-yl}-N,N-dimethyl-3-pyrrolidinamine
(3R)-1-{2-[(4aR,10bS)-3,4,4^{a},5,6,10b-hexahydro-1,10-phenanthrolin-1(2H)-ylmethyl]imidazo[1,2-a]pyridin-5-yl}-N,N-dimethyl-3-pyrrolidinamine
(4aR,10bS)-1-({5-[(8aS)-hexahydropyrrolo[1,2-a]pyrazin-2(1H)-yl]imidazo[1,2-a]pyridin-2-yl}methyl)-1,2,3,4,4^{a},5,6,10b-octahydro-1,10-phenanthroline
(3S)-1-{2-[(4aR,10bS)-3,4,4^{a},5,6,10b-hexahydro-1,10-phenanthrolin-1(2H)-ylmethyl]imidazo[1,2-a]pyridin-5-yl}-3-pyrrolidinamine
(3R)-1-{2-[(4aR,10bS)-3,4,4^{a},5,6,10b-hexahydro-1,10-phenanthrolin-1(2H)-ylmethyl]imidazo[1,2-a]pyridin-5-yl}-3-pyrrolidinamine
2-[(4aR,10bS)-3,4,4^{a},5,6,10b-hexahydro-1,10-phenanthrolin-1(2H)-ylmethyl]-N-[(3S)-3-piperidinyl]imidazo[1,2-a]pyridin-5-amine
(4aS,11bS)-1-{[5-(4-methyl-1-piperazinyl)imidazo[1,2-a]pyridin-2-yl]methyl}-2,3,4,4^{a},5,6,7,11b-octahydro-1H-pyrido[3′,2′:6,7]cyclohepta[1,2-b]pyridine
(4aR,11bS)-1-{[5-(4-methyl-1-piperazinyl)imidazo[1,2-a]pyridin-2-yl]methyl}-2,3,4,4^{a},5,6,7,11b-octahydro-1H-pyrido[3′,2′:6,7]cyclohepta[1,2-b]pyridine;
(4aR,10bS)-1-{[1-methyl-7-(4-methyl-1-piperazinyl)-1H-benzimidazol-2-yl]methyl}-1,2,3,4,4^{a},5,6,10b-octahydro-1,10-phenanthroline; and
1-{2-[(4aR,10bS)-3,4,4^{a},5,6,10b-hexahydro-1,10-phenanthrolin-1(2H)-ylmethyl]imidazo[1,2-a]pyridin-5-yl}-4-piperidinamine;
or salts solvates, or physiologically functional derivatives thereof.

40. A compound selected from:
(4aR,10bS)-1-{[5-(4-Methyl-1-piperazinyl)imidazo[1,2-a]pyridin-2-yl]methyl}-1,2,3,4,4a,5,6,10b-octahydro-1,10-phenanthroline
[2-[(4aR,10S)-3,4,4a,5,6,10b-hexahydro-1,10-phenanthrolin-1(2H)-ylmethyl]-5-(4-methyl-1-piperazinyl)imidazo[1,2-a]pyridin-3-yl]methanol
(4aR,10bS)-1-{[5-(4-ethyl-1-piperazinyl)imidazo[1,2-a]pyridin-2-yl]methyl}-1,2,3,4,4a,5,6,10b-octahydro-1,10-phenanthroline
(4aR,10bS)-1-({5-[4-(1-methylethyl)-1-piperazinyl]imidazo[1,2-a]pyridin-2-yl}methyl)-1,2,3,4,4a,5,6,10b-octahydro-1,10-phenanthroline
1-{2-[(4aR,10bS)-3,4,4a,5,6,10b-hexahydro-1,10-phenanthrolin-1(2H)-ylmethyl]imidazo[1,2-a]pyridin-5-yl}-N,N-dimethyl-3-pyrrolidinamine and
(3S)-1-{2-[(4aR,10bS)-3,4,4a,5,6,10b-hexahydro-1,10-phenanthrolin-1(2H)-ylmethyl]imidazo[1,2-a]pyridin-5-yl}-N,N-dimethyl-3-pyrrolidinamine;
or salts, solvates, or physiologically functional derivatives thereof.

41. A compound selected from:
(cis)-1-{[4-(4-Methyl-1-piperazinyl)-1H-benzimidazol-2-yl]methyl}-1,2,3,4,4a,5,6,10b-octahydro-1,10-phenanthroline
(4aR,10bS)-1-{[4-(4-methyl-1-piperazinyl)-1H-benzimidazol-2-yl]methyl}-1,2,3,4,4a,5,6,10b-octahydro-1,10-phenanthroline
(4aR,10S)-1-[(1-{[(3S)-1-(1-methylethyl)-3-piperidinyl]methyl}-1H-benzimidazol-2-yl)methyl]-1,2,3,4,4a,5,6,10b-octahydro-1,10-phenanthroline
(4aR,10bS)-1-[(1-{[(3S)-1-methyl-3-piperidinyl]methyl}-1H-benzimidazol-2-yl)methyl]-1,2,3,4,4a,5,6,10b-octahydro-1,10-phenanthroline; and
(4aR,10bS)-1-{[1-methyl-7-(4-methyl-1-piperazinyl)-1H-benzimidazol-2-yl]methyl}-1,2,3,4,4a,5,6,10b-octahydro-1,10-phenanthroline;
or salts, solvates, or physiologically functional derivatives thereof.

42. A pharmaceutical composition comprising a compound of claim 1 and a pharmaceutically acceptable carrier.

43-50. (canceled)

51. A method for the treatment of a condition or disease modulated by a chemokine receptor comprising the administration of an effective amount of a compound of claim 1.

52. The method of claim 51 wherein the chemokine receptor is CXCR4.

53. A method for the treatment of HIV infection, diseases associated with hematopoiesis, myocardial infarction, controlling the side effects of chemotherapy, enhancing the success of bone marrow transplantation, enhancing wound healing and burn treatment, combating bacterial infections in leukemia, inflammation, inflammatory or allergic diseases, asthma, allergic rhinitis, hypersensitivity lung diseases, hypersensitivity pneumonitis, eosinophilic pneumonitis, delayed-type hypersensitivity, interstitial lung disease (ILD), idiopathic pulmonary fibrosis, systemic lupus erythematosus, ankylosing spondylitis, systemic sclerosis, Sjogren's syndrome, polymyositis or dermatomyositis, systemic anaphylaxis or hypersensitivity responses, drug allergies, insect sting allergies, autoimmune diseases, rheumatoid arthritis, psoriatic arthritis, systemic lupus erythematosus, myastenia gravis, juvenile onset diabetes, glomerulonephritis, autoimmune throiditis, graft rejection, allograft rejection, graft-versus-host disease, inflammatory bowel diseases, Crohn's disease, ulcerative colitus; spondylo-arthropathies, scleroderma, psoriasis, T-cell-mediated psoriasis, inflammatory dermatoses, dermatitis, eczema, atopic dermatitis, allergic contact dermatitis, urticaria, vasculitis, necrotizing, cutaneous, hypersensitivity vasculitis, eosinophilic myotis, eosinophilic fasciitis, myocardial infarction, brain, breast, prostate, lung, or haematopoetic tissue cancers comprising the administration of an effective amount of a compound of claim 1.

54. A method for the treatment of HIV infection, rheumatoid arthritis, inflammation, or cancer comprising the administration of an effective amount of a compound of claim 1.

55. A method of treatment of HIV infection, rheumatoid arthritis, inflammation, or cancer comprising the administration of an effective amount of a pharmaceutical composition containing a compound of claim 1.

56. A method of treatment of HIV infection comprising the administration of an effective amount of a pharmaceutical composition containing of a compound of claim 1.

57. A pharmaceutical composition comprising a compound of claim 1 and one or more additional therapeutic agent selected from the group consisting of nucleotide reverse transcriptase inhibitors, non-nucleotide reverse transcriptase inhibitors, protease inhibitors, entry inhibitors, integrase inhibitors, budding inhibitors, CXCR4 inhibitors, and CCR5 inhibitors.

58. The pharmaceutical composition of claim 57 wherein:
the nucleotide reverse transcriptase inhibitor is selected from zidovudine, didanosine, lamivudine, zalcitabine, abacavir, stavidine, adefovir, adefovir dipivoxil, fozivudine, todoxil, emtricitabine, alovudine, amdoxovir, and elvucitabine;
the non-nucleotide reverse transcriptase inhibitor is selected from nevirapine, delavirdine, efavirenz, loviride, immunocal, oltipraz, capravirine, TMC-278, TMC-125, and etravirine;
the protease inhibitor is selected from saquinavir, ritonavir, indinavir, nelfinavir, amprenavir, fosamprenavir, brecanavir, palinavir, lasinavir, atazanavir, and tipranavir;
the entry inhibitor is selected from enfuvirtide (T-20), T-1249, PRO-542, PRO-140, TNX-355, BMS-806, and 5-Helix;
the integrase inhibitor is selected from L-870,180;
the budding inhibitors is selected from PA-344 and PA-457; and
the CXCR4/CCR5 inhibitor is selected from vicriviroc (Sch-C), Sch-D, TAK779, maraviroc (UK 427,857), and TAK449.

59. The pharmaceutical composition of claim 58 wherein the additional therapeutic agent is a CXCR4 inhibitor or CCR5 inhibitor.

60. An intermediate compound of formula (V): 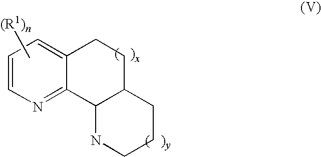
wherein x, y, n, and R¹ are as defined in claim 1.

61. A process for making a compound of formula (V) 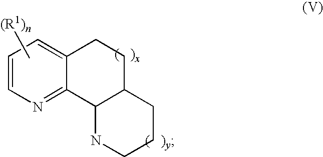
comprising:
i) reacting a compound of formula (II) 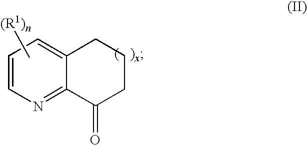
with a secondary amine to produce a compound of formula (III) 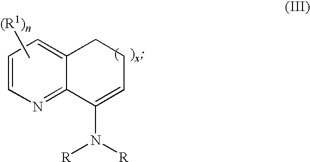
ii) reacting said compound of formula (III) with an alkylating agent to produce a compound of formula (IV) 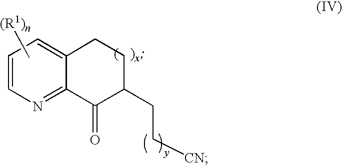
and
iii) transforming said compound of formula (IV) to a compound of formula (V) 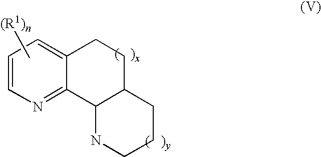
wherein x, y, n, R, and R¹ are as defined in claim 1.

62. A process for making a compound of formula (V) 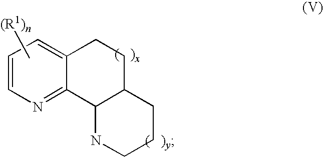
comprising;
i) treating a compound of formula (II) 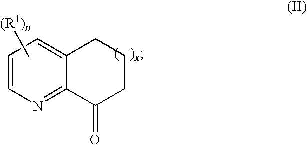
with a base and alkylating with an alkylating reagent to prepare a compound of formula (IV) 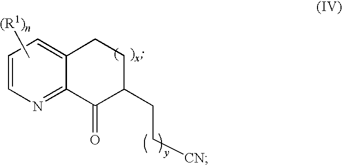
ii) transforming the compound of formula (IV) to a compound of formula (V) 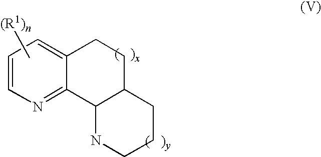
wherein x, v, n, R, and R¹ are as defined in claim 1.

63. A process for making a compound of formula (Va) 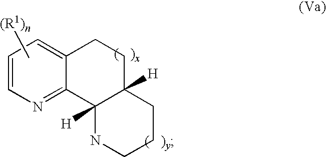
comprising:
i) reacting a compound of formula (II) 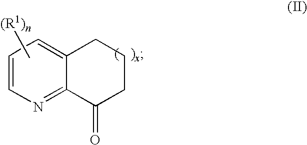
with a secondary amine to prepare a compound of formula (III) 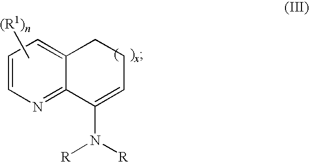
ii) reacting a compound of formula (III) with an alkylating reagent to prepare a compound of formula (VI) 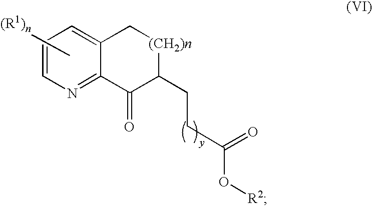
or
(ii-a) optionally forming a compound of formula (VI) by treating a compound of formula (II) with a base followed by treating with an alkylating agent;
iii) reacting a compound of formula (VI) with a chiral auxiliary amine and reducing reagent to prepare a compound of formula (VII) 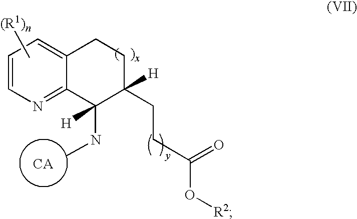
iv) treating the compound of formula (VII) with a reducing reagent to prepare a compound of formula (VII) 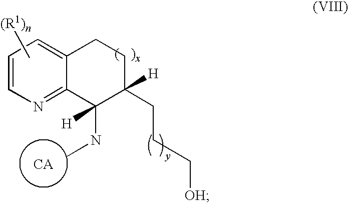
v) transforming the compound of formula (VII) to prepare a compound of formula (IX) 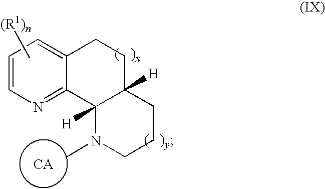
vi) cleaving the chiral auxiliary of compound (IX) to prepare a compound of formula (Va)
wherein x, y, n, R, R¹, and R² are as defined in claim 1 and CA is a chiral auxillary.

64. A process for making a compound of claim 1 comprising reacting a compound of formula (X) 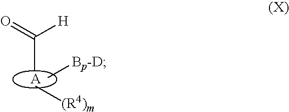
with a compound of formula (V) 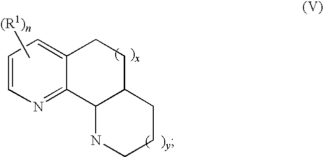
under reductive conditions to produce a compound of formula (I) 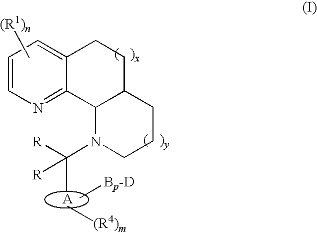
wherein x, V, m, n, p, A, B, D, R¹, and R⁴ are as defined in claim 1.

65. A process for making a compound of claim 1 comprising reacting a compound of formula (V) 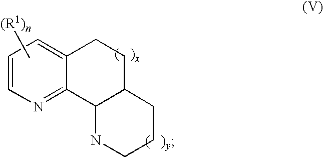
with a compound of formula (XI) 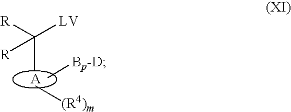
wherein LV is a leaving group under condensation conditions to produce a compound of formula (I) 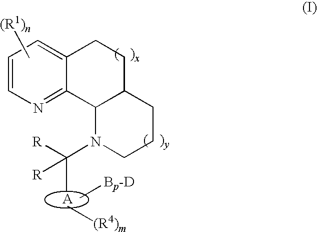
wherein x, y, m, n, p, A, B, D, R¹, and R⁴ are as defined in claim 1 and LV is a leaving group.

66. A process for making a compound of claim 1 wherein heterocycle A is a benzimidazole comprising treating a compound of formula (XV) 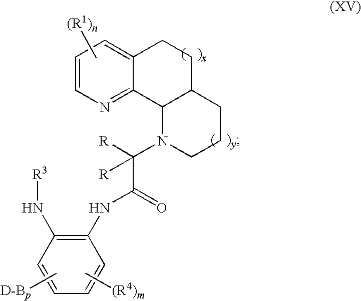
with an acid optionally with heating to produce a compound of formula (I) 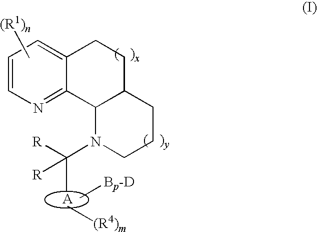
wherein x, y, m, n, p, A, B, D, R, R¹, R³, and R⁴ are as defined in claim 1.
